(19) United States
(12) Patent Application Publication
(10) Pub. No.: US 2006/0068496 A1
(43) Pub. Date: Mar. 30, 2006

(54) Differentiation of stem cells

(76) Inventor: James H. Kelly, Houston, TX (US)

Correspondence Address: NEEDLE & ROSENBERG, P.C., SUITE 1000, 999 PEACHTREE STREET, ATLANTA, GA, 30309-3915, US

(21) Appl. No.: 11/194,143

(22) Filed: Jul. 29, 2005

Related U.S. Application Data
(60) Provisional application No. 60/592,027, filed on Jul. 29, 2004.

(51) Int. Cl. C12N 5/08; C12N 15/85

(52) U.S. Cl. 435/455; 435/366

(57) ABSTRACT

Disclosed are compositions and methods for identifying specific cell types.

### I. CROSS-REFERENCE TO RELATED APPLICATIONS

This application claims benefit of U.S. Provisional Application No. 60/592,027, filed Jul. 29, 2004. Application Ser. No. 60/592,027, filed Jul. 29, 2004, is hereby incorporated herein by reference in its entirety.

### II. BACKGROUND

Pluripotent stem cells, such as human pluripotent stem cells, promise to dramatically alter and extend our ability to both understand and treat many of the chronic illnesses that define modern medicine. From drug discovery, to the generation of monoclonal antibodies, to the production of cell therapies, much of human cell biology expects to be transformed by the ability to generate specific cell types, such as human cell types at will. The medical and industrial application of pluripotent stem cells requires the ability to generate large numbers of a single cell type in vitro. Current strategies of directing cell differentiation through treatment with known morphogens, hormones or other chemicals have been successful in certain instances but in no case have they been able to generate the quality and volume of cells necessary for any practical application outside the laboratory. There is a tremendous need for being able to generate cell types in vitro. The production of monoclonal antibodies through in vitro immune systems, the production of islets for diabetes treatment, and the production of neural precursors for neural related dysfunction are just a few of the human disease areas needing a steady reliable production of specific cell types. The economic significance of this project is dramatic. The monoclonal antibody application alone is a multibillion dollar industry. The National Institutes of Health estimates that the annual cost of diabetes to the United States is $132 billion (http://diabetes.niddk.nih.gov/dm/pubs/statistics/index.htm#14). Estimates for the annual national cost of neurodegenerative disease is over $100 billion (http://www.alzheimers.org/pubs/prog00.htm#The%20Impact%2of%20Alzheimer/92s%20Di sease).

The practical application of embryonic stem cell biology will require the generation of large numbers of homogeneous cell types. Large scale culture of undifferentiated stem cells, followed by directed differentiation, presents a series of challenges that suggest a need for an alternative solution. ES and EG lines require the addition of expensive recombinant hormones to the cell culture medium to maintain their growth and maintenance of the undifferentiated state, such as Fibroblast Growth Factor and Leukemia Inhibitory Factor. In general, ES and EG lines are still cultured on feeder layers. They grow slowly, freeze and recover poorly and are difficult to passage. While progress is being made in making ES and EG cell culture easier, they will always require substantial resources and a knowledgeable and dedicated staff.

Directed differentiation presents additional problems. Differentiation can be initiated either by changing the hormonal milieu, forming embryoid bodies or a combination of both. Embryoid body formation is the most widely used and general process at present. This method appears to generate a wide variety of cells, resulting from the juxtaposition of the various tissue types within the embryoid body. Problems with this method revolve around homogenous formation. In a static culture, bodies of various sizes and shapes form, resulting in a variable differentiation process. Again, while laboratory scale methods, such as the hanging drop, can surmount these problems, they are problematic on a large scale. While the use of hormones and chemicals to direct differentiation, rather than embryoid body formation, seems a more attractive approach, our understanding of the complex interactions required for organogenesis is rudimentary. Filling in these gaps in our understanding will require painstaking and difficult analysis of embryological processes that are not easily accessible to experimentation.

Disclosed herein are methods that can generate virtually any cell type in vitro, as well as compositions used in the methods or derived from the methods. These cell lines which are generated can be cloned, characterized, frozen, and used in any quantity necessary while, for example, maintaining the advantages of a normal karyotype. The availability of these cells will enable the realization of many of the potential applications currently envisioned for human stem cells.

### III. SUMMARY

Disclosed are methods and compositions related to production of cells and cell lines.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic for an example of a cassette for reversible transformation using sequential expression of activated, dominant negative pairs of a transforming gene. Below the schematic there is a temporal progression of which parts of the cassette are activated during the progression from a pluripotent stem cell to a differentiated cell.

FIGS. 2A-2C show examples of plasmids that can be used for isolation of an hepatocyte derived cell line from ACTEG1, a gonadal ridge derived pluripotent stem cell.

FIG. 3 shows a schematic of an example of a cassette for reversible transformation using an excisable activated oncogene.

FIG. 4 shows the structure of ploxHBV-aRas, an example of a plasmid which can be used in the generation of a cassette as in FIG. 3.

FIG. 5 shows a schematic of an example of a cassette for reversible transformation using a temperature sensitive transforming gene.

FIG. 6 shows a schematic of the pEGSH plasmid, as indicated by Stratagene.

FIG. 7 shows a diagram of a form of the disclosed tissue specific reversible transformation (TSRT) method.

FIG. 8 shows a schematic of an example of a cassette for reversible transformation using a tetracycline regulated CMV promoter driving expression of a dominant negative ras and a tissue specific promoter driving expression of a-ras.

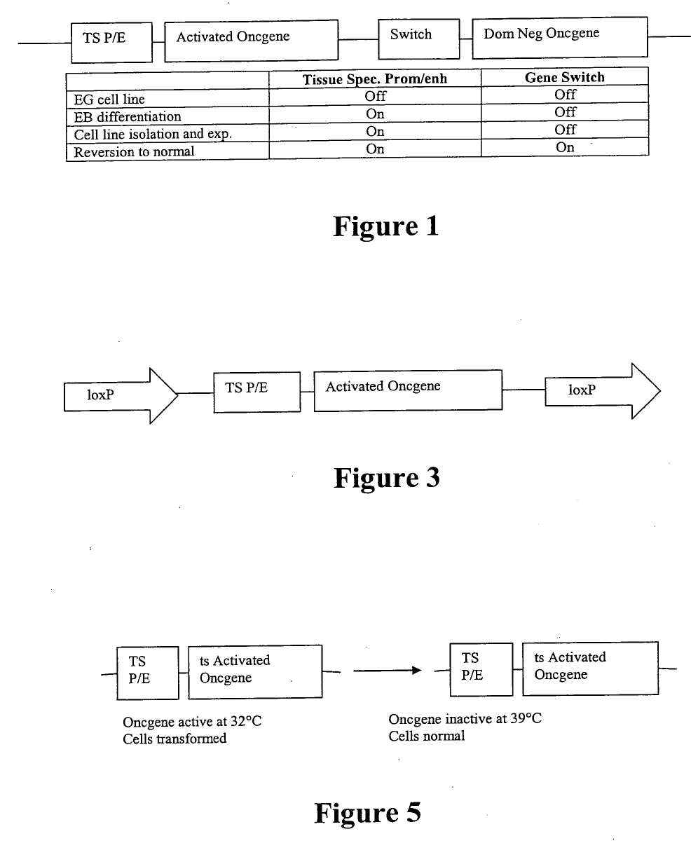

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Numerous authors have written about the possible applications of human pluripotent stem cells (for example, Gearhart, J (1998) Science 282, 1061-1062; Pera, M F, et al., (2000) J. Cell Sci. 113, 5-10; Trounson, A (2001) Reprod Fertil Dev. 2001; 13(7-8):523-32; Sussman, N L, Kelly, J H. (1994) U.S. Pat. No. 5,368,555). These range from target evaluation and toxicity testing in drug discovery to attempting to cure type I diabetes by implanting new beta cells into the pancreas. Each of these applications requires large quantities of differentiated cells from a controlled and renewable source. While previous technologies fail to meet this requirement, disclosed herein are compositions and methods capable of producing large quantities of a desired cell type in vitro in a controlled and reproducible way.

Human pluripotent stem cells promise to dramatically alter and extend our ability to treat many of the chronic illnesses that define modern medicine. Neurodegenerative disease, neuromuscular disease, diabetes, autoimmune disease, leukemia, and heart disease are all examples of targets for cell-based therapies aimed at replacing and regenerating damaged tissue.

This vision is primarily based on the success of using pluripotent stem cells to generate transgenic mice (Zambrowicz, B P, Sands, A T (2003) Nat. Rev. Drug Disc. 2, 38-51). The ability to alter stem cells in vitro and create mice with targeted mutations has led to rapid advancement in the understanding of gene regulation and function, as well as mammalian development. This, in turn, has led to an ability to mimic human disease in mouse models, facilitating the process of drug development. Work with pluripotent stem cells in mice has shown that they are capable of contributing to any tissue in the organism, and that genes of interest can be altered essentially at will, being turned off, deleted, activated or expressed in individual tissues, depending on the needs of the particular experiment.

While these results properly encourage enthusiasm for human pluripotent stem cell work, they also frame the central problem in generalizing this work from the mouse to the human. Because of the success of the transgenic mouse as a model, and its ability to replicate the complex interplay of tissues that leads to organotypic differentiation, substantially less attention has been devoted to defining conditions that reproduce differentiation in vitro. Yet, in order to realize the vision of cell-based therapies, substantial quantities of specific cell types or sets of cell types will need to be generated in vitro. It would be useful to have differentiated stem cells comprising an absolutely homogeneous population, that is, that they be clonal or semi-purified, in order to avoid the well documented propensity of pluripotent stem cells to form tumors when implanted in other than their normal environment (Andrew, P W (2002) Philos. Trans. R. Soc. Lond. B. Biol. Sci. 357, 405-417). Accordingly, disclosed are homogenous differentiated stem cells, clonal differentiated stem cells, semi-purified differentiated stem cells, and mixed differentiated stem cells. Also disclosed are populations of cells, which can, but need not be, clonal, can, but need not be, the same cell type, and can, but need not be, a subset of all cell types that could be produced. These populations can be used, for example, for therapy, in in vivo toxicity assays or in other types of in vitro assays such as drug screening. Also disclosed are semi-purified sets of a cell type which contain, at least 99, 98, 97, 96, 95, 94, 93, 92, 91, 90, 89, 88, 87, 86, 85, 84, 83 82, 81, 80, 79, 78, 77, 76, 75, 74, 73, 72, 71, 70, 65, 60, 55, 50, 45, 40, 35, 30, or 25% of a particular cell type, such as any combination of any cell disclosed herein, any cell disclosed herein, or a hepatocyte.

Disclosed is a method for producing differentiated stem cells and/or one or more types of cells. Also disclosed are cells and/or cell types produced by the disclosed method. The method generally can involve incubating stem cells under conditions that promote differentiation and selecting or screening for one or more cells and/or cell types. The stem cells used can comprise a nucleic acid segment comprising a transcriptional control element operably linked to a nucleic acid sequence encoding a marker. The selection or screening can be on the basis of the marker. The cells and/or cell types in which the marker is expressed can be selected or screened for, or the cells and/or cell types in which the marker is not expressed can be selected or screened for. In this way, particular cells and/or cell types can be obtained from stem cells.

The transcriptional control element can be a tissue-, cell-, cell type- and/or cell lineage-specific transcriptional control element, which means that the transcriptional control element allows or promotes expression of nucleic acid sequences operably linked to the transcriptional control element in specified tissues, cells, cell types and/or cell lineages, respectively. Thus, in the disclosed method, the marker can be expressed in tissues, cells, cell types and/or cell lineages for which the transcriptional control element is specific. In this way, particular cells, cells of particular tissues, particular cell types and/or cells of particular cell lineages can be obtained from stem cells.

The disclosed method has the advantage of providing a feature or characteristic (expression or non-expression of the marker) by which differentiated cells of interest can be selected or screened from stem cells and differentiated cells that are not of interest. The concept of the disclosed method is that the marker, operably linked to a transcriptional control element, will be expressed (or not expressed) only or primarily when starting stem cells have differentiated into a desired type of cell or tissue (the type of tissue or cell for which the transcriptional control element is specific). Any cell, cell type, cell lineage, and/or tissue of interest can be targeted by choosing a transcriptional control element relevant to the cell, cell type, cell lineage, and/or tissue of interest.

A useful type of marker is a transformation agent, such as an oncogene. In this case, expression of the transformation agent can cause transformation of the cell. The result can be growth and/or preferential growth of cells expressing the transformation agent. In the context of differentiated stem cells, transformation, and the associated growth, can allow selective and/or preferential growth of cells expressing the transformation agent because most other differentiated stem cells will grow slowly if at all. Cells expressing (or not expressing) the marker can be selected by applying selective pressure relevant to the marker. For example, many genes and proteins are known that can be used to give cells a selective advantage or disadvantage. Cells expressing (or not expressing) the marker can be screened by identifying cells expressing (or not expressing) the marker. For example, many enzymes and proteins are known that constitute and/or produce a signal that can be detected. Such a signal can be the basis of cell identification.

The method can also involve reversal of the marker expression. This can be accomplished by, for example, removal of all or part of the nucleic acid segment, such as by excision of all or part of the nucleic acid segment; inactivation of the nucleic acid segment, the transcriptional control element, and/or the marker; repression of the nucleic acid segment, the transcriptional control element, and/or the marker; and/or introduction and/or expression of a reversing agent. Excision of the nucleic acid segment can be accomplished in numerous ways. For example, the nucleic acid segment can be excised via site-specific recombination using a recombinase. A reversing agent can alter and/or reduce the effect of the marker. For example, where the marker is a transforming agent such as Ras, transformation of the cells (the effect of Ras) can be reversed by expression of a dominant negative Ras. Forms of the disclosed method that involve use of a transformation agent and subsequent reversal of transformation can be referred to as tissue specific reversible transformation (TSRT). Although TSRT refers to tissue specific reversible transformation, this is merely for convenience and it is intended that TSRT refers to tissue-, cell-, cell type- and/or cell lineage-specific expression of the transforming agent.

As indicated, combinations of reversal operations can be used to accomplish reversal. For example, excision of the nucleic acid segment and expression of a reversing agent can be used together in the disclosed method. Removal of the nucleic acid segment is a useful reversal operation when a cell having minimal genetic alteration (compared to a natural cell of the same type, for example) is desired. This is desirable, for example, if the cells are to be used therapeutically.

Disclosed herein are strategies involving tissue-specific reversible transformation for establishing differentiated cell lines of any particular cell type, using stem cells as a starting material. Disclosed are methods that employ tissue specific expression of a transforming gene, which can be used to identify and culture the particular cell type. This transforming event can, in some forms of the method, then be reversed, using one of a number of possible processes, leaving a clonal or semi-purified population of non-transformed, differentiated cells, including populations of different or semi-purified cells, or a clonal population of cells, as discussed herein.

Disclosed are compositions and methods involving modified stem cells, such as pluripotent stem cells, wherein the pluripotent stem cell contains, for example, a marker whose expression is controlled by a transcription control element, such as a tissue specific promoter, a cell type specific promoter, a cell specific promoter, and/or a cell lineage specific promoter. The modified pluripotent stem cell can then be grown under conditions that allow for cell proliferation or embryoid body (EB) and differentiated cell formation as discussed herein. When the stem cell is allowed to form an EB the EB produces many different cell types through spontaneous differentiation. In some forms of the disclosed method, after the EB is allowed to form for a desired time, a selective pressure can be applied by, for example, growing the cells in the cognate selection media for the marker. While at this point, there are many different cell types (the number depends on the length of time the EB is allowed to develop without selective pressure), the selective pressure causes cells having the expressed marker to be selectively amplified or visualized. The cells having the selective marker are a desired differentiated cell type or types, because the marker can be designed to be preferentially or selectively expressed in the desired cell type or types from the tissue specific promoter. It is also understood that in certain systems, there can be more than one tissue specific promoter driven marker. Having multiple markers driven by different promoters, the selective stringency can be increased for cell types where the tissue specific promoter is not expressed exclusively in a single tissue. It is also understood that there can an additional identification step after the selection step or steps in which the desired cell is identified. These identified cells can then be further isolated and cultured.

After a period of time under the selective conditions (selective pressure, for example) can be removed to allow for increased cell proliferation, and then the selective pressure can be reapplied. Thus, iterative rounds of selection can occur, increasing the stringency of selection. The iterative rounds of selection can also occur in systems with more than one type of marker being expressed from the same tissue specific promoter. In some forms of the method these iterative rounds of selection can occur such that, for example, a first marker is utilized and then a second marker is utilized and then the first marker is utilized and the second marker is utilized, and so forth. After the selective pressure is completed, the desired differentiated cells can be grown under non-selective conditions, at which point the marker and related DNA can be removed if desired. There are numerous ways for achieving this, including, for example, the use of recombinase technology, such as Cre-lox technology or temperature specific mutant markers. It is also understood that the marker can be integrated into the pluripotent stem cell chromosome or can be carried on extrachromosomal cassettes, such as a mammalian artificial chromosome.

Disclosed are methods and compositions for establishing differentiated cell lines of any particular cell type, using stem cells as a starting material. This mechanism can employ tissue specific expression of a marker, such as a transforming gene, which is used to identify and culture the particular cell type. This transforming event can then be reversed, using one of a number of possible processes, leaving a clonal or semi-purified population of nontransformed, differentiated cells.

For example, disclosed are compositions and methods related to the human liver specific promoter/enhancers from the hepatitis B virus core antigen driving different variations of the RAS gene. In some forms of the method, an activated RAS coupled to an ecdysone inducible dominant negative RAS as the reversing agent can be used. In some forms of the method, the HBV/RAS construct can be flanked with loxP sites that can be excised with CRE recombinase. Some forms of the method can use the generation of a temperature sensitive (ts), activated RAS.

Typically the marker construct can be transfected into a stem cell line, such as a human embryonal germ (EG) cell line. Differentiation of the resultant cell line can then be initiated, for example, by the formation of embryoid bodies. In this way, natural biological processes result in development of the appropriate cell type. When a cell becomes the desired cell type, such as an hepatocyte, the tissue or cell specific promoter, such as a liver specific construct, will be activated and the marker will be expressed. The cell is, for example, transformed or marked by expression of the marker. A selective media can be used, for example, such as soft agar for transformed cells, and when placed in the selective media only the appropriately differentiated transformed cells in the EB will survive or have selective advantage. Transformed cells will preferentially or selectively grow out and form colonies. Colonies can be picked and re-plated for cloning. For use, the cells can be grown by standard methods to the desired quantity and configuration. At the appropriate time, the reversing signal can be applied, for example, either ecdysone for gene switches, CRE recombinase for lox constructs or temperature shift for ts construct, leaving a population of cells functionally equivalent to primary cultures.

For example, disclosed are pluripotent stem cells containing a nucleic acid segment comprising the structure P-I, wherein: P is a transcriptional control element; and I is a sequence encoding a marker, wherein the marker can comprise a transformation agent.

Disclosed are cells, wherein the marker is expressed from a heterologous nucleic acid, wherein the nucleic acid further comprises a suicide gene, wherein P is a tissue specific transcriptional control element, wherein P causes I to be preferentially or selectively expressed, wherein the immortalization agent is a temperature permissive agent, wherein I comprises the SV40 large T antigen, wherein the nucleic acid segment is flanked by a site-specific excision sequence, wherein I is flanked by a site-specific excision sequence, wherein P is flanked by a site-specific excision sequence, and/or wherein P-I is flanked by a site-specific excision sequence, X, forming X-P-I-X.

Also disclosed are cells produced by excising the nucleic acid segment from the stem cells disclosed herein.

Disclosed are cells, wherein the nucleic acid segment comprising the structure P-I is excised using an adenovirus-mediated site-specific excision, and/or wherein the excision of the nucleic acid molecule comprising the structure P-I results in recombination of the non-excised nucleic acid molecule.

Disclosed are methods of deriving a population of conditionally immortal cell types from stem cells, comprising: transfecting a stem cell with a construct containing one of the nucleic acid molecules P-I disclosed herein, culturing the stem cells in an environment such that transcriptional control of element P is activated, whereby I is preferentially or selectively expressed, and selecting cell types expressing I.

Disclosed are methods, further comprising the step of increasing the purity of the population of cells expressing I, wherein the step of increasing the purity comprises creating a clonal or semi-purified population of cells, further comprising excising the nucleic acid, further comprising freezing the selected cell type, and/or further comprising adding a gene of interest to the population of cells.

Disclosed are methods of deriving conditionally immortal cell types, comprising transfecting pluripotent stem cells with a construct containing one of the nucleic acid molecules P-I disclosed herein, activating control element P, whereby I is preferentially or selectively expressed, selecting cell types expressing I and excising the construct containing the P-I nucleic acid molecule, contacting the selected cell types with an environment such that the ends of the nucleic acid formerly containing the construct containing the P-I nucleic acid molecule recombine; and freezing of the selected cell type.

Disclosed are methods wherein the stem cell culture is allowed to spontaneously differentiate into an embryoid body.

Also disclosed are methods of deriving a cell culture, comprising transfecting pluripotent stem cells with a construct containing one of the nucleic acid molecules P-I disclosed herein, contacting the stem cells with an environment such that transcriptional control element P is activated and I is preferentially or selectively expressed, culturing the cells expressing I.

Disclosed are methods, further comprising cloning the cultured cells expressing I.

Disclosed are methods of treating a patient comprising administering the cells disclosed herein, such as by transplanting the cells disclosed herein.

Disclosed are methods of assaying a composition for toxicity comprising incubating the composition with the cells produced by the method disclosed herein.

Disclosed are pluripotent stem cells containing a nucleic acid molecule construct comprising the structure P-I, wherein P is a tissue specific transcriptional control element, P causes I to be preferentially or selectively expressed; and I is a temperature permissive immortalization agent.

Disclosed are pluripotent stem cell containing a nucleic acid molecule construct comprising the structure X-P-I-X, wherein P is a tissue specific transcriptional control element, P causes I to be preferentially or selectively expressed, I is a temperature permissive immortalization agent; and X is a site-specific excision sequence.

Disclosed are cells, wherein P-I is excised, wherein P-I is excised at X by an adenovirus-mediated site-specific excision, and/or wherein the excision of P-I allows recombination of the nucleic acid formerly containing the construct containing the P-I nucleic acid molecule.

Derived are methods of deriving stem cell derived conditionally immortal cell types, comprising: transfecting pluripotent stem cells with a construct containing the nucleic acid molecule construct P-I disclosed herein, contacting the stem cells with an environment such that transcriptional control element P is activated and I is preferentially or selectively expressed, selection of stem cell derived cell types expressing I; and cloning and freezing of a selected cell type.

Disclosed are methods of deriving stem cell derived conditionally immortal cell types, comprising, transfecting pluripotent stem cells with a construct containing the nucleic acid molecule construct X-P-I-X disclosed herein contacting the stem cells with an environment such that transcriptional control element P is activated and I is preferentially or selectively expressed, selecting the stem cell derived cell types expressing I; and cloning and freezing of a selected cell type.

Disclosed are methods of deriving stem cell derived conditionally immortal cell types, comprising transfecting pluripotent stem cells with a construct containing the nucleic acid molecule construct X-P-I-X disclosed herein; contacting the stem cells with an environment such that transcriptional control element P is activated and I is preferentially or selectively expressed, selecting the stem cell derived cell types expressing I, excising of the construct containing the P-I nucleic acid molecule; and cloning and freezing of a selected cell type.

Disclosed are cells, wherein P and I are contained in the same vector or wherein P and I are contained in different vectors.

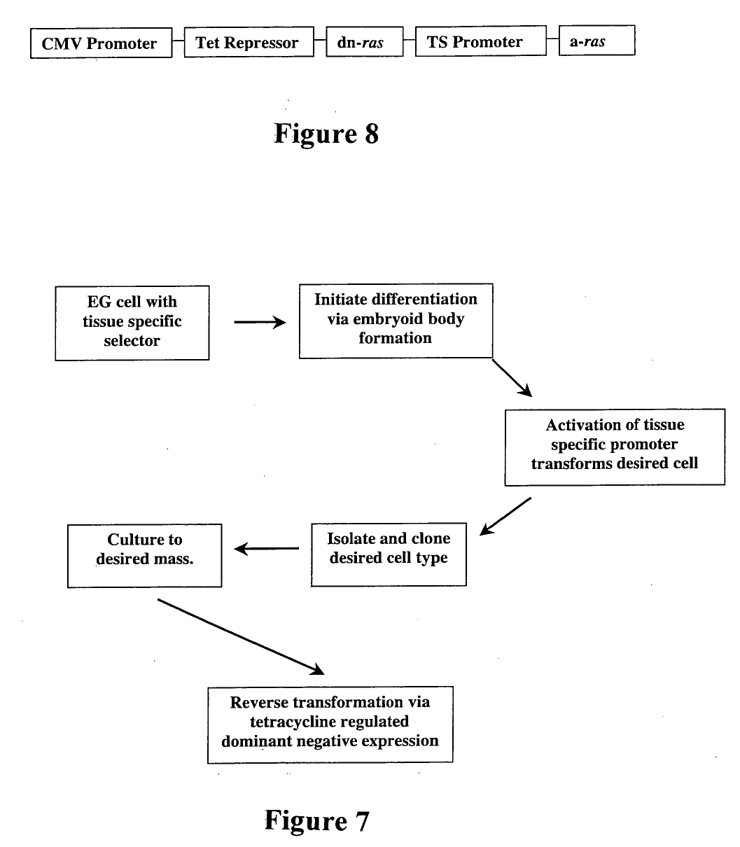

Disclosed are compositions and methods for generation of differentiated cells from stem cells. Particularly useful forms of the method involve site specific recombination and a tissue specific, reversible transformation (TSRT) process. The method can use, for example, flp/frt mediated recombination and a tissue specific promoter to activate, for example, ras transformation and identify the appropriate cell. Transformation can then be reversed, using, for example, tetracycline regulated expression of a dominant negative ras. Stepwise application of these techniques yields cells of any desired cell type that can be cloned, banked and cultured without extensive knowledge of their developmental program. Reversal of the transformation yields a verifiably uniform population of differentiated cells. The process is outlined in the FIG. 7 using, as an example, a nucleic acid segment diagramed in FIG. 8. Any cell type can be selected by switching out the tissue specific promoter (TS Promoter) in the nucleic acid segment. The α-MHC promoter is used in this example. The tissue specific selector in FIG. 8 consists of a tetracycline regulated CMV promoter driving dominant negative ras and a tissue specific promoter driving a-ras. Formation of the tissue type of interest activates the promoter and transforms the cell. When desired, transformation is reversed by the addition of tetracycline.

The method can use stem cells, such as human embryonic germ (EG) cell lines, that can be cultured under defined, feeder free conditions. In some forms of the method, TSRT process can be used in these cells can be used to identify and culture cell types formed during embryoid body differentiation and take advantage of the ability of a transforming gene, such as ras, expressed from a tissue specific promoter, to drive cell growth. These cells can then be cloned, characterized and frozen in Master Cell Banks for use as needed. When the cells are used, such as drug screening or cell therapy, the transformation process can be reversed through expression of a corresponding dominant negative ras. In this way, any required cell type can be identified, cultured to any desired mass, and quantitatively converted to an untransformed phenotype.

The disclosed method can involve, for example, the use of modified stem cells adapted for the method. For example, a frt recombination site can be inserted into a stem cell line, such as an EG cell line, to allow insertion of the tissue specific selectors into the same known site for each selection. The selectors can be nucleic acid segments containing, for example, expression-regulated transformation agent. Independent isolates can be characterized to identify a stem cell line with an optimal integration site. The resulting stem cell line can be referred to as a frt insertion (FI) line. The frt insertion lines can be used to create a tetracycline regulated insertion site. The resulting tetracycline operator frt insertion (TOFI) lines allow regulated expression of a dominant negative transformation agent to reverse the transformation.

Flp is a member of the lambda integrase family, named for its ability to flip a DNA segment in yeast (Branda and Dymecki, (2004) Talking about a revolution: the impact of site specific recombinases on genetic analyses in mice. Developmental Cell 6, 7-28). It mediates recombination through a specific recognition sequence, frt (flp recombinase target). Insertion of a frt sequence has been demonstrated to allow site specific integration of a plasmid containing a second frt sequence. Flp/frt has been demonstrated to work efficiently in embryonic stem cells (Dymecki, (1996) Flp recombinase promotes site specific DNA recombination in embryonic stem cells and transgenic mice. Proc. Natl. Acad. Sci. 93, 6191-6196).

By inserting a frt site (or other site specific recombination or insertion site) into stem cell lines, the selector construct, the tissue specific promoter attached to ras, can be targeted to the same site for any selection. This eliminates a problem with undirected insertion of DNA where the DNA integrates into a section of the genome that is turned on or off as differentiation progresses or into a functioning gene. Although not an insurmountable problem in traditional DNA insertion systems (it can generally be overcome by continued growth in the selection medium), the disclosed method provides an elegant solution. The disclosed method can use random insertion of the selector, but this requires more work since each insert might need to be assessed for insertional effects. Using a recombination site allows generation of appropriate cell once. This cell can then be used over and over, recombining into the same site repeatedly to select additional cell types. By recombining into an existing site, all transfectants will be the same and so an entire dish can be collected, avoiding the problems of repeated cloning. Use of a flp/frt system also maximizes the efficiency of transfection.

The disclosed method can be used to make any desired cell type based on, for example, the use of transcription control elements active in the desired cell type. For example, cardiomyocyte cells can be produced in the disclosed method by using, for example, the alpha myosin heavy chain (AMHC) promoter driving ras. An inserted tetracycline regulated, dominant negative ras can then be used to reverse the transformation of the cardiomyocyte cells. Temperature sensitive transformants or excision of the selector (nucleic acid segment containing the expression-regulated transformation agent) through regulated expression of the flp recombinase.

A. Compositions

1. Stem Cells

Stem cells are defined (Gilbert, (1994) DEVELOPMENTAL BIOLOGY, 4th Ed. Sinauer Associates, Inc. Sunderland, Mass., p. 354) as cells that are “capable of extensive proliferation, creating more stem cells (self-renewal) as well as more differentiated cellular progeny.” These characteristics can be referred to as stem cell capabilities. Pluripotential stem cells, adult stem cells, blastocyst-derived stem cells, gonadal ridge-derived stem cells, teratoma-derived stem cells, totipotent stem cells, multipotent stem cells, embryonic stem cells (ES), embryonic germ cells (EG), and embryonic carcinoma cells (EC) are all examples of stem cells.

Stem cells can have a variety of different properties and categories of these properties. For example in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express Oct 4 and Nanog (Rodda et al., J. Biol. Chem. 280, 24731-24737 (2005); Chambers et al., Cell 113, 643-655 (2003)). It is understood that some stem cells will express these at the mRNA level, and still others will also express them at the protein level, on for example, the cell surface or within the cell.

It is understood that stem cells can have any combination of any stem cell property or category or categories and properties discussed herein. For example, some stem cells can express alkaline phosphatase, not express SSEA-1, proliferate for at least 20 passages, and be capable of differentiating into any cell type. Another set of stem cells, for example, can express SSEA-1 on the cell surface, and be capable of forming endoderm, mesoderm, and ectoderm tissue and be cultured for over a year without differentiation. Another set of stem cells, for example, could be pluripotent stem cells that express SSEA-1. Another set of stem cells, for example, could be blastocyst-derived stem cells that express alkaline phosphatase.

Stem cells can be cultured using any culture means which promotes the properties of the desired type of stem cell. For example, stem cells can be cultured in the presence of basic fibroblast growth factor, leukemia inhibitory factor, membrane associated steel factor, and soluble steel factor which will produce pluripotential embryonic stem cells. See U.S. Pat. Nos. 5,690,926; 5,670,372, and 5,453,357, which are all incorporated herein by reference for material at least related to deriving and maintaining pluripotential embryonic stem cells in culture. Stem cells can also be cultured on embryonic fibroblasts and dissociated cells can be re-plated on embryonic feeder cells. See for example, U.S. Pat. Nos. 6,200,806 and 5,843,780 which are herein incorporated by reference at least for material related to deriving and maintaining stem cells.

One category of stem cells is a pluripotential embryonic stem cell. A pluripotential embryonic stem cell as used herein means a cell which can give rise to many differentiated cell types in an embryo or adult, including the germ cells (sperm and eggs). Pluripotent embryonic stem cells are also capable of self-renewal. Thus, these cells not only populate the germ line and give rise to a plurality of terminally differentiated cells which comprise the adult specialized organs, but also are able to regenerate themselves.

One category of stem cells are cells which are capable of self renewal and which can differentiate into cell types of the mesoderm, ectoderm, and endoderm, but which do not give rise to germ cells, sperm or egg.

Another category of stem cells are stem cells which are capable of self renewal and which can differentiate into cell types of the mesoderm, ectoderm, and endoderm, but which do not give rise to placenta cells.

Another category of stem cells is an adult stem cell which is any type of stem cell that is not derived from an embryo or fetus. Typically, these stem cells have a limited capacity to generate new cell types and are committed to a particular lineage, although adult stem cells capable of generating all three cell types have been described (for example, U.S. Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are referred to as “pluripotent hematopoietic stem cell” for its pluripotency within the hematopoietic lineage. A pluripotent adult stem cell is an adult stem cell having pluripotential capabilities (See for example, U.S. Patent Publication no. 20040107453, which is U.S. patent application Ser. No. 10/467,963.

Another category of stem cells is a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Another category of stem cells is a gonadal ridge-derived stem cell which is a pluripotent stem cell which was derived from a cell which was obtained from, for example, a human embryo or fetus at or after the 6, 7, 8, 9, or 10 week, post ovulation, developmental stage. Alkaline phosphatase staining occurs at the 5-6 week stage. Gonadal ridge-derived stem cell can be derived from the gonadal ridge of, for example, a 6-10 week human embryo or fetus from gonadal ridge cells.

Another category of stem cells are embryo derived stem cells which are derived from embryos of 150 cells or more up to 6 weeks of gestation. Typically embryo derived stem cells will be derived from cells that arose from the inner cell mass cells of the blastocyst or cells which will be come gonadal ridge cells, which can arise from the inner cell mass cells, such as cells which migrate to the gonadal ridge during development.

Other sets of stem cells are embryonic stem cells, (ES cells), embryonic germ cells (EG cells), and embryonic carcinoma cells (EC cells).

Also disclosed is another category of stem cells called teratoma-derived stem cells which are stem cells which was derived from a teratocarcinoma and can be characterized by the lack of a normal karyotype. Teratocarcinomas are unusual tumors that, unlike most tumors, are comprised of a wide variety of different tissue types. Studies of teratocarcinoma suggested that they arose from primitive gonadal tissue that had escaped the usual control mechanisms. Such properties and manipulations are generally applicable to teratoma-derived stem cells.

Stem cells can also be classified by their potential for development. One category of stem cells are stem cells that can grow into an entire organism. Another category of stem cells are stem cells (which have pluripotent capabilities as defined above) that cannot grow into a whole organism, but can become any other type of cell in the body. Another category of stem cells are stem cells that can only become particular types of cells: e.g. blood cells, or bone cells. Other categories of stem cells include totipotent, pluripotent, and multipotent stem cells.

The disclosed methods and compositions are generally described by reference to “stem cells” or “pluripotent stem cells.” However, the disclosed methods are not limited to use of stem cells and pluripotent stem cells. It is specifically contemplated that the disclosed methods and compositions can use or comprise any type or category of stem cell, such as adult stem cells, blastocyst-derived stem cells, gonadal ridge-derived stem cells, teratoma-derived stem cells, totipotent stem cells, and multipotent stem cells, or stem cells having any of the properties described herein. The use of any type or category of stem cell, both alone and in any combination, with or in the disclosed methods and compositions is specifically contemplated and described.

2. Differentiation of Stem Cells In Vitro

Until recently, pluripotent stem cell work was confined almost entirely to the mouse. Although lines had been derived from several other species, the experimental advantages of the mouse served to concentrate most of the work there. A secondary consequence of the mouse as an experimental model has been to deemphasize work on establishing conditions to facilitate in vitro differentiation. The relative simplicity of creating transgenic mice has discouraged the uncertain and serendipitous work of defining cell culture conditions that mimic the exceedingly complex interaction of cells that leads to organotypic differentiation. With the announcement of human pluripotent cell lines, the ability to modulate differentiation in vitro has taken on new prominence.

Pluripotent stem cells maintained, for example, on feeder layers and with appropriate culture medium remain undifferentiated indefinitely. Removal from the feeder layer and culture in suspension leads to the formation of aggregates and other differentiated cells (Kyba, M, (2003) Meth. Enzymol. 365, 114-129). These aggregates begin to organize and develop some of the characteristics of blastocysts. These protoblastocysts are called embryoid bodies (EB). Within the EB, progressive rounds of proliferation and differentiation occur, roughly following the pattern of development. While a wide variety of tissue types can be identified in EBs, without outside direction, differentiation is disorganized and does not lead to formation of significant quantities of any one cell type (Fairchild, P J, (2003) Meth. Enzymol. 365, 169-186). Numerous strategies have been devised to direct a larger proportion of cells down any particular developmental pathway (Wassarman, P M, Keller, G M. (2003) METHODS IN ENZYMOLOGY, Differentiation of Embryonic Stem Cells, vol. 365, Elsevier Academic Press, New York, N.Y., 510p.). These have taken the form of treatment with known morphogens, alteration of the hormonal environment, culture of the cells on particular substrata, and sequential application of chemicals known to affect differentiation in vitro. All of these strategies have been successful in certain applications but in no case have they been able to generate cells that are homogenously one cell type.

In addition to the problem of homogeneity, another problem arises when one considers the possibility of actually employing a particular cell type in a secondary application. For example, normal human hepatocytes for use in toxicity testing can be very useful in drug development. Human primary hepatocytes, cells derived directly from human livers, are in extremely short supply. Hepatocytes derived from a line of stem cells could solve this problem but would need to be available in significant numbers. Disclosed are compositions and methods capable of solving this problem.

In order for stem cell derived products to be applied in real applications, large quantities of identical cells need to be generated. Ideally, this can be a general process that could be applied broadly rather than necessitating tedious experimentation for each cell type.

3. Cell Specific Generation

Tissue specific reversible selection, such as transformation provides a useful process for generating differentiated stem cells. The disclosed method allows permanent lines of cells of any specific type to be identified and cultured, then allows the entire population to revert to the normal phenotype or be eliminated from the population.

Disclosed are compositions and methods for using tissue specific, reversible transformation of stem cell lines, which will develop into cell lines of any desired cell type. The disclosed methods use tissue specific expression of a transforming gene. Also disclosed are methods where the transformation is reversed via any number of strategies, such as expression of a dominant negative version of the transforming gene, depending on the context of the desired cell product. The disclosed compositions and methods avoid large scale cultivation of stem cells, as stem cells themselves need only be grown on a laboratory scale to isolate the desired cell type; they develop individual cell lines that can be cloned and characterized as is currently done in any large scale cell culture application and the lines can be characterized and frozen; they bypass pieces of biology that are poorly understood at present because the compositions and methods utilize the power of the biology as it is, rather than attempting to duplicate these complex processes on a large scale; and the cell lines will behave as most transformed lines in culture with general culture conditions, i.e., insulin, transferrin, selenium, ordinary cell culture medium, can be sufficient for most of these lines. It is understood that non-transformation methods as discussed herein can be used as well, and are interchangeable with transformation methods.

4. Modified Stem Cells

Disclosed are modified stem cells. A modified stem cell is a stem cell that has a genetic background different than the original background of the cell. For example, a modified stem cell can be a stem cell that expresses a marker from either an extra chromosomal nucleic acid or an integrated nucleic acid. The stem cell can be modified in a number of ways including through the expression of a marker. A marker can be anything that allows for selection or screening of the stem cell or a cell derived from the stem cell. For example, a marker can be a transformation gene, such as Ras, which provides a cell the ability to grow in conditions in which non-transformed cells cannot.

Cells can be put under a selective pressure which means that the cells are grown or placed under conditions designed to alter the cell population in some way which is related to the marker. For example, if the marker confers antibiotic resistance to the cells that express the marker, then the cell population can be put under conditions where the antibiotic was present. Only cells expressing the gene conveying antibiotic resistance can survive or can have a survival advantage relative to cells not expressing the antibiotic resistance gene. Cells that express the marker gene and have a selective advantage can in some forms of the method be selectively amplified relative to other cells not having the marker meaning they would grow at a rate or survive at a rate greater than the cells not having the marker. In some forms of the method the selection of the cells having the marker has a certain selective stringency. The selective stringency is the efficiency with which the marker identifies cells having the marker from cells that do not have the marker. For example, the selective stringency can be such that the marker producing cells have at least 2, 4, 8, 10, 15, 20, 25, 30, 40, 50, 75, 100, 200, 400, 500, 800, 1000, 2000, 4000, 10,000, 25000, 50,000 fold growth advantage over the non-marker expressing cells. In some forms of the method the selective stringency can be expressed as a selective ratio of the percent of cells expressing the marker that survive over a period of time, for example, a passage, over the percent of cells not expressing the marker that survive over the same time period. For example disclosed are markers that can confer a selective ratio of at least 1, 1.5, 2, 4, 8, 10, 15, 20, 25, 30, 40, 50, 75, 100, 200, 400, 500, 800, 1000, 2000, 4000, 10,000, 25000, 50,000, or 100,000. The markers allow the cells expressing the markers to be selectively grown or visualized which means that the cells expressing the marker can be preferentially or selectively grown or identified over the cells not expressing the marker.

a) Markers

The marker or marker product can used to determine if the marker or some other nucleic acid has been delivered to the cell and once delivered is being expressed. For example, the marker can be the expression product of a marker gene or reporter gene. Examples of useful marker genes include the E. Coli lacZ gene, which encodes β-galactosidase, adenosine phosphoribosyl transferase (APRT), and hypoxanthine phosphoribosyl transferase (HPRT). Fluorescent proteins can also be used as markers and marker products. Examples of fluorescent proteins include green fluorescent protein (GFP), green reef coral fluorescent protein (G-RCFP), cyan fluorescent protein (CFP), red fluorescent protein (RFP or dsRed2) and yellow fluorescent protein (YFP).

(1) Negative Selection Markers

The marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

(2) Dominant Selection Markers

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Other examples include the neomycin analog G418 and puromycin.

(3) Transforming Genes

A transforming gene can be used as a marker. A transforming gene is any sequence that encodes a protein or RNA that causes a cell to have at least one property of a cancer cell, such as the ability to grow in soft agar. Other properties include loss of contact inhibition and independence from growth factors, for example. Also, changes in morphology can occur in transformed cells, such as the cells become less round. Transforming genes can also be referred to as transformation genes. Transforming genes, transformation genes, and their products can be referred to as transforming agents or transformation agents. Transformation agents can also be referred to as immortalization agents.

An oncogene can be a transforming gene and typically a transforming gene will be an oncogene. An oncogene typically codes for a component of a signal transduction cascade. Typically the normal gene product of the oncogene regulates cell growth and a mutation in the protein or expression occurs which deregulates this activity or increases the activity. Oncogenes typically code for molecules in signal transduction pathways, such as the MAPK pathway or Ras pathway, and, for example, can be growth factors, growth factor receptors, transcription factors (erbA: codes a thyroid hormone receptor (steroid receptor), rel: form pairwise combinations that regulate transcription (NF-kB), v-rel: avian reticuloendotheliosis, jun & fos), protein kinases, signal transduction, serine/threonine kinases, nuclear proteins, growth factor receptor kinases, or cytoplasmic tyrosine kinases. It is understood that many oncogenes in combination can become transforming. All sets of combinations of the disclosed oncogenes and transforming genes specifically contemplated. Some oncogenes, such as Ras, are transforming by themselves.

Membrane associated transducing molecules can often be oncogenes. Membrane associated transducing molecules, such as Ras, are indirectly activated by the binding of other molecules to nearby receptors. The activation of the nearby receptors causes the oncogene to become active that starts a signaling cascade which leads to changes in the normal cell behavior. Receptor tyrosine kinases can also be oncogenes. Receptor tyrosine kinases are enzymes that are capable of transferring phosphate groups to target molecules. When a target molecule, such as a growth factor, binds to the extracellular portion of the kinase a signal is transmitted through the cell membrane causing a signal transduction cascade. An example of this type of oncogene is the HER2 protein. Receptor-associated kinases are also membrane associated enzymes but they are activated by binding other nearby receptors. This binding causes the kinase to phosphorylate a target protein causing signal transduction to the nucleus. Src is an example of this type of oncogene. Transcription factors are proteins that bind to specific sequences along the DNA helix causing the bound genes to be expressed in the nucleus. An example of this type of oncogene is myc. Some transcription factors are repressors, such as Rb. Telomerase is a protein-RNA complex that maintains the termini of chromosomes. If telomerase is not present or present in low amounts, chromosomes shorten with each cell division until serious damage occurs. Telomerase is not expressed or present or lowly expressed or present in most normal cells, but is present in concentrations, higher than in a cognate untransformed cell in most transformed cells. Apoptosis regulating proteins are proteins functioning to control programmed cell death. When DNA is damaged or other insults occur, apoptosis can occur. Many oncogenes in their normal state function to block cell death, such as Bcl-2.

A non-limiting list of oncogenes is abl (Tyrosine kinase activity); abl/bcr (New protein created by fusion); Af4/hrx (Fusion effects transcription factor product of hrx); akt-2 (Encodes a protein-serine/threonine kinase Ovarian cancer 1); alk (Encodes a receptor tyrosine kinase); ALK/NPM (New protein created by fusion); aml1 (Encodes a transcription factor); aml1/mtg8 (New protein created by fusion); axl (Encodes a receptor tyrosine kinase); bcl-2, 3, 6 (Block apoptosis (programmed cell death); bcr/abl (New protein created by fusion); c-myc (Cell proliferation and DNA synthesis); dbl (Guanine nucleotide exchange factor); dek/can (New protein created by fusion); E2A/pbx1 (New protein created by fusion); egfr (Tyrosine kinase); enl/hrx (New protein created by fusion); erg/c16 (New protein created by fusion); erbB (Tyrosine kinase); erbB-2 (originally neu) (Tyrosine kinase Breast); ets-1 (Transcription factor for some promoters); ews/fli-1 (New protein created by fusion); fms (Tyrosine kinase); fos (Transcription factor for API); fps (Tyrosine kinase); gip (Membrane associated G protein); gli (Transcription factor); gsp (Membrane associated G protein); HER2/neu (New protein created by gene fusion); hox11 (Over-expression of DNA binding protein); hrx/enl (New protein created by fusion); hrx/af4 (New protein created by fusion); hst (Encodes fibroblast growth factor); IL-3 (Over expression of protein); int-2 (Encodes a fibroblast growth factor); jun (Transcription factor); kit (Tyrosine kinase); KS3 (Growth factor); K-sam (Encodes growth factor receptors); Lbc (Guanine nucleotide exchange factor); Ick (Relocation of tyrosine kinase to the T-cell receptor gene); lmo-1, (2 Relocation of transcription factor near the T-cell receptor gene); L-myc (Cell proliferation and DNA synthesis); lyl-1 (Over-expression of DNA binding protein); lyt-10 (Relocation of transcription factor near the IgH gene); lt-10/C alpha1 (New protein created by fusion); mas (Angiotensin receptor); mdm-2 (Encodes a p53 inhibitor) Sarcomas 1; MLH1 (Mismatch repair in DNA); mll (New protein created by gene fusion); MLM (Encodes p16 a negative growth regulator that arrests the cell cycle); mos (Serine/threonine kinase); MSH2 (Mismatch repair in DNA); mtg8/aml1 (New protein created by fusion); myb (Encodes a transcription factor with DNA binding domain); MYH11/CBFB (New protein created by fusion); neu (now erb-2) (Tyrosine kinase); N-myc (Cell proliferation and DNA synthesis); NPM/ALK (New protein created by fusion); nrg/rel (New protein created by fusion); ost (Guanine nucleotide axchange factor); pax-5 (Relocation of transcription factor to the IgH gene); pbx1/E2A (New protein created by fusion); pim-1 (Serine/threonine kinase); PML/RAR (New protein created by fusion); PMS1, 2 (Mismatch repair in DNA); PRAD-1 (Encodes cyclin D1 that is important in G1 of the cell cycle); raf (Serine/threonine kinase); RAR/PML (New protein created by fusion); rasH (Involved in signal transduction of the cell); rasK (Involved in signal transduction of the cell); rasN (Involved in signal transduction of the cell); rel/nrg (New protein created by fusion); ret (DNA rearrangements that encode a receptor tyrosine kinase); rhom-1, 2 (Over-expression of DNA binding protein); ros (Tyrosine kinase); ski (Transcription factor); sis (Growth factor); set/can (New protein created by gene fusion); Src (Tyrosine kinase); tal-1, 2 (Over-expression of transcription factor); tan-1 (Over-expression of protein); Tiam-1 (Guanine nucleotide exchange factor); TSC2 (GTPase activator); trk (Recombinant fusion protein).

An example of a transforming gene is the Ras gene, an example of which is shown in SEQ ID NO:2. The ras family of oncogenes is comprises 3 main members:—K-ras, H-ras and N-ras. All of three of the oncogenes are involved in a variety of cancers. The K-ras oncogene is found on chromosome 12p12, encoding a 21-kD protein (p21ras). P21 is involved in the G-protein signal transduction pathway. Mutations of the K-ras oncogene produce constitutive activation of the G-protein transduction pathway which results in aberrant proliferation and differentiation.

Activating K-ras mutations are present in greater than 50% of colorectal adenomas and carcinomas, and the vast majority occur at codon 12 of the oncogene. K-ras mutations are one of the most common genetic abnormalities in pancreatic and bile duct carcinomas (greater than 75%). K-ras mutations are also frequent in adenocarcinomas of the lung.

Likewise, the disclosed transforming genes could be paired with other genes or sets of transforming genes that have desirable properties in the particular experiment. Different transformation strategies will be useful in different instances. For example, a cell transformed with an activated/dominant negative pair allows for multiple cycles of reversion. These cells then have the advantages of both primary cells and a cell line. Cells can be expanded, arrested, manipulated, then expanded again. Cells that are reverted using Cre/lox become analogs of primary cells, with only the 34 bp lox site remaining in the genome. These cells could be useful in a cell therapy setting.

b) Expression Systems

The nucleic acids that are delivered to cells typically contain expression controlling systems and often these expression controlling systems are tissues specific. The cells contain an expression controlling system which is tissue specific and possibly another which is not necessarily tissue specific. An expression controlling system is a system which causes expression of a target nucleic acid. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements. Sequences for affecting transcription can be referred to as transcription control elements.

(1) Tissue Specific and Cell Specific Promoters

Differentiation is the process whereby a cell is directed to express a particular set of transcription factors that transcribe the family of genes characteristic of that cell type. These transcription factors then act combinatorially at the promoters of the characteristic genes to bring about expression of the cognate mRNA and protein. In this way, a limited number of transcription factor genes can specifically regulate a much larger set of target genes (Alberts, B, Bray, D, Lewis, J, Raff, M, Roberts, K, Watson, J D. (1994) MOLECULAR BIOLOGY OF THE CELL, 3rd Ed., Garland Publishing, New York, N.Y., 1294p).

Tissue specific promoters function most effectively only in a particular biological context (Kelly, J H, Darlington, G J. (1985) Ann. Rev. Gen. 19, 273-296). For example, albumin is the major protein product of the adult hepatocyte and is expressed significantly only in that cell type. This is accomplished through expression of the human albumin gene, which has a promoter and enhancer that drive expression of the albumin gene only in the hepatocyte. Numerous experiments in transgenic mice have demonstrated that heterologous genes under the control of the albumin promoter/enhancer are expressed almost exclusively in the hepatocyte (Pinkert, C A, et al., (1987) Genes Dev. 3, 268-76). Since cell types are defined by the expression of particular genes and proteins, every specific type has a specific gene that is expressed exclusively, or nearly exclusively, in that cell type. Rhodopsin is expressed only in the cells of the retina, cardiac myosin is expressed only in cardiomyocytes, insulin is expressed only in the beta cells of the pancreas. Each of these genes is driven by a promoter which functions only in that cell type.

(a) Cell Specific Genes Have Cell Specific Promoters

In Table 3, there is an exemplary list of genes, which are expressed in whole or in part in the specific type of tissue indicated. It is understood that each of these genes has a 5′ upstream regions which contain regulatory elements which allow there specific expression patterns. Disclosed are nucleic acids comprising 100, 350, 500, 750, 1000, 1500, 2000, 2500, 3000, 4000, or 5000 bases of the 5′ upstream region of each of these genes, for example, linked operatively to a transformation gene disclosed herein. Also disclosed are methods of making and using the 5′ upstream regions of these genes including methods of identifying and isolating specific elements contained within these regions having the particular properties disclosed herein. Methods are well known, which allow for the identification of regulatory elements.

Table 3 attached to this application.

(b) Specific Promoters

There are a number of cell specific promoters that can be used in the disclosed methods and compositions. Promoters can also be identified by identifying regulatory regions associated with transcripts of genes that are cell type specific or occur in a subset of cell types.

For example for adipocyte regulatory sequences including promoters and enhancers, such as the sequences from the human adiponectin gene sequences from −908 to +14 can be used to identify adipocytes (SEQ ID NO:9) (Iwaki, M., et al. Diabetes 52, 1655-1663, 2003, Genbank nos. Q15848 and NM_{—}004797, all of which are herein incorporated at least for material related to the adiponectin gene and regulatory sequences including the sequences and methods of obtaining the same).

Another example are the hepatocyte cell regulatory sequences including promoters and enhancers, such as Human hepatitis B virus sequences from 1610 to 1810 (SEQ ID NO:22), Human alpha-1-antitrypsin promoter sequences from −137 to −37 (SEQ ID NO:10), and Human albumin gene sequences from −434 to +12 (SEQ ID NO:11). (Gabriela Kramer, M., et al. Molecular Therapy 7, 375-385 (2003) which is incorporated herein at least for material related to the hepatocyte regulatory sequences including the sequences and methods of obtaining the same).

Also disclosed heart cell regulatory sequences including promoters and enhancers. For example, Human myosin light chain gene VLC1 sequences from −357-+40 (SEQ ID NO:12) act in a heart cell specific way. (Kurabayashi, et al., J. Biol. Chem. 265, 19271-19278, (1990) which is incorporated herein at least for material related to the heart regulatory sequences including the sequences and methods of obtaining the same).

Also disclosed are retina regulatory sequences such as promoters and enhancers, such as the regulatory sequences for the human rhodopsin gene, such as sequences from −176 to +70 plus 246 bp from −2140 to −1894. (SEQ ID NO:13) (Nie et al., J. Biol. Chem. 271, 2667-2675, (1996) which is incorporated herein at least for material related to the retina regulatory sequences including the sequences and methods of obtaining the same).

Also disclosed are B cell regulatory sequences such as promoter and enhancer sequences, such as the sequences regulating the human immunoglobulin heavy chain promoter and enhancer elements (Maxwell, IH, et al. Cancer Res. 51, 4299-4304, (1991) which is incorporated herein at least for material related to the B cell regulatory sequences including the sequences and methods of obtaining the same).

Also disclosed are endothelial cell regulatory sequences such as promoter and enhancer sequences, such as the regulatory sequences for the human E selectin gene, such as sequences from −547 to +33. (SEQ ID NO:14) (Maxwell, IH, et al. Angiogenesis 6, 31-38, (2003) which is incorporated herein at least for material related to the endothelial regulatory sequences including the sequences and methods of obtaining the same).

Also disclosed are T cell regulatory sequences, such as promoter and enhancer sequences, such as the sequences for the human preT cell receptor, such as sequence from −279 to +5 (SEQ ID NO:15) and can include the upstream enhancer elements (Reizis and Leder, Exp. Med., 194, 979-990, (2001) which is incorporated herein at least for material related to the T cell regulatory sequences including the sequences and methods of obtaining the same).

Also disclosed are macrophage regulatory sequences, such as promoter and enhancer sequences, such as sequences for the human HCgp-39 gene from −308-+2. (SEQ ID NO:16) (Rehli, M., et al. J. Biol. Chem. 278, 44058-44067, (2003) which is incorporated herein at least for material related to the macrophage regulatory sequences including the sequences and methods of obtaining the same).

Also disclosed are regulatory sequences for kidney cells, such as promoter and enhancer sequences, such as regulatory sequences for the human uromodulin gene such as promoter sequences from −3.7 kb of the gene. (SEQ ID NO:17) (Zbikowska, H M, et al. Biochem. J. 365, 7-11, (2002) which is incorporated herein at least for material related to the kidney cell regulatory sequences including the sequences and methods of obtaining the same).

Also disclosed are brain regulatory sequences, such as promoter and enhancer sequences, such as regulatory sequences for the Human glutamate receptor 2 gene (GluR2), such as sequences from −302 to +320 of the gene. (SEQ ID NO:18) (Myers, S J, et al. J. Neuroscience 18, 6723-6739, (1998) which is incorporated herein at least for material related to the brain regulatory sequences including the sequences and methods of obtaining the same).

Also disclosed are regulatory sequences for lung cells, such as promoters and enhancers, such as regulatory sequences for the human surfactant protein A2 (SP-A2), such as sequences from −296 to +13 of the gene. (SEQ ID NO:19) (Young, P P, C R Mendelson Am. J. Physiol. 271, L287-289, (1996) which is incorporated herein at least for material related to the lung cell regulatory sequences including the sequences and methods of obtaining the same).

Also disclosed are pancreas cell regulatory sequences, such as promoters and enhancers, such as the regulatory sequences for the human insulin gene, such as sequences from −279 of the gene. (SEQ ID NO:20) (Boam, D S, et al. J. Biol. Chem. 265, 8285-8296, (1990) which is incorporated herein at least for material related to the pancreas cell regulatory sequences including the sequences and methods of obtaining the same).

Also disclosed are skeletal muscle regulatory sequences, such as promoters and enhancers, such as regulatory sequences for the human fast skeletal muscle troponin C gene, such as sequences from −978 to +1 of the gene. (SEQ ID NO:21) (Gahlmann, R, L. Kedes J. Biol. Chem. 265, 12520-12528, (1990) which is incorporated herein at least for material related to the skeletal muscle regulatory sequences including the sequences and methods of obtaining the same).

Also disclosed are nucleic acids that contain a suicide gene, such as those disclosed herein, wherein the gene will kill the cell if it is turned on, for example, and these genes can be regulated in their expression. For example, the suicide gene can also be included within a cre-lox recombination site, so that after transformation has taken place as disclosed herein, and after the cell or set of cells has been selectively grown in transformation media, and the transformation gene will be excised by a recombinase, such as Cre, the suicide gene will also be excised. Then in non-transformation media containing the appropriate conditions for turning the suicide gene on will allow only those cells in which a recombination event has occurred to survive. There are many variations and combinations of this result with the markers and compositions and methods disclosed herein in combination.

(2) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

The promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

c) Reversible Transformation

Transformation is the process whereby a cell loses its ability to respond to the signals that would normally regulate its growth. This can take the form of a loss of function mutation, such as results in loss of a repressor of cell growth such as PTEN, or a gain of function mutation whereby a gene becomes permanently activated such as occurs in many RAS mutations. Many laboratories have shown that insertion of one or more of these transforming genes into a normal cell can free it of the usual constraints on its growth and allow it to proliferate (Downward, J. (2002) Nat. Rev. Cancer 3, 11-22). Reversible transformation activates the transforming gene in one instance, then shuts it off in another. There are several means to accomplish this reversal.

The combination of tissue specific promoter/enhancers with reversible transforming genes allows the identification and culture of any specific cell type from differentiating stem cells. This system provides the dual advantages referred to above in that it is general and can be used to generate large quantities of specific cell types. In fact, it allows the establishment of permanent, clonal or semi-purified, differentiated cell lines that can be characterized and frozen. Upon reversal, the entire population reverts, providing an unlimited source of characterized, differentiated, normal cells.

(1) Dominant Negative Reversal

Many transforming genes, such as RAS, have another known mutant that is a dominant negative. For example, dominant negative RAS sequesters RAF, another protein necessary for propagation of the RAS signal, such that RAS signaling is turned off (Fiordalisi, (2002) J Biol. Chem. 29, 10813-23). Using such activated/dominant negative pairs of genes provides a reversible system. Such pairs are known for RAS, SRC and p53, for example (Barone and Courtneidge, (1995) Nature. 1995 Nov. 30; 378(6556):509-12; Willis A, et al., Oncogene. 2004 Mar. 25; 23(13):2330-8).

(2) Temperature Sensitive Mutant Reversal

Another mechanism to effect reversible transformation is with temperature sensitive mutants (Jat, P S, et al., (1991) Proc. Natl. Acad. Sci. 88, 5096-5100). Temperature sensitive (ts) proteins are stable at the permissive temperature but unstable at the restrictive temperature. T antigen (TAg), the well known transforming gene of the SV40 virus, has several ts mutants. When tsTAg is inserted into a normal cell, the cell is transformed and proliferates at 32° C. but arrests and reverts to normal at 39° C. Several such temperature sensitive mutants are known for SV40 T antigen and adenovirus E1A, for example (Fahnestock, M L, Lewis, J B. (1989) J. Virol. 63, 2348-2351).

(3) Recombinase Reversal

A third mechanism for reversible transformation is to, in fact, reversibly insert the transforming gene. Cre/lox and flp/frt are two such mechanisms for reversible insertion (Sauer. B. (2002) Endocrine 19, 221-228; Schaft, J, et al., (2001) Genesis 31, 6-10). If a gene is transfected into a target cell capped on each end by lox recombination sites, treatment of the cell with CRE recombinase will excise the inserted sequence, leaving only a single lox sequence. Likewise, if a gene is transfected into a target call capped on each end by frt treatment with flp will excise the inserted sequence, leaving only the flp sequence.

Disclosed are compositions including cells that comprise one or more of the sequences disclosed herein, such as a cell comprising a transformation sequence driven by the insulin promoter, such as a purified or semi-purified or clonal population of cells comprising the recombinase sequence, such as a lox or flp sequence, remaining after a recombination event, for example, wherein the cell was a cell previously containing one or more of the nucleic acids disclosed herein.

5. Cells Produced by the Disclosed Methods and Compositions

The adult human body produces many different cell types. Information on human cell types can be found at http://encyclopedia.thefreedictionary.com/List%20of%20distinct%20cell%20types%20in%20the%20adult%20human %20body). These different cell types include, but are not limited to, Keratinizing Epithelial Cells, Wet Stratified Barrier Epithelial Cells, Exocrine Secretory Epithelial Cells, Hormone Secreting Cells, Epithelial Absorptive Cells (Gut, Exocrine Glands and Urogenital Tract), Metabolism and Storage cells, Barrier Function Cells (Lung, Gut, Exocrine Glands and Urogenital Tract), Epithelial Cells Lining Closed Internal Body Cavities, Ciliated Cells with Propulsive Function, Extracellular Matrix Secretion Cells, Contractile Cells, Blood and Immune System Cells, Sensory Transducer Cells, Autonomic Neuron Cells, Sense Organ and Peripheral Neuron Supporting Cells, Central Nervous System Neurons and Glial Cells, Lens Cells, Pigment Cells, Germ Cells, and Nurse Cells. Also included are any stem cells and progenitor cells of the cells disclosed herein, as well as the cells they lead to. Cells and cell types of interest produced in the disclosed method can be identified by reference to one or more characteristics of such cells. Many such characteristics are known, some of which are described herein.

Cell Types

The usual estimate based on histological studies is that there are ˜200 distinct kinds of cells in an adult human body that show alternate structures and functions (David S. Goodsell, The Machinery of Life, Springer-Verlag, New York, 1993; Bruce Alberts, Dennis Bray, Julian Lewis, Martin Raff, Keith Roberts, James D. Watson, The Molecular Biology of the Cell, Second Edition, Garland Publishing, Inc., New York, 1989; Arthur J. Vander, James H. Sherman, Dorothy S. Luciano, Human Physiology: The Mechanisms of Body Function, Fifth Edition, McGraw-Hill Publishing Company, New York, 1990). These represent discrete categories of cell types of markedly different character, not arbitrary subdivisions along a morphological continuum. Traditional classification is based on microscopic shape and structure, and on crude chemical nature (e.g., affinity for various stains), but newer immunological techniques have revealed, for instance, that there are more than 10 distinct types of lymphocytes. Pharmacological and physiological tests have revealed many different varieties of smooth muscle cells—for example, uterine wall smooth muscle cells are highly sensitive to estrogen and (in late pregnancy) oxytocin, while gut wall smooth muscle cells are not.

Cells of the human body include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting melanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), Thymus epithelial cell

This list of cells is organized by cellular function and omits subdivisions of smooth muscle cells, neuron classes in the CNS, various related connective tissue and fibroblast types, and intermediate stages of maturing cells such as keratinocytes (only the stem cell and differentiated cell types are given). Otherwise, the catalog is represents an exhaustive listing of the ˜219 cell varieties found in the adult human phenotype (complexity theory and phylogenetic comparisons suggest that the maximum number of cell types N_{cell}˜N_{gene}^{1/2}=370 cell types for humans with N_{gene}˜10⁵ genes) (S. A. Kauffman, “Metabolic Stability and Epigenesis in Randomly Constructed Genetic Nets,” J. Theoret. Biol. 22(1969):437-467; Stuart A. Kauffman, The Origins of Order: Self-Organization and Selection in Evolution, Oxford University Press, New York, 1993).

Cell Markers

There are several identifying characteristics by which a cell can be distinguished and identified. Different cell types are unique in size, shape, density and have distinct expression profiles of intracellular, cell-surface, and secreted proteins. Described are markers that can be used to identify and define a differentiated cell provided herein. These markers can be evaluated using methods known in the art using antibodies, probes, primers, or other such targeting means known in the art. Examples of markers that are routinely used to identify and distinguish differentiated cell types are provided in Table 4.

TABLE 4
Markers Commonly Used to Identify and Characterize Differentiated Cell Types

| | | |
|---|---|---|
| Marker Name | Cell Type | Significance |

Blood Vessel

| | | |
|---|---|---|
| Fetal liver kinase-1 | Endothelial | Cell-surface receptor protein that identifies |
| (Flk1) | | endothelial cell progenitor; marker of cell-cell |
| | | contacts |
| Smooth muscle cell- | Smooth muscle | Identifies smooth muscle cells in the wall of blood |
| specific myosin heavy | | vessels |
| chain | | |
| Vascular endothelial | cadherin | Smooth muscle Identifies smooth muscle cells in |
| cell | | the wall of blood vessels |

Bone

| | | |
|---|---|---|
| Bone-specific alkaline | Osteoblast | Enzyme expressed in osteoblast; activity indicates |
| phosphatase (BAP) | | bone formation |
| Hydroxyapatite | Osteoblast | Minerlized bone matrix that provides structural |
| | | integrity; marker of bone formation |
| Osteocalcin (OC) | Osteoblast | Mineral-binding protein uniquely synthesized by |
| | | osteoblast; marker of bone formation |

Bone Marrow and Blood

| | | |
|---|---|---|
| Bone morphogenetic | Mesenchymal stem | Important for the differentiation of committed |
| protein receptor | and progenitor cells | mesenchymal cell types from mesenchymal stem |
| (BMPR) | | and progenitor cells; BMPR identifies early |
| | | mesenchymal lineages (stem and progenitor cells) |
| CD4 and CD8 | White blood cell | Cell-surface protein markers specific for mature T |
| | (WBC) | lymphocyte (WBC subtype) |
| CD34 | Hematopoietic stem | Cell-surface protein on bone marrow cell, |
| | cell (HSC), satellite, | indicative of a HSC and endothelial progenitor; |
| | endothelial | CD34 also identifies muscle satellite, a muscle |
| | progenitor | stem cell |
| CD34⁺Sca1⁺Lin⁻ | Mesencyhmal stem | Identifies MSCs, which can differentiate into |
| profile | cell (MSC) | adipocyte, osteocyte, chondrocyte, and myocyte |
| CD38 | Absent on HSC | Cell-surface molecule that identifies WBC lineages. |
| | Present on WBC | Selection of CD34⁺/CD38⁻ cells allows for |
| | lineages | purification of HSC populations |
| CD44 | Mesenchymal | A type of cell-adhesion molecule used to identify |
| | | specific types of mesenchymal cells |
| c-Kit | HSC, MSC | Cell-surface receptor on BM cell types that |
| | | identifies HSC and MSC; binding by fetal calf |
| | | serum (FCS) enhances proliferation of ES cells, |
| | | HSCs, MSCs, and hematopoietic progenitor cells |
| Colony-forming unit | HSC, MSC | CFU assay detects the ability of a single stem cell |
| (CFU) | progenitor | or progenitor cell to give rise to one or more cell |
| | | lineages, such as red blood cell (RBC) and/or white |
| | | blood cell (WBC) lineages |
| Fibroblast colony- | Bone marrow | An individual bone marrow cell that has given rise |
| forming unit (CFU-F) | fibroblast | to a colony of multipotent fibroblastic cells; such |
| | | identified cells are precursors of differentiated |
| | | mesenchymal lineages |
| Hoechst dye | Absent on HSC | Fluorescent dye that binds DNA; HSC extrudes the |
| | | dye and stains lightly compared with other cell |
| | | types |
| Leukocyte common | WBC | Cell-surface protein on WBC progenitor |
| antigen (CD45) | | |
| Lineage surface antigen | HSC, MSC | Thirteen to 14 different cell-surface proteins that |
| (Lin) | Differentiated RBC | are markers of mature blood cell lineages; detection |
| | and WBC lineages | of Lin-negative cells assists in the purification of |
| | | HSC and hematopoietic progenitor populations |
| Mac-1 | WBC | Cell-surface protein specific for mature granulocyte |
| | | and macrophage (WBC subtypes) |
| Muc-18 (CD146) | Bone marrow | Cell-surface protein (immunoglobulin superfamily) |
| | fibroblasts, | found on bone marrow fibroblasts, which may be |
| | endothelial | important in hematopoiesis; a subpopulation of |
| | | Muc-18+ cells are mesenchymal precursors |
| Stem cell antigen (Sca- | HSC, MSC | Cell-surface protein on bone marrow (BM) cell, |
| 1) | | indicative of HSC and MSC Bone Marrow and |
| | | Blood cont. |
| Stro-1 antigen | Stromal | Cell-surface glycoprotein on subsets of bone |
| | (mesenchymal) | marrow stromal (mesenchymal) cells; selection of |
| | precursor cells, | Stro-1+ cells assists in isolating mesenchymal |
| | hematopoietic cells | precursor cells, which are multipotent cells that |
| | | give rise to adipocytes, osteocytes, smooth |
| | | myocytes, fibroblasts, chondrocytes, and blood |
| | | cells |
| Thy-1 | HSC, MSC | Cell-surface protein; negative or low detection is |
| | | suggestive of HSC |

Cartilage

| | | |
|---|---|---|
| Collagen types II and | Chondrocyte | Structural proteins produced specifically by |
| IV | | chondrocyte |
| Keratin | Keratinocyte | Principal protein of skin; identifies differentiated |
| | | keratinocyte |
| Sulfated proteoglycan | Chondrocyte | Molecule found in connective tissues; synthesized |
| | | by chondrocyte |

Fat

| | | |
|---|---|---|
| Adipocyte lipid-binding | Adipocyte | Lipid-binding protein located specifically in |
| protein (ALBP) | | adipocyte |
| Fatty acid transporter | Adipocyte | Transport molecule located specifically in |
| (FAT) | | adipocyte |
| Adipocyte lipid-binding | Adipocyte | Lipid-binding protein located specifically in |
| protein (ALBP) | | adipocyte |

Liver

| | | |
|---|---|---|
| Albumin | Hepatocyte | Principal protein produced by the liver; indicates |
| | | functioning of maturing and fully differentiated |
| | | hepatocytes |
| B-1 integrin | Hepatocyte | Cell-adhesion molecule important in cell-cell |
| | | interactions; marker expressed during development |
| | | of liver |

Nervous System

| | | |
|---|---|---|
| CD133 | Neural stem cell, | Cell-surface protein that identifies neural stem |
| | HSC | cells, which give rise to neurons and glial cells |
| Glial fibrillary acidic | Astrocyte | Protein specifically produced by astrocyte |
| protein GFAP | | |
| Microtubule-associated | Neuron | Dendrite-specific MAP; protein found specifically |
| protein-2 (MAP-2) | | in dendritic branching of neuron |
| Myelin basic protein | Oligodendrocyte | Protein produced by mature oligodendrocytes; |
| (MPB) | | located in the myelin sheath surrounding neuronal |
| | | structures |
| Nestin | Neural progenitor | Intermediate filament structural protein expressed |
| | | in primitive neural tissue |
| Neural tubulin | Neuron | Important structural protein for neuron; identifies |
| | | differentiated neuron |
| Neurofilament (NF) | Neuron | Important structural protein for neuron; identifies |
| | | differentiated neuron |
| Noggin | Neuron | A neuron-specific gene expressed during the |
| | | development of neurons |
| O4 | Oligodendrocyte | Cell-surface marker on immature, developing |
| | | oligodendrocyte |
| O1 | Oligodendrocyte | Cell-surface marker that characterizes mature |
| | | oligodendrocyte |
| Synaptophysin | Neuron | Neuronal protein located in synapses; indicates |
| | | connections between neurons |
| Tau | Neuron | Type of MAP; helps maintain structure of the axon |

Pancreas

| | | |
|---|---|---|
| Cytokeratin 19 (CK19) | Pancreatic | CK19 identifies specific pancreatic epithehial cells |
| | epithelium | that are progenitors for islet cells and ductal cells |
| Glucagon | Pancreatic islet | Expressed by alpha-islet cell of pancreas |
| Insulin | Pancreatic islet | Expressed by beta-islet cell of pancreas |
| Pancreas Insulin- | Pancreatic islet | Transcription factor expressed by beta-islet cell of |
| promoting factor-1 | | pancreas |
| (PDX-1) | | |
| Nestin | Pancreatic | Structural filament protein indicative of progenitor |
| | progenitor | cell lines including pancreatic |
| Pancreatic polypeptide | Pancreatic islet | Expressed by gamma-islet cell of pancreas |
| Somatostatin | Pancreatic islet | Expressed by delta-islet cell of pancreas Pluripotent |
| | | Stem Cells |
| Alpha-fetoprotein | Endoderm | Protein expressed during development of primitive |
| (AFP) | | endoderm; reflects endodermal differentiation |

Pluripotent Stem Cells

| | | |
|---|---|---|
| Bone morphogenetic | Mesoderm | Growth and differentiation factor expressed during |
| protein-4 | | early mesoderm formation and differentiation |
| Brachyury | Mesoderm | Transcription factor important in the earliest phases |
| | | of mesoderm formation and differentiation; used as |
| | | the earliest indicator of mesoderm formation |
| GATA-4 gene | Endoderm | Expression increases as ES differentiates into |
| | | endoderm |
| Hepatocyte nuclear | Endoderm | Transcription factor expressed early in endoderm |
| factor-4 (HNF-4) | | formation |
| Nestin | Ectoderm, neural | Intermediate filaments within cells; characteristic |
| | and pancreatic | of primitive neuroectoderm formation |
| | progenitor | |
| Neuronal cell-adhesion | Ectoderm | Cell-surface molecule that promotes cell-cell |
| molecule (N-CAM) | | interaction; indicates primitive neuroectoderm |
| | | formation |
| Pax6 | Ectoderm | Transcription factor expressed as ES cell |
| | | differentiates into neuroepithelium |
| Vimentin | Ectoderm, neural | Intermediate filaments within cells; characteristic |
| | and pancreatic | of primitive neuroectoderm formation |
| | progenitor | |

Skeletal Muscle/Cardiac/Smooth Muscle

| | | |
|---|---|---|
| MyoD and Pax7 | Myoblast, myocyte | Transcription factors that direct differentiation of |
| | | myoblasts into mature myocytes |
| Myogenin and MR4 | Skeletal myocyte | Secondary transcription factors required for |
| | | differentiation of myoblasts from muscle stem cells |
| Myosin heavy chain | Cardiomyocyte | A component of structural and contractile protein |
| | | found in cardiomyocyte |
| Myosin light chain | Skeletal myocyte | A component of structural and contractile protein |
| | | found in skeletal myocyte |

Cell surface antigens are routinely used as markers to identify and distinguish cells. Antigenic specificities exist for species (xenotype), organ, tissue, or cell type for almost all cells—possibly involving as many as ˜10⁴ distinct antigens. Examples of cell surface antigens that can be used to distinguish cell types are provided in Table 5.

TABLE 5
Human Cell Surface Antigens

| | |
|---|---|
| B cell | CD1C, CHST10, HLA-A, HLA-DRA, NT5E |
| Activated B Cells | CD28, CD38, CD69, CD80, CD83, CD86, DPP4, FCER2, IL2RA, TNFRSF8, TNFSF7 |
| Mature B Cells | CD19, CD22, CD24, CD37, CD40, CD72, CD74, CD79A, CD79B, CR2, IL1R2, ITGA2, |
| | ITGA3, MS4A1, ST6GAL1 |
| T cell | CD160, CD28, CD37, CD3D, CD3G, CD3Z, CD5, CD6, CD7, FAS, KLRB1, KLRD1, |
| | NT5E, ST6GAL1 |
| Cytotoxic T Cells | CD8A, CD8B1 |
| Helper T Cells | CD4 |
| Activated T Cells | ALCAM, CD2, CD38, CD40LG, CD69, CD83, CD96, CTLA4, DPP4, HLA-DRA, IL12RB1, |
| | IL2RA, ITGA1, TNFRSF4, TNFRSF8, TNFSF7 |
| Natural Killer (NK) cell | CD2, CD244, CD3Z, CD7, CD96, CHST10, FCGR3B, IL12RB1, KLRB1, KLRC1, KLRD1, |
| | LAG3, NCAM1 |
| Monocyte/macrophage | ADAM8, C5R1, CD14, CD163, CD33, CD40, CD63, CD68, CD74, CD86, CHIT1, |
| | CHST10, CSF1R, DPP4, FABP4, FCGR1A, HLA-DRA, ICAM2, IL1R2, ITGA1, ITGA2, |
| | S100A8, TNFRSF8, TNFSF7 |
| Activated Macrophages | CD69, ENG, FCER2, IL2RA |
| Endothelial cell | ACE, CD14, CD34, CD31, CDH5, ENG, ICAM2, MCAM, NOS3, PECAM1, PROCR, |
| | SELE, SELP, TEK, THBD, VCAM1, VWF. |
| Smooth muscle cell | ACTA2, MYH10, MYH11, MYH9, MYOCD. |
| Dendritic cell | CD1A, CD209, CD40, CD83, CD86, CR2, FCER2, FSCN1 |
| Mast cell | C5R1, CMA1, FCER1A, FCER2, TPSAB1 |
| Fibroblast (stromal) | ALCAM, CD34, COL1A1, COL1A2, COL3A1, PH-4 |
| Epithelial cell | CD1D, K6IRS2, KRT10, KRT13, KRT17, KRT18, KRT19, KRT4, KRT5, KRT8, MUC1, |
| | TACSTD1. |
| Adipocyte | ADIPOQ, FABP4, RETN. |

In the case of red blood cells, antigens in the Rh, Kell, Duffy, and Kidd blood group systems are found exclusively on the plasma membranes of erythrocytes and have not been detected on platelets, lymphocytes, granulocytes, in plasma, or in other body secretions such as saliva, milk, or amniotic fluid (P. L. Mollison, C. P. Engelfriet, M. Contreras, Blood Transfusions in Clinical Medicine, Ninth Edition, Blackwell Scientific, Oxford, 1993). Thus detection of any member of this four-antigen set establishes a unique marker for red cell identification. MNSs and Lutheran antigens are also limited to erythrocytes with two exceptions: GPA glycoprotein (MN activity) also found on renal capillary endothelium (P. Hawkins, S. E. Anderson, J. L. McKenzie, K. McLoughlin, M. E. J. Beard, D. N. J. Hart, “Localization of MN Blood Group Antigens in Kidney,” Transplant. Proc. 17(1985):1697-1700), and Lu^{b}-like glycoprotein which appears on kidney endothelial cells and liver hepatocytes (D. J. Anstee, G. Mallinson, J. E. Yendle, et al., “Evidence for the occurrence of Lu^{b}-active glycoproteins in human erythrocytes, kidney, and liver,” International Congress ISBT-BBTS Book of Abstracts, 1988, p. 263). In contrast, ABH antigens are found on many non-RBC tissue cells such as kidney and salivary glands (Ivan M. Roitt, Jonathan Brostoff, David K. Male, Immunology, Gower Medical Publishing, New York, 1989). In young embryos ABH can be found on all endothelial and epithelial cells except those of the central nervous system (Aron E. Szulman, “The ABH antigens in human tissues and secretions during embryonal development,” J. Histochem. Cytochem. 13(1965):752-754). ABH, Lewis, I and P blood group antigens are found on platelets and lymphocytes, at least in part due to adsorption from the plasma onto the cell membrane. Granulocytes have I antigen but no ABH (P. L. Mollison, C. P. Engelfriet, M. Contreras, Blood Transfusions in Clinical Medicine, Ninth Edition, Blackwell Scientific, Oxford, 1993).

Platelets also express platelet-specific alloantigens on their plasma membranes, in addition to the HLA antigens they already share with body tissue cells. Currently there are five recognized human platelet alloantigen (HPA) systems that have been defined at the molecular level. The phenotype frequencies given are for the Caucasian population; frequencies in African and Asian populations may vary substantially. For instance, HPA-1b is expressed on the platelets of 28% of Caucasians but only 4% of the Japanese population (Thomas J. Kunicki, Peter J. Newman, “The molecular immunology of human platelet proteins,” Blood 80(1992):1386-1404).

Lymphocytes with a particular functional activity can be distinguished by various differentiation markers displayed on their cell surfaces. For example, all mature T cells express a set of polypeptide chains called the CD3 complex. Helper T cells also express the CD4 glycoprotein, whereas cytotoxic and suppressor T cells express a marker called CD8 (Wayne M. Becker, David W. Deamer, The World of the Cell, Second Edition, Benjamin/Cummings Publishing Company, Redwood City Calif., 1991). Thus the phenotype CD3⁺CD4⁺CD8⁻ positively identifies a helper T cell, whereas the detection of CD3⁺CD4⁻CD8⁺ uniquely identifies a cytotoxic or suppressor T cell. All B lymphocytes express immunoglobulins (their antigen receptors, or Ig) on their surface and can be distinguished from T cells on that basis, e.g., as Ig⁺ MHC Class II⁺.

Lymphocyte surfaces also display distinct markers representing specific gene products that are expressed only at characteristic stages of cell differentiation. For example, Stage I Progenitor B cells display CD34⁺PhiL⁻CD19⁻; Stage II, CD34⁺PhiL⁺CD19⁻; Stage III, CD34⁺PhiL⁺CD19⁺; and finally CD34⁻PhiL⁺CD19⁺ at the Precursor B stage (Una Chen, “Chapter 33. Lymphocyte Engineering, Its Status of Art and Its Future,” in Robert P. Lanza, Robert Langer, William L. Chick, eds., Principles of Tissue Engineering, R.G. Landes Company, Georgetown Tex., 1997, pp. 527-561).

There are neutrophil-specific antigens and various receptor-specific immunoglobulin binding specificities for leukocytes. For instance, monocyte FcRI receptors display the measured binding specificity IgG1⁺⁺⁺IgG2⁻IgG3⁺⁺⁺IgG4⁺, monocyte FcRIII receptors have IgG1⁺⁺IgG2⁻IgG3⁺⁺IgG4⁻, and FcRII receptors on neutrophils and eosinophils show IgG1⁺⁺⁺IgG2⁺IgG3⁺⁺⁺IgG4⁺. Neutrophils also have β-glucan receptors on their surfaces (Vicki Glaser, “Carbohydrate-Based Drugs Move CLoser to Market,” Genetic Engineering News, 15 Apr. 1998, pp. 1, 12, 32, 34).

Tissue cells display specific sets of distinguishing markers on their surfaces as well. Thyroid microsomal-microvillous antigen is unique to the thyroid gland (Ivan M. Roitt, Jonathan Brostoff, David K. Male, Immunology, Gower Medical Publishing, New York, 1989). Glial fibrillary acidic protein (GFAP) is an immunocytochemical marker of astrocytes (Carlos Lois, Jose-Manuel Garcia-Verdugo, Arturo Alvarez-Buylla, “Chain Migration of Neuronal Precursors,” Science 271(16 Feb. 1996):978-981), and syntaxin 1A and 1B are phosphoproteins found only in the plasma membrane of neuronal cells (Nicole Calakos, Mark K. Bennett, Karen E. Peterson, Richard H. Scheller, “Protein-Protein Interactions Contributing to the Specificity of Intracellular Vesicular Trafficking,” Science 263(25 Feb. 1994):1146-1149). Alpha-fodrin is an organ-specific autoantigenic marker of salivary gland cells (Norio Haneji, Takanori Nakamura, Koji Takio, et al., “Identification of alpha-Fodrin as a Candidate Autoantigen in Primary Sjogren's Syndrome,” Science 276(25 Apr. 1997):604-607). Fertilin, a member of the ADAM family, is found on the plasma membrane of mammalian sperm cells (Tomas Martin, Ulrike Obst, Julius Rebek Jr., “Molecular Assembly and Encapsulation Directed by Hydrogen-Bonding Preferences and the Filling of Space,” Science 281(18 Sep. 1998):1842-1845). Hepatocytes display the phenotypic markers ALB⁺⁺⁺GGT⁻CK19⁻ along with connexin 32, transferrin, and major urinary protein (MUP), while biliary cells display the markers AFP⁻GGT⁺⁺⁺CK19⁺⁺⁺ plus BD.1 antigen, alkaline phosphatase, and DPP4 (Lola M. Reid, “Chapter 31. Stem Cell/Lineage Biology and Lineage-Dependent Extracellular Matrix Chemistry: Keys to Tissue Engineering of Quiescent Tissues such as Liver,” in Robert P. Lanza, Robert Langer, William L. Chick, eds., Principles of Tissue Engineering, R.G. Landes Company, Georgetown Tex., 1997, pp. 481-514). A family of 100-kilodalton plasma membrane guanosine triphosphatases implicated in clathrin-coated vesicle transport include dynamin I (expressed exclusively in neurons), dynamin II (found in all tissues), and dynamin III (restricted to the testes, brain, and lungs), each with at least four distinct isoforms; dynamin II also exhibits intracellular localization in the trans-Golgi network (Martin Schnorf, Ingo Potrykus, Gunther Neuhaus, “Microinjection Technique: Routine System for Characterization of Microcapillaries by Bubble Pressure Measurement,” Experimental Cell Research 210(1994):260-267). Table 6 lists numerous unique antigenic markers of hepatopoietic (e.g., hepatoblast) and hemopoietic (e.g., erythroid progenitor) cells.

TABLE 6
Unique antigenic markers of hepatopoietic and hemopoietic human cells.

| | |
|---|---|
| Hepatopoietic Cells | α-fetoprotein, albumin, stem cell factor, hepatic heparin sulfate-PGs |
| (e.g., Hepatoblasts) | (syndecan/perlecans), IGF I, IGF II, TGF-α, TGF-α receptor, α1 integrin, α5 integrin, |
| | connexin 26, and connexin 32 |
| Hematopoietic Cells | OX43 (MCA 276), OX44 (MCA 371, CD37), OX42 (MCA 275, CD118), c-Kit, stem cell |
| (e.g., Erythroid Progenitors) | factor receptor, hemopoietic heparin sulfate-PG (serglycin), GM-CSF, CSF, α4 integrin, |
| | and red blood cell antigen |

At least four major families of cell-specific cell adhesion molecules had been identified by 1998—the immunoglobulin (Ig) superfamily (including N-CAM and ICAM-1), the integrin superfamily, the cadherin family and the selectin family (see below).

Integrins are ˜200 kilodalton cell surface adhesion receptors expressed on a wide variety of cells, with most cells expressing several integrins. Most integrins, which mediate cellular connection to the extracellular matrix, are involved in attachments to the cytoskeletal substratum. Cell-type-specific examples include platelet-specific integrin (α_{IIb}β₃), leukocyte-specific β2 integrins, late-activation (α_{L}β₂) lymphocyte antigens, retinal ganglion axon integrin (α₆β₁) and keratinocyte integrin (α₅β₁) (Richard O. Hynes, “Integrins: Versatility, Modulation, and Signaling in Cell Adhesion,” Cell 69(3 Apr. 1992):11-25). At least 20 different heterodimer integrin receptors were known in 1998.

The cadherin molecular family of 723-748-residue transmembrane proteins provides yet another avenue of cell-cell adhesion that is cell-specific (Masatoshi Takeichi, “Cadherins: A molecular family important in selective cell-cell adhesion,” Ann. Rev. Biochem. 59(1990):237-252). Cadherins are linked to the cytoskeleton. The classical cadherins include E-(epithelial), N-(neural or A-CAM), and P-(placental) cadherin, but in 1998 at least 12 different members of the family were known (Elizabeth J. Luna, Anne L. Hitt, “Cytoskeleton-Plasma Membrane Interactions,” Science 258 (1992):955-964). They are concentrated (though not exclusively found) at cell-cell junctions on the cell surface and appear to be crucial for maintaining multicellular architecture. Cells adhere preferentially to other cells that express the identical cadherin type. Liver hepatocytes express only E-; mesenchymal lung cells, optic axons and neuroepithelial cells express only N-; epithelial lung cells express both E- and P-cadherins. Members of the cadherin family also are distributed in different spatiotemporal patterns in embryos, with the expression of cadherin types changing dynamically as the cells differentiate (Masatoshi Takeichi, “Cadherins: A molecular family important in selective cell-cell adhesion,” Ann. Rev. Biochem. 59(1990):237-252).

Carbohydrates are crucial in cell recognition. All cells have a thin sugar coating (the glycocalyx) consisting of glycoproteins and glycolipids, of which 3000 different motifs had been identified by 1998. The repertoire of carbohydrate cell surface structures changes characteristically as the cell develops, differentiates, or sickens. For example, a unique trisaccharide (SSEA-1 or L^{ex}) appears on the surfaces of cells of the developing embryo exactly at the 8- to 16-cell stage when the embryo compacts from a group of loose cells into a smooth ball.

Carbohydrate motifs are in theory more combinatorially diverse than nucleotide or protein-based structures. While nucleotides and amino acids can interconnect in only one way, the monosaccharide units in oligosaccharides and polysaccharides can attach at multiple points. Thus two amino acids can make only two distinct dipeptides, but two identical monosaccharides can bond to form 11 different disaccharides because each monosaccharide has 6 carbons, giving each unit 6 different attachment points for a total of 6+5=11 possible combinations. Four different nucleotides can make only 24 distinct tetranucleotides, but four different monosaccharides can make 35,560 unique tetrasaccharides, including many with branching structures (Nathan Sharon, Halina Lis, “Carbohydrates in Cell Recognition,” Scientific American 268(January 1993):82-89). A single hexasaccharide can make ˜10¹² distinct structures, vs. only 6.4×10⁷ structures for a hexapeptide; a 9-mer carbohydrate has a mole of isomers (Roger A. Laine. Glycobiology 4(1994):1-9).

The CD44 family of transmembrane glycoproteins are 80-95 kilodalton cell adhesion receptors that mediate ECM binding, cell migration and lymphocyte homing. CD44 antigen shows a wide variety of cell-specific and tissue-specific glycosylation patterns, with each cell type decorating the CD44 core protein with its own unique array of carbohydrate structures (Jayne Lesley, Robert Hyman, Paul W. Kincade, “CD44 and Its Interaction with Extracellular Matrix,” Advances in Immunology 54(1993):271-335; Tod A. Brown, Todd Bouchard, Tom St. John, Elizabeth Wayner, William G. Carter, “Human Keratinocytes Express a New CD44 Core Protein (CD44E) as a Heparin-Sulfate Intrinsic Membrane Proteoglycan with Additional Exons,” J. Cell Biology 113(April 1991):207-221). Distinct CD44 cell surface molecules have been found in lymphocytes, macrophages, fibroblasts, epithelial cells, and keratinocytes. CD44 expression in the nervous system is restricted to the white matter (including astrocytes and glial cells) in healthy young people, but appears in gray matter accompanying age or disease (Jayne Lesley, Robert Hyman, Paul W. Kincade, “CD44 and Its Interaction with Extracellular Matrix,” Advances in Immunology 54(1993):271-335). A few tissues are CD44 negative, including liver hepatocytes, kidney tubular epithelium, cardiac muscle, the testes, and portions of the skin.

The selectin family of ˜50 kilodalton cell adhesion receptor glycoprotein molecules (Ajit Varki, “Selectin ligands,” Proc. Natl. Acad. Sci. USA 91(August 1994):7390-7397; Masatoshi Takeichi, “Cadherins: A molecular family important in selective cell-cell adhesion,” Ann. Rev. Biochem. 59(1990):237-252) can recognize diverse cell-surface antigen carbohydrates and help localize leukocytes to regions of inflammation (leukocyte trafficking). Selectins are not attached to the cytoskeleton (Elizabeth J. Luna, Anne L. Hitt, “Cytoskeleton-Plasma Membrane Interactions,” Science 258(6 Nov. 1992):955-964). Leukocytes display L-selectin, platelets display P-selectin, and endothelial cells display E-selectin (as well as L and P) receptors. Cell-specific molecules recognized by selectins include tumor mucin oligosaccharides (recognized by L, P, and E), brain glycolipids (P and L), neutrophil glycoproteins (E and P), leukocyte sialoglycoproteins (E and P), and endothelial proteoglycans (P and L) (Ajit Varki, (1994). The related MEL-14 glycoprotein homing receptor family allows lymphocyte homing to specific lymphatic tissues coded with “vascular addressin”—cell-specific surface antigens found on cells in the intestinal Peyer's patches, the mesenteric lymph nodes, lung-associated lymph nodes, synovial cells and lactating breast endothelium. Homing receptors also allow some lymphocytes to distinguish between colon and jejunum (Ted A. Yednock, Steven D. Rosen, “Lymphocyte Homing,” Advances in Immunology 44(1989):313-378; Lloyd M. Stoolman, “Adhesion Molecules Controlling Lymphocyte Migration,” Cell 56(24 Mar. 1989):907-910). Selectin-related interactions, along with chemoattractant receptors and with integrin-Ig, regulate leukocyte extravasation in series, establishing a three-digit “area code” for cell localization in the body (Timothy A. Springer, “Traffic Signals on Endothelium for Lymphocyte Recirculation and Leukocyte Emigration,” Annu. Rev. Physiol. 57(1995):827-872).

Finally, cells may be typed according to their indigenous transmembrane cytoskeleton-related proteins. For example, erythrocyte membranes contain glycophorin C (˜25 kilodaltons, ˜3000 molecules/micron²) and band 3 ion exchanger (90-100 kilodaltons, ˜10,000 molecules/micron²) (Elizabeth J. Luna, Anne L. Hitt, “Cytoskeleton-Plasma Membrane Interactions,” Science 258(6 Nov. 1992):955-964; M. J. Tanner, “The major integral proteins of the human red cell,” Baillieres Clin. Haematol. 6(June 1993):333-356); platelet membranes incorporate the GP Ib-IX glycoprotein complex (186 kilodaltons); cell membrane extensions in neutrophils require the transmembrane protein ponticulin (17 kilodaltons); and striated muscle cell membranes contain a specific laminin-binding glycoprotein (156 kilodaltons) at the outermost part of the transmembrane dystrophin-glycoprotein complex (Elizabeth J. Luna, Anne L. Hitt, “Cytoskeleton-Plasma Membrane Interactions,” Science 258(6 Nov. 1992):955-964). There are also a variety of carbohydrate-binding proteins (lectins) that appear frequently on cell surfaces, and can distinguish different monosaccharides and oligosaccharides (Nathan Sharon, Halina Lis, “Carbohydrates in Cell Recognition,” Scientific American 268(January 1993):82-89). Cell-specific lectins include the galactose (asialoglycoprotein)-binding and fucose-binding lectins of hepatocytes, the mannosyl-6-phosphate (M6P) lectin of fibroblasts, the mannosyl-N-acetylglucosamine-binding lectin of alveolar macrophages, the galabiose-binding lectins of uroepithelial cells, and several galactose-binding lectins in heart, brain and lung (Nathan Sharon, (1993); Mark J. Poznansky, Rudolph L. Juliano, “Biological Approaches to the Controlled Delivery of Drugs: A Critical Review,” Pharmacological Reviews 36(1984):277-336; Karl-Anders Karlsson, “Glycobiology: A Growing Field for Drug Design,” Trends in Pharmacological Sciences 12(July 1991):265-272; N. Sharon, H. Lis, “Lectins—proteins with a sweet tooth: functions in cell recognition,” Essays Biochem. 30(1995):59-75).

Further description of cell types that can be produced in the disclosed method is provided below and elsewhere herein.

a) Keratinizing Epithelial Cells

Keratinizing Epithelial Cells include which includes Epidermal keratinocytes ((differentiating epidermal cell)). The keratinocyte makes up approximately 90% of the cells of the epidermis. The epidermis is divided into four layers based on keratinocyte morphology: which includes the basal layer (at the junction with the dermis), the stratum granulosum, the stratum spinosum, and the stratum corneum. Keratinocytes begin their development in the basal layer through keratinocyte stem cell differentiation. They are pushed up through the layers of the epidermis, undergoing gradual differentiation until they reach the stratum corneum where they form a layer of dead, flattened, highly keratinised cells called squames. This layer forms an effective barrier to the entry of foreign matter and infectious agents into the body and minimizes moisture loss. Keratinizing Epithelial Cells also include Epidermal basal cells which are epidermal stem cells. Keratinizing Epithelial Cells also include Keratinocytes of fingernails and toenails, Nail bed basal cells (a stem cell), Medullary hair shaft cells, Cortical hair shaft cells, Cuticular hair shaft cells, Cuticular hair root sheath cells, Hair root sheath cells of Huxley's layer, Hair root sheath cells of Henle's layer, External hair root sheath cells, and Hair matrix cells (a stem cell). Also included are any stem cells and progenitor cells of the cells disclosed herein, as well as the cells they lead to.

b) Wet Stratified Barrier Epithelial Cells

The human Wet Stratified Barrier Epithelial Cells include surface epithelial cells of the stratified squamous epithelium of the cornea, tongue, oral cavity, esophagus, anal canal, distal urethra, and vagina, as well as basal cells (stem cells) of the epithelia of cornea, tongue, oral cavity, esophagus, anal canal, distal urethra and vagina, and urinary epithelium cells (lining the bladder and urinary tracks. Also included are any stem cells and progenitor cells of the cells disclosed herein, as well as the cells they lead to.

In zootomy, epithelium is a tissue composed of epithelial cells. Such tissue typically covers parts of the body, like a cell membrane covers a cell. It is also used to form glands. The outermost layer of human skin and mucous membranes of mouths and body cavities are made up of dead squamous epithelial cells. Epithelial cells also line the insides of the lungs, the gastrointestinal tract, the reproductive and urinary tracts, and make up the exocrine and endocrine glands. Also included are any stem cells and progenitor cells of the cells disclosed herein, as well as the cells they lead to.

c) Exocrine Secretory Epithelial Cells

Exocrine secretory epithelial cells include Salivary gland mucous cells (which produce polysaccharide-rich secretions), Salivary gland serous cell (glycoprotein-enzyme rich secretion), Von Ebner's gland cell in tongue (washes taste buds), Mammary gland cells (milk secretion), Lacrimal gland cell (tear secretion), and Ceruminous gland cell in ear (wax secretion), Eccrine sweat gland dark cells, (Glycoprotein secretion) Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose, Brunner's gland cell in duodenum (enzymes and alkaline mucus), Seminal vesicle cell (secretes seminal fluid components), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), and Clara cell of lung. Also included are any stem cells and progenitor cells of the cells disclosed herein, as well as the cells they lead to.

d) Hormone Secreting Cells

Hormone secreting cells include Anterior pituitary cells, Somatotropes, Lactotropes, Thyrotropes, Gonadotropes, Corticotropes, Intermediate pituitary cell, secreting melanocyte-stimulating hormone, Magnocellular neurosecretory cells, secreting oxytocin, secreting vasopressin, Gut and respiratory tract cells secreting serotonin, secreting endorphin, secreting somatostatin, secreting gastrin, secreting secretin, secreting cholecystokinin, secreting insulin, secreting glucagon, secreting bombesin, Thyroid gland cells, thyroid epithelial cell, parafollicular cell, Parathyroid gland cells, Parathyroid chief cell, oxyphil cell, Adrenal gland cells, chromaffin cells, secreting steroid hormones (mineralcorticoids and glucocorticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, and Mesangial cell of kidney. Also included are any stem cells and progenitor cells of the cells disclosed herein, as well as the cells they lead to.

e) Epithelial Absorptive Cells (Gut, Exocrine Glands and Urogenital Tract)

Epithelial Absorptive Cells include, Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, and Epididymal basal cell. Also included are any stem cells and progenitor cells of the cells disclosed herein, as well as the cells they lead to.

f) Metabolism and Storage Cells

Metabolism and Storage cells include, Hepatocyte (liver cell), White fat cell, Brown fat cell, and Liver lipocyte. Also included are any stem cells and progenitor cells of the cells disclosed herein, as well as the cells they lead to.

g) Barrier Function Cells (Lung, Gut, Exocrine Glands and Urogenital Tract)

Barrier Function Cells include Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, and Duct cell (of seminal vesicle, prostate gland, etc.). Also included are any stem cells and progenitor cells of the cells disclosed herein, as well as the cells they lead to.

h) Epithelial Cells Lining Closed Internal Body Cavities

Epithelial Cells Lining Closed Internal Body Cavities include Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, and Corneal endothelial cell. Also included are any stem cells and progenitor cells of the cells disclosed herein, as well as the cells they lead to.

i) Ciliated Cells with Propulsive Function

Ciliated Cells with Propulsive Function include, Respiratory tract ciliated cell, Oviduct ciliated cell (in female), Uterine endometrial ciliated cell (in female), Rete testis cilated cell (in male), Ductulus efferens ciliated cell (in male), and Ciliated ependymal cell of central nervous system (lining brain cavities). Also included are any stem cells and progenitor cells of the cells disclosed herein, as well as the cells they lead to.

j) Extracellular Matrix Secretion Cells

Extracellular Matrix Secretion Cells include Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other nonepithelial fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, and Stellate cell of perilymphatic space of ear. Also included are any stem cells and progenitor cells of the cells disclosed herein, as well as the cells they lead to.

k) Contractile Cells

Contractile Cells include Red skeletal muscle cell (slow), White skeletal muscle cell (fast), Intermediate skeletal muscle cell, nuclear bag cell of Muscle spindle, nuclear chain cell of Muscle spindle, Satellite cell (stem cell), Ordinary heart muscle cell, Nodal heart muscle cell, Purkinje fiber cell, Smooth muscle cell (various types), Myoepithelial cell of iris, and Myoepithelial cell of exocrine glands. Also included are any stem cells and progenitor cells of the cells disclosed herein, as well as the cells they lead to.

l) Blood and Immune System Cells

Blood and Immune System Cells include, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, B cells, Natural killer cell, Reticulocyte, and Stem cells and committed progenitors for the blood and immune system (various types). Also included are any stem cells and progenitor cells of the cells disclosed herein, as well as the cells they lead to.

m) Sensory Transducer Cells

Sensory Transducer Cells include Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory receptor neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), and Proprioceptive primary sensory neurons (various types). Also included are any stem cells and progenitor cells of the cells disclosed herein, as well as the cells they lead to.

n) Autonomic Neuron Cells

Autonomic Neuron Cells include Cholinergic neural cell (various types), Adrenergic neural cell (various types), and Peptidergic neural cell (various types). Also included are any stem cells and progenitor cells of the cells disclosed herein, as well as the cells they lead to.

o) Sense Organ and Peripheral Neuron Supporting Cells

Sense Organ and Peripheral Neuron Supporting Cells include Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), and Enteric glial cell. Also included are any stem cells and progenitor cells of the cells disclosed herein, as well as the cells they lead to.

p) Central Nervous System Neurons and Glial Cells

Central Nervous System Neurons and Glial Cells include Neuron cells (large variety of types), Astrocyte glial cell (various types), and Oligodendrocyte glial cell. Also included are any stem cells and progenitor cells of the cells disclosed herein, as well as the cells they lead to.

q) Lens Cells

Lens Cells include Anterior lens epithelial cell, and Crystallin-containing lens fiber cell. Also included are any stem cells and progenitor cells of the cells disclosed herein, as well as the cells they lead to.

r) Pigment Cell

Pigment Cells include Melanocyte and Retinal pigmented epithelial cell. Also included are any stem cells and progenitor cells of the cells disclosed herein, as well as the cells they lead to.

s) Germ Cells

Germ Cells include Oogonium/oocyte, Spermatocyte, and Spermatogonium cell (stem cell for spermatocyte). Also included are any stem cells and progenitor cells of the cells disclosed herein, as well as the cells they lead to.

t) Nurse Cells

Nurse Cells include Ovarian follicle cell, Sertoli cell (in testis), and Thymus epithelial cell. Also included are any stem cells and progenitor cells of the cells disclosed herein, as well as the cells they lead to.

6. Characteristics and Techniques for Compositions and Methods

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that can arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences can be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions can be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example, Ras, as well as any other proteins disclosed herein, as well as various functional nucleic acids. The disclosed nucleic acids are made up of, for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to, for example, Ras, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and Probes

Disclosed are compositions including primers and probes, which are capable of interacting with the genes disclosed herein. The primers can be used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. The primers can be used for the DNA amplification reactions, such as PCR or direct sequencing. It is understood that the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

(4) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Ras or the genomic DNA of Ras or they can interact with the polypeptide Ras. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule can be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of Ras aptamers, the background protein could be Serum albumin. Representative examples of how to make and use aptamers to bind a variety of different target molecules can be found in the following non-limiting list of U.S. Pat. Nos. 5,476,766, 5,503,978, 5,631,146, 5,731,424 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

It is also understood that the disclosed nucleic acids can be used for RNAi or RNA interference. It is thought that RNAi involves a two-step mechanism for RNA interference (RNAi): an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that can act in RNAi. For description of making and using RNAi molecules see See, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least form material related to delivery and making of RNAi molecules.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

d) Delivery of Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as a Ras expressing nucleic acid, into the cell without degradation and include a promoter yielding expression of the gene in the cells into which it is delivered. The vectors can be derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A viral vector can be used which has been engineered so as to suppress the immune response of the host organism, elicited by the viral antigens. Preferred vectors of this type will carry coding regions for Interleukin 8 or 10.

Viral vectors can have higher transaction abilities (ability to introduce genes) than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, Science 260:926-932 (1993); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Both the E1 and E3 genes can be removed from the adenovirus genome.

(c) Adeno-associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An useful form of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(d) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (QIAGEN, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described herein, the compositions can be administered in a pharmaceutically acceptable carrier and can be delivered to the subject cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Peptides

(1) Protein Variants

There are numerous variants of the disclosed proteins that are known and herein contemplated. In addition, to the known functional strain variants there are derivatives of the proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues can be deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular cell from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations (Rizo and Gierasch, Ann. Rev. Biochem. 61:387 (1992), incorporated herein by reference).

f) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone can range from about 1 μg/kg to up to 100 mg/kg of body weight or more per day, depending on the factors mentioned above.

g) Chips and Microarrays

Disclosed are chips where at least one address is the sequences or part of the sequences set forth in any of the nucleic acid sequences, peptides, or cells disclosed herein. Also disclosed are chips where at least one address is the sequences or portion of sequences set forth in any of the peptide sequences disclosed herein. For example, one could have different 96 well plates, one of which has liver cells, one of which has lung cells, and one of which has heart cells heart cells, for example, and ship these as a kit with reagents and media. The end user, would then add things to be tested, for example, into the wells. Another example includes screening using a high density array of chemicals on a film which is then washed with various solutions containing compositions, such as cells or other things, which then give an indicator if they interact with something on the chip.

Also disclosed are chips where at least one address is a variant of the sequences or part of the sequences set forth in any of the nucleic acid sequences, peptides, or cells disclosed herein. Also disclosed are chips where at least one address is a variant of the sequences or portion of sequences set forth in any of the peptide sequences disclosed herein.

h) Computer Readable Media

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable media comprising the sequences and information regarding the sequences set forth herein.

i) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include nucleic acids encoding the desired molecules or modified ES cells discussed in certain forms of the methods, as well as the buffers and enzymes required to use them. Other examples of kits, include cells derived by the methods described herein useful for toxicity screening. These cells can represent a variety of terminally differentiated cells that give a relevant profile of the drug being screened. The cells could, for example, still comprise the marker or could have the marker excised. Since the methods allow the use of a pluripotent cell as the starting cell, multiple cell types all derived from a common pluripotent cell and thus sharing a common genotype can be generated. Kits, can include, for example, plates, such as 96 well plates, which can be coated with the compositions disclosed herein.

B. Methods

1. Methods of Using Modified Stem Cells

The modified stem cells can be used to identify and select desired cell types and cultures of desired cell types. In general, the modified stem cells can be cultured under conditions allowing all cells to grow. Then the modified stem cells can then be put under a selective pressure, such as movement into soft agar which will select for the presence of a transforming gene. Those cells which are expressing the selection gene, such as transforming gene, will continue to grow or can be identified. Because the modified stem cell has been engineered so that the selection gene is only expressed in a single cell type or subset of cell types only these cells will continue to proliferate or remains identifiable. Further or alternative steps of identification, such as through cell sorting for particular cell type markers or visualization and subsequent sub-culturing and cloning can produce a population of cells which are a single cell type and which if cloned, arose from a single ancestor cell, When the modified stem cell is a cell which can form an embryoid body under the appropriate conditions, then since an embryoid body can give rise to any cell type spontaneously, any desired cell type can be obtained by allowing the modified stem cell to go through spontaneous embryoid body formation, with subsequent selection, such as for a transforming gene, as discussed herein. It is understood that these methods and those disclosed herein, along with the compositions disclosed can produce any desired cell type, such as those disclosed herein. To initiate the formation of embryoid bodies, typically undifferentiated stem cells are passaged, via trypsin or some other dissociation method, into untreated plastic dishes in the absence of a feeder layer. Without special treatment, cells typically do not readily attach to plastic. In these condition, the stem cells will divide to form individual balls of cells with a hollow cavity.

2. Methods of Using Differentiated Cells

The methods for making the modified stem cells as disclosed herein can produce cells which are suitable for in vivo methods and/or ex vivo methods and/or in vitro methods. For example, the activated/dominant negative transforming gene strategy, for example, can be best suited to in vitro applications but would not be as desirable for cell therapy because the marker, such as the transforming gene, would remain within the cell. On the other hand CRE/lox is suitable for cell therapy because the marker, such as a transforming gene, is excised from the final cell. Furthermore, for in vivo mechanisms the marker can be placed on an extrachromosomal cassette, such as a mammalian artificial chromosome, which can then be removed entirely from the final cells using a variety of mechanisms.

a) Methods of Identifying Conditions for Differentiation

Disclosed are methods of using the disclosed cells in methods for identifying and optimizing conditions to differentiate stem cells. The process of differentiation proceeds in a stepwise fashion with cells progressing from one precursor cell to the next before their final cell type. An example can be found in the hematopoietic system where the primordial stem cell gives rise to various precursors which in turn generate additional precursors before the appearance of the final B cell or T cell. Disclosed are methods and compositions which can be used to define this progression, or any other, from precursor to final product, and include the disclosed reversible transformation system.

Most genes whose function is well understood are genes expressed in the final tissue. These genes are genes whose promoters would be useful in the disclosed methods and compositions, as they are terminal cell type promoters. A terminal cell type is a cell type which is no longer differentiates. Albumin is a good example of a gene expressed in a terminal cell type. Albumin is expressed only in the hepatocyte. Its promoter is driven by a series of known transcription factors, such as the CAAT/Enhancer binding protein (C/EBP) and the forkhead family of proteins (Schrem, H., et al. Pharmacol. Rev. 54, 129-158, 2002.) Using the disclosed methods and compositions, such as the tissue specific reversible transformation procedure, one can identify cells that become hepatocytes within the mixture of other cells derived from the embryoid body. One can use the promoter from one of the albumin-controlling transcription factors as the tissue specific selector, and identify the cell immediately preceding the hepatocyte. This cell can then be isolated and using standard genomic techniques, genes expressed in that cell can be identified and additional selectors, genes which are uniquely expressed in the cell, can be identified. Repeating this procedure with each additional selector, we can trace a lineage back to the origin.

A variation on this can be used to define cell culture conditions for each step in the progression. Using, for example, a transforming gene, such as the activated Ras gene, as the marker, one can quantitate how many colonies appear in soft agar under various culture conditions. Using green fluorescent protein or lactate dehydrogenase would also allow quantitation. By varying the conditions of culture along with the selectors, cell or linage specific promoters, one can maximize the number of cells that follow a particular pathway at each stage, or identify any other desired characteristic. Maximizing the yield at each stage can allow, for example, one to design a differentiation protocol that would lead to the desired cell type without the use of the selector.

b) Reconstituted Immune System

Disclosed herein are methods and compositions capable of generating and modifying any desired human cell type. For example, disclosed is the in vitro reconstitution of the human immune system. Monoclonal antibodies currently are produced in mice by a three-step process. The mouse is first inoculated with the desired antigen. After a few days, its spleen is removed and the immune cells residing in the spleen are fused with a mouse B cell lymphoma line. This serves to immortalize the B cells in the spleen. These are then cultured and the fusion that is producing the appropriate antibody is selected.

Mouse monoclonal antibodies are poor therapeutics in humans since they are recognized as foreign and destroyed. Monoclonal antibodies that are currently being used for therapies, such as Herceptin® for breast cancer, are humanized or chimerized to minimize these problems, but they are not completely eliminated. Fully human monoclonal antibodies are the solution. Unfortunately, this would mean inoculating people with the antigen. This has been both unpopular and unsuccessful, in the few instances where it has been attempted. As disclosed herein, tissue specific, reversible transformation of stem cells will allow the selection of a matched set of human immune cells: B, T and macrophage lines. This can only be accomplished from stem cells since the B, T, and macrophage cells should be from the same genetic background in order to function correctly. When the appropriate cells are established, they can be cultured together to produce an in vitro immune system. Antigen incubated in the system can be processed and presented to the B cells correctly, expanding the cognate cells. With time in culture, these cells can proliferate preferentially or selectively, comprising a larger percentage of the total B cell population. These cells can then be cloned and the appropriate antibody producing cell can be selected. Because they are transformed, they can be characterized, frozen, and then expanded indefinitely, producing fully human monoclonal antibodies. This system can dramatically expand the applicability of monoclonal antibodies for therapy.

c) Toxicology Testing

The desire of the pharmaceutical industry to drive down the staggering cost of new drug discovery and development has forced an examination of the factors that cause drug candidates to fail. After efficacy problems, the most common reason for failure is toxicity (van de Waterbeemd, H, Gifford, E. (2003) Nat. Rev. Drug Disc. 2, 192-204). Even more problematic are compounds that go onto the market, only to be withdrawn due to unrecognized toxicities. Troglitazone and trovafloxacin are well known examples of compounds which were pulled or whose use was severely curtailed due to liver toxicity, grepafloxacin had problems with muscle toxicity, terfenadine and astemizole were pulled due to cardiac toxicity (Suchard, J. (2001) Int. J. Med. Toxicol. 4, 15-20).

Ideally, the toxic properties of new compounds can be recognized and avoided early in development. ACTIVTox, based on a human liver cell line, is designed to provide a high throughput, metabolically active platform for the development of structure toxicity relationships. Compounds are screened through a battery of tests at multiple concentrations to develop a structural ranking that can be used by the chemists to direct the next round of synthesis. In this way, the toxic properties of a compound can be minimized while the therapeutic properties are maximized.

By developing a panel of related cell lines, the idea of ACTIVTox can be generalized. New compounds can be tested against a panel of matched, non-transformed cell lines in a high throughput system, raising the probability of success in clinical trials. Using the methods described herein, the panel can consist of cell lines, representing a number of tissues, matched as closely as possible. This could be accomplished by derivation of the cells used in the assay from the same parental stem cell line, e.g. an EG line, and reversibly transformed by the same mechanism. These cells would constitute a set of tissue samples from a single individual, minimizing problems with differences in genetic background.

Predictive toxicology using the disclosed method can also be performed with a larger cell collection. Disclosed are methods of toxicology testing on heart, neuron, intestine, kidney, liver, muscle, or lung lines. These lines can be produced and screened in the same toxicity assays using the same compounds, as those which are used for liver.

An example is beating heart cell cultures. A major concern among pharmaceutical companies is the phenomenon known as QT prolongation, which can lead to heart arrythmias and possibly death (Belardinelli, L., et al. Trends in Pharmocol. Sci. 24, 619-625, 2003). Several compounds, such as terfenadine, were withdrawn from the market for this serious side effect. Currently, it is difficult to test for QT prolongation except in animals or people, since it is an electrical phenomenon. Beating heart cell cultures would allow a direct test for this problem.

By testing the same compounds in the same assays using many different cell types, a clear picture of the toxic potential of new compounds can be determined before testing in humans. This will have a dramatic effect on the cost and speed of new drug development since clinical testing is by far the most expensive phase.

d) Specific Target Cells for Discovery Applications

(1) Dopamine Specific Neurons

Tissue specific reversible transformation also allows the development of specific cell types for drug discovery applications. Currently, new drugs are frequently tested on cells that have been genetically manipulated to contain the target of interest because the natural target-containing cell is unavailable. An example is dopaminergic neurons. Many neuroactive drugs are directed against the dopamine receptor, such as the tricyclic antidepressants or dopamine reuptake inhibitors for drug addiction. The availability of an unlimited and reproducible supply of the specific cell type of interest, such as dopaminergic neurons uncontaminated by any other cell type, are disclosed herein.

e) Knockouts for Target Validation

The use of the disclosed methods and compositions, such as tissue specific reversible transformation, in combination with gene targeted, homologous recombination allows the development of cells with a particular gene deleted or modified. A central problem in drug development is the validation of therapeutic targets. This is the determination of whether a particular protein, when blocked or activated by a drug, will in fact deliver the desired therapeutic effect. Knockout or knock in mice are frequently used in this application (Zambrowicz, B P, et al. Nat. Rev. Drug Disc. 2, 38-51, 2003). The disclosed cells and cell lines, which have been produced as disclosed herein, will provide similar validation opportunities in vitro. A specific example is the knockout of the human low density lipoprotein receptor. The LDL receptor is used as an entryway for a number of human viruses, including the human hepatitis B virus. Using the techniques of homologous recombination in the cells disclosed herein, such as stem cells, the LDL receptor gene can be damaged, such that no LDL receptor protein is synthesized. Using tissue specific reversible transformation in these cells, human hepatocytes without the LDL receptor can be created. These cells can be used to examine the role of the LDL receptor in HBV infection. If, for example, these cells were uninfectable with HBV, the LDL receptor would be declared to be a validated target for anti HBV therapies. Similar strategies could be devised to create gain of function or loss of function mutations for other purposes. Using the same example as above, the LDL receptor could be activated in cells that normally do not express this protein.

f) Ex Vivo Cell Therapy

(1) Liver Assist Device

Disclosed is a liver assist device based on the liver cell lines disclosed herein. There are about 5,000 liver transplantations carried out in the United States each year. There are currently about 17,000 on the waiting list. About 1500 die on the list each year.

Currently, there is no means to support a patient who has entered into end stage liver disease, such as hemodialysis for kidney patients. Because of the liver's ability to regenerate, support for this short, crucial period can allow the patient to survive, either until a suitable organ is available or, in the best of circumstances, with their own liver.

A liver assist device in animals and on 52 patients in the United States and Great Britain has been developed and tested (Sussman, N L, et al., (1992) Hepatology 16, 60-65; Sussman, N L, et al., (1994) Artificial Organs 18, 390-396; Millis, J M, et al., (2002) Transplantation 74, 1735-1746). In this device, a hollow fiber cartridge, as is used in kidney dialysis, is filled with a human liver cell line that carries out the function of the liver. The cells are separated from the patient's immune system by the cellulose acetate fibers. Blood is pumped through the lumen of the fibers, small molecules diffuse through the fibers to the cells, where they are appropriately metabolized. The device is safe and while trials of sufficient power to prove its effectiveness have not been carried out, anecdotal evidence suggests that it is able to save lives. Other similar devices, using animal hepatocytes, also appear to be effective (Hui, T, et al., (2001) J. Hepatobiliary Pancreat Surg. 8, 1-15).

A practical problem arises in the source of the hepatocytes to fill the device. In order to be effective, each device requires about 200 g of cells, 15 to 20% of the total liver mass. Hepatocytes, despite their regenerative capabilities in vivo, do not divide to any extent in culture, even after decades of research on this topic. The statistics described in the opening paragraph are not encouraging in using human livers to supply cells for support devices. Transplantation is totally organ limited. The use of animal livers can supply sufficient cells but requires the constant harvest of new organs and presents problems of reproducibility and quality control. This problem has been approached by employing a human liver cell line, which is immortalized and could be frozen in cell banks (Sussman, N L & Kelly, J H. (1995) Scientific American: Science and Medicine 2, 68-77). These cells can supply a constantly renewable, reproducible and unlimited supply of devices.

Unfortunately, the tumor-derived source of these cells has presented acceptance and regulatory problems for its use in human therapy. The disclosed hepatocytes produced from the compositions and methods disclosed herein can circumvent this hurdle, because after reversion, they are no longer a cell line.

g) Genetically Matched Cell Lines

Genetically matched cell lines can be used for gene expression studies and proteomic studies since the genetic noise level can be dramatically reduced.

A major drawback to use of cells in culture, prior to the disclosed cells, to study gene expression is that the cells do not have the same genetic background. Different sets of genes are expressed at different levels in different individuals. This has both a genetic and environmental component. Moreover, most cells in culture are derived from tumors, which are, by definition, genetically abnormal and usually contain multiple inversions, duplications and completely duplicated or missing chromosomes.

A set of cells that were isolated from the same stem cell would be that same as having tissue samples from an individual. The genetic background of cells from the liver and the intestine, for example, would be the same. This allows for a much clearer determination of tissue specific expression of genes and proteins, since individual variability is eliminated. The disclosed methods and compositions can be used to produce genetically matched cells of a specific cell type from any cell disclosed herein, such as stem cells, from any source, such as any unique individual.

h) Identification of Developmental Pathways and Control

As described earlier, transcription factors act combinatorially to effect tissue specific gene expression. The disclosed compositions and methods can be used to identify cell stages that activate certain genes specific for a given cell type. Using the hepatocyte as an example, albumin is primarily a product of the adult hepatocyte. Several transcription factors are known to regulate its expression. One such factor is C/EBP, a factor in the regulation of many genes involved in intermediary metabolism (Darlington, G J, (1998) J. Biol. Chem. 273, 30057-30060). Using the promoter for C/EBP in the EG system, for example, one can identify cells that activate this gene. One of these is the hepatoblast, a precursor to the hepatocyte. By then selecting a gene whose expression regulates C/EBP, we can follow the developmental pathway backwards to the origin, stepwise.

C. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular modified ES cell is disclosed and discussed and a number of modifications that can be made to a number of molecules including the modified ES cell are discussed, specifically contemplated is each and every combination and permutation of modified ES cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that there are many different compositions and method steps disclosed herein and each and every combination and permutation for each composition and method as disclosed herein is contemplated and disclosed. For example, there are lists of transformation genes, promoters, cell types, recombinase combinations, modified stem cells, markers, cell specific genes, and each combination of each of these singularly or in total, is disclosed, which provides many thousands of specific embodiments and sets of embodiments. Once the lists and pieces are disclosed, the combinations are also disclosed without specifically reciting each combination.

Furthermore, it is understood that unless specifically indicated to the contrary or unless understood as being contrary to the skilled artisan, where one specific embodiment is discussed, such as a Ras transformation gene, then all other transformation genes are also disclosed for that recitation or embodiment, and likewise for each composition and method step disclosed herein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, primary culture, or cultures derived from such cells unless specifically indicated. A “culture” refers to a composition comprising isolated cells of the same or a different type.

A cell line is a culture of a particular type of cell that can be reproduced indefinitely, thus making the cell line “immortal.”

A cell culture is a population of cells grown on a medium such as agar.

A primary cell culture is a culture from a cell or taken directly from a living organism, which is not immortalized.

The term “pro-drug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

The term “metabolite” refers to active derivatives produced upon introduction of a compound into a biological milieu, such as a patient.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Nucleic acid segments for use in the disclosed method can also be referred to as nucleic acid sequences and nucleic acid molecules. Unless the context indicates otherwise, reference to a nucleic acid segment, nucleic acid sequence, and nucleic acid molecule is intended to refer to an oligo- or polynucleotide chain having specified sequence and/or function which can be separate from or incorporated into or a part of any other nucleic acid.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System iPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide can be independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. For example, disclosed are the cells produced by the disclosed methods. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequences disclosed herein and a sequence controlling the expression of the nucleic acid.

Also disclosed are nucleic acid molecules produced by the process comprising linking in an operative way a nucleic acid molecule comprising a sequence having 80% identity to the sequences disclosed herein, and a sequence controlling the expression of the nucleic acid.

Disclosed are nucleic acid molecules produced by the process comprising linking in an operative way a nucleic acid molecule comprising a sequence that hybridizes under stringent hybridization conditions to the disclosed sequences and a sequence controlling the expression of the nucleic acid.

Disclosed are nucleic acid molecules produced by the process comprising linking in an operative way a nucleic acid molecule comprising a sequence encoding a peptide disclosed herein and a sequence controlling an expression of the nucleic acid molecule.

Disclosed are nucleic acid molecules produced by the process comprising linking in an operative way a nucleic acid molecule comprising a sequence encoding a peptide having 80% identity to a peptide disclosed herein and a sequence controlling an expression of the nucleic acid molecule.

Disclosed are nucleic acids produced by the process comprising linking in an operative way a nucleic acid molecule comprising a sequence encoding a peptide having 80% identity to a peptide disclosed herein, wherein any change from the peptide sequence are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids. Combinations of different cells produced by the methods described herein are also disclosed. Also combinations of cells produced by the methods described herein mixed with other cells are also provided. These cells can have various purities based on the particular need or application.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

Disclosed are any of the stem cells disclosed herein produced by transforming the cells with the nucleic acids disclosed herein. Also disclosed are any of the cells produced by the methods disclosed herein, such as the methods for isolating selecting a specific cell type and using the disclosed modified stem cells.

E. Methods of Using the Compositions

1. Methods of Using the Compositions as Research Tools

The disclosed compositions can be used in a variety of ways as research tools.

The compositions can be used for example as targets in combinatorial chemistry protocols or other screening protocols to isolate molecules that possess desired functional properties related to the specific cell type.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, a particular gene in a particular cell type disclosed herein. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein. Similarly, a stem cell, such as a pluripotent stem cell, can be used to knock out a gene to create a transgenic animal and the same cell can be used in methods described herein to create cell lines that can be compared to the animal in various assays.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### F. Specific Embodiments

Disclosed is a pluripotent stem cell containing a nucleic acid segment, wherein the nucleic acid segment comprises the structure P-I, wherein P is a transcriptional control element and I is a sequence encoding a marker, wherein the marker comprises a transformation agent.

Also disclosed is a differentiated cell produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method comprising introducing the differentiated cell into a subject, wherein the differentiated cell is produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method of assaying a composition for toxicity, the method comprising incubating the composition with a differentiated cell, and assessing the differentiated cell for toxic effects, wherein the differentiated cell is produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method of assaying a compound for toxicity, the method comprising incubating the compound with a differentiated cell, and assessing the differentiated cell for toxic effects, wherein the differentiated cell is produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method of assaying a composition for an effect of interest on a cell, the method comprising incubating the composition with a differentiated cell, and assessing the differentiated cell for the effect of interest, wherein the differentiated cell is produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method of assaying a compound for an effect of interest on a cell, the method comprising incubating the compound with a differentiated cell, and assessing the differentiated cell for the effect of interest, wherein the differentiated cell is produced by culturing a pluripotent stem cell under conditions in which the transcriptional control element is activated, whereby I is preferentially or selectively expressed, wherein the pluripotent stem cell contains a nucleic acid segment, wherein the nucleic acid segment comprises the structure P-I, wherein P is a transcriptional control element and I is a sequence encoding a marker, wherein the marker comprises a transformation agent.

Also disclosed is a method of deriving differentiated cells from stem cells, the method comprising culturing stem cells under conditions in which the transcriptional control element is activated, whereby I is preferentially or selectively expressed, thereby deriving differentiated cells, wherein the stem cells contain a nucleic acid segment, wherein the nucleic acid segment comprises the structure P-I, wherein P is a transcriptional control element and I is a sequence encoding a marker, wherein the marker comprises a transformation agent, wherein I is a heterologous nucleic acid sequence.

Also disclosed is a method of deriving stem cell derived conditionally immortal cell types, the method comprising culturing stem cells under conditions in which the transcriptional control element is activated, whereby I is preferentially or selectively expressed, thereby deriving stem cell derived conditionally immortal cell types, wherein the stem cells contain a nucleic acid segment, wherein the nucleic acid segment comprises the structure P-I, wherein P is a transcriptional control element and I is a sequence encoding a marker, wherein the marker comprises a transformation agent, wherein I is a heterologous nucleic acid sequence.

Also disclosed is a method of deriving stem cell derived conditionally immortal cell types, the method comprising transfecting stem cells with a nucleic acid segment comprising the structure P-I, wherein P is a transcriptional control element and I is a sequence encoding a marker, wherein the marker comprises a transformation agent; culturing the stem cells under conditions in which the transcriptional control element is activated, whereby I is preferentially or selectively expressed, thereby deriving stem cell derived conditionally immortal cell types.

Also disclosed is a method of deriving differentiated cells from stem cells, the method comprising transfecting stem cells with a nucleic acid segment comprising the structure P-I, wherein P is a transcriptional control element and I is a sequence encoding a marker, wherein the marker comprises a transformation agent; and culturing the stem cells under conditions in which the transcriptional control element is activated, whereby I is preferentially or selectively expressed, thereby deriving differentiated cells.

Also disclosed is a method of deriving differentiated cells from stem cells, the method comprising transfecting stem cells with a nucleic acid segment comprising the structure P-I, wherein P is a transcriptional control element and I is a sequence encoding a marker; and culturing the stem cells under conditions in which the transcriptional control element is activated, whereby I is preferentially or selectively expressed, wherein the conditions in which the transcriptional control element is activated are conditions in which the stem cells differentiate thereby deriving differentiated cells.

Also disclosed is a pluripotent stem cell containing a nucleic acid molecule comprising the structure P-I, wherein: P is a transcriptional control element; and I is a sequence encoding a marker, wherein the marker comprises a transformation agent. Also disclosed is a cell produced by excising a nucleic acid from a stem cell, wherein the stem cell contains a nucleic acid molecule comprising the structure P-I, wherein: P is a transcriptional control element; and I is a sequence encoding a marker, wherein the marker comprises a transformation agent.

Also disclosed is a method of deriving a population of conditionally immortal cell types from stem cells, comprising transfecting a stem cell with a construct containing one of the nucleic acid molecules P-I recited in claim 1; culturing the stem cells in an environment such that transcriptional control of element P is activated, whereby I is preferentially or selectively expressed; and selecting cell types expressing I.

Also disclosed is a method of deriving a population of conditionally immortal cell types from stem cells, comprising transfecting a stem cell with a construct containing one of the nucleic acid molecules P-I recited in claim 1; culturing the stem cells in an environment such that transcriptional control of element P is activated, whereby I is preferentially or selectively expressed; and selecting cell types expressing I.

Also disclosed is a method of deriving conditionally immortal cell types, comprising transfecting pluripotent stem cells with a construct containing one of the nucleic acid molecules P-I; activating control element P, whereby I is preferentially or selectively expressed; selecting cell types expressing I and; excising the construct containing the P-I nucleic acid molecule; contacting the selected cell types with an environment such that the ends of the nucleic acid formerly containing the construct containing the P-I nucleic acid molecule recombine; and freezing of the selected cell type.

Also disclosed is a method of deriving a cell culture, comprising transfecting pluripotent stem cells with a construct containing one of the nucleic acid molecules P-I; contacting the stem cells with an environment such that transcriptional control element P is activated and I is preferentially or selectively expressed; and culturing the cells expressing I, wherein P is a transcriptional control element; and I is a sequence encoding a marker, wherein the marker comprises a transformation agent.

Also disclosed is a pluripotent stem cell containing a nucleic acid molecule construct comprising the structure P-I, wherein P is a tissue specific transcriptional control element; P causes I to be preferentially or selectively expressed; and I is a temperature permissive immortalization agent.

Also disclosed is a pluripotent stem cell containing a nucleic acid molecule construct comprising the structure X-P-I-X, wherein P is a tissue specific transcriptional control element; P causes I to be preferentially or selectively expressed; I is a temperature permissive immortalization agent; and X is a site-specific excision sequence.

Also disclosed is a method of deriving stem cell derived conditionally immortal cell types, comprising transfecting pluripotent stem cells with a construct containing the nucleic acid molecule construct P-I; contacting the stem cells with an environment such that transcriptional control element P is activated and I is preferentially or selectively expressed; selecting of stem cell derived cell types expressing I; and cloning and freezing of a selected cell type, wherein P is a transcriptional control element; and I is a sequence encoding a marker, wherein the marker comprises a transformation agent.

Also disclosed is a method of deriving stem cell derived conditionally immortal cell types, comprising transfecting pluripotent stem cells with a construct containing the nucleic acid molecule construct X-P-I-X; contacting the stem cells with an environment such that transcriptional control element P is activated and I is preferentially or selectively expressed; selecting of stem cell derived cell types expressing I; and cloning and freezing of a selected cell type, wherein X is a site-specific recombination site, P is a transcriptional control element; and I is a sequence encoding a marker, wherein the marker comprises a transformation agent.

Also disclosed is a method of deriving stem cell derived conditionally immortal cell types, comprising transfecting pluripotent stem cells with a construct containing the nucleic acid molecule construct X-P-I-X recited in claim 11; contacting the stem cells with an environment such that transcriptional control element P is activated and I is preferentially or selectively expressed; selecting of stem cell derived cell types expressing I; excising of the construct containing the P-I nucleic acid molecule; and cloning and freezing of a selected cell type, wherein X is a site-specific recombination site, P is a transcriptional control element; and I is a sequence encoding a marker, wherein the marker comprises a transformation agent.

Also disclosed is a method of treating a patient comprising transplanting cell types derived from stem cells. Also disclosed is a method of treating a patient comprising transplanting cell types derived form stem cells. Also disclosed is a method of assaying a composition for toxicity comprising incubating the composition with cells derived from stem cells.

The nucleic acid segment can be a heterologous nucleic acid segment. The nucleic acid segment can be an exogenous nucleic acid segment. The marker can be heterologous. I can be a heterologous nucleic acid sequence. P and I can be contained in the same vector. P and I can be contained in different vectors. The nucleic acid segment can further comprise a suicide gene. P can be a tissue specific transcriptional control element. P can be a cell type specific transcriptional control element. P can be a cell lineage specific transcriptional control element. P can be a cell specific transcriptional control element. P can causes I to be preferentially or selectively expressed.

The marker can comprise a temperature permissive immortalization agent. The transformation agent can be a temperature permissive agent. I can comprises the SV40 large T antigen. The nucleic acid segment can be flanked by a site-specific excision sequence. I can be flanked by a site-specific excision sequence. P can be flanked by a site-specific excision sequence. The nucleic acid segment can further comprise X, wherein X can be a site-specific excision sequence, wherein X flanks P-I, wherein the nucleic acid segment comprises the structure X-P-I-X. The nucleic acid segment can be excised at X. X can be a loxP site.

The conditions in which the transcriptional control element can be activated can be conditions in which the stem cell differentiates. The stem cell can differentiate under the conditions in which the transcriptional control element can be activated. The transcriptional control element can be activated by allowing the stem cells to spontaneously differentiate into an embryoid body. The nucleic acid segment can be excised from the differentiated cell. The nucleic acid segment can be excised using an adenovirus-mediated site-specific excision. The nucleic acid segment can be excised using a recombinase. The recombinase can be Cre. The excision of the nucleic acid segment results in recombination of the nucleic acid molecule from which the nucleic acid segment can be excised.

The effect of the expression of I can be reversed. The effect of expression of I can be transformation of the differentiated cell, wherein reversal of the effect of the expression of I can be reversal of transformation of the differentiated cell. The effect of the expression of I can be reversed by expression of a dominant negative transformation agent. The effect of the expression of I can be reversed by excision of the nucleic acid segment. The differentiated cell can be a hepatocyte. The differentiated cell can be a stem cell derived conditionally immortal cell.

The differentiated cell can be introduced by administering the differentiated cell to the subject. The differentiated cell can be introduced by transplanting the differentiated cell into the subject. The conditions in which the transcriptional control element can be activated can be conditions in which the stem cells differentiate. The stem cells can differentiate under the conditions in which the transcriptional control element can be activated. The transcriptional control element can be activated by allowing the stem cells to spontaneously differentiate into an embryoid body.

The method can further comprise selecting cells expressing I. The method can further comprise increasing the purity of the cells expressing I. Increasing the purity can comprise creating a clonal or semi-purified population of cells. The method can further comprise excising the nucleic acid segment. The method can further comprise cloning the differentiated cells. The method can further comprise culturing the differentiated cells. The method can further comprise freezing the differentiated cells. The method can further comprise adding a gene of interest to the selected cells. The method can further comprise excising the nucleic acid segment; and freezing of the selected cells. The ends of the nucleic acid formerly containing the nucleic acid segment can recombine when the nucleic acid segment is excised. The method can further comprise culturing the cells expressing I. The method can further comprise cloning the cultured cells expressing I. The method can further comprise introducing the differentiated cells into a subject.

The differentiated cell can be introduced by administering the differentiated cell to the subject. The differentiated cell can be introduced by transplanting the differentiated cell into the subject. The method can further comprise incubating a composition with the differentiated cells, and assessing the differentiated cells for toxic effects. The method can further comprise incubating a compound with the differentiated cells, and assessing the differentiated cells for toxic effects. The method can further comprise incubating a composition with the differentiated cells, and assessing the differentiated cells for an effect of interest. The method can further comprise incubating a compound with the differentiated cells, and assessing the differentiated cells for an effect of interest. The method can further comprise selecting the differentiated cells by selecting for the marker. The method can further comprise screening for the differentiated cells be identifying cells expressing the marker. The stem cells can differentiate under the conditions in which the transcriptional control element can be activated. The transcriptional control element can be activated by allowing the stem cells to spontaneously differentiate into an embryoid body.

The marker can be expressed from a heterologous nucleic acid. The nucleic acid can further comprise a suicide gene. P can be a tissue specific transcriptional control element. P can cause I to be preferentially or selectively expressed. The immortalization agent can be a temperature permissive agent. I can comprise the SV40 large T antigen. The nucleic acid molecule can be flanked by a site-specific excision sequence. I can be flanked by a site-specific excision sequence. P can be flanked by a site-specific excision sequence. P-I can be flanked by a site-specific excision sequence, X, forming X-P-I-X. The nucleic acid molecule comprising the structure P-I can be excised using an adenovirus-mediated site-specific excision. The excision of the nucleic acid molecule comprising the structure P-I can result in recombination of the non-excised nucleic acid molecule.

The method can further comprise increasing the purity of the population of cells expressing I. Increasing the purity can comprise creating a clonal or semi-purified population of cells. The method can further comprise excising the nucleic acid. The method can further comprise freezing the selected cell type. The method can further comprise adding a gene of interest to the population of cells. Activating control element P can comprise allowing the stem cell culture to spontaneously differentiate into an embryoid body. The method can further comprise cloning the cultured cells expressing I.

P-I can be excised. P-I can be excised at X by an adenovirus-mediated site-specific excision. The excision of P-I can allow recombination of the nucleic acid formerly containing the construct containing the P-I nucleic acid molecule. P and I can be contained in the same vector. P and I can be contained in different vectors.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Identification of a Human Hepatocyte Cell Line Using an Activated/Dominant Negative Transforming Gene Pair

Identification of a human hepatocyte cell line starting from human EG cells using sequential expression of an activated and a dominant negative transforming gene can be performed as follows. Human EG cells can be transfected with a construct containing the human hepatitis B virus core promoter/enhancer (SEQ ID NO:1) driving an activated H-RAS gene (SEQ ID NO:2) and also optionally containing an ecdysone inducible gene switch promoter (SEQ ID NO:3) driving a dominant negative H-RAS gene (SEQ ID NO:4) (Sandig et al., (1996) Gene Therapy 3, 1002-1009; Saez et al., (2000) Proc. Natl. Acad. Sci. 97, 14512-14517). The activated H-RAS can be transcribed after differentiation of the EG cells. Transformed hepatocytes can be isolated in soft agar, cloned, expanded and frozen. Cultures can be plated at low density then treated with ponasterone A to induce the dominant negative RAS and reverse transformation. Cells are expected to arrest growth at subconfluent densities. Their identity as hepatocytes can be confirmed by production of albumin, cyp1A and cyp3A.

This transformation can be performed using pHBV-aRAS and ACTEG1 cells to produce hepatocyte cell lines that can be identified from embryoid bodies.

a) Methods

(1) Plasmids

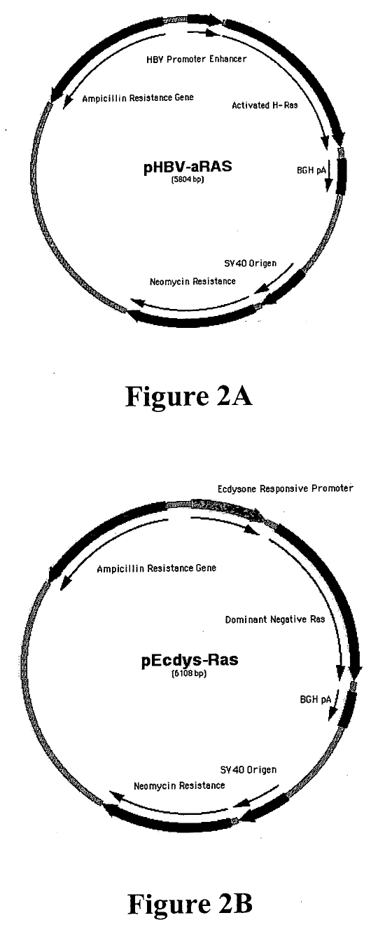

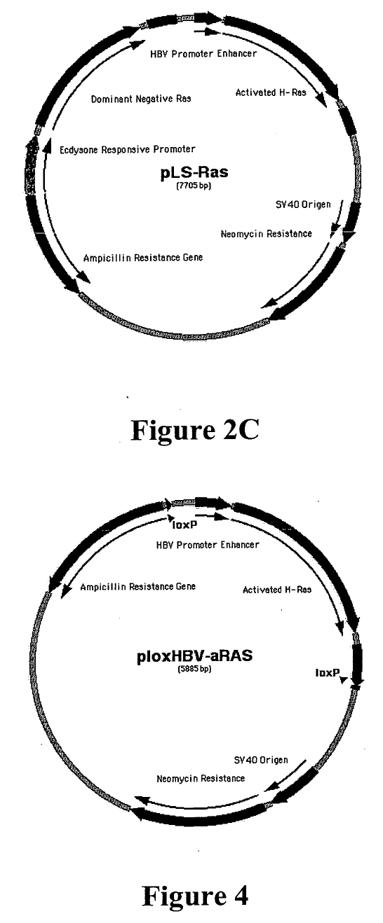

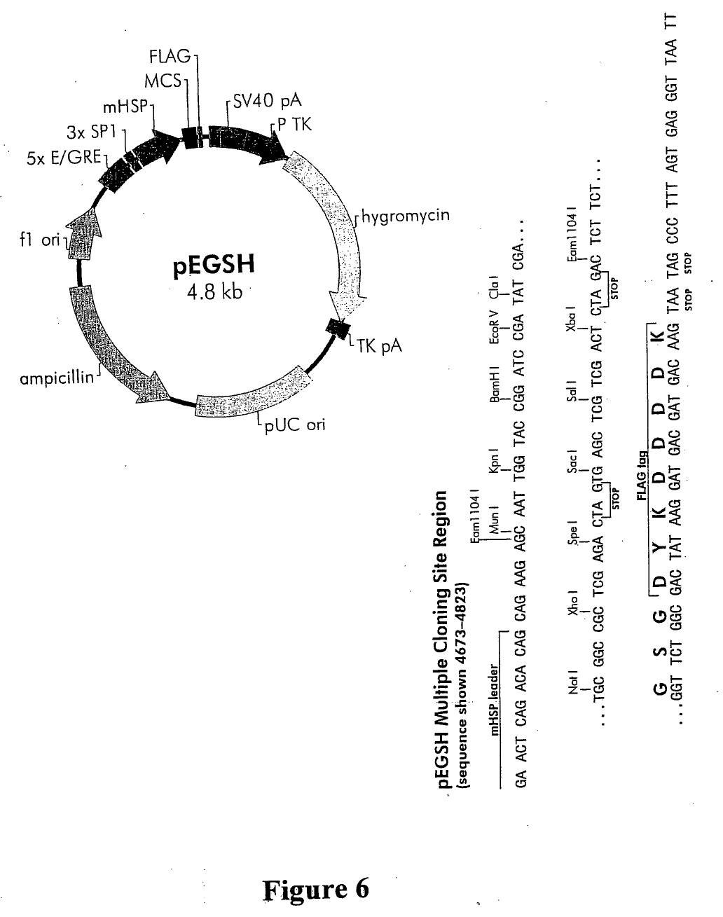

The plasmid shown in FIG. 2, pLS-RAS, contains a promoter enhancer from the hepatitis B virus driving transcription of an activated H-Ras and an ecdysone inducible promoter driving a dominant negative H-Ras. The Ras containing plasmids can be obtained from Upstate, Inc. Both the activated Ras and the dominant negative Ras plasmids can be digested with BglII and BamHI to remove the CMV promoter enhancer. Sequences corresponding to nucleotides 1610 to 1810 in the human hepatitis B virus can be isolated via PCR amplification from pEco63 (ATCC). This segment can be ligated into the BglII/BamHI cut, activated Ras containing plasmid to create pHBV-Ras (FIG. 2). The sequence corresponding to the ecdysone inducible promoter of pEGSH (Stratagene, under license from Salk Institute), when desired to be part of the construct, can be obtained by PCR amplification and ligated into the BglII/BamHI cut, dominant negative Ras containing plasmid to create pEcdys-Ras (FIG. 2).

The sequences containing the ecdysone inducible promoter, the dominant negative Ras and the polyA addition site can be amplified from pEcdys-Ras by PCR. The plasmid pLS-Ras can be constructed by blunt end ligating the PCR amplification product into pHBV-Ras linearized between the ampicillin resistance gene and the HBV promoter/enhancer by SspI digestion.

(2) Cell Culture

The human EG cell line ACTEG1 can be cultured on mouse STO feeder layers in KnockOut DMEM, 15% Knockout serum substitute (both from Invitrogen) supplemented with glutamine, mercaptoethanol, nonessential amino acids, forskolin or LIF, basic fibroblast growth factor and leukemia inhibitory factor as described for other EG cell lines (U.S. Pat. Nos. 5,690,926; 5,670,372, and 5,453,357, de Miguel and Donovan, (2002) Meth. Enzymol. 365, 353-363). Isolation of specific cell lines from EG cell lines can be achieved by transfecting pHBV-aRAS into ACTEG1 (A human gonadal ridge derived stem cell which is a pluripotent stem cell) via electroporation. Colonies can be selected for G418 resistance on Matrigel plates. ACTEG-RAS will be selected for further study.

To induce differentiation, cells can be removed from the Matrigel coated plates and aggregates can be formed via hanging drop culture. After two days, embryoid bodies can be collected and re-plated in Petri dishes that are not coated for cell culture. Cultures can be re-fed every two days. On day twelve, EBs can be collected, suspended in soft agar containing Amphioxus Cell Technologies Med3 with 5% defined calf serum. Within one week, colonies can be visible in the agar. Colonies can be picked, dispersed into Med3, 5% serum and plated into 24 well plates. Transformed colonies can form from most embryoid bodies. These colonies can be positive for markers of hepatocyte differentiation such as albumin, cyp1A, and cyp3A.

Medium from confluent cultures can be assayed for human albumin production. Cells can be trypsinized and counted using a hemocytometer. Cells can then be suspended in sufficient cell culture medium such that the density of the cells in the suspension is approximately three cells per milliliter. This suspension can then be aliquoted into the wells of a 96 well plate, using 200 microliters per well. The resulting culture will have less than one cell per well. In this way, colonies that appear are known to have arisen from a single cell. This clonal population is then assured to have a homogeneous genetic background.

This same cloning step can be used to isolate cells of a particular cell type from a mixed population. If the colony arising in the soft agar is of mixed lineage, cloning the cells as described above will separate them into individual homogeneous populations. These clones can then be examined for the cell type off interest by any of a variety of mechanisms. A usual method is to measure a known secreted protein in the supernate of the culture. For example, albumin would be measured to assay for hepatocyte colonies. Other methods to identify specific cell types are visual examination of morphology, staining with an antibody specific to a protein produced by that cell type or measurement of a specific RNA produced by that cell type.

(3) Generation of Gene Switch Competent Line

To generate the gene switch competent line, ACTEG1 cells can be transfected with pERV3 (Stratagene Corp) to insert the ecdysone receptor using electroporation. The plasmid pERV3 (or pVgRXR from Invitrogen) encodes a hybrid ecdysone receptor that is necessary for expression of the ecdysone sensitive promoter. Colonies will be selected for hygromycin resistance on Matrigel coated plates. ACTEG1-Hyg1 can be chosen for further study. Colonies can be selected for Zeocin resistance on Matrigel coated plates if using pVgRXR). ACTEG1-Zeo1 can be chosen for further study. Apoptosis of the cell line after shutting off the transforming gene can be addressed. (Hilger, R A, et al., (2002) Onkologie 25, 511-518). The ecdysone promoter system can prevent apoptosis because the amount of dominant negative produced can be modulated or titrated using differing concentrations of hormone.

If pERV3 used then ACTEG1-Hyg1 can be transfected with pLS-Ras using electroporation. Colonies resistant to G418 can be selected and expanded. ACTEG1-HygNeo can be selected. If pVgRXR used then ACTEG1-Zeo1 can be transfected with pLS-Ras using electroporation. Colonies resistant to G418 can be selected and expanded. ACTEG1-ZeoNeo (AZN) can be selected.

To induce differentiation, cells can be removed from the Matrigel coated plates and aggregates can be formed via hanging drop culture. After two days, embryoid bodies can be collected and re-plated in Petri dishes that are not coated for cell culture. Cultures can be re-fed every two days. On day twelve, EBs can be collected, suspended in soft agar containing Amphioxus Cell Technologies Med3 with 5% defined calf serum. Within one week, colonies can be visible in the agar. Colonies can be picked, dispersed into Med3, 5% serum and plated into 24 well plates.

Medium from confluent cultures can be assayed for human albumin production. Colonies should be positive. Several cultures can be selected and cloned via limiting dilution in 96 well plates. Cell lines ACTHep1 through ACTHep6 can be grown to confluence in 75 cm² plates, trypsinized and frozen in a controlled rate freezer, then stored in liquid nitrogen vapor phase.

ACTHep1-6 can be further characterized. Individual vials can be thawed and plated in Med3, 5% serum as described above. Cells can be expanded, then plated at a density of 10,000 cells per well in a 96 well plate. After overnight incubation, medium can be changed to Med3, 5% serum plus 10 μM ponasterone A. Cells should stop growing over the next 24 hours and arrest at subconfluent densities. Cells are selected having the cuboidal appearance of hepatocytes with a prominent nucleus. Their identity as hepatocytes can be confirmed by albumin production, metabolism of ethoxyresorufin to resorufin (cyp1A activity), and formation of 6 beta hydroxy testosterone from testosterone (cyp3A activity) (Kelly, J H, Sussman, N L (2000) J. Biomol. Scr. 5, 249-253).

### 2. Example 2

### Identification of a Human Hepatocyte Cell Line Using CRE/lox Recombination to Revert

Identification of a human hepatocyte cell line using tissue specific expression of an activated transforming gene followed by Cre recombinase excision can be produced. Human gonadal derived stem cells can be transfected with a construct containing the human hepatititis B virus promoter/enhancer driving an activated H-RAS gene, flanked by loxP sites. Cell lines of the hepatocyte lineage can be isolated as described above. Cells can be transfected with a plasmid expressing Cre recombinase to excise the activated oncogene. Cre-recombinase treated cells should cease division and express markers of the differentiated hepatocyte such as albumin production, cyp1 and cyp3 expression.

a) Methods

(1) Plasmids

The hepatocyte specific selection plasmid, pHBV-aRas, described above can be used for construction of ploxHBV-aRas by insertion of synthetic loxP oligomers (SEQ ID NO:5 and 6. SspI can be used to linearize pHBV-aRas between the ampicillin resistance gene and the HBV promoter/enhancer. The oligomer 5′ ATT ATA ACT TCG TAT AAT GTA TGC TAT ACG AAG TTA T 3′ (SEQ ID NO:5) can be ligated in to reconstruct the Ssp1 site on the 5′ side. This plasmid can then be linearized with BbsI and the oligomer 5′ ATA ACT TCG TAT AAT GTA TGC TAT ACG AAG TTA TGA AGA C 3′ (SEQ ID NO:6) can be ligated in to reconstruct the BbsI site on the 3′ side. The resulting plasmid, ploxHBV-aRas is shown in FIG. 4.

(2) Cell Culture

The human EG cell line ACTEG-1 is cultured as described above. The plasmid ploxHBV-aRas can be transfected into ACTEG-1 using electroporation and colonies will be selected using G418 resistance.

Hepatocyte colonies can be isolated as described above after differentiation and selection in soft agar. Cell lines Heplox1 through Heplox6 can be expanded and frozen.

Heplox1 can be expanded. Cells can be plated at a density of 10,000 cells/cm² in Med3, 5% defined calf serum. The plasmid pBS185, containing the Cre recombinase gene under the control of the CMV promoter, can be introduced into Heplox1 by electroporation. Over two days, the bulk of the cells should cease division. The cultures will be assayed for albumin production, cyp1A and cyp3A activity as described above.

Excision of the ploxHBV-aRas is unlikely to be 100% efficient. With time in culture, colonies that have not excised the transforming plasmid should become apparent. Other strategies, such as secondary selection in gancyclovir, can be employed to gain a 100% selection of excised cells. The herpes simplex virus thymidine kinase gene confers sensitivity to gancyclovir on human cells. If the HSV-TK gene was included in the original selection plasmid, then cells retaining the plasmid would die in the presence of gancyclovir. By reversing the transformation using CRE recombinase, then culturing in gancyclovir, only cells that had deleted the ploxHBV-aRAS would survive. Transformation is reversible. Characteristics to be reviewed can be the arrest of cells at subconfluent densities, amplification of expression of liver specific characteristics. Measurement of cell division via PCNA and BrdU staining; Albumin ELIS A, ethoxyresorufin metabolism, dibenzylfluorescein metabolism can occur.

### 3. Example 3

### Identification of a Human Hepatocyte Cell Line Using a Temperature Sensitive Transforming Gene

Identification of a human hepatocyte cell line using a tissue specific promoter and expression of a temperature sensitive transforming gene can be performed. Human gonadal derived pluripotent stem cells can be transfected with a plasmid containing the human hepatitis B virus promoter driving a temperature sensitive, activated RAS gene (SEQ ID NO:7) (DeClue et al., (1991) Mol. Cell. Biol. 11, 3132-3138). After differentiation of embryoid bodies at 37° C. for twelve days, the colonies can be dispersed in soft agar and incubated at 32° C. Cells of the hepatocyte lineage can be isolated as described above. When cultures of these cells are replated and shifted to 39° C., they cease division and express markers of the human hepatocyte such as albumin, cyp1A and cyp3A.

a) Methods

(1) Plasmids

Serine39 of the aRAS can be mutated to a Cys39 by oligonucleotide directed mutagenesis (Promega). Activated RAS can be excised from pHBV-aRAS by EcoRI and subcloned into the selectable plamid pALTER1. The oligonucleotide 5′-GAATACGACCCCACTATAGAGGATTGCTACCGGAAGCAGGTGGTCATTGAT-3′ can be used to change Serine 39 to Cysteine 39 (SEQ ID NO:8). The appropriate plasmid will be rescued via antibiotic selection and sequenced across the insert to insure accuracy. The mutated aRAS, now termed tsaRAS, will be excised from the pALTER plasmid with EcoR1 and inserted into EcoR1 cleaved pHBV-aRAS to generate pHBV-tsaRAS.

(2) Cell culture

The human gonadal ridge derived pluripotent stem cell line ACTEG-1 can be cultured as described above. The plasmid pHBV-tsaRAS can be transfected using electroporation and G418 resistant colonies can be selected. After differentiation as described above, soft agar plates can be incubated at 32° C. for isolation of transformed human hepatocytes lines. ACTtsHep1 though 6 can be isolated, cloned and frozen. ACTtsHep1 can be chosen for futher characterization. Cells cultured at 32° C. can be trypsinized and plated at 10,000 cells/cm², then incubated at 39° C. Cells cease division within two days, arrest at subconfluent densities and express markers of the human hepatocyte such as albumin, cyp1A and cyp3A.

Multiple cell types can be selected using tissue specific expression of reversible transforming genes. Isolation of several other cell types using RAS or some other transforming gene can be achieved. Analysis of isolated cells can include analyzing expression of markers characteristic of the cell type under selection.

### 4. Example 4

### Culture of the One of the Hepatocyte Lines Disclosed Herein in Hollow Fiber Bioreactors to Form the Basis of a Liver Assist Device

a) Methods

ACTHep1 and ACTtsHep1 can be cultured in hollow fiber bioreactors essentially as described for culture of the Amphioxus Cell Technologies human liver cell line HepG2/C3A (Sussman et al, Hepatology 16, 60-65, 1992. Briefly, cells are cultured in roller bottles using serum containing medium. Two bottles of cells containing about 1 g of cells each, are tryspinized, suspended in 50 ml of medium and inoculated into the extracapillary side of a hollow fiber cartridge. These cartridges are maintained in an automated system such as the Cellex Maximizer system. After inoculation, these cartridges are cultured in a serum free, insulin containing medium for approximately two weeks, during which time they multiply to fill the culture space. Glucose consumption and albumin production are monitored daily, peaking at about 12 g of glucose consumption and the production of over 1 gram of human albumin per day (Kelly, (1997) IVD Technology 3, 30-37).

Using HepG2/C3A in these devices, their ability to replicate liver specific biochemistry has been extensively characterized. Similar analysis on devices filled with the ACTHep1 and ACTtsHep1 cell lines can be performed. These studies will begin with the basics such as growth curves and medium consumption rates. One can determine how similar they are to the tumor derived line. For example, HepG2/C3A can be maintained in these devices essentially indefinitely. It is clear that with the tumor derived line, there was a certain steady state established where cell death was replaced by new cells. The amount of ACTHep1 cells needed to achieve a steady state can be determined and new cells can be added since the cells are not transformed and will not divide indefinitely in the device after reversion. The ability of these devices to metabolize ammonia via urea production, to metabolize drugs such as lidocaine, caffeine and midazolam, to synthesize glucose from pyruvate and lactate and to produce serum proteins, such as albumin, transferrin and factor IX can be determined.

### 5. Example 5

### Production of a Panel of Matched Lines Comprising Multiple Tissue Types for Use in Toxicology Testing

a) Methods

The plasmids constructed above can form the basis for the selection of new cell lines. Tissue specific promoter/enhancers can be chosen for the appropriate tissue then spliced into the plasmids in place of the HBV sequences. The tissues that can be represented include, for example, liver, kidney, heart, brain, muscle and intestine. Where multiple cell type are involved, such as the brain, several lines will be selected such as neuron, oligodendrocyte, etc. Each of these cell line can, for example, be produced from the same pluripotent cell line, e.g. human EG cell line ACTEG1 as described above. Thus, the panel of cells can have the same genotype providing multiple advantages.

### 6. Example 6

### Production of In Vitro Immune System (IVIS)

Monoclonal antibody (MAB) technology was developed by Kohler and Milstein over twenty five years ago (Kohler and Milstein, (1975) Nature 256, 495-497). Nonetheless, there are still relatively few MABs in therapeutic use. The main problem is that mouse monoclonal antibodies are recognized as foreign and so have a short useful lifetime as a therapeutic. MABs that are currently on the market are “humanized” by introduction of mutations into the antibody gene that substitute amino acids found in human antibodies for those of the mouse.

The production of fully human monoclonal antibodies has been hindered by several problems. Mouse monoclonal antibodies are produced by injecting an antigen into the mouse then removing its spleen several days later for fusion with a mouse myeloma for immortalization. Injection of antigen into humans is not generally feasible and has failed in the few instances where it has been attempted. Additionally, technology currently prevents removing a person's spleen and so one needs to use peripheral blood cells. Finally, suitable human myelomas have been very difficult to isolate.

IVIS will circumvent these problems by moving the entire human antibody production system into the test tube. Starting with a stem cell as discussed herein, such as a pluripotential embryonic stem cell or EG cell, matched T cell, B cell and macrophage lines can be developed. The B and T cells can be chosen to be at the appropriate stage of differentiation to be primed with the antigen. Because the three cell lines will have been developed from the same parental line, they will have an identical genetic background, exactly analogous to a person's own immune system. The cells can recognize each other and behave in the complex, cooperative way that stimulates B cell proliferation and antibody synthesis. Since the isolation procedure conditionally immortalizes the B cell, the antibody producing cell can be isolated then grown in any quantity necessary, from lab to production scale.

a) Methods

(1) Plasmids

Each of the necessary plasmids can be constructed from pLS-RAS, containing the activated ras and the dominant negative ras. To select for B cells, pB-RAS can be constructed by first excising the HBV promoter/enhancer using BamHI. The human immunoglobulin heavy chain promoter can be ligated into the site to form pB-RAS. Similar constructs can be made using the preT cell promoter to select for T cells (pT-RAS) and using the human CHI 3L1 gene promoter to select for macrophages. The bone marrow stromal cell line, needed for directed differentiation of B, T and macrophage lines, cam be selected using the promoter from the bone marrow stromal cell antigen 1 (BST1) gene.

(2) Bone Marrow Stromal Cell Selection

The BST1 promoter can be ligated into Bam/BglII cut pLS-RAS to make pBST-RAS. This can be transfected into ACTEG-1 and differentiation can be triggered via EB formation. The resulting bone marrow stromal cell line, ACT-BMST1, arising after day 5 of EB formation (Kramer et al, Meth. Enzymol. 365, 251-268, 2003), can be characterized by expression of BST1.

(3) B Cell Selection

B cells can be developed from ACTEG-1. The plasmid pB-RAS can be transfected into the stem cells as described above. B cell differentiation from the transfected stem cell line can be initiated as described (Cho, S K, Zuniga-Pflucker, J C Meth. Enzymol. 365, 158-169, 2003). The human ACT-BMST1 can be substituted for the mouse OP9 stromal line. The human Ig heavy chain promoter can select for a B cell at any stage of development. Several lines will be characterized for Ig light chain production to isolate a B cell of the appropriate developmental stage.

(4) T Cell Selection

T cells can be developed from ACTEG-1 by transfection of a plasmid containing the promoter of the preT cell receptor. After isolation of this stem cell line, differentiation of T cells can be carried out as described (Schmitt et al. Nat. Immunol. 5, 410-417, 2004). ACT-BMST 1 can be substituted for the mouse OP9 stromal line. Mature T cells can be characterized by the expression of CD4 and CD8 antigens.

(5) Macrophage Selection

A human macrophage line can be developed from ACTEG-1 by transfection of a plasmid containing the promoter for the CHI 3L1 gene driving ras. Macrophage colonies are abundant in day 6 embryoid bodies (Kennedy and Keller, Meth. Enzymol. 365, 39-59, 2003).

(6) In Vitro Immune System

Each of the individual lines can be cloned, characterized and frozen. The immortalized and matched B, T and macrophage lines can be cultured on the matched ACT-BMST1 line in 24 well plates. Antigen cam be added along with the fresh cell culture medium every three days for two weeks. At that time, and for two weeks longer, supernate can be assayed for the presence of antigen specific antibody by enzyme linked immunoassay. After antibody has been detected, the individual cells in the well can be diluted and cloned. Once established, antibody production from each B cell clone can continue. Clones expressing the appropriate antigen can be frozen for further characterization or production.

### 7. Example 7

### Establishment of the Human Embryonic Germ Cell Line Hay1

Using the techniques defined by Matsui, et al. ((1992) Cell 70, 841-847), a human EG line was established. Briefly, the gonadal ridges were dissected from a 10 week male fetus, dissociated with trypsin-EDTA and plated onto irradiated STO feeder layers. Cells were fed daily with DMEM, 15% fetal bovine serum, supplemented with non-essential amino acids and □-mercaptoethanol, 60 ng/ml human Stem Cell Factor (SCF), 10 ng/ml human Leukemia Inhibitory Factor (LIF) and 10 ng/ml human basic Fibroblast Growth Factor (FGF). On day 5, one of the two flasks was stained for alkaline phosphatase. Many positive cells were observed. Cells were passaged with trypsin-EDTA on day 6 and split 1 to 4 onto fresh irradiated STO layers. This process was repeated, following alkaline phosphatase at each passage. At passage 5, several vials of cells were frozen in DMEM, 15% fetal bovine serum, 10% dimethylsulfoxide, using a controlled rate freezer. Cells are routinely passaged now on mitomycin C treated STO layers.

a) Characteristics of Hay1

Hay1 cells, both on feeder layers and on plastic, as described below, grow as elongated cells resembling migratory primordial germ cells (Shamblott et al. (1998) Proc. Natl. Acad. Sci. 95, 13726-13731; Turnpenny et al. (2003) Stem Cells 21, 598-609). Hay1 displays morphology identical to the cells described by Tumpenny, et al. In addition to alkaline phosphatase, the cells stain positively for SSEA-1, TRA 1-60 and TRA 1-80. It is characteristic of human EG cells, unlike human ES cells, to express SSEA-1. Determination of karyotype and multi-tissue tumor formation is underway. When switched to low adherence plastic in the absence of feeders or hormone supplements, they readily form cystic embryoid bodies. When these embryoid bodies are re-plated in tissue culture plastic, the cells exhibit dramatically different morphology and lose expression of alkaline phosphatase.

b) Culture of Hay1 in Defined Conditions

The use of feeder layers complicates the use of stem cells for a variety of applications. Use of feeder layers dramatically raise the background in standard in vitro toxicology assays, such as MTT or resazurin reductions confounding the results. Hay1 can be grown routinely under defined conditions. Standard medium consists of KO-DMEM, 15% KO-serum replacement, glutamine, nonessential amino acids, β-MeSH, 10 ng/ml oncostatin M, 10 ng/ml SCF and 25 ng/ml bFGF. Using this medium, Hay1 continues to express the markers listed above and doubles approximately every three to four days. This is slightly slower than their doubling on feeder layers.

c) Hay1 Expresses Oct 4 and Nanog

While surface markers and alkaline phosphatase are convenient markers for stem cells, it has become clear that expression of the transcription factors Oct 4 and Nanog are fundamental characteristics of stem cells (Rodda et al. (2005) J. Biol. Chem. 280, 24731-24737; Chambers et al. (2003) Cell 113, 643-655). Hay1 was examined for expression of these factors using real time RT-QPCR. Expression of cells under standard defined conditions was compared to that in cells that have been subjected to differentiation via EB formation followed by culture in Med3 (Kelly and Sussman, (2000) J. Biomol. Screen. 5, 249-254), a medium that is a mixture of Weymouth's MAB, Ham's F12 and William's E. It also contains 5% defined calf serum (Hyclone). Actin was used as a standard. The results show that both Oct 4 and Nanog are expressed in Hay1 and that expression falls dramatically upon differentiation.

d) Hay1 is Dependent on gp130 Signaling for Growth

Growth of Hay1 was examined under various conditions known to affect stem cell growth and differentiation. Mouse and human EG cells require a source of gp130 signaling for growth in culture (Shamblott et al. (1998); Koshimuzu et al. (1996) Development 122, 1235-1242). When each of the three peptide hormone factors (Onc M, SCF, bFGF) was removed individually from the medium, each had some effect on growth. However, removal of oncostatin M completely arrested the growth of the cultures and they became alkaline phosphatase negative within several days.

e) FGF Induces Oct 4 and Nanog

Removal of FGF from the culture had a slight negative effect on growth of the culture and an effect on morphology, with the cells becoming flatter and more spread out on the dish. Cultures were examined for Oct 4 and Nanog expression after FGF withdrawal and a dramatic reduction in expression was observed. Replacement of FGF returned Oct 4 expression to its former level. Since Oct 4 controls Nanog expression (Rodda et al. (2005)), it was expected that induction of Oct 4 would also raise nanog, and this is what was observed.

f) Zeocin Sensitivity

In preparation for the establishment of the frt insert line, the sensitivity of Hay1 to zeocin was tested. A standard titration curve indicated that a concentration of 75 μg/ml will be an effective selection concentration.

### 8. Example 8

### Derivation of Cardiomyocytes

a) Creation of frt Insertion (FI) Cell Line FI Hay1

The plasmid pFrt/lac/Zeo (Invitrogen) can be transfected into Hay1 using Lipofectamine 2000. After 48 hrs, resistant cells can be selected by changing to medium containing 75 μg/ml Zeocin (Invitrogen). Non-resistant cells are dead in about seven days. An efficiency of about 1×10⁻⁵/μg is expected. Approximately ten individual transfectants can be selected and tested for expression of lacZ. Copy number of the plasmid can be evaluated via Southern blotting. Transfectants with single insertions can be chosen for further analysis. To examine the behavior of the insert during differentiation, cells can be subjected to EB formation, followed by culture in Med3, 5% defined calf serum for one week. They can be reevaluated for lacZ expression. Since Zeo selection can be maintained, it is expected that all surviving cells will retain lacZ expression. It is a general strategy to maintain selective pressure on the inserts to insure expression of the surrounding DNA, as has been successfully employed in a number of other studies (Zweigerdt et al., (2001) Cytotherapy 5, 399-413; Liu et al. (2004) Stem Cells Dev. 13, 636-645; Schuldiner et al., (2003) Stem Cells 21, 257-265).

The ten clones can then be evaluated for their insertion site. The ideal clone will have incorporated the DNA into some redundant or non functional segment of the genome. While in the end this may be a somewhat subjective evaluation, it is important that the site not be incorporated into a functioning gene that might interfere with later isolation of differentiated clones. DNA can be isolated from the cells and the inserted DNA, along with some surrounding sequences, can be recovered by plasmid rescue and sequenced (Organ et al., (2004) BMC Cell Biology 5, 41). The site of incorporation can be determined by comparison with human sequence databases.

b) Creation of Tetracycline Operator frt Insertion Cell Line TOFI Hay1

The cell line produced as described above can be transfected with pcDNA6/TR© (Invitrogen) using Lipofectamine as described above and selected for blasticidin resistance. This plasmid expresses the tetracycline repressor under the control of the CMV promoter. Multiple clones can be evaluated for continued expression under selective pressure as described above. As above, the insertion site can be evaluated to choose an appropriate clone for further evaluation.

The efficiency of the frt insertion cloning can be evaluated using pcDNA5/Frt/TO/CAT, a control plasmid supplied with the kit. The plasmid pcDNA5/Frt/TO (Invitrogen) is the frt targeting plasmid to be used in later selection studies. It contains a cloning site immediately 3′ of a tetracycline regulated CMV promoter. Chloramphenicol acetyl transferase (CAT) has been inserted into this plasmid to serve as a control. Plasmid pcDNA/Frt/TO/CAT can be cotransfected into the TOFI Hay1 line along with pOG44 (Invitrogen) to transiently express the flp recombinase. The frt-CAT plasmid will target the frt insertion site in TOFI Hay1, recombine and incorporate. The insertion is arranged such that it disrupts the Zeo resistance gene but carries with it hygromycin resistance. Successfully targeted clones will be hygromycin and blasticidin resistant but Zeo sensitive.

The efficiency of frt mediated recombination can be evaluated by examining the number of hygromycin resistant, blasticidin resistant clones that are obtained per microgram of pcDNA/Frt/TO/CAT. The efficiency of expression of the inserted CAT gene can be evaluated using the differentiation protocol described above. Two variations of the protocol can be carried out, one with tetracycline present throughout the procedure, one where tetracycline is added only after differentiation has occurred.

c) Construction of Selector Plasmid

The selector plasmids can be constructed using the Multisite Gateway three fragment vector construction system from Invitrogen (Hartley et al., (2000) Genome Res. 10, 1788-1795). This system uses site specific lambda integrase sequences and proteins to clone and recombine fragments in an ordered sequence. Activated ras and dominant negative ras were obtained from Upstate Biotechnology. Specific primers incorporating the lambda integrase sites can be used to amplify the a-ras and dn-ras sequences. These will then be cloned into specific plasmids in the kit using the integrase system.

Sequences extending from −454 to +32 of the human α-MHC promoter have been shown to direct high level, tissues specific expression (Yamauchi-Takihara et al. (1989) Proc. Natl, Acad. Sci. 86, 3504-3508; Sucharov et al. (2004) Mol. Cell. Biol. 24, 8705-8715). This sequence, along with the integrase sites, can be cloned into the third plasmid in the Multisite Gateway kit. These sequences can then be recombined into a fourth plasmid to create a clone with the gene order “dn-ras—α-MHC promoter—a-ras”.

Sequences extending from the dn-ras across the promoter to the end of the a-ras gene can be amplified via PCR and cloned into pcDNA5/Frt/TO using topoisomerase cloning to generate the selector plasmid ready for insertion into the frt recombination site in TOFI Hay1 site. This is termed the cardiac selector plasmid.

d) Creation of Cardiac Selective Stem Cell Line

The cardiac selector plasmid can be transfected into TOFI Hay1, along with pOG44 to transiently express the flp recombinase. As mentioned above, recombination into the frt site inserts a hygromycin resistance gene and disrupts Zeocin resistance. Appropriate recombinants will be blasticidin resistant, hygromycin resistant and Zeo sensitive. Clones can be selected in blasticidin/hygromycin then tested for Zeocin sensitivity. Plasmid rescue and sequencing can be used to verify that the correct DNA sequence has been constructed. This cell should now have an insert of the gene order “CMV Promoter—TO Regulated Repressor—dn-ras—α-MHC Promoter—a-ras.” The cell line can be termed Hay1-cardio.

e) Identification and Cloning of Cardiomyocyte Cell Line

Differentiation can be initiated in Hay1-cardio by formation of embryoid bodies in Med3, 5% defined calf serum plus hygromycin/blasticidin. After four days, the embryoid bodies can be placed back into tissue culture plastic for attachment and fed with the same medium. Patches of beating cells appear in such differentiating Hay1 approximately 14 days later. Cultures can be observed for appearance of beating areas but ras transformation of cardiomyocytes has been shown to block beating (Engelmann et al. (1993) J. Mol. Cell. Cardiol. 25, 197-213). Matched cultures of TOFI Hay1 without the selector can be carried along in parallel as indicators of the onset of cardiac differentiation.

When cardiac differentiation is detected in the cultures, cells can be trypsinized and plated into soft agar, made up in the same Med3 based medium. Control experiments with other a-ras transformed lines suggest that colonies should be identifiable within one week. Colonies can be picked, dispersed into fresh medium and re-plated in tissue culture plastic. Cells can be analyzed for expression of cardiomyocyte specific markers, such as authentic α-MHC, as well as expression of a-ras.

f) Reversion to “Normal” Cardiomyocytes

Addition of 1 μg/ml tetracycline to the medium will release the tetracycline repressor and activate transcription of the dn-ras. Exploratory experiments can be used to determine the effect of the dn-ras and the appropriate amount of tetracycline to add to the cultures in order to reverse the transformation but not kill the cells or disrupt cardiac function. A clear indicator of the appropriate regulation will be the onset of synchronized beating within the cultures.

### H. REFERENCES

Gearhart, J (1998) New potential for human embryonic stem cells. Science 282, 1061-1062.
Pera, M F, Reubinoff, B, and Trounson, A (2000) Human embryonic stem cells. J. Cell Sci. 113, 5-10.
Trounson, A (2001) Human embryonic stem cells: mother of all cell and tissue types. Reprod Fertil Dev. 2001; 13(7-8):523-32.
Zambrowicz, B P, Sands, A T (2003) Knockouts model the 100-best selling drugs—will they model the next 100? Nat. Rev. Drug Disc. 2, 38-51.
Andrew, P W (2002) From teratocarcinomas to embryonic stem cells. Philos. Trans. R. Soc. Lond. B. Biol. Sci. 357, 405-417.
Gilbert, S F. (1994) DEVELOPMENTAL BIOLOGY, 4^{th} Ed. Sinauer Associates, Inc. Sunderland, Mass., p 354.
Hogan, B L M, Costantini, F, Lacy, E. (1986) MANIPULATING THE MOUSE EMBRYO, Cold Spring Harbor Laboratory Press, Cold Spring Harbor, N.Y., 332p.
Mintz, B, Illmensee, K. (1975) Normal genetically mosaic mice produced from malignant teratocarcinoma cells. Proc. Natl. Acad. Sci. 72, 3585-3589.
Evans, M J, Kaufman, M H (1981) Establishment in culture of pluripotential cells from mouse embryos. Nature 292, 154-156.
Martin, G R (1981) Isolation of a pluripotent cell line from early mouse embryos cultured in medium conditioned by teratocarcinoma stem cells. Proc. Natl. Acad. Sci. 78, 7634-7638.
Misra, R P, Duncan, S A (2002) Gene targeting in the mouse: advances in introduction of transgenes into the genome by homologous recombination. Endocrine 19, 229-238.
Matsui, Y, Zsebo, K, Hogan, B L M (1992) Derivation of pluripotential embryonic stem cells from murine primordial germ cells in culture. Cell 70, 841-847.
Labosky, P A, Barlow, D P, Hogan B L M (1994) Mouse embryonic germ (EG) cell lines: transmission through the germline and differences in the methylation imprint of insulin-like growth factor 2 receptor (Igfr2) gene compared with embryonic stem (ES) cell lines. Development 120, 3197-3204.
Thomson, J A, Itskovitz-Eldor, J, Shapiro, S S, Waknitz, M A, Swiergiel, J J, Marshall, V S, Jones, J M. (1998) Embryonic stem cell lines derived from human blastocysts. Science 282, 1145-1147.
Shamblott, M J, Axelman, J, Wang, S, Bugg, E M, Littlefield, J W, Donovan, P J, Blumenthal, P D, Huggins, G R, Gearhart, J D. (1998) Derivation of pluripotent stem cells from cultured human primordial germ cells. Proc. Natl, Acad. Sci. 95, 13726-13731.
Kyba, M, Perlingeiro, R C R, Daley, G Q (2003) Development of hematopoietic repopulating cells from embryonic stem cells. Meth. Enzymol. 365, 114-129.
Fairchild, P J, Nolan, K F, Waldmann, H. (2003) Probing dendritic cell function by guiding the differentiation of embryonic stem cells. Meth. Enzymol. 365, 169-186.
Wassarman, P M, Keller, G M. (2003) METHODS IN ENZYMOLOGY, Differentiation of Embryonic Stem Cells, vol. 365, Elsevier Academic Press, New York, N.Y., 510p.
Alberts, B, Bray, D, Lewis, J, Raff, M, Roberts, K, Watson, J D. (1994) MOLECULAR BIOLOGY OF THE CELL, 3^{rd} Ed., Garland Publishing, New York, N.Y., 1294p.
Kelly, J H, Darlington, G J. (1985) Hybrid genes: molecular approaches to tissue specific gene regulation. Ann. Rev. Gen. 19, 273-296.
Pinkert, C A, Ornitz, D M, Brinster, R L, Palmiter, R D. (1987) An albumin enhancer located 10 kb upstream functions along with its promoter to direct efficient, liver-specific expression in transgenic mice. Genes Dev. 3, 268-76.
Downward, J. (2002) Targeting RAS signalling pathways in cancer therapy. Nat. Rev. Cancer 3, 11-22.
Fiordalisi, J J, Holly, S P, Johnson, R L, Parise, L V, Cox A D. (2002) A distinct class of dominant negative Ras mutants: cytosolic GTP-bound Ras effector domain mutants that inhibit Ras signaling and transformation and enhance cell adhesion. J Biol. Chem. 29, 10813-23.
Barone, M V, Courtneidge, S A. (1995) Myc but not fos rescue of PDGF signaling block by kinase inactive src. Nature. 1995 Nov. 30; 378(6556):509-12
Willis A, et al., Mutant p53 exerts a dominant negative effect by preventing wild-type p53 from binding to the promoter of its target genes, Oncogene. 2004 Mar. 25; 23(13):2330-8.
Jat, P S, Noble, M D, Ataliotis, P, Tanaka, Y, Yannoutsos, N, Larsen, L, Kioussis, D. (1991) Direct derivation of conditionally immortal cell lines from an H-2 Kb-tsA58 transgenic mouse. Proc. Natl. Acad. Sci. 88, 5096-5100.
Fahnestock, M L, Lewis, J B. (1989) Limited temperature sensitive transactivation by adenovirus type 2 E1a proteins. J. Virol. 63, 2348-2351.
Sauer. B. (2002) Cre/lox: one more step in the taming of the genome. Endocrine 19, 221-228.
Schaft, J, Ashery-Padan, R, van der Houven, F, Gruss, P, Stewart, A F. (2001) Efficient FLP recombination in mouse ES cells and oocytes. Genesis 31, 6-10.
Sandig, V, Loser, P, Lieber, A, Kay, M A, Strauss, M. (1996) HBV-derived promoters direct liver-specific expression of an adenovirally transduced LDL receptor gene. Gene Therapy 3, 1002-1009.
Saez, E, Nelson, M C, Eshelman, B, Banayo, E, Koder, A, Cho, G J, Evans, R M. (2000) Identification of ligands and coligands for the ecdysone-regulated gene switch. Proc. Natl. Acad. Sci. 97, 14512-14517.
de Miguel, M P, Donovan, P J. (2002) Isolation and culture of embryonic germ cells. Meth. Enzymol. 365, 353-363.
Kelly, J H, Sussman, N L (2000) A fluorescent cell-based assay for cyp1A2 induction and inhibition. J. Biomol. Scr. 5, 249-253.
DeClue, J E, Stone, J C, Blanchard, R A, Papageorge, A G, Martin, P, Zhang, K, Lowy, D R. (1991) A ras efffector domain mutant which is temperature sensitivie for cellular transformation: interactions wsith GTPase-activating protein and NF-1. Mol. Cell. Biol. 11, 3132-3138.
Hilger, R A, Scheulen, M E, Strumberg, D. (2002) The Ras-raf-mek-erk pathway in the treatment of cancer. Onkologie 25, 511-518.
Sussman, N L, Chong, M G, Koussayer, T, He, D, Shang, T A, Whisennand, H H & Kelly, J H. (1992) Reversal of fulminant hepatic failure using an extracorporeal liver assist device. Hepatology 16, 60-65.
Sussman, N L, Gislason, G T, Conlin, C A, & Kelly, J H. (1994) The Hepatix extracorporeal liver assist device: initial clinical experience. Artificial Organs 18, 390-396.
Millis, J M, Cronin D C, Johnson, R, Conjeevarum, H, Conlin, C, Trevino, S, & Maguire, P. (2002) Initial experience with the modified extracorporeal liver assist device for patients with fulminant hepatic failure: system modificiations and clinical impact. Transplantation 74, 1735-1746.
Hui, T, Rozga, J, & Demetriou, A A (2001) Bioartificial liver support. J. Hepatobiliary Pancreat Surg. 8, 1-15.
Sussman, N L & Kelly, J H. (1995) The Artificial Liver. Scientific American: Science and Medicine 2, 68-77.
Kelly, J H, Spiering, A L, Sussman, N L (1997) Pathogen free human serum protein production using a hollow-fiber bioreactor system. IVD Technology 3, 30-37.
van de Waterbeemd, H, Gifford, E. (2003) ADMET in silico modeling: towards prediction paradise. Nat. Rev. Drug Disc. 2, 192-204.
Suchard, J. (2001) Review: wherefore withdrawal, The science behind recent drug withdrawals and warnings. Int. J. Med. Toxicol. 4, 15-20.
Rambhatla, L, Chiu, C P, Kundu, P, Peng, Y, Carpenter, M K. (2003) Generation of hepatocyte-like cells from human embryonic stem cells. Cell Transplant. 12, 1-11.
Hogan, B L M. (1995) Pluripotential embryonic stem cells and methods of making same. U.S. Pat. No. 5,453,357.
Hogan, B L M. (1997) Pluripotential embryonic stem cells and methods of making same. U.S. Pat. No. 5,670,372.
Hogan, B L M. (1997) Pluripotential embryonic cells and methods of making same. U.S. Pat. No. 5,690,926.
Thomson, J A (1998) Primate embryonic stem cells. U.S. Pat. No. 5,843,780.
Gearhart, J D. (2000) Human embryonic pluripotent germ cells. U.S. Pat. No. 6,090,622.
Darlington, G J, Ross, S E, MacDougald, O A. (1998) the role of C/EBP genes in adipocyte differentiation. J. Biol. Chem. 273, 30057-30060.
Kelly, J H. (1994) Permanent human hepatocyte cell line and its use in a liver assist device (LAD). U.S. Pat. No. 5,290,684.
Sussman, N L, Kelly, J H. (1994) Organ support system. U.S. Pat. No. 5,368,555.
Macneish, J. (2004) Stem cells in drug discovery. Nat. Rev. Drug Disc. 3, 70-80.
U.S. Pat. No. 5,849,553 Mammalian multipotent neural stem cells.
U.S. Pat. No. 5,811,281 Immortalized intestinal epithelial cell lines.
U.S. Pat. No. 5,672,499 Immoralized neural crest stem cells and methods of making.
Wasserman, P. M., Keller, G. M. (2003) “DIFFERENTIATION OF EMBRYONIC STEM CELLS”. Methods Enzymol., Vol. 235, Elsevier Academic Press, Amsterdam.
Zuniga-Pflucker, J. C. (2004) T-cell development made simple. Nat. Rev. Immunol. 4, 67-72.
Li, H., Roblin, G., Liu, H, Heller, S. (2003) Generation of hair cells by stepwise differentiation of embryonic stem cells. Proc. Natl. Acad. Sci. 100, 13495-13500.
Mitalipova, M. M., et al. (2005) preserving the genetic integrity of human embryonic stem cells. Nat. Biotechnol. 23, 19-20.
Muraca, M., Neri, D., et al. (2002) Intraportal hepatocyte transplantation in the pig: hemodynamic and histopathological study. Transplantation 73, 890-896.
Matsui, Y., Zsebo, K., Hogan, B. L. (1992) Derivation of pluripotent embryonic stem cells from murine primordial germ cells in culture. Cell 70, 841-847.
Resnick, J. L., Bixler, L. S., Cheng, L., and Donovan, P. J. (1992) Long-term proliferation of mouse primordial germ cells in culture. Nature 359, 550-551.
Pettite, J. N., Liu, G., Yang, Z. (2004) Avian pluripotent stem cells. Mech. Dev. 121, 1159-1168.
Tsung, H. C., et al. (2003) The culture and establishment of embryonic germ (EG) cell lines from Chinese mini swine. Cell Res. 13, 195-202.
Shamblott, M. J., Axelman, J., et al. (1998) Derivation of pluripotent stem cells from cultured human primordial germ cells. Proc. Natl. Acad. Sci. 95, 13726-13731.
Turnpenny, L., Brickwood, S., et al. (2003) Derivation of human embryonic germ cells: an alternative source of pluripotent stem cells. Stem Cells 21, 598-609.
Branda, C. S., Dymecki, S. M. (2004) Talking about a revolution: the impact of site specific recombinases on genetic analyses in mice. Developmental Cell 6, 7-28.
Dymecki, S (1996) Flp recombinase promotes site specific DNA recombination in embryonic stem cells and transgenic mice. Proc. Natl. Acad. Sci. 93, 6191-6196.
Kelly, J. H., Darlington, G. J. (1985) Hybrid genes: molecular approaches to tissue specific gene regulation. Ann. Rev. Gen. 19, 273-296.
Asahina, K., Fujimora, H., et al. (2004) Expression of the liver specific gene cyp7A1 reveals hepatic differentiation in embryoid bodies derived from mouse embryonic stem cells. Genes Cells 9, 1297-1308.
Aubert, J., Stavridis, M. P. (2003) Screening for mammalian neural genes via fluorescence activated cell sorter purification of neural precursors from Sox 1-gfp knock-in mice. Proc. Natl. Acad. Sci. 100, Suppl. 1, 11836-11841.
Zweigerdt, R., Burg, M., Willbold, E., Abts, H. F., Ruediger, M. (2001) Generation of confluent cardiomyocyte monolayers derived from embryonic stem cells in suspension: a cell source for new therapies and screening strategies. Cytotherapy 5, 399-413.
Ray, M. K., Fagan, S. P., Brunicardi, F. c. (2000) The Cre-loxP system: a versatile tool for targeting genes in a cell and stage specific manner. Cell Transplant. 9, 805-815.
Lewandowski, M. (2001) Conditional control of gene expression in the mouse. Nat. Rev. Genet. 2, 743-755.
Downward, J. (2003) Targeting ras signaling pathways in cancer therapy. Nat. Rev. Cancer 3, 11-22.
Engelmann, G. L., et al. (1993) Formation of fetal rat cardiac cell clones by retroviral transformation: retention of select myocyte characteristics. J. Mol. Cell. Cardiol. 25, 197-213.
Sugden, P. H. (2003) Ras, Akt and mechanotransduction in the cardiac myocyte. Circ. Res. 93, 1179-1192.
Kelly, J. H., Sussman, N. L. (2000) A fluorescent cell based assay for cytochrome P450 isozyme 1A2 induction and inhibition. J. Biomol. Screen. 5, 249-254.
Rodda, D. J., et al. (2005) Transcriptional regulation of nanog by OCT4 and SOX2. J. Biol. Chem. 280, 24731-24737.
Chambers, I., et al. (2003) Functional expression cloning of Nanog, a pluripotency sustaining factor in embryonic stem cells. Cell 113, 643-655
Koshimuzu, U., et al. (1996) Functional requirement of gp130-mediated signaling for growth and survival of mouse primordial germ cells in vitro and derivation of embryonic germ (EG) cells. Development 122, 1235-1242.
Liu, Y. P., et al. (2004) Maintenance of pluripotentcy in human embryonic stem cells stably over-expressing enhanced green fluorescent protein. Stem Cells Dev. 13, 636-645.
Schuldiner, M., Itskovitz-Elder, J., Benvenisty, N. (2003) Selective ablation of human embryonic stem cells expressing a “suicide” gene. Stem Cells 21, 257-265.
Organ, E. L., Sheng, J., Ruley, H. E., Rubin, D. H. (2004) Discovery of mammalian genes that participate in virus infection. BMC Cell Biology 5, 41.
Hartley, J. L., Temple, G. F., Brasch, M. A. (2000) DNA cloning using in vitro site-specific recombination. Genome Res. 10, 1788-1795.
Yamauchi-Takihara, K., et al. (1989) Characterization of human cardiac myosin heavy chain genes. Proc. Natl, Acad. Sci. 86, 3504-3508.
Sucharov, C. C., et al. (2004) The Ku protein complex interacts with YY1, is up-regulated in human heart failure, and represses a myosin heavy chain gene expression. Mol. Cell. Biol. 24, 8705-8715.

Sequences. For SEQ ID NOs 9-23, references refer to the structure of the promoter. All actual sequences are from The University of California Santa Cruz Genome Bioinformatics website at: http://genome.ucsc.edu/index.html?org=Human&db=hg15&hgsid=34607112. SEQ ID NO:1 is human hepatitis B virus core promoter/enhancer. SEQ ID NO:2 is activated H-RAS gene. SEQ ID NO:3 is ecdysone inducible gene switch promoter. SEQ ID NO:4 is dominant negative H-RAS gene. SEQ ID NO:5 is used to construct Cre-lox site. SEQ ID NO:6 is used to construct the Cre-lox site. SEQ ID NO:7 is temperature sensitive, activated RAS gene. SEQ ID NO:8 is oligo to change Serine 39 to Cysteine 39 of activated ras. SEQ ID NO:9 is Adipocyte Human adiponectin gene sequences from −908 to +14. Iwaki, M., et al. Diabetes 52, 1655-1663, 2003. SEQ ID NO:10 is Human alpha-1-antitrypsin promoter sequences from −137 to −37. SEQ ID NO:11 is Human albumin gene sequences from −434 to +12. SEQ ID NO:12 is Human myosin light chain gene VLC1 sequences from −357-+40 Kurabayashi, M., et al. J. Biol. Chem. 265, 19271-19278, 1990. SEQ ID NO:13 is Human rhodopsin gene sequences from −176 to +70 plus 246 bp from −2140 to −1894, Nie, Z., et al. J. Biol. Chem. 271, 2667-2675, 1996. SEQ ID NO:14 is Human E selectin gene sequences from −547 to +33. Maxwell, 1H, et al. Angiogenesis 6, 31-38, 2003. SEQ ID NO:15 is Human preT cell receptor sequence from −279 to +5 plus upstream enhancer element. Reizis, B, P. Leder. J. Exp. Med., 194, 979-990, 2001. SEQ ID NO:16 is Human CHI 3L1 gene from −308-+2. Rehli, M., et al. J. Biol. Chem. 278, 44058-44067, 2003. SEQ ID NO:17 is Human uromodulin gene promoter sequences from −3.7 kb. Zbikowska, H M, et al. Biochem. J. 365, 7-11, 2002. SEQ ID NO:18 is Human glutamate receptor 2 gene (GluR2) sequences from −302 to +320 Myers, S J, et al. J. Neuroscience 18, 6723-6739, 1998. SEQ ID NO:19 is Human surfactant protein A2 (SP-A2) sequences from −296 to +13 Young, P P, C R Mendelson Am. J. Physiol. 271, L287-289, 1996. SEQ ID NO:20 is Human insulin gene sequences from −279. Boam, D S, et al. J. Biol. Chem. 265, 8285-8296, SEQ ID NO:21 is Human fast skeletal muscle troponin C gene sequences from −978 to +1 Gahlmann, R, L. Kedes J. Biol. Chem. 265, 12520-12528, 1990. SEQ ID NO:22 is Gabriela Kramer, M., et al. Molecular Therapy 7, 375-385. Human hepatitis B virus sequences from 1610 to 1810. SEQ ID NO:23 is B Cells Human immunoglobulin heavy chain promoter Staudt, L. M., Lenardo, M. J. Ann. Rev. Immunol. 9, 373-398, 1991 Gene name: IGH@ Genbank: None. SEQ ID NO:24 is Lox sequence, sequence left behind after recombination. SEQ ID NO:25 is frt sequence. SEQ ID NO:26 is pEGSH, 4829 bp. SEQ ID NO:27 is pERV3, 8433 bp.

TABLE 3

| | Gene | | Transcript | Genome | |
|---|---|---|---|---|---|
| Tissue Type | Abbrev. | Gene Name | Number | Location | Promoter Region |
| Adipocyte | ACDC | Adipocyte, C1Q and collagen | NM_004797.2 | Chr 3: 187.962-187.978 Mbp | http://genome.ucsc.edu/cgi-bin/hgc?hgsid= |
| | | domain containing | | (+) | 34522615&g= |
| | | | | | htcDnaNearGene&i= |
| | | | | | NM_004797&c= |
| | | | | | chr3&l= |
| | | | | | 187880375&r= |
| | | | | | 187898165&o= |
| | | | | | refGene&hgSeq.promoter= |
| | | | | | on&boolshad.hgSeq.promoter= |
| | | | | | 1&hgSeq.promoterSize= |
| | | | | | 5000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | COL6AI | Collagen, type VI, alpha 1 | NM_001848.1 | Chr 21: 46.258-46.281 Mbp | http://genome.ucsc.edu/cgi-bin/hgc?hgsid= |
| | | | | (+) | 34524523&g= |
| | | | | | htcDnaNearGene&i= |
| | | | | | NM_001063&c= |
| | | | | | chr3&l= |
| | | | | | 134745845&r= |
| | | | | | 134780246&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | COMP | Cartilage oligomericmatrix | NM_000095.2 | Chr 19: 18.738-18.747 Mbp | http://genome.ucsc.edu/cgi-bin/hgc?hgsid= |
| | | protein | | (−) | 34603833&g= |
| | | | | | htcDnaNearGene&i= |
| | | | | | NM_001442&c= |
| | | | | | chr8&l= |
| | | | | | 82113111&r= |
| | | | | | 82119635&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | FABP4 | Fatty acid binding | NM_001442.1 | Chr 8: 82.114-82.118 Mbp | http://genome.ucsc.edu/cgi-bin/hgc?hgsid= |
| | | protein4, adipocyte | | (−) | 34603921&g= |
| | | | | | htcDnaNearGene&i= |
| | | | | | NM_001442&c= |
| | | | | | chr8&l= |
| | | | | | 82113111&r= |
| | | | | | 82119635&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | FADS1 | Fatty acid desaturase 1 | NM_013402.3 | Chr 11: 61.817-61.835 Mbp | http://genome.ucsc.edu/cgi-bin/hgc?hgsid= |
| | | | | (−) | 34603932&g= |
| | | | | | htcDnaNearGene&i= |
| | | | | | NM_013402&c= |
| | | | | | chr11&l= |
| | | | | | 61816983&r= |
| | | | | | 61836195&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GPAM | Glycerol-3-phosphate | NM_020918.2 | Chr 10: 114.04-114.074 Mbp | http://genome.ucsc.edu/cgi-bin/hgc?hgsid= |
| | | acyltransferase, mitochondrial | | (−) | 34603949&g= |
| | | | | | htcDnaNearGene&i= |
| | | | | | NM_020918&c= |
| | | | | | chr10&l= |
| | | | | | 114039847&r= |
| | | | | | 114075744&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GPD1 | Glycerol-3-phosphate | NM_005276.2 | Chr 12: 50.214-50.221 Mbp | http://genome.ucsc.edu/cgi-bin/hgc?hgsid= |
| | | dehydrogenase 1 (soluable) | | (+) | 34603967&g= |
| | | | | | htcDnaNearGene&i= |
| | | | | | NM_005276&c= |
| | | | | | chr12&l= |
| | | | | | 50213547&r= |
| | | | | | 50222843&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LPL | Lipoprotein lipase | NM_000237.1 | Chr 8: 19.606-19.634 Mbp | http://genome.ucsc.edu/cgi-bin/hgc?hgsid= |
| | | | | (+) | 34603977&g= |
| | | | | | htcDnaNearGene&i= |
| | | | | | NM_000237&c= |
| | | | | | chr8&l= |
| | | | | | 19605081&r= |
| | | | | | 19635073&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MFAP5 | Microfibrillar associated | NM_003480.2 | Chr 12: 8.698-8.715 Mbp | http://genome.ucsc.edu/cgi-bin/hgc?hgsid= |
| | | protein 5 | | (−) | 34603991&g= |
| | | | | | htcDnaNearGene&i= |
| | | | | | NM_003480&c= |
| | | | | | chr12&l= |
| | | | | | 8697806&r= |
| | | | | | 8716700&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RBP4 | Retinol binding protein 4, | NM_006744.2 | Chr 10: 95.482-95.492 Mbp | http://genome.ucsc.edu/cgi-bin/hgc?hgsid= |
| | | plasma | | (+) | 34604016&g= |
| | | | | | htcDnaNearGene&i= |
| | | | | | NM_006744&c= |
| | | | | | chr10&l= |
| | | | | | 95481826&r= |
| | | | | | 95493223&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SCD | Stearoyl-CoA desaturase | NM_005063.3 | Chr 10: 102.238-102.255 Mbp | http://genome.ucsc.edu/cgi-bin/hgc?hgsid= |
| | | (delta-9-desaturase) | | (+) | 34604048&g= |
| | | | | | htcDnaNearGene&i= |
| | | | | | NM_005063&c= |
| | | | | | chr10&l= |
| | | | | | 102237106&r= |
| | | | | | 102256817&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Adrenal Gland | AADAC | Arylacetamide deacetylase | NM_001086.1 | Chr 3: 152.813-152.827 Mbp | http://genome.ucsc.edu/cgi-bin/hgc?hgsid= |
| | | (esterase) | | (+) | 34604278&g= |
| | | | | | htcDnaNearGene&i= |
| | | | | | NM_001086&c= |
| | | | | | chr3&l= |
| | | | | | 152812476&r= |
| | | | | | 152828885&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CYP11B1 | Cytochrome P450, family 11, | NM_000497.2 | Chr 8: 143.758-143.765 Mbp | http://genome.ucsc.edu/cgi-bin/hgc?hgsid= |
| | | subfamily B, polypeptide 1 | | (−) | 34604360&g= |
| | | | | | htcDnaNearGene&i= |
| | | | | | NM_000497&c= |
| | | | | | chr8&l= |
| | | | | | 143758681&r= |
| | | | | | 143766702&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CYP17A1 | Cytochrome P450, family 17, | NM_000102.2 | Chr 10: 104.721-104.728 Mbp | http://genome.ucsc.edu/cgi-bin/hgc?hgsid= |
| | | subfamily A, polypeptide 1 | | (−) | 34604080&g= |
| | | | | | htcDnaNearGene&i= |
| | | | | | NM_000102&c= |
| | | | | | chr10&l= |
| | | | | | 104720517&r= |
| | | | | | 104729404&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CYP21A2 | Cytochrome P450, family 21, | NM_000500.4 | Chr 6: 32.032-32.035 Mbp | http://genome.ucsc.edu/cgi-bin/hgc?hgsid= |
| | | subfamily A, polypeptide 2 | | (+) | 34604103&g= |
| | | | | | htcDnaNearGene&i= |
| | | | | | NM_000500&c= |
| | | | | | chr6&l= |
| | | | | | 32031087&r= |
| | | | | | 32036423&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GSTA2 | Glutathione S-transferase A2 | NM_000846.3 | Chr 6: 52.615-52.629 Mbp | http://genome.ucsc.edu/cgi-bin/hgc?hgsid= |
| | | | | (+) | 34604434&g= |
| | | | | | htcDnaNearGene&i= |
| | | | | | NM_000846&c= |
| | | | | | chr6&l= |
| | | | | | 52615576&r= |
| | | | | | 52630720&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HSD3B2 | Hydroxy-delta-5-steroid | NM_000198.1 | Chr 1: 119.104-119.112 Mbp | http://genome.ucsc.edu/cgi-bin/hgc?hgsid= |
| | | dehydrogenase, 3 beta- and | | (+) | 34604155&g= |
| | | steroid delta isomerase 2 | | | htcDnaNeatGene&i= |
| | | | | | NM_000198&c= |
| | | | | | chr1&l= |
| | | | | | 119103821&r= |
| | | | | | 119113700&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | STAR | Steroidogenic acute regulator | NM_000349.1 | Chr 8: 37.742-37.749 Mbp | http://genome.ucsc.edu/cgi-bin/hgc?hgsid= |
| | | | | (−) | 34604210&g= |
| | | | | | htcDnaNearGene&i= |
| | | | | | NM_000349&c= |
| | | | | | chr8&l= |
| | | | | | 38017537&r= |
| | | | | | 38026839&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Wholeblood | AIF1 | Allograft infloammatory factor 1 | NM_032955.1 | Chr 6: 31.643-31.642 Mbp | http://genome.ucsc.edu/cgi-bin/hgc?hgsid= |
| | | | | (+) | 34604590&g= |
| | | | | | htcDnaNearGene&i= |
| | | | | | NM_001623&c= |
| | | | | | chr6&l= |
| | | | | | 31641632&r31644642&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | AQP9 | Aquaporin 9 | NM_020980.2 | Chr 15: 56.009-56.057 Mbp | http://genome.ucsc.edu/cgi-bin/hgc?hgsid= |
| | | | | (+) | 34604619&g= |
| | | | | | htcDnaNearGene&i= |
| | | | | | NM_020980&c= |
| | | | | | chr15&l= |
| | | | | | 56008616&r= |
| | | | | | 56058247&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ARHGAP25 | Rho GTPase activating protein | ENST00000295381 | Chr 2: 68.919-69.011 Mbp | |
| | | 25 | | (+) | |
| | CCL5 | Chemokine (C—C motif) ligand 5 | NM_002985.2 | Chr 17: 34.047-34.056 Mbp | http://genome.ucsc.edu/cgi-bin/hgc?hgsid= |
| | | | | (−) | 34604667&g= |
| | | | | | htcDnaNearGene&i= |
| | | | | | NM_002985&c= |
| | | | | | chr17&l= |
| | | | | | 34046151&r= |
| | | | | | 34057034&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CDW52 | CDW52 antigen (CAMPATH- | NM_001803.1 | Chr 1: 25.877-25.88 Mbp | http://genome.ucsc.edu/cgi-bin/hgc?hgsid= |
| | | 1 antigen) | | (+) | 34604691&g= |
| | | | | | htcDnaNearGene&i= |
| | | | | | NM_001803&c= |
| | | | | | chr1&l= |
| | | | | | 25876528&r= |
| | | | | | 25881054&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | DPEP2 | Dipeptidase2 | NM_022355.1 | Chr 16: 67.756-67.769 Mbp | http://genome.ucsc.edu/cgi-bin/hgc?hgsid= |
| | | | | (−) | 34604726&g= |
| | | | | | htcDnaNearGene&i= |
| | | | | | NM_022355&c= |
| | | | | | chr16&l= |
| | | | | | 67755760&r= |
| | | | | | 67769820&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GNLY | Granulysin | NM_012483.1 | Chr 2: 85.879-85.883 Mbp | http://genome.ucsc.edu/cgi-bin/hgc?hgsid= |
| | | | | (+) | 34604755&g= |
| | | | | | htcDnaNearGene&i= |
| | | | | | NM_006433&c= |
| | | | | | chr2&l= |
| | | | | | 85878124&r= |
| | | | | | 85884591&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GPR86 | G protein-coupled receptor 86 | NM_023914.2 | Chr 3: 152.325-152.328 Mbp | http://genome.ucsc.edu/cgi-bin/hgc?hgsid= |
| | | | | (−) | 34604779&g= |
| | | | | | htcDnaNearGene&i= |
| | | | | | NM_053002&c= |
| | | | | | chr3&l= |
| | | | | | 152324706&r= |
| | | | | | 152329946&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ICAM3 | intercellular adhesion | NM_002162.2 | Chr 19: 10.289-10.295 Mbp | http://genome.ucsc.edu/cgi-bin/hgc?hgsid= |
| | | | | (−) | 34604808&g= |
| | | | | | htcDnaNearGene&i= |
| | | | | | NM_002162&c= |
| | | | | | chr19&l= |
| | | | | | 10288660&r= |
| | | | | | 10296509&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | IL8RB | interleuk in 8 receptor, beta | NM_001557.2 | Chr 2: 218.954-218.965 Mbp | http://genome.ucsc.edu/cgi-bin/hgc?hgsid= |
| | | | | (+) | 34604831&g= |
| | | | | | htcDnaNearGene&i= |
| | | | | | NM_001557&c= |
| | | | | | chr2&l= |
| | | | | | 218953767&r= |
| | | | | | 218966997&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LST1 | Leukocyte specific transcript 1 | NM_007161.2 | Chr 6: 31.612-31.615 Mbp | http://genome.ucsc.edu/cgi-bin/hgc?hgsid= |
| | | | | (+) | 34604866&g= |
| | | | | | htcDnaNearGene&i= |
| | | | | | NM_007161&c= |
| | | | | | chr6&l= |
| | | | | | 31611834&r= |
| | | | | | 31616550&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LYZ | Lysozyme (renal amyloidosis) | NM_000239.1 | Chr 12: 69.458-69.464 Mbp | http://genome.ucsc.edu/cgi-bin/hgc?hgsid= |
| | | | | (+) | 34604885&g= |
| | | | | | htcDnaNearGene&i= |
| | | | | | NM_000239&c= |
| | | | | | chr12&l= |
| | | | | | 69457910&r= |
| | | | | | 69465760&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MGAM | Maltase-Glucomamylase | NM_004668.1 | Chr 7: 141.026-141.136 Mbp | http://genome.ucsc.edu/cgi-bin/hgc?hgsid= |
| | | (alpha-glucosidase) | | (+) | 34604916&g= |
| | | | | | htcDnaNearGene&i= |
| | | | | | NM_004668&c= |
| | | | | | chr7&l= |
| | | | | | 141025099&r= |
| | | | | | 141137968&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MNDA | Myeloid cell nuclear | NM_002432.1 | Chr 1: 155.579-155.597 Mbp | http://genome.ucsc.edu/cgi-bin/hgc?hgsid= |
| | | differentiation antigen | | (+) | 34604938&g= |
| | | | | | htcDnaNearGene&i= |
| | | | | | NM_002432&c= |
| | | | | | chr1&l= |
| | | | | | 155578041&r= |
| | | | | | 155598144&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | NCF1 | Neutrophil cytosolic factor 1 | NM_000265.1 | Chr 7: 73.586-73.986 Mbp | http://genome.ucsc.edu/cgi-bin/hgc?hgsid= |
| | | (47 kDa, chronic granulomatous | | (+) | 34604966&g= |
| | | disease, autosomal 1) | | | htcDnaNearGene&i= |
| | | | | | NM_000265&c= |
| | | | | | chr7&l= |
| | | | | | 73969732&r= |
| | | | | | 73987046&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | NKG7 | natural killer cell group 7 | NM_005601.2 | Chr 19: 56.55-56.551 Mbp | http://genome.ucsc.edu/cgi-bin/hgc?hgsid= |
| | | sequence | | (−) | 34604988&g= |
| | | | | | htcDnaNearGene&i= |
| | | | | | NM_005601&c= |
| | | | | | chr19&l= |
| | | | | | 56549894&r= |
| | | | | | 56552910&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | NCR3 | natural cytotoxicity triggering | NM_147130.1 | Chr 6: 31.615-31.619 Mbp | |
| | | receptor 3 | | (−) | |
| | PFC | Properdin Pfactor, complement | NM_002621.1 | Chr X: 46.309-46.316 Mbp | http://genome.ucsc.edu/cgi-bin/hgc?hgsid= |
| | | | | (−) | 34605014&g= |
| | | | | | htcDnaNearGene&i= |
| | | | | | NM_002621&c= |
| | | | | | chrX&l= |
| | | | | | 46308953&r= |
| | | | | | 46317033&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PPBP | pro-platelet basic | NM_002704.2 | Chr 4: 75.253-75.254 Mbp | http://genome.ucsc.edu/cgi-bin/hgc?hgsid= |
| | | protein(chemokine(C—X—C | | (−) | 34605036&g= |
| | | motif) ligand 7) | | | htcDnaNearGene&i= |
| | | | | | NM_002704&c= |
| | | | | | chr4&l= |
| | | | | | 75318005&r= |
| | | | | | 75321151&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | S100A8 | S100 calcium binding protein | NM_002964.3 | Chr 1: 150.137-150.138 Mbp | http://genome.ucsc.edu/cgi-bin/hgc?hgsid= |
| | | A8 (calgranulin A) | | (−) | 34605075&g= |
| | | | | | htcDnaNearGene&i= |
| | | | | | NM_002964&c= |
| | | | | | chr1&l= |
| | | | | | 150578089&r= |
| | | | | | 150581131&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | S100A9 | S100 calcium binding protein | NM_002965.2 | Chr 1: 150.105-150.108 Mbp | http://genome.ucsc.edu/cgi-bin/hgc?hgsid= |
| | | A9 (calgranulin B) | | (+) | 34605111&g= |
| | | | | | htcDnaNearGene&i= |
| | | | | | NM_002965&c= |
| | | | | | chr1&l= |
| | | | | | 150545911&r= |
| | | | | | 150551081&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | S100P | S100 calcium binding protein P | NM_005980.2 | Chr 4: 6.688-6.691 Mbp | http://genome.ucsc.edu/cgi-bin/hgc?hgsid= |
| | | | | (+) | 34605130&g= |
| | | | | | htcDnaNearGene&i= |
| | | | | | NM_005980&c= |
| | | | | | chr4&l= |
| | | | | | 6687292&r= |
| | | | | | 6692624&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SEPX1 | Selenoprotein X, 1 | NM_016332.2 | Chr 16: 1.928-1.933 Mbp | http://genome.ucsc.edu/cgi-bin/hgc?hgsid= |
| | | | | (−) | 34605153&g= |
| | | | | | htcDnaNearGene&i= |
| | | | | | NM_016332&c= |
| | | | | | chr16&l= |
| | | | | | 1927234&r= |
| | | | | | 1934295&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | VNN2 | Vanin 2 | NM_078488.1 | Chr 6: 133.0-133.019 Mbp | http://genome.ucsc.edu/cgi-bin/hgc?hgsid= |
| | | | | (−) | 34605180&g= |
| | | | | | htcDnaNearGene&i= |
| | | | | | NM_004665&c= |
| | | | | | chr6&l= |
| | | | | | 132999138&r= |
| | | | | | 133020728&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Bone Marrow | ALAS2 | Aminolevulinate, delta-, | NM_000032.1 | Chr X: 53.64-53.662 Mbp | http://genome.ucsc.edu/cgi-bin/hgc?hgsid= |
| | | synthase 2 | | (−) | 34605244&g= |
| | | (sideroblastic/hypochromic | | | htcDnaNearGene&i= |
| | | anemia) | | | NM_000032&c= |
| | | | | | chrX&l= |
| | | | | | 53639861&r= |
| | | | | | 53663781&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | AZU1 | Azurocidin 1 (cationic | NM_001700.3 | Chr 19: 0.765-0.772 Mbp | http://genome.ucsc.edu/cgi-bin/hgc?hgsid= |
| | | antimicrobial protein 37) | | (+) | 34605294&g= |
| | | | | | htcDnaNearGene&i= |
| | | | | | NM_001700&c= |
| | | | | | chr19&l= |
| | | | | | 766830&r= |
| | | | | | 773017&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CAMP | Cathelicidin antimicrobial | NM_004345.3 | Chr 3: 48.084-48.086 Mbp | http://genome.ucsc.edu/cgi-bin/hgc?hgsid= |
| | | peptide | | (+) | 34605366&g= |
| | | | | | htcDnaNearGene&i= |
| | | | | | NM_004345&c= |
| | | | | | chr3&l= |
| | | | | | 48083094&r= |
| | | | | | 48087208&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CEACAM8 | Carcinoembryonic antigen- | NM_001816.2 | Chr 19: 47.76-47.775 Mbp | http://genome.ucsc.edu/cgi-bin/hgc?hgsid= |
| | | related cell adhesion molecule 8 | | (−) | 34605434&g= |
| | | | | | htcDnaNearGene&i= |
| | | | | | NM_001816&c= |
| | | | | | chr19&l= |
| | | | | | 47759443&r= |
| | | | | | 47776099&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CLC | Charcot-Leyden crystal protein | NM_001828.4 | Chr 19: 44.897-44.904 Mbp | http://genome.ucsc.edu/cgi-bin/hgc?hgsid= |
| | | | | (+) | 34605548&g= |
| | | | | | htcDnaNearGene&i= |
| | | | | | NM_001828&c= |
| | | | | | chr19&l= |
| | | | | | 44896943&r= |
| | | | | | 44905717&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | DEFA1 | Defensin, alpha 1, corticostatin | NM_004084.2 | Chr 8: 7.014-7.016 Mbp | http://genome.ucsc.edu/cgi-bin/hgc?hgsid= |
| | | | | (+) | 34605625&g= |
| | | | | | htcDnaNearGene&i= |
| | | | | | NM_004084&c= |
| | | | | | chr8&l= |
| | | | | | 7013400&r= |
| | | | | | 7017825&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | DEFA4 | Defensin, alpha 4, corticostatin | NM_001925.1 | Chr 8: 6.953-6.956 Mbp | http://genome.ucsc.edu/cgi-bin/hgc?hgsid= |
| | | | | (+) | 34605720&g= |
| | | | | | htcDnaNearGene&i= |
| | | | | | NM_001925&c= |
| | | | | | chr8&l= |
| | | | | | 6952503&r= |
| | | | | | 6956945&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ELA2 | Elastase 2, neutrophil | NM_001972.1 | Chr 19: 0.792-0.796 Mbp | http://genome.ucsc.edu/cgi-bin/hgc?hgsid= |
| | | | | (+) | 34605796&g= |
| | | | | | htcDnaNearGene&i= |
| | | | | | NM_001972&c= |
| | | | | | chr19&l= |
| | | | | | 791290&r= |
| | | | | | 797242&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HBD | Hemoglobin, delta | NM_000519.2 | Chr 11: 5.213-5.214 Mbp | http://genome.ucsc.edu/cgi-bin/hgc?hgsid= |
| | | | | (+) | 34605890&g= |
| | | | | | htcDnaNearGene&i= |
| | | | | | NM_000519&c= |
| | | | | | chr11&l= |
| | | | | | 5212100&r= |
| | | | | | 5215750&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HBG1 | Hemoglobin, gammin A | NM_000559.2 | Chr 11: 5.228-5.23 Mbp | http://genome.ucsc.edu/cgi-bin/hgc?hgsid= |
| | | | | (−) | 34605986&g= |
| | | | | | htcDnaNearGene&i= |
| | | | | | NM_000559&c= |
| | | | | | chr11&l= |
| | | | | | 5227538&r= |
| | | | | | 5231124&o= |
| | | | | | refGene&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&hgSeq.cdsExon= |
| | | | | | on&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | Hs.356861 | CDNA FLJ26905 fis, clone | | Chr 22: 21.56-21.562 Mbp | |
| | | RCTO1427, highly similar to | | (+) | |
| | | lg lambda chain C regions | | | |
| | IGHG1 | Immunoglobulin heavy | | Chr 14: 104.202-104.211 Mbp | |
| | | constant gamma 1 (G1m | | (−) | |
| | | marker) | | | |
| | IGL@ | Immunoglobulin lambda locus | | Chr 22: 21.425-21.568 Mbp | |
| | | | | (+) | |
| | IGLJ3 | Immunoglobulin lambda | | Chr 22: 20.977-21.568 Mbp | |
| | | joining 3 | | (+) | |
| | LCN2 | Lipocalin 2 (oncongene 24p3) | NM_005564.2 | Chr 9: 124.365-124.369 Mbp | http://genome.ucsc.edu/cgi-bin/hgc?hgsid= |
| | | | | (+) | 34606119&g= |
| | | | | | htcDnaNearGene&i= |
| | | | | | NM_005564&c= |
| | | | | | chr9&l= |
| | | | | | 124364387&r= |
| | | | | | 124370404&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LTF | Lactotransferrin | NM_002343.1 | Chr 3: 46.296-46.345 Mbp | http://genome.ucsc.edu/cgi-bin/hgc?hgsid= |
| | | | | (−) | 34606155&g= |
| | | | | | htcDnaNearGene&i= |
| | | | | | NM_002343&c= |
| | | | | | chr3&l= |
| | | | | | 46295736&r= |
| | | | | | 46326886&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MPO | Myeloperoxidase | NM_000250.1 | Chr 17: 56.689-56.7 Mbp | http://genome.ucsc.edu/cgi-bin/hgc?hgsid= |
| | | | | (−) | 34606311&g= |
| | | | | | htcDnaNearGene&i= |
| | | | | | NM_000250&c= |
| | | | | | chr17&l= |
| | | | | | 56688295&r= |
| | | | | | 56701375&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | OLFM2 | Olfactomedin 4 | NM_006418.3 | Chr 13: 52.539-52.562 Mbp | http://genome.ucsc.edu/cgi-bin/hgc?hgsid= |
| | | | | (+) | 34606391&g= |
| | | | | | htcDnaNearGene&i= |
| | | | | | NM_006418&c= |
| | | | | | chr13&l= |
| | | | | | 52538608&r= |
| | | | | | 52563829&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PRG2 | Proteoglycan 2, bone marrow | NM_002728.4 | Chr 11: 57.405-57.409 Mbp | http://genome.ucsc.edu/cgi-bin/hgc?hgsid= |
| | | (natural killer cell activator, | | (−) | 34606424&g= |
| | | eosinphil granule major basic | | | htcDnaNearGene&i= |
| | | protein) | | | NM_002728&c= |
| | | | | | chr11&l= |
| | | | | | 57404716&r= |
| | | | | | 57410013&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RNASE3 | Ribonuclease, Rnase A family, | NM_002935.2 | Chr 14: 19.349-19.35 Mbp | http://genome.ucsc.edu/cgi-bin/hgc?hgsid= |
| | | 3 (eosinophil cationic protein) | | (+) | 34606450&g= |
| | | | | | htcDnaNearGene&i= |
| | | | | | NM_002935&c= |
| | | | | | chr14&l= |
| | | | | | 19348689&r= |
| | | | | | 19351635&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Amygdala | APLP1 | Amyloid beta (A4) precursor- | NM_005166.2 | Chr 19: 41.035-41.046 Mbp | http://genome.ucsc.edu/cgi-bin/hgc?hgsid= |
| | | like protein 1 | | (+) | 34606560&g= |
| | | | | | htcDnaNearGene&i= |
| | | | | | NM_005166&c= |
| | | | | | chr19&l= |
| | | | | | 41034518&r= |
| | | | | | 41047740&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CaMKINalpha | Calcium/calmodulin-dependent | NM_018584.4 | Chr 1: 19.955-19.958 Mbp | http://genome.ucsc.edu/cgi-bin/hgc?hgsid= |
| | | protein kinase II | | (−) | 34606589&g= |
| | | | | | htcDnaNearGene&i= |
| | | | | | NM_018584&c= |
| | | | | | chr1&l= |
| | | | | | 19954898&r= |
| | | | | | 19959252&o= |
| | | | | | refGene&hgSeq.promoter= |
| | | | | | on&boolshad.hgSeq.promoter= |
| | | | | | 1 &hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GPM6B | Glycoprotein M6B | NM_005278.2 | Chr X: 12.994-13.037 Mbp | http://genome.ucsc.edu/cgi-bin/hgc?hgsid= |
| | | | | (−) | 34606607&g= |
| | | | | | htcDnaNearGene&i= |
| | | | | | NM_005278&c= |
| | | | | | chrX&l= |
| | | | | | 12993126&r= |
| | | | | | 13038158&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1 &boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GRIA2 | Glutamate receptor, ionotropic, | NM_000826.1 | Chr 4: 158.608-158.751 Mbp | http://genome.ucsc.edu/cgi-bin/hgc?hgsid= |
| | | AMPA2 | | (+) | 34606642&g= |
| | | | | | htcDnaNearGene&i= |
| | | | | | NM_000826&c= |
| | | | | | chr4&l= |
| | | | | | 158607221&r= |
| | | | | | 158752289&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | OLFM1 | Olfactonmedin 1 | NM_006334.2 | Chr 9: 131.49-131.536 Mbp | http://genome.ucsc.edu/cgi-bin/hgc?hgsid= |
| | | | | (+) | 34606662&g= |
| | | | | | htcDnaNearGene&i= |
| | | | | | NM_006334&c= |
| | | | | | chr9&l= |
| | | | | | 131489268&r= |
| | | | | | 131537122&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | STMN2 | Stathmin-like 2 | NM_007029.2 | Chr 8: 80.246-80.3 Mbp | http://genome.ucsc.edu/cgi-bin/hgc?hgsid= |
| | | | | (+) | 34606685&g= |
| | | | | | htcDnaNearGene&i= |
| | | | | | NM_007029&c= |
| | | | | | chr8&l= |
| | | | | | 80245565&r= |
| | | | | | 80301429&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Thalamus | GFAP | Glialfibrillary acidic protein | NM_002055.2 | Chr 17: 42.993-43.003 Mbp | http://genome.ucsc.edu/cgi-bin/hgc?hgsid= |
| | | | | (−) | 34606809&g= |
| | | | | | htcDnaNearGene&i= |
| | | | | | NM_002055&c= |
| | | | | | chr17&l= |
| | | | | | 42992757&r= |
| | | | | | 43004633&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HTN3 | Histatin3 | NM_000200.1 | Chr 4: 71.144-71.152 Mbp | http://genome.ucsc.edu/cgi-bin/hgc?hgsid= |
| | | | | (+) | 34606828&g= |
| | | | | | htcDnaNearGene&i= |
| | | | | | NM_000200&c= |
| | | | | | chr4&l= |
| | | | | | 71143105&r= |
| | | | | | 71153177&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MBP | Myelin basic product | NM_002385.1 | Chr 18: 74.454-74.491 Mbp | http://genome.ucsc.edu/cgi-bin/hgc?hgsid= |
| | | | | (+) | 34606859&g= |
| | | | | | htcDnaNearGene&i= |
| | | | | | NM_002385&c= |
| | | | | | chr18&l= |
| | | | | | 74453704&r74492956&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PLP1 | Proteolipid protein 1 | NM_199478.1 | Chr X: 101.064-101.08 Mbp | http://genome.ucsc.edu/cgi-bin/hgc?hgsid= |
| | | (Pelizaeous-Merzbacher | | (+) | 34606886&g= |
| | | disease, spastic parapeligia 2, | | | htcDnaNearGene&i= |
| | | uncomplicated) | | | NM_000533&c= |
| | | | | | chrX&l= |
| | | | | | 101063720&r= |
| | | | | | 101081515&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PRH1 | Proline-rich protein Haelll | NM_006250.1 | Chr 12: 10.933-11.224 Mbp | http://genome.ucsc.edu/cgi-bin/hgc?hgsid= |
| | | subfamily 1 | | (−) | 34606910&g= |
| | | | | | htcDnaNearGene&i= |
| | | | | | NM_006250&c= |
| | | | | | chr12&l= |
| | | | | | 10932826&r= |
| | | | | | 10938121&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PRH2 | Proline-rich protein Haelll | NM_005042.1 | Chr 12: 10.982-10.986 Mbp | http://genome.ucsc.edu/cgi-bin/hgc?hgsid= |
| | | subfamily 2 | | (+) | 34606929&g= |
| | | | | | htcDnaNearGene&i= |
| | | | | | NM_005042&c= |
| | | | | | chr12&l= |
| | | | | | 10981106&r= |
| | | | | | 10986184&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | TTR | Transythretin (prealbumin, | NM_000371.1 | Chr 18: 29.059-29.066 Mbp | http://genome.ucsc.edu/cgi-bin/hgc?hgsid= |
| | | amyloidosis type 1) | | (+) | 34606955&g= |
| | | | | | htcDnaNearGene&i= |
| | | | | | NM_000371&c= |
| | | | | | chr18&l= |
| | | | | | 29058831&r= |
| | | | | | 29067775&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ZIC1 | Zic family member 1 (odd- | NM_000371.1 | Chr 18: 29.059-29.066 Mbp | http://genome.ucsc.edu/cgi-bin/hgc?hgsid= |
| | | paired homolog), Drosphilia | | (+) | 34606980&g= |
| | | | | | htcDnaNearGene&i= |
| | | | | | NM_003412&c= |
| | | | | | chr3&l= |
| | | | | | 148447089&r= |
| | | | | | 148454257&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | 32512_at | Homo sapiens clone BAC | | Chr 8: 24.596-24.597 Mbp | |
| | | 72m22 chromosome 8 map | | (+) | |
| | | 8p21, complete sequence | | | |
| Cuadatenucleus | ARPP-21 | cyclic AMP-regulated | NM_016300.3 | Chr 3: 35.556-35.671 Mbp | http://genome.ucsc.edu/cgi-bin/hgc?hgsid= |
| | | phosphoprotein, 21 kD | | (+) | 34607030&g= |
| | | | | | htcDnaNearGene&i= |
| | | | | | NM_016300&c= |
| | | | | | chr3&l= |
| | | | | | 35555575&r= |
| | | | | | 35672448&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeg.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HPCA | Hippocalcin | NM_002143.2 | Chr 1: 32.781-32.786 Mbp | http://genome.ucsc.edu/cgi-bin/hgc?hgsid= |
| | | | | (+) | 34607057&g= |
| | | | | | htcDnaNearGene&i= |
| | | | | | NM_002143&c= |
| | | | | | chr1&l= |
| | | | | | 32780120&r= |
| | | | | | 32787890&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | 38291_at | Human enkephalin gene | | Chr 8: 57.076-57.077 Mbp | |
| | | | | (−) | |
| | 41602_at | Homo sapiens gene for | | Chr 1: 32.786-32.786 Mbp | |
| | | hippocalcin | | (+) | |
| PrefrontalCortex | CHN1 | Chimerin (Chimaerin) 1 | NM_001822.2 | Chr 2: 175.628-175.833 Mbp | http://genome.ucsc.edu/cgi-bin/hgc?hgsid= |
| | | | | (−) | 34607105&g= |
| | | | | | htcDnaNearGene&i= |
| | | | | | NM_001822&c= |
| | | | | | chr2&l= |
| | | | | | 175627257&r= |
| | | | | | 175834978&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Olfactory Bulb | S100B | S100B calcium binding | NM_006272.1 | Chr 21: 46.875-46.881 Mbp | http://genome.ucsc.edu/cgi-bin/hgc?hgsid= |
| | | protein, beta (neural) | | (−) | 34607140&g= |
| | | | | | htcDnaNearGene&i= |
| | | | | | NM_006272&c= |
| | | | | | chr21&l= |
| | | | | | 46874172&r= |
| | | | | | 46882638&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1 &hgSeq.casing= |
| | | | | | exon&boolshad.hgSeq.maskRepeats=1&hgSeq.repMasking=lower&submit= |
| | | | | | submit |
| | PCP4 | Purkinje cell protein 4 | NM_006198.2 | Chr 21: 40.191-40.222 Mbp | http://genome.ucsc.edu/cgi-bin/hgc?hgsid= |
| | | | | (−) | 34607176&g= |
| | | | | | htcDnaNearGene&i= |
| | | | | | NM_006198&c= |
| | | | | | chr21&l= |
| | | | | | 40158742&r= |
| | | | | | 40222718&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Hypothalamus | PMCH | pro-melanin-concentrating | NM_002674.1 | Chr 12: 102.523-102.524 Mbp | http://genome.ucsc.edu/cgi-bin/hgc?hgsid= |
| | | hormone | | (−) | 34608309&g= |
| | | | | | htcDnaNearGene&i= |
| | | | | | NM_002674&c= |
| | | | | | chr12&l= |
| | | | | | 102522185&r= |
| | | | | | 102525549&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Cortex | 33925_at | H. sapiens NRGN gene, exons | | Chr 11: 124.65-102.651 Mbp | |
| | | 2, 3 & 4 (joined CDS) | | (+) | |
| | 38699_at | Human beta-tubulin gene (5- | | Chr 19: 6.434-6.434 Mbp | |
| | | beta) with ten Alu family | | (−) | |
| | | members | | | |
| | 40995_at | Human gene for neurofilament | | Chr 8: 24.63-24.63 Mbp | |
| | | subunit NF-L | | (−) | |
| | GPR51 | G protein-coupled receptor 51 | NM_005458.5 | Chr 9: 94.507-94.928 Mbp | |
| | | | | (−) | |
| | SLC17A7 | solute carrier family 17 | NM_020309.2 | Chr 19: 54.608-54.62 Mbp | |
| | | (sodium-dependent inorganic | | (−) | |
| | | phosphate cotransporter), | | | |
| | | member 7 | | | |
| | SNAP91 | Synaptosomal-associated | | Chr 6: 84.212-84.368 Mbp | |
| | | protein, 91 kDa homolog | | (−) | |
| | | (mouse) | | | |
| Brain | CA11 | Carbonic anhydrase XI | NM_001217.2 | Chr 19: 53.817-53.825 Mbp | |
| | | | | (−) | |
| | DDN | Dendrin | | Chr 12: 49.105-49.109 Mbp | |
| | | | | (−) | |
| Corpus_Callosum | BCAS1 | breast carcinoma amplified | NM_003657.1 | Chr 20: 53.198-53.325 Mbp | |
| | | sequence 1 | | (−) | |
| | UGT8 | UDP glycosyltransferase 8 | NM_003360.2 | Chr 4: 115.936-115.99 Mbp | |
| | | (UDP-galactose ceramide | | (+) | |
| | | galactosyltransferase) | | | |
| Cerebellum | NEUROD1 | neurogenic differentiation 1 | NM_002500.1 | Chr 2: 182.505-182.509 Mbp | |
| | | | | (−) | |
| Bronchialepi- | CDH1 | Cadherin 1, type 1, E-cadherin | NM_004360.2 | Chr 16: 68.506-68.604 Mbp | http://genome.ucsc.edu/cgi-bin/hgc?hgsid= |
| | | (epithelial) | | (+) | 34608402&g= |
| | | | | | htcDnaNearGene&i= |
| | | | | | NM_004360&c= |
| | | | | | chr16&l= |
| | | | | | 68505610&r= |
| | | | | | 68605860&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.Padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| thelialcells | CDH3 | Cadherin 3, type 1, P-cadherin | NM_001793.3 | Chr 16: 68.414-68.468 Mbp | http://genome.ucsc.edu/cgi-bin/hgc?hgsid= |
| | | (placental) | | (+) | 34608426&g= |
| | | | | | htcDnaNearGene&i= |
| | | | | | NM_001793&c= |
| | | | | | chr16&l= |
| | | | | | 68453934&r= |
| | | | | | 68510130&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.Padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CSTA | Cystatin A (stefin A) | NM_005213.2 | Chr 3: 123.325-123.341 Mbp | http://genome.ucsc.edu/cgi-bin/hgc?hgsid= |
| | | | | (+) | 34608458&g= |
| | | | | | htcDnaNearGene&i= |
| | | | | | NM_005213&c= |
| | | | | | chr3&l= |
| | | | | | 123324311&r= |
| | | | | | 123342740&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | FXYD3 | FXYDdomain containing ion | NM_005971.2 | Chr 19: 40.282-40.291 Mbp | http://genome.ucsc.edu/cgi-bin/hgc?hgsid= |
| | | transport regulator 3 | | (+) | 34608472&g= |
| | | | | | htcDnaNearGene&i= |
| | | | | | NM_005213&c= |
| | | | | | chr3&l= |
| | | | | | 123324311&r= |
| | | | | | 123342740&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT14 | Keratin 14 (epidermolysis | NM_000526.3 | Chr 17: 39.647-39.651 Mbp | http://genome.ucsc.edu/cgi-bin/hgc?hgsid= |
| | | bullosa simplex, Dowling- | | (−) | 34608502&g= |
| | | Meara, Koebner) | | | htcDnaNearGene&i= |
| | | | | | NM_005971&c= |
| | | | | | chr19&l= |
| | | | | | 40281847&r= |
| | | | | | 40292276&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT17 | Keratin 17 | NM_000422.1 | Chr 17: 39.684-39.689 Mbp | http://genome.ucsc.edu/cgi-bin/hgc?hgsid= |
| | | | | (−) | 34608554&g= |
| | | | | | htcDnaNearGene&i= |
| | | | | | NM_000422&c= |
| | | | | | chr17&l= |
| | | | | | 39683457&r= |
| | | | | | 39690573&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT19 | Keratin 19 | NM_002276.3 | Chr 17: 39.588-39.593 Mbp | http://genome.ucsc.edu/cgi-bin/hgc?hgsid= |
| | | | | (−) | 34608593&g= |
| | | | | | htcDnaNearGene&i= |
| | | | | | NM_002276&c= |
| | | | | | chr17&l= |
| | | | | | 39587632&r= |
| | | | | | 39594398&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT5 | Keratin 5 | NM_000424.2 | Chr 12: 52.625-52.63 Mbp | http://genome.ucsc.edu/cgi-bin/hgc?hgsid= |
| | | (epidermolysisbullosa simplex, | | (−) | 34608628&g= |
| | | Dowling- | | | htcDnaNearGene&i= |
| | | Maera/Koebner/Weber- | | | NM_002276&c= |
| | | Cockayne types) | | | chr17&l= |
| | | | | | 39587632&r= |
| | | | | | 39594398&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT6A | Keratin 6A | NM_005554.2 | Chr 12: 52.597-52.603 Mbp | http://genome.ucsc.edu/cgi-bin/hgc?hgsid= |
| | | | | (−) | 34608657&g= |
| | | | | | htcDnaNearGene&i= |
| | | | | | NM_005554&c= |
| | | | | | chr12&l= |
| | | | | | 52596723&r= |
| | | | | | 52604767&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT6B | Keratin 6B | NM_005555.2 | Chr 12: 52.557-52.562 Mbp | http://genome.ucsc.edu/cgi-bin/hgc?hgsid= |
| | | | | (−) | 34608690&g= |
| | | | | | htcDnaNearGene&i= |
| | | | | | NM_000424&c= |
| | | | | | chr12&l= |
| | | | | | 52624107&r= |
| | | | | | 52631990&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT6E | Keratin 6E | NM_173086.2 | Chr 12: 52.579-52.584 Mbp | http://genome.ucsc.edu/cgi-bin/hgc?hgsid= |
| | | | | (−) | 34608994&g= |
| | | | | | htcDnaNearGene&i= |
| | | | | | NM_173086&c= |
| | | | | | chr12&l= |
| | | | | | 52578341&r= |
| | | | | | 52585304&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | KRT7 | Keratin 7 | NM_005556.2 | Chr 12: 52.343-52.359 Mbp | http://genome.ucsc.edu/cgi-bin/hgc?hgsid= |
| | | | | (+) | 34644330&g= |
| | | | | | htcDnaNearGene&i= |
| | | | | | NM_005556&c= |
| | | | | | chr12&l= |
| | | | | | 52343784&r= |
| | | | | | 52359456&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LAMA3 | Laminin, alpha3 | NM_198129.1 | Chr 18: 21.157-21.423 Mbp | http://genome.ucsc.edu/cgi-bin/hgc?hgsid= |
| | | | | (+) | 34644330&g= |
| | | | | | htcDnaNearGene&i= |
| | | | | | NM_000227&c= |
| | | | | | chr18&l= |
| | | | | | 21332738&r= |
| | | | | | 21422895&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | LGALS7 | Lectin, galactoside-binding, | NM_002307.1 | Chr 19: 43.955-43.958 Mbp | http://genome.ucsc.edu/cgi-bin/hgc?hgsid= |
| | | soluable 7 (galectin 7) | | (+) | 34644330&g= |
| | | | | | htcDnaNearGene&i= |
| | | | | | NM_002307&c= |
| | | | | | chr19&l= |
| | | | | | 43955900&r= |
| | | | | | 43958443&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | S100A2 | S100 calcium binding protein | NM_005978.3 | Chr 1: 150.36-150.365 Mbp | http://genome.ucsc.edu/cgi-bin/hgc?hgsid= |
| | | A2 | | (+) | 34644330&g= |
| | | | | | htcDnaNearGene&i= |
| | | | | | NM_005978&c= |
| | | | | | chr1&l= |
| | | | | | 150360914&r= |
| | | | | | 150365412&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SERPINB5 | Serine (or cysteine) proteinase | NM_002639.1 | Chr 18: 60.929-60.957 Mbp | http://genome.ucsc.edu/cgi-bin/hgc?hgsid= |
| | | inhibitor, clade B (ovalbumin), | | (+) | 34644330&g= |
| | | member 5 | | | htcDnaNearGene&i= |
| | | | | | NM_002639&c= |
| | | | | | chr18&l= |
| | | | | | 60929192&r= |
| | | | | | 60957291&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SFN | Stratifin | NM_006142.3 | Chr 1: 26.422-26.423 Mbp | http://genome.ucsc.edu/cgi-bin/hgc?hgsid= |
| | | | | (+) | 34644330&g= |
| | | | | | htcDnaNearGene&i= |
| | | | | | NM_006142&c= |
| | | | | | chr1&l= |
| | | | | | 26422672&r= |
| | | | | | 26423992&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | TACSTD2 | tumor-associated calcium | NM_006142.3 | Chr 1: 58.398-58.401 Mbp | http://genome.ucsc.edu/cgi-bin/hgc?hgsid= |
| | | signal transducer 2 | | (−) | 34644330&g= |
| | | | | | htcDnaNearGene&i= |
| | | | | | NM_002353&c= |
| | | | | | chr1&l= |
| | | | | | 58398350&r= |
| | | | | | 58401153&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | TFP12 | tissue factor pathway inhibitor 2 | NM_006528.2 | Chr 7: 93.113-93.118 Mbp | |
| | | | | (−) | |
| Colorectal- | CST1 | Cystatin SN | NM_001898.2 | Chr 20: 23.676-23.679 Mbp | http://genome.ucsc.edu/cgi-bin/hgc?hgsid= |
| | | | | (−) | 34644330&g= |
| | | | | | htcDnaNearGene&i= |
| | | | | | NM_001898&c= |
| | | | | | chr20&l= |
| | | | | | 23676189&r= |
| | | | | | 23681199&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Adenocarcinoma | SERPINE1 | Serine (or cysteine) proteinase | NM_000602.1 | Chr 7: 100.316-100.328 Mbp | http://genome.ucsc.edu/cgi-bin/hgc?hgsid= |
| | | inhibitor, clade E (nexin, | | (+) | 34644330&g= |
| | | plasminogen activator inhibitor | | | htcDnaNearGene&i= |
| | | type 1), member 1 | | | NM_000602&c= |
| | | | | | chr7&l= |
| | | | | | 100318110&r= |
| | | | | | 100328878&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| PB-BDCA4+ | 216401_x_at | Homo sapiens partial IGKV | | Chr 2: 89.482-89.482 Mbp | |
| | | gene for immunoglobulin | | (−) | |
| | | kappa chain variable region, | | | |
| | | clone 38 | | | |
| Dentritic_Cells | 216491_x_at | Human immunoglobulin heavy | | Chr 14: 104.449-104.45 Mbp | |
| | | chain variable region (V4-4) | | (−) | |
| | | gene, partial cds | | | |
| | CLIC3 | Chloride intracellular channel 2 | NM_004669.2 | Chr 9: 133.33-133.332 Mbp | http://genome.ucsc.edu/cgi-bin/hgc?hgsid= |
| | | | | (−) | 34644330&g= |
| | | | | | htcDnaNearGene&i= |
| | | | | | NM_004669&c= |
| | | | | | chr9&l= |
| | | | | | 133330155&r= |
| | | | | | 133332086&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | DOCK2 | dedicator of cytokinesis 2 | NM_004946.1 | Chr 5: 168.999-169.445 Mbp | |
| | | | | (+) | |
| | HLA-DQB1 | major histocompatibility | NM_002123.2 | Chr 6: 32.628-32.635 Mbp | http://genome.ucsc.edu/cgi-bin/hgc?hgsid= |
| | | complex, class II, DQ beta II | | (−) | 34644330&g= |
| | | | | | htcDnaNearGene&i= |
| | | | | | NM_002123&c= |
| | | | | | chr6_random&l= |
| | | | | | 8324503&r= |
| | | | | | 8331637&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HLA-DRA | major histocompatibility | NM_019111.2 | Chr 6: 32.433-32.438 Mbp | http://genome.ucsc.edu/cgi-bin/hgc?hgsid= |
| | | complex, class II, DR alpha | | (+) | 34644330&g= |
| | | | | | htcDnaNearGene&i= |
| | | | | | NM_019111&c= |
| | | | | | chr6_random&1= |
| | | | | | 8129918&r= |
| | | | | | 8134989&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | HLA-DRB3 | major histocompatibility | NM_022555.3 | Chr 6: 32.489-32.502 Mbp | |
| | | complex, class II, DR beta 3 | | (−) | |
| | Hs.383169 | Partial mRNA for | | Chr 22: 21.56-21.562 Mbp | |
| | | immunoglobulin heavy chain | | (+) | |
| | | variable region (IGHV32-D- | | | |
| | | JH-Cmu gene), clone ET39 | | | |
| | IGH@ | Immunoglobulin heavy locus | | Chr 14: 104.077-104.45 Mbp | |
| | | | | (−) | |
| | ILT7 | Leukocyte immunoglobulin- | NM_012276.3 | Chr 19: 59.52-59.526 Mbp | http://genome.ucsc.edu/cgi-bin/hgc?hgsid= |
| | | like receptor, subfamily A | | (−) | 34644330&g= |
| | | (without TM domain), member 4 | | | htcDnaNearGene&i= |
| | | | | | NM_012276&c= |
| | | | | | chr19&l= |
| | | | | | 59520712&r= |
| | | | | | 59610729&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PACAP | Proapoptotic caspase adaptor | NM_016459.2 | Chr 5: 138.754-138.756 Mbp | |
| | | protein | | (−) | |
| | RNASE6 | Ribonuclease, Rnase A family, | NM_005615.2 | Chr 14: 19.239-19.24 Mbp | http://genome.ucsc.edu/cgi-bin/hgc?hgsid= |
| | | k6 | | (+) | 34644330&g= |
| | | | | | htcDnaNearGene&i= |
| | | | | | NM_005615&c= |
| | | | | | chr14&l= |
| | | | | | 19239337&r= |
| | | | | | 19240752&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | TNFRSF17 | tumor necrosis factor receptor | NM_001192.2 | Chr 16: 12.025-12.028 Mbp | http://genome.ucsc.edu/cgi-bin/hgc?hgsid= |
| | | superfamily, member 17 | | (+) | 34644330&g= |
| | | | | | htcDnaNearGene&i= |
| | | | | | NM_001192&c= |
| | | | | | chr16&l= |
| | | | | | 12025398&r= |
| | | | | | 12028355&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Pancreas | 216470_x_at | T cell receptor beta locus | | Chr 7: 141.854-141.855 Mbp | |
| | | | | (+) | |
| | AMY2A | Amylase, alpha 2A; pancreatic | NM_000699.2 | Chr 1: 103.342-103.351 Mbp | http://genome.ucsc.edu/cgi-bin/hgc?hgsid= |
| | | | | (+) | 34644330&g= |
| | | | | | htcDnaNearGene&i= |
| | | | | | NM_001192&c= |
| | | | | | chr16&l= |
| | | | | | 12025398&r= |
| | | | | | 12028355&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | ARFGEF2 | ADP-ribosylation factor | NM_006420.1 | Chr 20: 48.176-48.288 Mbp | http://genome.ucsc.edu/cgi-bin/hgc?hgsid= |
| | | guanine nucleotide-exchange | | (+) | 34644330&g= |
| | | factor 2 (brefeldin A-inhibited) | | | htcDnaNearGene&i= |
| | | | | | NM_006420&c= |
| | | | | | chr20&l= |
| | | | | | 48176848&r= |
| | | | | | 48288660&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CEL | Carboxyl ester lipase (bile salt- | NM_001807.2 | Chr 9: 129.291-129.3 Mbp | http://genome.ucsc.edu/cgi-bin/hgc?hgsid= |
| | | stimulated lipase) | | (+) | 34644330&g= |
| | | | | | htcDnaNearGene&i= |
| | | | | | NM_001807&c= |
| | | | | | chr9&l= |
| | | | | | 129291039&r= |
| | | | | | 129300849&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | CELP | Carboxyl ester lipase | NM_001808 | Chr 9: 129.311-129.316 Mbp | http://genome.ucsc.edu/cgi-bin/hgc?hgsid= |
| | | pseudogene | | (+) | 34644330&g= |
| | | | | | htcDnaNearGene&i= |
| | | | | | NM_173692&c= |
| | | | | | chr9&l= |
| | | | | | 129311595&r= |
| | | | | | 129316412&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CLPS | Colipase, pancreatic | NM_001832.2 | Chr 6: 35.764-35.766 Mbp | http://genome.ucsc.edu/cgi-bin/hgc?hgsid= |
| | | | | (−) | 34644330&g= |
| | | | | | htcDnaNearGene&i= |
| | | | | | NM_001832&c= |
| | | | | | chr6&l= |
| | | | | | 35764174&r= |
| | | | | | 35766515&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | CPA1 | Carboxy-peptidase A1 | NM_001868.1 | Chr 7: 129.559-129.567 Mbp | http://genome.ucsc.edu/cgi-bin/hgc?hgsid= |
| | | (pancreatic) | | (+) | 34644330&g= |
| | | | | | htcDnaNearGene&i= |
| | | | | | NM_001868&c= |
| | | | | | chr7&l= |
| | | | | | 129559540&r= |
| | | | | | 129567150&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CPA2 | Carboxypeptidase A2 | NM_001869.1 | Chr 7: 129.445-129.468 Mbp | http://genome.ucsc.edu/cgi-bin/hgc?hgsid= |
| | | (pancreatic) | | (+) | 34644330&g= |
| | | | | | htcDnaNearGene&i= |
| | | | | | NM_001869&c= |
| | | | | | chr7&l= |
| | | | | | 129445905&r= |
| | | | | | 129468834&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | CPB1 | Carboxy-peptidase B1 (tissue) | NM_001871.1 | Chr 3: 149.827-149.859 Mbp | http://genome.ucsc.edu/cgi-bin/hgc?hgsid= |
| | | | | (+) | 34644330&g= |
| | | | | | htcDnaNearGene&i= |
| | | | | | NM_001871&c= |
| | | | | | chr3&l= |
| | | | | | 149827217&r= |
| | | | | | 149859585&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CTRB1 | Chymotrypsinogen B1 | NM_001906.1 | Chr 16: 74.976-74.997 Mbp | http://genome.ucsc.edu/cgi-bin/hgc?hgsid= |
| | | | | (+) | 34644330&g= |
| | | | | | htcDnaNearGene&i= |
| | | | | | NM_001906&c= |
| | | | | | chr16&l= |
| | | | | | 74976827&r= |
| | | | | | 74979862&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | CTRC | Chymotrypsin C (caldecrin) | NM_007272.1 | Chr 1: 15.032-15.041 Mbp | http://genome.ucsc.edu/cgi-bin/hgc?hgsid= |
| | | | | (+) | 34644330&g= |
| | | | | | htcDnaNearGene&i= |
| | | | | | NM_007272&c= |
| | | | | | chr1&l= |
| | | | | | 15032850&r= |
| | | | | | 15041061&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CTRL | Chymotrypsin-like | NM_001907.1 | Chr 16: 67.698-67.701 Mbp | http://genome.ucsc.edu/cgi-bin/hgc?hgsid= |
| | | | | (−) | 34644330&g= |
| | | | | | htcDnaNearGene&i= |
| | | | | | NM_001907&c= |
| | | | | | chr16&l= |
| | | | | | 67698980&r= |
| | | | | | 67705384&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | CUZD1 | CUBand zona pellucida-like | NM_022034.3 | Chr 10: 124.598-124.617 Mbp | http://genome.ucsc.edu/cgi-bin/hgc?hgsid= |
| | | domains 1 | | (−) | 34646048&g= |
| | | | | | htcDnaNearGene&i= |
| | | | | | NM_022034&c= |
| | | | | | chr10&l= |
| | | | | | 124597641&r= |
| | | | | | 124618281&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | o&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ELA2A | Elastase 2A | NM_033440.1 | Chr 1: 15.051-15.066 Mbp | http://genome.ucsc.edu/cgi-bin/hgc?hgsid= |
| | | | | (+) | 34644330&g= |
| | | | | | htcDnaNearGene&i= |
| | | | | | NM_033440&c= |
| | | | | | chr1&l= |
| | | | | | 15051139&r= |
| | | | | | 15066498&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | ELA2B | Pancreatic elastase IIB | NM_015849.1 | Chr 1: 15.07-15.085 Mbp | http://genome.ucsc.edu/cgi-bin/hgc?hgsid= |
| | | | | (+) | 34644330&g= |
| | | | | | htcDnaNearGene&i= |
| | | | | | NM_015849&c= |
| | | | | | chr1&l= |
| | | | | | 15070511&r= |
| | | | | | 15085810&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ELA3A | Elastase 3A, pancreatic | NM_005747.2 | Chr 1: 21.474-21.485 Mbp | http://genome.ucsc.edu/cgi-bin/hgc?hgsid= |
| | | | | (+) | 34645455&g= |
| | | | | | htcDnaNearGene&i= |
| | | | | | NM_005747&c= |
| | | | | | chr1&l= |
| | | | | | 21473132&r= |
| | | | | | 21486009&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | ELA3B | Elastase 3B, pancreatic | NM_007352.1 | Chr 1: 21.449-21.47 Mbp | http://genome.ucsc.edu/cgi-bin/hgc?hgsid= |
| | | | | (+) | 34645480&g= |
| | | | | | htcDnaNearGene&i= |
| | | | | | NM_007352&c= |
| | | | | | chr1&l= |
| | | | | | 21448494&r= |
| | | | | | 21462817&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | FABP1 | fatty acid binding protein 1, | NM_001443.1 | Chr 2: 88.307-88.312 Mbp | http://genome.ucsc.edu/cgi-bin/hgc?hgsid= |
| | | liver | | (−) | 34645611&g= |
| | | | | | htcDnaNearGene&i= |
| | | | | | NM_001443&c= |
| | | | | | chr2&l= |
| | | | | | 88306824&r= |
| | | | | | 88313893&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | GCG | Glucagon | NM_002054.2 | Chr 2: 162.963-162.972 Mbp | http://genome.ucsc.edu/cgi-bin/hgc?hgsid= |
| | | | | (−) | 34645642&g= |
| | | | | | htcDnaNearGene&i= |
| | | | | | NM_002054&c= |
| | | | | | chr2&l= |
| | | | | | 162962411&r= |
| | | | | | 162973781&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.Padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GP2 | Glycoprotein 2 (zymogen | NM_001502.1 | Chr 16: 20.248-20.266 Mbp | http://genome.ucsc.edu/cgi-bin/hgc?hgsid= |
| | | granule membrane) | | (−) | 34645676&g= |
| | | | | | htcDnaNearGene&i= |
| | | | | | NM_001502&c= |
| | | | | | chr16&l= |
| | | | | | 20248517&r= |
| | | | | | 20266229&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | INS | Insulin | NM_000207.1 | Chr 11: 2.14-2.141 Mbp | http://genome.ucsc.edu/cgi-bin/hgc?hgsid= |
| | | | | (−) | 34645704&g= |
| | | | | | htcDnaNearGene&i= |
| | | | | | NM_000207&c= |
| | | | | | chr11&l= |
| | | | | | 2139295&r= |
| | | | | | 2142711&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | MT1G | Metallothionein 1G | NM_005950.1 | Chr 16: 56.435-56.436 Mbp | |
| | | | | (−) | |
| | PDIP | Protein disulfide isomerase, | NM_006849.1 | Chr 16: 0.273-0.277 Mbp | |
| | | pancreatic | | (+) | |
| | PLA2G1B | Phosphlipase A2, group IB | NM_000928.2 | Chr 12: 120.542-120.548 Mbp | http://genome.ucsc.edu/cgi-bin/hgc?hgsid= |
| | | (pancreas) | | (−) | 34645736&g= |
| | | | | | htcDnaNearGene&i= |
| | | | | | NM_000928&c= |
| | | | | | chr12&l= |
| | | | | | 120541766&r= |
| | | | | | 120549445&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | PNLIP | Pancreatic lipase | NM_000936.1 | Chr 10: 118.436-118.458 Mbp | http://genome.ucsc.edu/cgi-bin/hgc?hgsid= |
| | | | | (+) | 34645761&g= |
| | | | | | htcDnaNearGene&i= |
| | | | | | NM_000936&c= |
| | | | | | chr10&l= |
| | | | | | 118435684&r= |
| | | | | | 118459593&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PNILPRP1 | Pancreatic lipase-related | NM_006229.1 | Chr 10: 118.481-118.499 Mbp | http://genome.ucsc.edu/cgi-bin/hgc?hgsid= |
| | | protein 2 | | (+) | 34645797&g= |
| | | | | | htcDnaNearGene&i= |
| | | | | | NM_006229&c= |
| | | | | | chr10&l= |
| | | | | | 118480715&r= |
| | | | | | 118500912&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1 &boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | PNLIPRP2 | Pancreatic lipase-related | NM_005396.3 | Chr 10: 118.512-118.535 Mbp | http://genome.ucsc.edu/cgi-bin/hgc?hgsid= |
| | | protein 1 | | (+) | 34645829&g= |
| | | | | | htcDnaNearGene&i= |
| | | | | | NM_005396&c= |
| | | | | | chr10&l= |
| | | | | | 118511043&r= |
| | | | | | 118536878&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1 &boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PRSS2 | Protease, serine, 2 (trypsin 2) | NM_002770.2 | Chr 7: 141.822-141.866 Mbp | http://genome.ucsc.edu/cgi-bin/hgc?hgsid= |
| | | | | (+) | 34645849&g= |
| | | | | | htcDnaNearGene&i= |
| | | | | | NM_002770&c= |
| | | | | | chr7&l= |
| | | | | | 141861729&r= |
| | | | | | 141867315&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1 &boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | PRSS3 | Protease, serine, 3 | NM_002771.2 | Chr 9: 33.74-33.789 Mbp | http://genome.ucsc.edu/cgi-bin/hgc?hgsid= |
| | | (mesotrypsin) | | (+) | 34645872&g= |
| | | | | | htcDnaNearGene&i= |
| | | | | | NM_002771&c= |
| | | | | | chr9&l= |
| | | | | | 33784559&r= |
| | | | | | 33790229&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1 &boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | REG1A | regenerating islet-derived 1 | NM_002909.3 | Chr 2: 79.305-79.305 Mbp | http://genome.ucsc.edu/cgi-bin/hgc?hgsid= |
| | | alpha (pancreatic stone protein, | | (+) | 34645890&g= |
| | | pancreatic thread protein) | | | htcDnaNearGene&i= |
| | | | | | NM_002909&c= |
| | | | | | chr2&l= |
| | | | | | 79304291&r= |
| | | | | | 79309253&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | REG1B | regenerating islet-derived 1 | NM_006507.2 | Chr 2: 79.269-79.272 Mbp | http://genome.ucsc.edu/cgi-bin/hgc?hgsid= |
| | | beta (pancreatic thread protein,) | | (−) | 34645907&g= |
| | | pancreatic stone protein) | | | htcDnaNearGene&i= |
| | | | | | NM_006507&c= |
| | | | | | chr2&l= |
| | | | | | 79268858&r= |
| | | | | | 79273827&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolShad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SERPIN12 | Serine (or cysteine) proteinase | NM_006217.2 | Chr 3: 168.561-168.591 Mbp | |
| | | inhibitor, clade I (neuroserpin), | | (−) | |
| | | member 2 | | | |
| | SPINK1 | Serine protease inhibitor, Kazal | NM_003122.2 | Chr 5: 147.187-147.195 Mbp | http://genome.ucsc.edu/cgi-bin/hgc?hgsid= |
| | | type 1 | | (−) | 34645943&g= |
| | | | | | htcDnaNearGene&i= |
| | | | | | NM_003122&c= |
| | | | | | chr5&l= |
| | | | | | 147186303&r= |
| | | | | | 147195418&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | SYCN | Syncollin | | Chr 19: 44.369-44.37 Mbp | |
| | | | | (−) | |
| | TRY6 | Trypsinogen C | NM_139000 | Chr 7: 141.842-141.845 Mbp | http://genome.ucsc.edu/cgi-bin/hgc?hgsid= |
| | | | | (+) | 34646018&g= |
| | | | | | htcDnaNearGene&i= |
| | | | | | NM_139000&c= |
| | | | | | chr7&l= |
| | | | | | 141841283&r= |
| | | | | | 141846943&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Pancreaticislets | IAPP | Islet amyloid polypeptide | NM_000415.1 | Chr 12: 21.426-21.432 Mbp | http://genome.ucsc.edu/cgi-bin/hgc?hgsid= |
| | | | | (+) | 34646120&g= |
| | | | | | htcDnaNearGene&i= |
| | | | | | NM_000415&c= |
| | | | | | chr12&l= |
| | | | | | 21425084&r= |
| | | | | | 21433683&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | PAP | Pancreatitis-associated protein | NM_002580.1 | Chr 2: 79.341-79.344 Mbp | http://genome.ucsc.edu/cgi-bin/hgc?hgsid= |
| | | | | (−) | 34646153&g= |
| | | | | | htcDnaNearGene&i= |
| | | | | | NM_002580&c= |
| | | | | | chr2&l= |
| | | | | | 79340840&r= |
| | | | | | 79345587&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | PCSK1 | Proprotein convertase | NM_000439.3 | Chr 5: 95.754-95.797 Mbp | http://genome.ucsc.edu/cgi-bin/hgc?hgsid= |
| | | subtilisen/kexin type 1 | | (−) | 34646184&g= |
| | | | | | htcDnaNearGene&i= |
| | | | | | NM_000439&c= |
| | | | | | chr5&l= |
| | | | | | 95753830&r= |
| | | | | | 95798664&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | SST | Somatostatin | NM_001048.2 | Chr 3: 188.788-188.79 Mbp | http://genome.ucsc.edu/cgi-bin/hgc?hgsid= |
| | | | | (−) | 34646232&g= |
| | | | | | htcDnaNearGene&i= |
| | | | | | NM_001048&c= |
| | | | | | chr3&l= |
| | | | | | 188787726&r= |
| | | | | | 188791133&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | UNQ429 | LLM429 | NM_198448.1 | Chr 2: 79.21-79.213 Mbp | |
| | | | | (+) | |
| BM-CD105+ | CA1 | Carbonic anhydrase 1 | NM_001738.1 | Chr 8: 86.019-86.071 Mbp | http://genome.ucsc.edu/cgi-bin/hgc?hgsid= |
| | | | | (−) | 34646365&g= |
| | | | | | htcDnaNearGene&i= |
| | | | | | NM_001738&c= |
| | | | | | chr8&l= |
| | | | | | 86019484&r= |
| | | | | | 86071370&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| Endothelial | GYPA | Glycophorin A (includes MN | NM_002099.2 | Chr 4: 145.496-145.528 Mbp | http://genome.ucsc.edu/cgi-bin/hgc?hgsid= |
| | | blood group) | | (−) | 34646395&g= |
| | | | | | htcDnaNearGene&i= |
| | | | | | NM_002099&c= |
| | | | | | chr4&l= |
| | | | | | 145495643&r= |
| | | | | | 145529031&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HBG2 | Hemoglobin, gamma G | NM_000184.2 | Chr 11: 5.233-5.235 Mbp | http://genome.ucsc.edu/cgi-bin/hgc?hgsid= |
| | | | | (−) | 34646434&g= |
| | | | | | htcDnaNearGene&i= |
| | | | | | NM_000184&c= |
| | | | | | chr11&l= |
| | | | | | 5232457&r= |
| | | | | | 5236048&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | HEMGN | Hemogen | NM_197978.1 | Chr 9: 94.146-94.164 Mbp | http://genome.ucsc.edu/cgi-bin/hgc?hgsid= |
| | | | | (−) | 34646448&g= |
| | | | | | htcDnaNearGene&i= |
| | | | | | NM_018437&c= |
| | | | | | chr9&l= |
| | | | | | 94145526&r= |
| | | | | | 94165588&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | NMU | Neuromedin U | NM_006681.1 | Chr 4: 56.311-56.352 Mbp | http://genome.ucsc.edu/cgi-bin/hgc?hgsid= |
| | | | | (−) | 34646473&g= |
| | | | | | htcDnaNearGene&i= |
| | | | | | NM_006681&c= |
| | | | | | chr4&l= |
| | | | | | 56310320&r= |
| | | | | | 56353388&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SLC4A1 | solute carrier family 4, anion | NM_000342.1 | Chr 17: 42.802-42.82 Mbp | http://genome.ucsc.edu/cgi-bin/hgc?hgsid= |
| | | exchanger, member 1 | | (−) | 34646489&g= |
| | | (erythrocyte membrane protein | | | htcDnaNearGene&i= |
| | | band 3, Diego blood group) | | | NM_000342&c= |
| | | | | | chr17&l= |
| | | | | | 42801204&r= |
| | | | | | 42821632&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | TOP2A | topoisomerase (DNA) II alpha | NM_001067.2 | Chr 17: 38.453-38.482 Mbp | http://genome.ucsc.edu/cgi-bin/hgc?hgsid= |
| | | 170 kDa | | (−) | 34646510&g= |
| | | | | | htcDnaNearGene&i= |
| | | | | | NM_001067&c= |
| | | | | | chr17&l= |
| | | | | | 38452558&r= |
| | | | | | 38483933&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hegSeq.maskRepeats=1&hgSeq.repMasking=lower&submit= |
| | | | | | submit |
| BM-CD34+ | DNTT | Deoxynucleotidyltransferase, | NM_004088.2 | Chr 10: 98.195-98.229 Mbp | http://genome.ucsc.edu/cgi-bin/hgc?hgsid= |
| | | terminal | | (+) | 34646546&g= |
| | | | | | htcDnaNearGene&i= |
| | | | | | NM_004088&c= |
| | | | | | chr10&l= |
| | | | | | 98194437&r= |
| | | | | | 98230547&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | FOSB | FBJ murine osteosarcoma viral | NM_006732.1 | Chr 19: 50.647-50.654 Mbp | http://genome.ucsc.edu/cgi-bin/hgc?hgsid= |
| | | oncogene homolog B | | (+) | 34646569&g= |
| | | | | | htcDnaNearGene&i= |
| | | | | | NM_006732&c= |
| | | | | | chr19&l= |
| | | | | | 50646301&r= |
| | | | | | 50655485&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ITGA2B | Integrin, alpha 2b (platelet | NM_000419.2 | Chr 17: 42.46-42.477 Mbp | http://genome.ucsc.edu/cgi-bin/hgc?hgsid= |
| | | glycoprotein IIB/IIA complex, | | (−) | 34646591&g= |
| | | antigen CD41B) | | | htcDnaNearGene&i= |
| | | | | | NM_000419&c= |
| | | | | | chr17&l= |
| | | | | | 42459314&r= |
| | | | | | 42478638&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| BM-CD71 + Early | ANK1 | Ankyrin 1, erythrocytic | NM_000037.2 | Chr 8: 41.251-41.396 Mbp | http://genome.ucsc.edu/cgi-bin/hgc?hgsid= |
| | | | | (−) | 34646690&g= |
| | | | | | htcRefMrna&i= |
| | | | | | NM_000037&c= |
| | | | | | chr8&l= |
| | | | | | 41250690&r= |
| | | | | | 41397087&o= |
| | | | | | refGene&table= |
| | | | | | refGene |
| Erythroid | CA2 | Carbonic anhydrase II | NM_000067.1 | Chr 8: 86.156-86.173 Mbp | http://genome.ucsc.edu/cgi-bin/hgc?hgsid= |
| | | | | (+) | 34646734&g= |
| | | | | | htcDnaNearGene&i= |
| | | | | | NM_000067&c= |
| | | | | | chr8&l= |
| | | | | | 86155273&r= |
| | | | | | 86174749&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | CLIC2 | Chloride intracellular channel 2 | NM_001289.3 | Chr X: 152.023-152.081 Mbp | http://genome.ucsc.edu/cgi-bin/hgc?hgsid= |
| | | | | (−) | 34646754&g= |
| | | | | | htcDnaNearGene&i= |
| | | | | | NM_001289&c= |
| | | | | | chrX&l= |
| | | | | | 152022518&r= |
| | | | | | 152082024&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | EPB42 | Erythrocyte membrane protein | NM_000119.1 | Chr 15: 41.068-41.092 Mbp | http://genome.ucsc.edu/cgi-bin/hgc?hgsid= |
| | | band 4.2 | | (−) | 34646796&g= |
| | | | | | htcDnaNearGene&i= |
| | | | | | NM_000119&c= |
| | | | | | chr15&l= |
| | | | | | 41067565&r= |
| | | | | | 41093619&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ERAF | Erythroid associated factor | NM_016633.1 | Chr 16: 31.536-31.537 Mbp | http://genome.ucsc.edu/cgi-bin/hgc?hgsid= |
| | | | | (+) | 34646847&g= |
| | | | | | htcDnaNearGene&i= |
| | | | | | NM_016633&c= |
| | | | | | chr16&l= |
| | | | | | 31535165&r= |
| | | | | | 31538069&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | FOXO3A | forkhead box O3A | NM_001455.2 | Chr 6: 108.881-109.002 Mbp | http://genome.ucsc.edu/cgi-bin/hgc?hgsid= |
| | | | | (+) | 34646917&g= |
| | | | | | htcDnaNearGene&i= |
| | | | | | NM_001455&c= |
| | | | | | chr6&l= |
| | | | | | 108880155&r= |
| | | | | | 109003098&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | GYPB | Glycophorin B (includes Ss | NM_002100.2 | Chr 4: 145.383-145.406 Mbp | http://genome.ucsc.edu/cgi-bin/hgc?hgsid= |
| | | blood group) | | (+) | 34646972&g= |
| | | | | | htcDnaNearGene&i= |
| | | | | | NM_002100&c= |
| | | | | | chr4&l= |
| | | | | | 145493904&r= |
| | | | | | 145519123&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | HBQ1 | Hemoglobin, theta 1 | NM_005331.3 | Chr 16: 0.17-0.171 Mbp | http://genome.ucsc.edu/cgi-bin/hgc?hgsid= |
| | | | | (+) | 34647029&g= |
| | | | | | htcDnaNearGene&i= |
| | | | | | NM_005331&c= |
| | | | | | chr16&l= |
| | | | | | 169334&r= |
| | | | | | 172178&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | MSCP | Mitochondrial solute carrier | NM_016612.1 | Chr 8: 23.207-23.25 Mbp | http://genome.ucsc.edu/cgi-bin/hgc?hgsid= |
| | | protein | | (+) | 34647097&g= |
| | | | | | htcDnaNearGene&i= |
| | | | | | NM_016612&c= |
| | | | | | chr8&l= |
| | | | | | 23206033&r= |
| | | | | | 23251305&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | NFE2 | nuclear factor (erythroid- | NM_006163.1 | Chr 12: 54.402-54.406 Mbp | http://genome.ucsc.edu/cgi-bin/hgc?hgsid= |
| | | derived2), 45 kDa | | (−) | 34647151&g= |
| | | | | | htcDnaNearGene&i= |
| | | | | | NM_006163&c= |
| | | | | | chr12&l= |
| | | | | | 54401641&r= |
| | | | | | 54407291&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | NUSAP1 | nucleolar and spindle | NM_016359.1 | Chr 15: 39.204-39.252 Mbp | http://genome.ucsc.edu/cgi-bin/hgc?hgsid= |
| | | | | | 34647189&g= |
| | | | | | htcDnaNearGene&i= |
| | | | | | NM_016359&c= |
| | | | | | chr15&l= |
| | | | | | 39203225&r= |
| | | | | | 39253382&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RHAG | Rhesus blood group-associated | NM_000324.1 | Chr 6: 49.574-49.605 Mbp | http://genome.ucsc.edu/cgi-bin/hgc?hgsid= |
| | | glycoprotein | | (−) | 34647276&g= |
| | | | | | htcDnaNearGene&i= |
| | | | | | NM_000324&c= |
| | | | | | chr6&l= |
| | | | | | 49573283&r= |
| | | | | | 49606948&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RHCE | Rhesus blood group, CcEe | NM_020485.2 | Chr 1: 24.597-24.931 Mbp | http://genome.ucsc.edu/cgi-bin/hgc?hgsid= |
| | | antigens | | (−) | 34647306&g= |
| | | | | | htcDnaNearGene&i= |
| | | | | | NM_020485&c= |
| | | | | | chr1&l= |
| | | | | | 24596833&r= |
| | | | | | 24657408&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RHD | Rhesus blood group, D antigen | NM_001034.1 | Chr 2: 10.267-10.275 Mbp | http://genome.ucsc.edu/cgi-bin/hgc?hgsid= |
| | | | | (+) | 34647365&g= |
| | | | | | htcDnaNearGene&i= |
| | | | | | NM_016124&c= |
| | | | | | chr1&l= |
| | | | | | 24667748&r= |
| | | | | | 24860158&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | RRM2 | Ribonucleoltide reductase M2 | NM_001034.1 | Chr 2: 10.267-10.275 Mbp | http://genome.ucsc.edu/cgi-bin/hgc?hgsid= |
| | | polypeptide | | (+) | 34647447&g= |
| | | | | | htcDnaNearGene&i= |
| | | | | | NM_001034&c= |
| | | | | | chr2&l= |
| | | | | | 10266649&r= |
| | | | | | 10276538&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | SELENBP1 | Selenium binding protein 1 | NM_003944.2 | Chr 1: 148.111-148.12 Mbp | http://genome.ucsc.edu/cgi-bin/hgc?hgsid= |
| | | | | (−) | 34647489&g= |
| | | | | | htcDnaNearGene&i= |
| | | | | | NM_003944&c= |
| | | | | | chr1&l= |
| | | | | | 148110874&r= |
| | | | | | 148121259&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| Fetalliver | 1232_s_at | Human insulin-like growth | | Chr 7: 45.639-45.639 Mbp | |
| | | factor binding protein | | (+) | |
| | | (hIGFBP1) gene, complete cds | | | |
| | 31506_s_at | Human neutrophil peptide-3 | | Chr 8: 7.033-7.034 Mbp | |
| | | gene, complete cds | | (−) | |
| | 33487_at | Human gene for 4- | | Chr 12: 122.046-122.054 Mbp | |
| | | hydroxyphenylpyruvic acid | | (−) | |
| | | dioxygenase (HPD), comlete | | | |
| | | cds | | | |
| | 33703_f_at | Human phosphoenolpyruvate | | Chr 20: 56.779-56.779 Mbp | |
| | | carboxykinase (PCK1) gene, | | (+) | |
| | | complete cds with repeats | | | |
| | 33990_at | Human mRNA clone with | | Chr 4: 74.687-74.687 Mbp | |
| | | similarity to L-glycerol-3- | | (+) | |
| | | phosphate-NAD | | | |
| | | oxidoreductase and albumin | | | |
| | | gene sequences | | | |
| | 33991_g_at | Human mRNA clone with | | Chr 4: 74.75-74.753 Mbp | |
| | | similarity to L-glycerol-3- | | (+) | |
| | | phosphate-NAD | | | |
| | | oxidoreductase and albumin | | | |
| | | gene sequences | | | |
| | 33992_at | Human serum albumin (ALB) | | Chr 4: 74.685-74.685 Mbp | |
| | | gene, complete cds | | (+) | |
| | 36646_at | Human plasminogen gene | | Chr 6: 160.995-161.007 Mbp | |
| | | | | (+) | |
| | 36995_at | Human inter-alpha-trypsin | | Chr 9: 110.276-110.278 Mbp | |
| | | inhibitor light chain (ITI) gene | | (−) | |
| | 37175_at | Human antithrombin III | | Chr 1: 170.453-170.459 Mbp | |
| | | (ATIII) gene | | (−) | |
| | 38585_at | H. sapiens G-gamma globin and | | Chr 11: 5.233-5.235 Mbp | |
| | | A-gamma globin genes, | | (−) | |
| | | complete cdss | | | |
| | 38825_at | Human fibrinogen alpha chain | | Chr 4: 155.97-155.97 Mbp | |
| | | gene, complete mRNAs | | (−) | |
| | 38890_at | Homo sapiens gene for serum | | Chr 1: 156.335-156.336 Mbp | |
| | | amyloid P component, | | (+) | |
| | | complete cds | | | |
| | 39763_at | Human hemopexin gene | | Chr 11: 6.411-6.412 Mbp | |
| | | | | (−) | |
| | 40114_at | Human alpha-fetoprotein | | Chr 4: 74.718-74.722 Mbp | |
| | | (AFP) mRNA, complete cds | | (+) | |
| | 926_at | HUMMT2A Human (clone | | Chr 16: 56.435-56.436 Mbp | |
| | | 14VS) metallothionein-IG | | (−) | |
| | | (MT1G) gene; complete cds | | | |
| | AFP | alpha-fetoprotein | NM_001134.1 | Chr 4: 74.702-74.22 Mbp | http://genome.ucsc.edu/cgi-bin/hgc?hgsid= |
| | | | | (+) | 34521952&g= |
| | | | | | htcDnaNearGene&i= |
| | | | | | NM_001134&c= |
| | | | | | chr4&l= |
| | | | | | 74701568&r= |
| | | | | | 74723128&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | AHSG | alpha-2-HS-glycoprotein | NM_001622.1 | Chr 3: 187.732-187.741 Mbp | |
| | | | | (+) | |
| | ALB | Albumin | NM_000477.3 | Chr 4: 74.67-74.687 Mbp | http://genome.ucsc.edu/cgi-bin/hgc?hgsid= |
| | | | | (+) | 34521814&g= |
| | | | | | htcDnaNearGene&i= |
| | | | | | NM_000477&c= |
| | | | | | chr4&l= |
| | | | | | 74669641&r= |
| | | | | | 74688768&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats=1&hgSeq.repMasking=lower&submit= |
| | | | | | submit |
| | ALDOB | Aldolase B, fructose- | NM_000035.2 | Chr 9: 97.641-97.655 Mbp | |
| | | bisphosphate | | (−) | |
| | AMBP | alpha-1-microglobulin/bikunin | NM_001633.2 | Chr 9: 110.276-110.294 Mbp | |
| | | precursor | | (−) | |
| | APOA1 | Apolipoprotein A-1 | NM_000039.1 | Chr 11: 116.74-116.742 Mbp | |
| | | | | (−) | |
| | APOA2 | Apolipoprotein A-II | NM_001643.1 | Chr 1: 157.969-157.971 Mbp | |
| | | | | (−) | |
| | APOB | Apolipoprotein B (including | NM_000384.1 | Chr 2: 21.182-21.224 Mbp | |
| | | Ag(x) antigen) | | (−) | |
| | APOC2 | Apolipoprotein C-II | NM_000483.3 | Chr 19: 50.125-50.128 Mbp | |
| | | | | (+) | |
| | APOC3 | Apolipoprotein C-III | NM_000040.1 | Chr 11: 116.734-116.737 Mbp | |
| | | | | (+) | |
| | APOH | Apolipoprotein H (beta-2- | NM_000042.1 | Chr 17: 64.625-64.643 Mbp | |
| | | glycoprotein 1) | | (−) | |
| | CPS1 | Carbamoyl-phosphate | NM_001875.2 | Chr 2: 211.385-211.507 Mbp | |
| | | synthetase 1, mitochondrial | | (+) | |
| | CYP3A7 | Cytochrome P450, family 3, | NM_000765.2 | Chr 7: 98.9-98.93 Mbp | |
| | | subfamily A, polypeptide 7 | | (−) | |
| | FGA | Fibrinogen, A alpha | NM_000508.2 | Chr 4: 155.97-155.978 Mbp | |
| | | polypeptide | | (−) | |
| | FGB | Fibrinogen, B beta polypeptide | NM_005141.1 | Chr 4: 155.95-155.958 Mbp | |
| | | | | (+) | |
| | FGG | Fibrinogen, gamma | NM_000509.3 | Chr 4: 155.991-155.999 Mbp | |
| | | polypeptide | | (−) | |
| | GC | group-specific compnent | NM_000583.2 | Chr 4: 73.008-73.05 Mbp | |
| | | (vitamin D binding protein) | | (−) | |
| | HBZ | Hemoglobin, zeta | NM_005332.2 | Chr 16: 0.142-0.144 Mbp | |
| | | | | (+) | |
| | Hs.407269 | Clone FLB5539 PRO1454 | | Chr 12: 69.001-69.004 Mbp | |
| | | mRNA, complete cds | | (−) | |
| | IGF2 | Insulin-like growth factor 2 | NM_000612.2 | Chr 11: 2.113-2.119 Mbp ( | |
| | | (somatomedin A) | | | |
| | LIPC | Lipase, hepatic | NM_000236.1 | Chr 15: 56.303-56.44 Mbp | |
| | | | | (+) | |
| | ORM1 | Ororomucoid1 | NM_000607.1 | Chr 9: 110.538-110.542 Mbp | |
| | | | | (+) | |
| | PLG | Plasminogen | NM_000301.1 | Chr 6: 160.956-161.007 Mbp | |
| | | | | (+) | |
| | PRTN3 | Proteinase 3 (serine proteinase, | NM_002777.2 | Chr 19: 0.78-0.788 Mbp | |
| | | neutrophil, Wegener | | (+) | |
| | | granulomatosis autoantigen) | | | |
| | SERPINA1 | Serine (or cysteine) proteinase | NM_000295.2 | Chr 14: 92.834-92.845 Mbp | |
| | | inhibitor, clade A (alpha-1 | | (− | |
| | | antiproteinase, antitrypsin), | | | |
| | | member 1 | | | |
| | SERPINC1 | Serine (or cysteine) proteinase | NM_000488.1 | Chr 1: 170.453-170.467 Mbp | |
| | | inhibitor, clade C | | (−) | |
| | | (antithrombin), member 2 | | | |
| | SLC2A2 | solute carrier family 2 | NM_000340.1 | Chr 3: 172.116-172.146 Mbp | |
| | | (facilitated glucose | | (−) | |
| | | transporter), member 2 | | | |
| | SPP2 | secreted phosphoprotein 2, | NM_006944.1 | Chr 2: 234.975-234.994 Mbp | |
| | | 24 kDa | | (+) | |
| | TM4SF4 | transmembrane 4 superfamily | NM_004617.2 | Chr 3: 150.474-150.502 Mbp | |
| | | member 4 | | (+) | |
| | UGT2B4 | UDP glycosyltransferase 2 | NM_021139.1 | Chr 4: 70.595-70.611 Mbp | |
| | | family, polypeptide B4 | | (−) | |
| Fetalbrain | CHL1 | Cell adhesion molecule with | NM_006614.2 | Chr 3: 0.213-0.426 Mbp | |
| | | homology to L1CAM (close | | (+) | |
| | | homolog of L1) | | | |
| | FABP7 | fatty acid binding protein 7, | NM_001446.3 | Chr 6: 123.035-123.04 Mbp | |
| | | brain | | (+) | |
| | FOX1B | forkhead box G1B | NM_005249.3 | Chr 14: 27.225-27.228 Mbp | |
| | | | | (+) | |
| | GPM6A | Glycoprotein M6A | NM_005277.3 | Chr 4: 177.138-177.508 Mbp | |
| | | | | (−) | |
| | Hs.4267 | Clones 24714 and 24715 | | Chr 18: 29.58-29.582 Mbp | |
| | | mRNA sequence | | (+) | |
| | MGC8685 | Tubulin, beta polypeptide | NM_178012.3 | Chr 6: 3.214-3.217 Mbp | |
| | | paralog | | (−) | |
| | RTN1 | Transcribed sequences | NM_021136.2 | Chr 14: 58.052-58.327 Mbp | |
| | | | | (−) | |
| | TUBB | Tubulin, beta polypeptide | NM_001069.1 | Chr 6: 3.143-3.147 Mbp | |
| | | | | (−) | |
| Fetalthyroid | ACTA1 | Actin, alpha 1, skeletal muscle | NM_001100.3 | Chr 1: 225.966-225.969 Mbp | |
| | | | | (−) | |
| | SLC26A7 | solute carrier family 26, | NM_134266.1 | Chr 8: 91.93-92.079 Mbp | |
| | | member 7 | | (+) | |
| | TG | Thyroglubulin | NM_052832.2 | Chr 8: 91.93-92.079 Mbp | |
| | | | | (+) | |
| | TPO | Thyroid peroxidase | NM_175719.1 | Chr 2: 1.49-1.619 Mbp | |
| | | | | (+) | |
| | TSHR | Thyroid stimulating horomone | NM_000369.1 | Chr 14: 79.411-79.6 Mbp | |
| | | receptor | | (+) | |
| Fetallung | HPR | Haptoglobin-related protein | NM_020995.3 | Chr 16: 71.832-71.846 Mbp | |
| | | | | (+) | |
| | SFTPB | Surfactant, pulmonary- | NM_198843.1 | Chr 2: 85.842-85.853 Mbp | |
| | | associated protein B | | (−) | |
| | SFTPC | Surfactant, pulmonary- | NM_003018.2 | Chr 8: 21.839-21.842 Mbp | |
| | | associated protein C | | (+) | |
| DRG | 40657_at | yl28b07.s1 Homo sapiens | | Chr 3: 187.978-187.978 Mbp | |
| | | cDNA, 3′ end | | (+) | |
| | FABP4 | fatty acid binding protein 4, | NM_001442.1 | Chr 8: 82.114-82.118 Mbp | |
| | | adipocyte | | (−) | |
| | NEF3 | Neurofilament 3 (150 kDa | NM_005382.1 | Chr 8: 24.591-24.597 Mbp | |
| | | medium) | | (+) | |
| | NEFL | Neurofilament, light | NM_006158.1 | Chr 8: 24.63-24.634 Mbp | |
| | | polypeptide 68 kDa | | (−) | |
| | TAC1 | Tachykinin, precursor 1 | NM_003182.1 | Chr 7: 96.959-96.967 Mbp | |
| | | (substance K, substance P, | | (+) | |
| | | neurokinin 1, neurokinin 2, | | | |
| | | neuromedin L, neurokinin | | | |
| | | alpha, neuropeptide K, | | | |
| | | neuropeptide gamma) | | | |
| Prostate | 1197_at | Human enteric smooth muscle | | Chr 2: 74.098-74.104 Mbp | |
| | | gamma-actin gene, 5′ flank and | | (+) | |
| | 33767_at | H. sapiens NF-H gene, exon 1 | | Chr 22: 28.211-28.211 Mbp | |
| | | (and joined CDS) | | (+) | |
| | ACPP | Acid phosphate, prostate | NM_001099.2 | Chr 3: 133.317-133.359 Mbp | |
| | | | | (+) | |
| | AZGP1 | alpha-2-glycoprotein 1, zinc | NM_001185.2 | Chr 7: 99.161-99.171 Mbp | |
| | | | | (−) | |
| | CLDN | Claudin 3 | NM_001306.2 | Chr 7: 72.581-72.582 Mbp | |
| | | | | (−) | |
| | FOXA1 | Forkhead box A1 | NM_004496.2 | Chr 14: 36.049-36.054 Mbp | |
| | | | | (−) | |
| | KLK2 | Kallikrein 2, Prostatic | NM_005551.2 | Chr 19: 56.052-56.059 Mbp | |
| | | | | (+) | |
| | KLK3 | Kallikrein 3, (prostate specific | NM_001648.2 | Chr 19: 56.034-56.04 Mbp | |
| | | antigen) | | (+) | |
| | KRT15 | Keratin 15 | NM_002275.2 | Chr 17: 39.578-39.587 Mbp | |
| | | | | (−) | |
| | MSMB | Microseminoprotein, beta- | NM_002443.2 | Chr 10: 51.441-51.455 Mbp | |
| | | | | (+) | |
| | MYH11 | Myosin, heavy polypeptide 11, | NM_002474.1 | Chr 16: 15.724-15.878 Mbp | |
| | | smooth muscle | | (−) | |
| | NEFH | Neurofilament, heavy | NM_021076.2 | Chr 22: 28.191-28.211 Mbp | |
| | | polypeptide 200 kDa | | (+) | |
| | TGM4 | Transglutaminase 4 (prostate) | NM_003241.1 | Chr 3: 44.735-44.775 Mbp | |
| | | | | (+) | |
| | TMPRSS2 | Transmembrane protease, | NM_005656.2 | Chr 21: 41.757-41.8 Mbp | |
| | | serine 2 | | (−) | |
| Uterus | 40776_at | Human desmin gene, complete | | Chr 2: 220.254-220.255 Mbp | |
| | | cds | | (+) | |
| | CNN1 | Calponin 1, basic, smooth | NM_001299.3 | Chr 19: 11.494-11.506 Mbp | |
| | | muscle | | (+) | |
| | PAEP | Progestagen-associated | NM_002571.1 | Chr 9: 131.976-131.981 Mbp | |
| | | endometrial protein (placental | | (+) | |
| | | protein 14, pregnancy- | | | |
| | | associated endometrial alpha- | | | |
| | | 2-globulin, alpha uterine | | | |
| | | protein) | | | |
| Testis | 34658_at | Human protamine 1 (PRM1), | | Chr 16: 11.335-11.336 Mbp | |
| | | protamine 2 (PRM2) and | | (−) | |
| | | transition protein 2 (TNP2) | | | |
| | | genes, complete cds | | | |
| | 36301_at | Homo sapiens chromosome 19, | | Chr 19: 17.772-17.773 Mbp | |
| | | cosmid F19847 | | (−) | |
| | 37008_r_at | Human protein C inhibitor | | Chr 14: 93.049-93.049 Mbp | |
| | | gene, complete cds | | (+) | |
| | 39156_at | dJ149A16.3 (Ret finger | | Chr 22: 31.08-31.08 Mbp | |
| | | protein-like 3 antisense) | | (−) | |
| | 41149_at | Homo sapiens Chromosome 16 | | Chr 16: 20.783-20.788 Mbp | |
| | | BAC clone CIT987SK-44M2 | | (+) | |
| | AKAP4 | A kinase (PRKA) anchor | NM_003886.2 | Chr X: 48.653-48.663 Mbp | |
| | | protein 4 | | (−) | |
| | ART3 | ADP-ribosyltransferase 3 | NM_001179.2 | Chr 4: 77.388-77.426 Mbp | |
| | | | | (+) | |
| | CDKN3 | Cyclin-dependent kinase | NM_005192.2 | Chr 14: 52.853-52.876 Mbp | |
| | | inhibitor 3 (CDK2-associated | | (+) | |
| | | dual specificity phosphatase) | | | |
| | GAGE4 | G antigen 5 | NM_001475.1 | Chr X: 48.023-48.04 Mbp | |
| | | | | (+) | |
| | GK2 | Glycerol kinase 2 | NM_033214.2 | Chr 4: 80.72-80.722 Mbp | |
| | | | | (−) | |
| | Insl3 | Insulin-like 3 (Leydig cell) | NM_005543.2 | Chr 19: 17.772-17.777 Mbp | |
| | | | | (−) | |
| | LDHC | Lactate dehydrogenase C | NM_002301.2 | Chr 11: 18.473-18.511 Mbp | |
| | | | | (+) | |
| | LOC81691 | Exonuclease NEF-sp | NM_030941.1 | Chr 16: 20.745-20.788 Mbp | |
| | | | | (+) | |
| | ODF2 | outer dense fiber of sperm tails 2 | NM_002540.3 | Chr 9: 124.672-124.716 Mbp | |
| | | | | (+) | |
| | PRM1 | Protamine 1 | NM_002761.1 | Chr 16: 11.341-11.341 Mbp | |
| | | | | (−) | |
| | PRM2 | Protamine 2 | NM_002762.1 | Chr 16: 11.335-11.336 Mbp | |
| | | | | (−) | |
| | SPINK2 | Serine protease inhibitor, Kazal | NM_021114.1 | Chr 4: 57.525-57.537 Mbp | |
| | | type 2 (acrosin-trypsin | | (−) | |
| | | inhibitor) | | | |
| | TKTL1 | Transketolase-like 1 | NM_012253.1 | Chr X: 151.109-151.144 Mbp | |
| | | | | (+) | |
| | TNP1 | transition protein 1 (during | NM_003284.2 | Chr 2: 217.688-217.688 Mbp | |
| | | histone to protamine | | (−) | |
| | | replacement) | | | |
| | TSPY2 | Testis specific protein, Y- | NM_022573.1 | Chr Y: 9.14-9.143 Mbp | |
| | | linked 2 | | (+) | |
| | ZPBP | zona pellucida binding protein | NM_007009.1 | Chr 7: 49.687-49.843 Mbp | |
| | | | | (−) | |
| TestisSeminiferousTubule | ANKRD7 | Ankyrin repeat domain 7 | NM_019644.1 | Chr 7: 117.405-117.423 Mbp | |
| | | | | (+) | |
| Placenta | 1332_f_at | Human germ line gene for | | Chr 17: 62.335-62.336 Mbp | |
| | | growth hormone | | (−) | |
| | | (presomatotropin) | | | |
| | 1691_g_at | ovary- and prostate-specific | | Chr 15: 49.114-49.114 Mbp | |
| | | exon 1 from Human | | (−) | |
| | | cytochrome P-450 aromatase | | | |
| | | gene, multiple exons 1 and | | | |
| | | exon 2 | | | |
| | 203807_x_at | chorionic somatomammotropin | | Chr 17: 62.29-62.291 Mbp | |
| | | hormone 2 | | (−) | |
| | 208294_x_at | chorionic somatomammotropin | | Chr 17: 62.327-62.329 Mbp | |
| | | hormone-like 1 | | (−) | |
| | 31493_s_at | Human growth hormone (GH-1 | | Chr 17: 62.29-62.314 Mbp | |
| | | and GH-2) and chorionic | | (−) | |
| | | somatomammotropin (CS-1, | | | |
| | | CS-2 and CS-5) genes, | | | |
| | | complete cds | | | |
| | 35721_at | Human 3-beta-hydroxysteroid | | Chr 1: 119.204-119.204 Mbp | |
| | | dehydrogenase/delta-5-delta-4- | | (+) | |
| | | isomerase (3-beta-HSD) gene, | | | |
| | | complete cds | | | |
| | 36784_at | human growth horomone (GH- | | Chr 17: 62.328-62.328 Mbp | |
| | | 1 and GH-2) and chorionic | | (−) | |
| | | somato mammotropin (CS- | | | |
| | | 1, CS-2, and CS-5) genes, | | | |
| | | complete cds | | | |
| | 39352_at | thyroid-stimulating hormone | | Chr 6: 87.745-87.748 Mbp | |
| | | alpha subunit [human, | | (−) | |
| | | Genomic, 1327 nt 4 segments] | | | |
| | 40316_at | Human growth hormone | | Chr 17: 62.298-62.299 Mbp | |
| | | variant (HGH-V) gene, | | (−) | |
| | | complete cds | | | |
| | ABP1 | Amiloride binding protein 1 | NM_001091.1 | Chr 7: 149.864-149.873 Mbp | |
| | | (amine oxidase(copper- | | (+) | |
| | | containing)) | | | |
| | ADAM12 | a disintegrin and | NM_003474.2 | Chr 10: 127.744-128.118 Mbp | |
| | | metalloproteinase domain 12 | | (−) | |
| | | (meltrin alpha) | | | |
| | ALPP | Alkaline phosphatase, placental | NM_001632.2 | Chr 2: 233.207-233.211 Mbp | |
| | | (Reganisozyme) | | (+) | |
| | ALPPL2 | Alkaline phosphatase, | NM_031313.1 | Chr 2: 233.235-233.239 Mbp | |
| | | placental-like 2 | | (+) | |
| | CAPN6 | Calpain 6 | NM_014289.2 | Chr X: 108.513-108.538 Mbp | |
| | | | | (−) | |
| | CGA | Glycoprotein horomones, apha | NM_000735.2 | Chr 6: 87.745-87.754 Mbp | |
| | | polypeptide | | (−) | |
| | CGB | Chorionic gonadotropin, beta | NM_000737.2 | Chr 19: 54.202-54.203 Mbp | |
| | | polypeptide | | (−) | |
| | CGB2 | Chorionic gonadotropin, beta | NM_033378.1 | Chr 19: 54.211-54.212 Mbp | |
| | | polypeptide 2 | | (+) | |
| | CRH | Corticotropin releasing | NM_000756.1 | Chr 8: 66.811-66.813 Mbp | |
| | | horomone | | (−) | |
| | CSH1 | Chorionic | NM_001317.3 | Chr 17: 62.313-62.314 Mbp | |
| | | somatomammotropin | | (−) | |
| | | horomone 1 (placental | | | |
| | | lactogen) | | | |
| | CSH2 | Chorionic | NM_020991.3 | Chr 17: 62.29-62.291 Mbp | |
| | | somatomammotropin | | (−) | |
| | | horomone 2 | | | |
| | CSHL1 | Chorionic | NM_001318.2 | Chr 17: 62.327-62.329 Mbp | |
| | | Sommatomammotropin | | (−) | |
| | | horomone-like 1 | | | |
| | CYP19A1 | Cytochrome P450, family 19, | NM_000103.2 | Chr 15: 49.08-49.209 Mbp | |
| | | subfamily A, polypeptide 1 | | (−) | |
| | DLK1 | delta-like 1 homolog | NM_003836.3 | Chr 14: 99.183-99.191 Mbp | |
| | | (Drosophilia) | | (+) | |
| | EB13 | Epstein-Barr virus induced | NM_005755.2 | Chr 19: 4.169-4.177 Mbp | |
| | | gene 3 | | (+) | |
| | FBLN1 | Fibulin 1 | NM_001996.2 | Chr 22: 44.175-44.273 Mbp | |
| | | | | (+) | |
| | GAGEC1 | G antigen, family C, 1 | NM_007003.2 | Chr X: 48.291-48.296 Mbp | |
| | | | | (+) | |
| | GDF15 | growth differentiation factor 15 | NM_004864.1 | Chr 19: 18.324-18.345 Mbp | |
| | | | | (+) | |
| | GH1 | growth horomone 1 | NM_000515.3 | Chr 17: 62.335-62.337 Mbp | |
| | | | | (−) | |
| | GH2 | growth horomone 2 | NM_002059.3 | Chr 17: 62.298-62.314 Mbp | |
| | | | | (−) | |
| | HSD17B1 | Hydroxysteroid (17-beta) | NM_000413.1 | Chr 17: 40.612-40.615 Mbp | |
| | | dehydrogenase 1 | | (+) | |
| | HSD3B1 | Hydroxy-delta-5-steroid | NM_000862.1 | Chr 1: 119.196-119.204 Mbp | |
| | | dehyrogenase, 3 beta- and | | (+) | |
| | | steroid delta-isomerase | | | |
| | Hs.231971 | MRNA full length insert cDNA | | Chr 9: 106.585-106.628 Mbp | |
| | | clone EUROIMAGE 248114 | | (−) | |
| | IGFBP1 | Insulin-like growth factor | NM_000596.1 | Chr 7: 45.634-45.64 Mbp | |
| | | binding protein 1 | | (+) | |
| | KISS1 | KISS-1 metastasis-suppressor | NM_002256.2 | Chr 1: 200.52-200.526 Mbp | |
| | | | | (−) | |
| | PAPPA | pregnancy-associated plasma | NM_002581.3 | Chr 9: 112.369-112.618 Mbp | |
| | | protein A | | (+) | |
| | PSG1 | pregnancy specific beta-1- | NM_006905.2 | Chr 19: 48.047-48.059 Mbp | |
| | | glycoprotein 1 | | (−) | |
| | PSG2 | pregnancy specific beta-1- | NM_031246.1 | Chr 19: 48.244-48.262 Mbp | |
| | | glycoprotein 2 | | (−) | |
| | PSG3 | pregnancy specific beta-1- | NM_021016.2 | Chr 19: 47.901-47.92 Mbp | |
| | | glycoprotein 3 | | (−) | |
| | PSG4 | pregnancy specific beta-1- | NM_002780.3 | Chr 19: 48.372-48.385 Mbp | |
| | | glycoprotein 4 | | (−) | |
| | PSG5 | pregnancy specific beta-1- | NM_002781.2 | Chr 19: 48.347-48.366 Mbp | |
| | | glycoprotein 5 | | (−) | |
| | PSG7 | pregnancy specific beta-1- | NM_002783.1 | Chr 19: 48.104-48.117 Mbp | |
| | | glycoprotein 7 | | (−) | |
| | PSG9 | pregnancy specific beta-1- | NM_002784.2 | Chr 19: 48.433-48.449 Mbp | |
| | | glycoprotein 9 | | (−) | |
| | TFAP2A | transcription factor AP-2 alpha | NM_003220.1 | Chr 6: 10.46-10.477 Mbp | |
| | | (activating enhancer binding | | (−) | |
| | | protein 2 alpha) | | | |
| | TGM2 | Transglutaminase 2 (C | NM_004613.2 | Chr 20: 37.395-37.432 Mbp | |
| | | polypeptide, protein-glutamine- | | (−) | |
| | | gamma-glutamyltransferase) | | | |
| | TIMP2 | tissue inhibitor of | NM_003255.2 | Chr 17: 77.312-77.382 Mbp | |
| | | metalloproteinase 2 | | (−) | |
| | VGLL1 | vestigal-like 1 (drosphilia) | NM_016267.2 | Chr X: 133.559-133.583 Mbp | |
| | | | | (+) | |
| TestisGermCell | CRISP2 | Cysteine-rich secretory protein 2 | NM_003296.1 | Chr 6: 49.661-49.682 Mbp | |
| | | | | (−) | |
| | 203861_s_at | actinin, alpha 2 | | Chr 1: 233.217-233.222 Mbp | |
| | | | | (+) | |
| Heart | 32485_at | Human myoglobin gene (exon | | Chr 22: 34.274-34.275 Mbp | |
| | | 1) (and joined CDS) | | (−) | |
| | 36477_at | Homo sapiens TNNI3 gene | | Chr 19: 60.339-60.341 Mbp | |
| | | | | (−) | |
| | 39063_at | Human alpha-cardiac actin | | Chr 15: 32.661-32.662 Mbp | |
| | | gene, 5 flank | | (−) | |
| | 39085_at | Human slow twitch skeletal | | Chr 3: 52.341-52.341 Mbp | |
| | | muscle/cardiac muscle troponin | | (−) | |
| | | C gene, complete cds | | | |
| | ACTN2 | Actinin, alpha 2 | NM_001103.1 | Chr 1: 233.146-233.223 Mbp | |
| | | | | (+) | |
| | CASQ2 | Calsequestrin 2 (cardiac | NM_001232.1 | Chr 1: 115.39-115.459 Mbp | |
| | | muscle) | | (−) | |
| | CKM | Creatine kinase, muscle | NM_001824.2 | Chr 19: 50.485-50.502 Mbp | |
| | | | | (−) | |
| | COX6A2 | Cytochrome c oxidase subunit | NM_005205.2 | Chr 16: 31.435-31.436 Mbp | |
| | | VIa polypeptide 2 | | (−) | |
| | CSRP3 | Cysteine and glycine-rich | NM_003476.2 | Chr 11: 19.245-19.262 Mbp | |
| | | protein 3 (cardiac LIM protein) | | (−) | |
| | DES | Desamin | NM_001927.2 | Chr 2: 220.247-220.255 Mbp | |
| | | | | (+) | |
| | HRC | Histidine rich calcium binding | NM_002152.1 | Chr 19: 54.33-54.334 Mbp | |
| | | protein | | (−) | |
| | LDB3 | LIM domain binding 3 | NM_007078.1 | Chr 10: 88.559-88.625 Mbp | |
| | | | | (+) | |
| | MB | Myglobin | NM_005368.2 | Chr 22: 34.274-34.291 Mbp | |
| | | | | (−) | |
| | MYH6 | Myosin, heavy polypeptide 6, | NM_002471.1 | Chr 14: 21.841-21.866 Mbp | |
| | | cardiac muscle, alpha | | (−) | |
| | | (cardiomyopathy, hypertrophic | | | |
| | | 1) | | | |
| | MYH7 | Myosin, heavy polypeptide 7, | NM_000257.1 | Chr 14: 21.872-21.893 Mbp | |
| | | cardiac muscle, beta | | (−) | |
| | MYL2 | Myosin, light polypeptide 2, | NM_000432.1 | Chr 12: 111.131-111.141 Mbp | |
| | | regulatory, cardia, slow | | (−) | |
| | MYL3 | Myosin, light polypeptide 3, | NM_000258.1 | Chr 3: 46.718-46.724 Mpb | |
| | | alkali; ventricular, skeletal, | | (+) | |
| | | slow | | | |
| | MYL7 | Myosin, light polypeptide 7, | NM_021223.1 | Chr 7: 43.885-43.888 Mbp | |
| | | regulatory | | (+) | |
| | MYOZ2 | Myozenin 2 | NM_016599.2 | Chr 4: 120.45-120.502 Mbp | |
| | | | | (+) | |
| | PGAM | Phosphoglycerate mutase 2 | NM_000290.1 | Chr 7: 43.809-43.811 Mbp | |
| | | (muscle) | | (−) | |
| | SLC4A3 | solute carrier family 4, anion | NM_005070.1 | Chr 2: 220.456-220.47 Mbp | |
| | | exchanger, member 3 | | (+) | |
| | TCAP | Titin-cap (telethonin) | NM_003673.2 | Chr 17: 37.73-37.733 Mbp | |
| | | | | (+) | |
| | TNNC1 | Troponin C, slow | NM_003280.1 | Chr 3: 52.341-52.344 Mbp | |
| | | | | (+) | |
| | TNN13 | Troponin 1, cardiac | NM_000363.3 | Chr 19: 60.339-60.345 Mbp | |
| | | | | (−) | |
| | TNNT2 | Troponin T2, cardiac | NM_000364.2 | Chr 1: 198.616-198.635 Mbp | |
| | | | | (−) | |
| | TPM1 | Tropomyosin 1 (alpha) | NM_000366.4 | Chr 15: 60.913-60.937 Mbp | |
| | | | | (+) | |
| 17369_THY− | IGLL1 | Immunoglobulin lambda-like | NM_020070.2 | Chr 22: 22.239-22.247 Mbp | |
| | | polypeptide 1 | | (−) | |
| | MYB | V-MYB myeloblastosis viral | NM_005375.2 | Chr 6: 135.437-135.475 Mbp | |
| | | oncogene homolog (avian) | | (+) | |
| 17299_THY+ | 1369_s_at | Human interleukin 8 (IL8) | | Chr 4: 75.009-75.009 Mbp | |
| | | gene, complete cds | | (+) | |
| | CACNA1E | Calcium channel, voltage- | NM_000721.1 | Chr 1: 177.972-178.288 Mbp | |
| | | dependent, alpha 1E subunit | | (+) | |
| | HIST1H2AE | Histone 1, H2ae | NM_021052.2 | Chr 6: 26.279-26.28 Mbp | |
| | | | | (+) | |
| | HIST2H2AA | Histone 2, H2aa | NM_003516.2 | Chr 1: 146.588-146.598 Mbp | |
| | | | | (+) | |
| 17440_THY− | EREG | Epiregulin | NM_001432.1 | Chr 4: 75.631-75.655 Mbp | |
| | | | | (+) | |
| HL60 | 33641_g_at | Homo sapiens DNA, cosmid | | Chr 6: 31.643-31.643 Mbp | |
| | | clones TN62 and TN82 | | (+) | |
| | 40019_at | Human EVI2B3P gene, exon | | Chr 17: 29.48-29.481 Mbp | |
| | | and complete cds | | (−) | |
| | CLC | Charcot-Leyden crystal protein | NM_001828.4 | Chr 19: 44.897-44.904 Mbp | |
| | | | | (−) | |
| | LILRB1 | Leukocyte immunoglobulin- | NM_006669.2 | Chr 19: 59.804-59.825 Mbp | |
| | | like receptor, subfamily B | | (+) | |
| | | (with TM and ITIM domains), | | | |
| | | member 1 | | | |
| | MPO | Myeloperoxidase | NM_000250.1 | Chr 17: 56.689-56.7 Mbp | |
| | | | | (−) | |
| | RNASE2 | Ribonuclease, RNase A family, | NM_002934.2 | Chr 14: 19.413-19.414 Mbp | |
| | | 2 (liver, eosinophil-derived | | (+) | |
| | | neurotoxin) | | | |
| | SERPINB10 | Serine (or cysteine) proteinase | NM_005024.1 | Chr 18: 61.367-61.387 Mbp | |
| | | inhibitor, clade B (ovalbumin), | | (+) | |
| | | member 10 | | | |
| MOL4 | 217028_at | Chemokine (C—X—C motif), | | Chr 2: 136.894-136.894 Mbp | |
| | | receptor 4 (fusin) | | (−) | |
| | 33238_at | Human T-lymphocyte specific | | Chr 1: 32.177-32.178 Mbp | |
| | | protein tyrosine kinase p56lck | | (+) | |
| | | (lck) abberant mRNA, | | | |
| | | complete cds | | | |
| | 37861_at | Human CD1 R2 gene for | | Chr 1: 155.104-155.105 Mbp | |
| | | MHC-related antigen | | (+) | |
| | 40775_at | Human DNA sequence from | | Chr X: 76.657-76.657 Mbp | |
| | | PAC 696H22 on chromosome | | (−) | |
| | | Xq21.1-21.2. Contains a mouse | | | |
| | | E25 like gene, a Kinesin like | | | |
| | | pseudogene and ESTs | | | |
| | ALDH1A2 | Aldehyde dehydrogenase 1 | NM_003888.2 | Chr 15: 55.824-55.937 Mbp | |
| | | family, member A2 | | (−) | |
| | ARHGDIB | Rho GDP dissociation inhibitor | NM_001175.1 | Chr 12: 14.995-15.014 Mbp | |
| | | (GDI) beta | | (−) | |
| | CD1B | CD1B antigen, b polypeptide | NM_001764.1 | Chr 1: 155.075-155.079 Mbp | |
| | | | | (−) | |
| | CFTR | Cystic fibrosis transmembrane | NM_000492.2 | Chr 7: 116.66-116.849 Mbp | |
| | | conductance regulator, ATP- | | (+) | |
| | | binding cassette (sub-family C, | | | |
| | | member 7) | | | |
| | CORO1A | Coronin, actin binding protein, | NM_007074.1 | Chr 16: 30.192-30.197 Mbp | |
| | | 1A | | (+) | |
| | CXCR4 | Chemokine (C—X—C motif) | NM_003467.1 | Chr 2: 137.082-137.086 Mbp | |
| | | receptor 4 | | (−) | |
| | ITM2A | integral membrane protein 2A | NM_004867.2 | Chr X: 76.657-76.664 Mbp | |
| | | | | (−) | |
| | LEF1 | Lymphoid enhancer-binding | NM_016269.2 | Chr 4: 109.361-109.482 Mbp | |
| | | factor 1 | | (−) | |
| | NINJ2 | Ninjurin 2 | NM_016533.4 | Chr 12: 0.552-0.652 Mbp | |
| | | | | (−) | |
| | hIAN2 | human immune associated | NM_024711.2 | Chr 7: 149.637-149.644 Mbp | |
| | | nucleotide 2 | | (−) | |
| | RHOH | Ras homolog gene family, | NM_004310.2 | Chr 4: 40.033-40.08 Mbp | |
| | | member H | | (+) | |
| K562 | 217414_x_at | Hemoglobin, alpha 2 | | Chr 16: 0.162-0.163 Mbp | |
| | | | | (+) | |
| | GAGE2 | G antigen 2 | NM_001472.1 | Chr X: 47.994-48.059 Mbp | |
| | | | | (+) | |
| | HBA1 | Hemoglobin, alpha 1 | NM_000558.3 | Chr 16: 0.166-0.167 Mbp | |
| | | | | (+) | |
| | HBE1 | Hemoglobin, epsilon 1 | NM_005330.3 | Chr 11: 5.248-5.25 Mbp | |
| | | | | (−) | |
| | PRAME | preferentially expressed | NM_006115.3 | Chr 22: 21.214-21.226 Mbp | |
| | | antigen in melanoma | | (−) | |
| | SCG3 | Secretogranin III | NM_013243.2 | Chr 15: 49.552-49.592 Mbp | |
| | | | | (+) | |
| | SSX2 | Synovial sarcoma, X | NM_003147.4 | Chr X: 51.377-51.442 Mbp | |
| | | breakpoint 2 | | (+) | |
| TestisLeydigCell | SPAG11 | sperm associated antigen 11 | NM_016512.2 | Chr 8: 7.468-7.592 Mbp | |
| | | | | (+) | |
| TestisInterstitial | MCSP | Mitochondrial capsule | NM_030663.2 | Chr 1: 149.625-149.632 Mbp | |
| | | selenoprotein | | (+) | |
| Leukemialympho | DNTT | Deoxynucleotidyltransferase, | NM_004088.2 | Chr 10: 98.195-98.229 Mbp | |
| | | terminal | | (+) | |
| blastic(molt 4) | | | | | |
| Leukemiaprom- | CR2 | complement component | NM_001877.2 | Chr 1: 204.271-204.306 Mbp | |
| | | (3d/Epstein Barr virus) | | (+) | |
| | | receptor 2 | | | |
| yelocytic(h160) | RGS13 | regulator of G-protein | NM_002927.3 | Chr 1: 189.071-189.095 Mbp | |
| | | signalling 13 | | (+) | |
| PB- | 203828_s_at | natural killer cell transcript 4 | | Chr 16: 3.118-3.119 Mbp | |
| CD56+NKCells | | | | (+) | |
| | 37145_at | Homo sapiens NKG5 gene, | | Chr 2: 85.879-85.883 Mbp | |
| | | complete cds | | (+) | |
| | AKNA | AT-hook transcription factor | NM_030767.2 | Chr 9: 110.552-110.603 Mbp | |
| | | | | (−) | |
| | BIN2 | bridging integrator | NM_016187.1 | Chr 12: 51.391-51.434 Mbp | |
| | | | | (−) | |
| | CD3Z | CD32 antigen, zeta polypeptide | NM_002985.2 | Chr 17: 34.047-34.056 Mbp | |
| | | (TiT3 complex) | | (−) | |
| | CD7 | CD7 antigen (p41) | NM_006137.5 | Chr 17: 80.802-80.805 Mbp | |
| | | | | (−) | |
| | CMRF-35H | Leukocyte membrane antigen | NM_007261.1 | Chr 17: 72.926-72.945 Mbp | |
| | | | | (+) | |
| | CST7 | Cystatin F (leukocystatin) | NM_003650.2 | Chr 20: 24.877-24.888 Mbp | |
| | | | | (+) | |
| | CTSW | Cathespin W (lymphopain) | NM_001335.2 | Chr 11: 65.897-65.901 Mbp | |
| | | | | (+) | |
| | CX3CR1 | Chemokine (C-X3-C motif) | NM_001337.2 | Chr 3: 39.118-39.134 Mbp | |
| | | receptor 1 | | (−) | |
| | EDG8 | Endothelial differentiation, | NM_030760.3 | Chr 19: 10.468-10.473 Mbp | |
| | | sphingolipid G-protein-coupled | | (−) | |
| | | receptor 8 | | | |
| | GNLY | Granulysin | NM_006433.2 | Chr 2: 85.879-85.883 Mbp | |
| | | | | (+) | |
| | GZMH | Granzyme H (cathepsin G-like | NM_033423.2 | Chr 14: 23.065-23.068 Mbp | |
| | | 2, protein h-CCPX) | | (−) | |
| | HA-1 | minor histocompatibility | NM_012292.2 | Chr 19: 1.018-1.037 Mbp | |
| | | antigen HA-1 | | (+) | |
| | KLRB1 | killer cell lectin-like receptor | NM_002258.1 | Chr 12: 9.647-9.66 Mbp | |
| | | subfamily B, mamber 1 | | (−) | |
| | KLRD1 | killer cell lectin-like receptor | NM_002262.2 | Chr 12: 10.36-10.369 Mbp | |
| | | subfamily D, member 1 | | (+) | |
| | KLRF1 | killer cell lectin-like receptor | NM_016523.1 | Chr 12: 9.88-9.897 Mbp | |
| | | subfamily F, member 1 | | (+) | |
| | MYOM2 | Myomesin (M-protein) 2, | NM_003970.1 | Chr 8: 2.143-2.243 Mbp | |
| | | 165 kDa | | (+) | |
| | NK4 | natural killer cell transcript 4 | NM_004221.3 | Chr 16: 3.115-3.119 Mbp | |
| | | | | (+) | |
| | PRF1 | Perforin 1 (pore froming | NM_005041.3 | Chr 10: 72.249-72.254 Mbp | |
| | | protein) | | (−) | |
| | PSMB8 | Proteasome (prosome, | NM_004159.3 | Chr 6: 32.81-32.814 Mbp | |
| | | macropain) sunbunit, beta type, | | (−) | |
| | | 8 (large multifunctional | | | |
| | | protease7) | | | |
| | PTPRC | protein tyrosine phosphatase, | NM_002838.2 | Chr 1: 195.074-195.192 Mbp | |
| | | receptor type, C | | (+) | |
| | RAC2 | Ras-related C3 botulinum toxin | NM_002872.3 | Chr 22: 35.864-35.883 Mbp | |
| | | substrate 2 (rho family, small | | (−) | |
| | | GTP binding protein Rac2) | | | |
| | RUNX3 | Runt-related transcription | NM_004350.1 | Chr 1: 24.205-24.235 Mbp | |
| | | factor 3 | | (−) | |
| | SH2D1A | SH2 domain protein 1A, | NM_002351.1 | Chr X: 121.432-121.459 Mbp | |
| | | Duncan's disease | | (+) | |
| | | (lymphoproliferative | | | |
| | | syndrome) | | | |
| | STK10 | Serine/threonine kinase 10 | NM_005990.2 | Chr 5: 171.406-171.55 Mbp | |
| | | | | (−) | |
| | T3JAM | TRAF3-interacting Jun N- | NM_025228.1 | Chr 1: 206.568-206.594 Mbp | |
| | | terminal kinase (JNK)- | | (+) | |
| | | activating modulator | | | |
| | TRD@ | T cell receptor delta locus | | Chr 14: 20.908-20.925 Mbp | |
| | | | | (+) | |
| | TRGV9 | T cell receptor gamma variable 9 | | Chr 7: 38.004-38.1 Mbp | |
| | | | | (−) | |
| | XCL1 | Chemokine (C motif) ligand 1 | NM_002995.1 | Chr 1: 165.241-165.247 Mbp | |
| | | | | (+) | |
| | XCL2 | Chemokine (C motif) ligand 2 | NM_003175.2 | Chr 1: 165.206-165.209 Mbp | |
| | | | | (−) | |
| | ZAP70 | zeta-chain (TCR) associated | NM_001079.3 | Chr 2: 97.934-97.96 Mbp | |
| | | protein kinase 70 kDa | | (+) | |
| 721_B_lympho | CTAG1B | cancer/testis antigen 1 | NM_001327.1 | Chr X: 151.398-151.432 Mbp | |
| | | | | (+) | |
| blasts | CTAG2 | cancer/testis antigen 2 | NM_020994.1 | Chr X: 151.465-151.467 Mbp | |
| | | | | (+) | |
| | FCER2 | Fc fragment of lgE, low affinity | NM_002002.3 | Chr 19: 7.648-7.661 Mbp | |
| | | II, receptor for (CD23A) | | (−) | |
| | HLA-DQA1 | major histocompatibility | NM_002122.2 | Chr 6: 32.656-32.662 Mbp | |
| | | complex, class II DQ alpha 1 | | (+) | |
| | MAP4K1 | Mitogen-activated protein | NM_007181.3 | Chr 19: 43.754-43.784 Mbp | |
| | | kinase 1 | | (−) | |
| | UNC13C | unc-13 homolog C (C. elgans) | | Chr 15: 51.878-52.499 Mbp | |
| | | | | (+) | |
| PB-CD19+Bcells | ADAM28 | a disintegrin and | NM_014265.1 | Chr 8: 23.972-24.033 Mbp | |
| | | metalloproteinase domain 28 | | (+) | |
| | BLK | B lymphoid tyrosine kinase | NM_001715.2 | Chr 8: 11.222-11.293 Mbp | |
| | | | | (+) | |
| | C14orf110 | Chromosome 14 open | | Chr 14: 104.355-104.363 Mbp | |
| | | readingfram 110 | | (+) | |
| | CD22 | CD22 antigen | NM_001771.1 | Chr 19: 40.498-40.514 Mbp | |
| | | | | (+) | |
| | CD37 | CD37 antigen | NM_001774.1 | Chr 19: 54.514-54.519 Mbp | |
| | | | | (+) | |
| | HLA-DOB | major histocompatibility | NM_002120.2 | Chr | |
| | | complex, class II, DO beta | | 6_random: 4.083-4.088 Mbp | |
| | | | | (−) | |
| | HLA-DQB2 | major histocompatibility | NM_182549.1 | Chr 6: 32.725-32.732 Mbp | |
| | | complex, class II, DQ beta 2 | | (−) | |
| | ISG20 | Interferon stimulated fene 20 kDa | NM_002201.4 | Chr 15: 86.769-86.786 Mbp | |
| | | | | (+) | |
| | LTB | Lymphotoxin beta (TNF | NM_002341.1 | Chr 6: 31.607-31.609 Mbp | |
| | | superfamily, member 3) | | (−) | |
| | P2RX5 | Purinergic receptor P2X, | NM_002561.2 | Chr 17: 3.527-3.55 Mbp | |
| | | ligand-gated ion channel 5 | | (−) | |
| | POU2AFI | POU domain, class 2, | NM_006235.1 | Chr 11: 111.256-111.284 Mbp | |
| | | associating factor 1 | | (−) | |
| | TOSO | regulator of Fas-induced | NM_005449.3 | Chr 1: 203.721-203.738 Mbp | |
| | | apoptosis | | (−) | |
| Liver | 1103_at | Human angiogenin gene, | | Chr 14: 19.152-19.152 Mbp | |
| | | complete cds, and three Alu | | (+) | |
| | | repetitive sequences | | | |
| | 1431_at | Human cytochrome P450IIE1 | | Chr 10: 135.263-135.268 Mbp | |
| | | (ethanol-inducible) gene, | | (+) | |
| | | complete cds | | | |
| | 203722_at | aldehyde dehydrogenase 4 | | Chr 1: 18.344-18.344 Mbp | |
| | | family, member A1 | | (−) | |
| | 31825_at | Human heparin cofactor II | | Chr 22: 19.466-19.466 Mbp | |
| | | (HCF2) gene, exons 1 through 5 | | (+) | |
| | 33487_at | Human gene for 4- | | Chr 12: 122.046-122.054 Mbp | |
| | | hydroxyphenylpyruvic acid | | (−) | |
| | | dioxygenase (HPD), comlete | | | |
| | | cds | | | |
| | 33703_f_at | Human phosphoenolpyruvate | | Chr 20: 56.779-56.779 Mbp | |
| | | carboxykinase (PCK1) gene, | | (+) | |
| | | complete cds with repeats | | | |
| | 33990_at | Human mRNA clone with | | Chr 4: 74.687-74.687 Mbp | |
| | | similarity to L-glycerol-3- | | (+) | |
| | | phosphate-NAD | | | |
| | | oxidoreductase and albumin | | | |
| | | gene sequences | | | |
| | 33991_g_at | Human mRNA clone with | | Chr 4: 74.684-74.687 Mbp | |
| | | similarity to L-glycerol-3- | | (+) | |
| | | phosphate-NAD | | | |
| | | oxidoreductase and albumin | | | |
| | | gene sequences | | | |
| | 33992_at | Human serum albumin (ALB) | | Chr 4: 74.685-74.685 Mbp | |
| | | gene, complete cds | | (+) | |
| | 34298_at | H. sapiens gene for inter-alpha- | | Chr 3: 52.679-52.68 Mbp | |
| | | trypsin inhibitor heavy chain | | (+) | |
| | | H1, exons 1-3 | | | |
| | 36646_at | Human plasminogen gene | | Chr 6: 160.995-161.007 Mbp | |
| | | | | (+) | |
| | 36995_at | Human inter-alpha-trypsin | | Chr 9: 110.276-110.278 Mbp | |
| | | inhibitor light chain (ITI) gene | | (−) | |
| | 37175_at | Human antithrombin III | | Chr 1: 170.453-170.459 Mbp | |
| | | (ATIII) gene | | (−) | |
| | 39763_at | human hemopexingene | | Chr 11: 6.411-6.412 Mbp | |
| | | | | (−) | |
| | A1BG | alpha-1-B glycoprotein | NM_130786.2 | Chr 19: 63.532-63.54 Mbp | |
| | | | | (−) | |
| | AADAC | Arylacetamide deacetylase | NM_001086.1 | Chr 3: 152.813-152.827 Mbp | |
| | | (esterase) | | (+) | |
| | ADH1A | alcohol dehydrogenase 1A | NM_000667.2 | Chr 4: 100.59-100.604 Mbp | |
| | | (class I), alpha polypeptide | | (−) | |
| | ADH1C | alcohol dehydrogenase 1C | NM_000669.2 | Chr 4: 100.65-100.666 Mbp | |
| | | (class I), gamma polypeptide | | (−) | |
| | AGXT | Alanine- | NM_000030.1 | Chr 2: 241.827-241.838 Mbp | |
| | | glyoxylateaminotransferase | | (+) | |
| | | (oxalosis 1; hyperoxaluria 1; | | | |
| | | glycolicaciduria; serine- | | | |
| | | pyruvate aminotransferase | | | |
| | AKR1C4 | Aldo-keto reductase family 1, | NM_001818.2 | Chr 10: 5.339-5.361 Mbp | |
| | | member C4 (chlordecone | | (+) | |
| | | reductase; 3-alpha | | | |
| | | hydroxysteroid dehydrogenase, | | | |
| | | type I; dihydrodiol | | | |
| | | dehydrogenase 4) | | | |
| | AKR7A3 | Aldo-keto reductase family 7, | NM_012067.2 | Chr 1: 18.755-18.761 Mbp | |
| | | member A3 (aflatoxin aldehyde | | (−) | |
| | | reductase) | | | |
| | ALDH4A1 | Aldehyde dehydrogenase4 | NM_003748.2 | Chr 1: 18.343-18.375 Mbp | |
| | | family, member A1 | | (−) | |
| | ALDOB | Aldolase B, fructose- | NM_000035.2 | Chr 9: 97.641-97.655 Mbp | |
| | | bisphosphate | | (−) | |
| | AMBP | alpha-1-microglobulin/bikunin | NM_001633.2 | Chr 9: 110.276-110.294 Mbp | |
| | | precursor | | (−) | |
| | APOC1 | Apolipoprotein C-1 | NM_001645.2 | Chr 19: 50.094-50.098 Mbp | |
| | | | | (+) | |
| | ASGR2 | Asialoglycoprotein receptor 2 | NM_001181.2 | Chr 17: 6.949-6.961 Mbp | |
| | | | | (−) | |
| | C8G | complement component 8, | NM_000606.1 | Chr 9: 133.28-133.282 Mbp | |
| | | gamma polypeptide | | (+) | |
| | CES1 | Carboxylesterase 1 | NM_001266.3 | Chr 16: 55.536-55.597 Mbp | |
| | | (monocyte/macrophage serine | | (+) | |
| | | esterase 1) | | | |
| | CYP2A6 | Cytochrome P450, family 2, | NM_000762.4 | Chr 19: 46.025-46.209 Mbp | |
| | | subfamily A, polypeptide 6 | | (−) | |
| | CYP2A7 | Cytochrome P450, family 2, | NM_000764.2 | Chr 19: 46.057-46.064 Mbp | |
| | | subfamily A, polypeptide 7 | | (−) | |
| | CYP2D6 | Cytochrome P450, family 2, | NM_000106.3 | Chr 22: 40.767-40.771 Mbp | |
| | | subfamily D, polypeptide 6 | | (−) | |
| | CYP2E1 | Cytochrome P450, family 2, | NM_000773.2 | Chr 10: 135.256-135.268 Mbp | |
| | | subfamily E, polypeptide 1 | | (+) | |
| | DP1L1 | Polyposis locus protein 1-like 1 | NM_138393.1 | Chr 19: 1.431-1.437 Mbp | |
| | | | | (+) | |
| | F12 | Coagulation factor XII | NM_000505.2 | Chr 5: 176.764-176.772 Mbp | |
| | | (Hageman factor) | | (−) | |
| | F2 | Coagulation factor II | NM_000506.2 | Chr 11: 46.772-46.792 Mbp | |
| | | (thrombin) | | (+) | |
| | G6PC | Glucose-6-phosphatase, | NM_000151.1 | Chr 17: 40.961-40.974 Mbp | |
| | | catalytic (glycogen storage | | (+) | |
| | | disease type 1, von Glerke | | | |
| | | disease) | | | |
| | HAMP | Hepicidin antimicrobial | NM_021175.1 | Chr 19: 40.449-40.452 Mbp | |
| | | peptide | | (+) | |
| | HMGCS2 | 3-hydroxy-3-methylglutaryl- | NM_005518.1 | Chr 1: 119.438-119.458 Mbp | |
| | | Coenzyme A synthase 2 | | (−) | |
| | | (mitochondrial) | | | |
| | HP | Haptoglobin | NM_005143.1 | Chr 16: 71.824-71.83 Mbp | |
| | | | | (+) | |
| | HPD | 4-hydroxyphenylpyruvate | NM_002150.2 | Chr 12: 122.046-122.065 Mbp | |
| | | dioxygenase | | (−) | |
| | HPX | Hemopexin | NM_000613.1 | Chr 11: 6.411-6.421 Mbp | |
| | | | | (−) | |
| | ITIH1 | Inter-alpha (globulin) inhibitor | NM_002215.1 | Chr 3: 52.666-52.68 Mbp | |
| | | H1 | | (+) | |
| | ITIH4 | Inter-alpha (globulin inhibitor | NM_002218.3 | Chr 3: 52.701-52.719 Mbp | |
| | | H4 (plasma Kallikrein- | | (−) | |
| | | sensitive glycoprotein)) | | | |
| | LBP | Lipopolysaccharide binding | NM_004139.2 | Chr 20: 37.66-37.691 Mbp | |
| | | protein | | (+) | |
| | LCAT | Lecithin-cholesterol | NM_000229.1 | Chr 16: 67.708-67.713 Mbp | |
| | | acyltransferase | | (+) | |
| | MAT1A | Methionine | NM_000429.1 | Chr 10: 82.162-82.18 Mbp | |
| | | adenosyltransferase 1, alpha | | (−) | |
| | MUCDHL | Mucin and cadherin-like | NM_017717.3 | Chr 11: 0.573-0.583 Mbp | |
| | | | | (+) | |
| | NNMT | Nicotinamide N- | NM_006169.1 | Chr 11: 114.201-114.217 Mbp | |
| | | methyltransferase | | (+) | |
| | ORM2 | Orosomucoid 2 | NM_000608.2 | Chr 9: 110.545-110.55 Mbp | |
| | | | | (+) | |
| | PCK1 | Phosphoenolpyruvate | NM_002591.2 | Chr 20: 56.774-56.779 Mbp | |
| | | carboxykinase 1 (soluble) | | (+) | |
| | PPP1R1A | Protein phosphatase 1, | NM_006741.2 | Chr 12: 54.685-54.699 Mbp | |
| | | regulatory (inhibitor) sunbunit | | (−) | |
| | | 1A | | | |
| | PRAP1 | Proline-rich acidic protein 1 | NM_145202.3 | Chr 10: 135.079-135.082 Mbp | |
| | | | | (+) | |
| | PROC | Protein C (inactivator of | NM_000312.1 | Chr 2: 128.08-128.091 Mbp | |
| | | coagulation factors Va and | | (+) | |
| | | VIIIa) | | | |
| | RARRES2 | Retinoic acid receptor | NM_002889.2 | Chr 7: 149.35-149.353 Mbp | |
| | | responder (tazarotene induced) 2 | | (−) | |
| | RNASE4 | Ribonuclease, Rnase A family, 4 | NM_002937.3 | Chr 14: 19.142-19.158 Mbp | |
| | | | | (+) | |
| | SERPINA6 | Serine (or cysteine) proteinase | NM_001756.2 | Chr 14: 92.76-92.779 Mbp | |
| | | inhibitor, clade A (alpha-1 | | (−) | |
| | | antiproteinase, antitrypsin), | | | |
| | | member 6 | | | |
| | SERPIND1 | Serine (or cysteine) proteinase | NM_000185.2 | Chr 22: 19.452-19.466 Mbp | |
| | | inhibitor, clade D (heparin | | (+) | |
| | | cofactor), member 1 | | | |
| | SLC22A1 | solute carrier family 22 | NM_003057.2 | Chr 6: 160.376-160.413 Mbp | |
| | | (organic cation transporter), | | (+) | |
| | | member 1 | | | |
| | SLC27A5 | solute carrier family 27 (fatty | NM_012254.1 | Chr 19: 63.685-63.699 Mbp | |
| | | acid transporter), member 5 | | (−) | |
| | TAT | Tyrosine aminotransferase | NM_000353.1 | Chr 16: 71.336-71.346 Mbp | |
| | | | | (−) | |
| | TF | Transferrin | NM_001063.2 | Chr 3: 134.746-134.779 Mbp | http://genome.ucsc.edu/cgi-bin/hgc?hgsid= |
| | | | | | 34524523&g= |
| | | | | | htcDnaNearGene&i= |
| | | | | | NM_001063&c= |
| | | | | | chr3&l= |
| | | | | | 134745845&r= |
| | | | | | 134780246&o= |
| | | | | | refGene&hgSeq.promoter= |
| | | | | | on&boolshad.hgSeq.promoter= |
| | | | | | 1&hgSeq.promoterSize= |
| | | | | | 1000&hgSeq.utrExon5= |
| | | | | | on&boolshad.hgSeq.utrExon5= |
| | | | | | 1&boolshad.hgSeq.cdsExon= |
| | | | | | 1&boolshad.hgSeq.utrExon3= |
| | | | | | 1&boolshad.hgSeq.intron= |
| | | | | | 1&boolshad.hgSeq.downstream= |
| | | | | | 1&hgSeq.downstreamSize= |
| | | | | | 1000&hgSeq.granularity= |
| | | | | | gene&hgSeq.Padding5= |
| | | | | | 0&hgSeq.padding3= |
| | | | | | 0&boolshad.hgSeq.splitCDSUTR= |
| | | | | | 1&hgSeq.casing= |
| | | | | | exon&boolshad.hgSeq.maskRepeats= |
| | | | | | 1&hgSeq.repMasking= |
| | | | | | lower&submit= |
| | | | | | submit |
| | | | | (+) | |
| | TFR2 | Transferrin receptor 2 | NM_003227.2 | Chr 7: 99.815-99.836 Mbp | |
| | | | | (−) | |
| | TST | Thiosulfate sulfurtransferase | NM_003312.4 | Chr 22: 35.649-35.658 Mbp | |
| | | (rhodanase) | | (−) | |
| | TTR | Transthyretin (prealbumin, | NM_000371.1 | Chr 18: 29.059-29.066 Mbp | |
| | | amyloidosis type I) | | (+) | |
| | VTN | Vitronectin (serum spreadin | NM_000638.2 | Chr 17: 26.546-26.549 Mbp | |
| | | factor < somatomedin V, | | (−) | |
| | | complement S-protein) | | | |
| HepG2 | 261_s_at | Human apolipoprotein B-100 | | Chr 2: 21.182-21.182 Mbp | |
| | | (apoB) gene | | (−) | |
| | ABCC2 | ATP-binding cassette, sub- | NM_000392.1 | Chr 10: 101.673-101.742 Mbp | |
| | | family C (CFTR/MRP), | | (+) | |
| | | member 2 | | | |
| Lung | C20orf114 | Chromosome 20 open reading | NM_033197.2 | Chr 20: 32.539-32.566 Mbp | |
| | | frame 114 | | (+) | |
| | LAMP3 | Lysosomal-associated | NM_014398.2 | Chr 3: 184.242-184.282 Mbp | |
| | | membrane protein 3 | | (−) | |
| | MUC1 | Mucin 1, transmembrane | NM_002456.3 | Chr 1: 151.933-151.94 Mbp | |
| | | | | (−) | |
| | SCGB1A1 | Secretoglobin, family 1A, | NM_003357.3 | Chr 11: 62.437-62.441 Mbp | |
| | | member 1 (uteroglobin) | | (+) | |
| | SFTPA2 | Surfactant, pulmonary- | NM_006926.1 | Chr 10: 81.208-81.212 Mbp | |
| | | associated protein A2 | | (−) | |
| | SFTPD | Surfactant, pulmonary- | NM_003019.3 | Chr 10: 81.828-81.84 Mbp | |
| | | associated protein D | | (−) | |
| Daudi | BMP7 | bone morphogenetic protein 7 | NM_001719.1 | Chr 20: 56.383-56.479 Mbp | |
| | | (osteogenic protein 1) | | (−) | |
| | CD19 | CD19 antigen | NM_001770.3 | Chr 16: 28.941-28.949 Mbp | |
| | | | | (+) | |
| | CD53 | CD53 antigen | NM_000560.2 | Chr 1: 110.517-110.544 Mbp | |
| | | | | (+) | |
| | CD79A | CD79A antigen | NM_001783.1 | Chr 19: 47.057-47.061 Mbp | |
| | | (immunoglobulin-associated | | (+) | |
| | | alpha) | | | |
| | CD79B | CD79B antigen | NM_000626.1 | Chr 17: 62.346-62.35 Mbp | |
| | | (immunoglobulin-associated | | (−) | |
| | | beta) | | | |
| | CDKN3 | Cyclin-dependent kinase | NM_005192.2 | Chr 14: 52.853-52.876 Mbp | |
| | | inhibitor 3 (CDK2-associated | | (+) | |
| | | dual specificity phosphatase) | | | |
| | CDW52 | CDW52 antigen (CAMPATH- | NM_001803.1 | Chr 1: 25.877-25.88 Mbp | |
| | | 1 antigen) | | (+) | |
| | DDX3Y | DEAD (Asp-Glu-Ala-Asp) box | NM_004660.2 | Chr Y: 14.326-14.342 Mbp | |
| | | polypeptide 3, Y-linked | | (+) | |
| | EVI2B | Ecotropic viral integration site | NM_006495.2 | Chr 17: 29.48-29.49 Mbp | |
| | | 2B | | (−) | |
| | HHL | expressed in hematopoietic | NM_014857.2 | Chr 1: 170.709-171.508 Mbp | |
| | | cells, heart, liver | | (+) | |
| | HLA-DPB1 | major histocompatibility | NM_002121.4 | Chr 6: 33.045-33.056 Mbp | |
| | | complex, class II, DP beta 1 | | (+) | |
| | HLA-DRA | major histocompatibility | NM_019111.2 | Chr 6: 32.433-32.438 Mbp | |
| | | complex, class II, DR alpha | | (+) | |
| | IGJ | Immunoglobulin J polypeptide, | NM_144646.2 | Chr 4: 71.922-71.932 Mbp | |
| | | linker protein for | | (−) | |
| | | immunoglobulin alpha and mu | | | |
| | | polypeptides | | | |
| | IGKC | Immunoglobulin kappa | | Chr 2: 89.058-89.18 Mbp | |
| | | constant | | (−) | |
| | IGLJ3 | Immunoglobulin lambda | | Chr 22: 20.977-21.573 Mbp | |
| | | joining 3 | | (+) | |
| | LAPTM5 | Lysosomal-associated | NM_006762.1 | Chr 1: 30.631-30.657 Mbp | |
| | | multispanning membrane | | (−) | |
| | | protein-5 | | | |
| | LCP1 | Lymphocyte cytosolic protein 1 | NM_002298.2 | Chr 13: 45.636-45.693 Mbp | |
| | | (L-plastin) | | (−) | |
| | MS4A1 | Membrane-spanning 4- | NM_021950.2 | Chr 11: 60.474-60.487 Mbp | |
| | | domains, subfamily A, member 1 | | (+) | |
| | PTPN22 | protein tyrosine phosphatase, | NM_012411.2 | Chr 1: 113.475-113.514 Mbp | |
| | | non-receptor type 22 | | (−) | |
| | | (lymphoid) | | | |
| | TCL1A | T-cell leukemia/lymphoma 1A | NM_021966.1 | Chr 14: 94.166-94.17 Mbp | |
| | | | | (−) | |
| | TNFRSF7 | tumor necrosis factor receptor | NM_001242.3 | Chr 12: 6.433-6.44 Mbp | |
| | | superfamily, member 7 | | (+) | |
| Raji | CD48 | CD48 antigen (B-cell | NM_001778.2 | Chr 1: 157.426-157.459 Mbp | |
| | | membrane protein) | | (−) | |
| | CD74 | CD74 antigen (invariant | NM_004355.1 | Chr 5: 149.764-149.775 Mbp | |
| | | polypeptide of major | | (−) | |
| | | histocompatibility complex, | | | |
| | | class II antigen-associated) | | | |
| | HLA-DQB1 | major histocompatibility | NM_002123.2 | Chr 6: 32.628-32.635 Mbp | |
| | | complex, class II, DQ beta 1 | | (−) | |
| | HLA-DRB3 | major histocompatibility | NM_022555.3 | Chr 6: 32.489-32.502 Mbp | |
| | | complex, class II, DR beta 3 | | (−) | |
| | KLK1 | Kallikrein 1, | NM_002257.2 | Chr 19: 55.998-56.003 Mbp | |
| | | renal/pancreas/salivary | | (−) | |
| | PLEK | Pleckstrin | NM_002664.1 | Chr 2: 68.55-68.582 Mbp | |
| | | | | (+) | |
| | SPARCL1 | SPARC-like 1 (mast9, hevin) | NM_004684.2 | Chr 4: 88.787-88.843 Mbp | |
| | | | | (−) | |
| Lymphnode | 217378_x_at | immunoglobulin kappa | | Chr 2: 114.07-114.071 Mbp | |
| | | variable 1OR2-108 | | (+) | |
| | CCL21 | Chemokine (C—C motif) ligand | NM_002989.2 | Chr 9: 34.699-34.7 Mbp | |
| | | 21 | | (−) | |
| LymphomaburkettsDaudi | LRMP | Lymphoid-restricted membrane | NM_006152.2 | Chr 12: 25.105-25.161 Mbp | |
| | | protein | | (+) | |
| PB_CD14 + Monocytes | CD14 | CD14 antigen | NM_000591.1 | Chr 5: 139.994-139.995 Mbp | |
| | | | | (−) | |
| | CTSS | Cathespin S | NM_004079.3 | Chr 1: 147.477-147.513 Mbp | |
| | | | | (−) | |
| | DUSP1 | Dual specifity phosphatase 1 | NM_004417.2 | Chr 5: 172.13-172.133 Mbp | |
| | | | | (−) | |
| | DUSP6 | Dual specifity phosphatase 6 | NM_001946.2 | Chr 12: 89.674-89.679 Mbp | |
| | | | | (−) | |
| | FCN1 | Ficolin (collagen/fibrinogen | NM_002003.2 | Chr 9: 131.324-131.332 Mbp | |
| | | domain containing) 1 | | (−) | |
| | GMFG | Gila maturation factor, gamma | NM_004877.1 | Chr 19: 44.495-44.502 Mbp | |
| | | | | (−) | |
| | HK3 | Hexokinase 3(white cell) | NM_002115.1 | Chr 5: 176.243-176.261 Mbp | |
| | | | | (−) | |
| | IFI30 | Interferon, gamma-inducible | NM_006332.3 | Chr 19: 18.129-18.134 Mbp | |
| | | protein 30 | | (+) | |
| | LILRB2 | Leukocyte immunoglobulin- | NM_005874.1 | Chr 19: 59.454-59.46 Mbp | |
| | | like receptor | | (−) | |
| | RGS2 | regulator of G-protein | NM_002923.1 | Chr 1: 189.244-189.247 Mbp | |
| | | signalling 2, 24 kDa | | (+) | |
| | TYROBP | TYRO protein tyrosine kinase | NM_003332.2 | Chr 19: 41.071-41.075 Mbp | |
| | | binding protein | | (−) | |
| Smooth Muscle | CCL2 | Chemokine (C—C motif) ligand 2 | NM_002982.2 | Chr 17: 32.43-32.432 Mbp | |
| | | | | (+) | |
| | COL1A1 | Collagen, type 1, alpha 1 | NM_000088.2 | Chr 17: 48.603-48.621 Mbp | |
| | | | | (−) | |
| | CXCL1 | Chemokine (C—X—C motif) | NM_001511.1 | Chr 4: 75.135-75.137 Mbp | |
| | | ligand 6 (granulocyte | | (+) | |
| | | chemotactic protein 2) | | | |
| | CXCL6 | Chemokine (C—X—C motif) | NM_002993.1 | Chr 4: 75.103-75.105 Mbp | |
| | | ligand 1 (melanoma growth | | (+) | |
| | | stimulating activity, alpha) | | | |
| | IL8 | Interleukin 8 | NM_000584.2 | Chr 4: 75.006-75.01 Mbp | |
| | | | | (+) | |
| | LOXL1 | Lysyl oxidase-like 1 | NM_005576.1 | Chr 15: 71.794-71.82 Mbp | |
| | | | | (+) | |
| | MMP1 | Matrix metalloproteinase 1 | NM_002421.2 | Chr 11: 102.694-102.702 Mbp | |
| | | (interstitial collagenase) | | (−) | |
| | PTX3 | Pentaxin-related gene, rapidly | NM_002852.2 | Chr 3: 158.436-158.442 Mbp | |
| | | induced by IL-1 beta | | (+) | |
| | SERPINE1 | Serine (or cysteine) proteinase | NM_000602.1 | Chr 7: 100.316-100.328 Mbp | |
| | | inhibitor, clade E (nexin, | | (+) | |
| | | plasminogen activator inhibitor | | | |
| | | type 1), member 1 | | | |
| | SERPINH1 | Serine (or cysteine) proteinase | NM_001235.2 | Chr 11: 75.495-75.506 Mbp | |
| | | inhibitor, clade H (heat shock | | (+) | |
| | | protein 47), member 1 | | | |
| | | (collagen binding protein 1) | | | |
| | TFP12 | tissue factor pathway inhibitor 2 | NM_006528.2 | Chr 7: 93.113-93.118 Mbp | |
| | | | | (−) | |
| Skeletal Muscle | 213201_s_at | Troponin T1, skeletal, slow | | Chr 19: 60.32-60.328 Mbp | |
| | | | | (−) | |
| | ENO3 | Enolase 3, (beta, muscle) | NM_001976.2 | Chr 17: 4.799-4.805 Mbp | |
| | | | | (+) | |
| | HUMMLC2B | Myosin light chain 2 | NM_013292.2 | Chr 16: 30.383-30.386 Mbp | |
| | | | | (+) | |
| | MYBPC2 | Myosin binding protein C, fast | NM_004533.1 | Chr 19: 55.612-55.645 Mbp | |
| | | type | | (+) | |
| | MYL1 | Myosin, light polypeptide 1, | NM_079420.1 | Chr 2: 211.118-211.143 Mbp | |
| | | alkali; skeletal, fast | | (−) | |
| | TNNC2 | Troponin C2, fast | NM_003279.2 | Chr 20: 45.09-45.094 Mbp | |
| | | | | (−) | |
| | TNNI1 | Troponin 1, skeletal, slow | NM_003281.2 | Chr 1: 197.84-197.857 Mbp | |
| | | | | (−) | |
| | TNNI2 | Troponin 1, skeletal, fast | NM_003282.1 | Chr 11: 1.82-1.822 Mbp | |
| | | | | (+) | |
| | TTN | Titin | NM_003319.2 | Chr 2: 179.354-179.636 Mbp | |
| | | | | (−) | |
| CardiacMyocytes | POSTN | Periostin, osteoblast specific | NM_006475.1 | Chr 13: 37.073-37.109 Mbp | |
| | | factor | | (−) | |
| BM-CD33+Mye | AIF1 | Allograft inflammatory factor 1 | NM_001623.3 | Chr 6: 31.642-31.643 Mbp | |
| | | | | (+) | |
| loid | COPEB | core promoter element binding | NM_001300.3 | Chr 10: 3.921-3.927 Mbp | |
| | | protein | | (−) | |
| | CSPG2 | Chondroitin sulfate | NM_004385.2 | Chr 5: 82.806-82.915 Mbp | |
| | | proteoglycan 2 (versican) | | (+) | |
| | FOSB | FBJ murine osteosarcoma viral | NM_006732.1 | Chr 19: 50.647-50.654 Mbp | |
| | | oncogene homolog B | | (+) | |
| Salivary Gland | AMY2B | Amylase, alpha 2B; pancreatic | NM_020978.2 | Chr 1: 103.28-103.305 Mbp | |
| | | | | (+) | |
| | AZGP1 | Alpha-2-glycoprotein 1, zinc | NM_001185.2 | Chr 7: 99.161-99.171 Mbp | |
| | | | | (−) | |
| | C20orf70 | Chromosome 20 open reading | NM_080574.2 | Chr 20: 32.424-32.437 Mbp | |
| | | frame 70 | | (+) | |
| | CA6 | Carbonic anhydrase VI | NM_001215.1 | Chr 1: 8.602-8.631 Mbp | |
| | | | | (+) | |
| | CRISP3 | Cysteine-rich secretory protein 3 | NM_006061.1 | Chr 6: 49.696-49.713 Mbp | |
| | | | | (−) | |
| | CST1 | Cystatin SN | NM_001898.2 | Chr 20: 23.676-23.679 Mbp | |
| | | | | (−) | |
| | CST2 | Cystatin SA | NM_001322.2 | Chr 20: 23.752-23.755 Mbp | |
| | | | | (−) | |
| | CST4 | Cystatin S | NM_001899.2 | Chr 20: 23.614-23.617 Mbp | |
| | | | | (−) | |
| | HTN1 | Histatin 1 | NM_002159.2 | Chr 4: 71.166-71.174 Mbp | |
| | | | | (+) | |
| | HTN3 | Histatin 3 | NM_000200.1 | Chr 4: 71.144-71.152 Mbp | |
| | | | | (+) | |
| | LOC124220 | similar to common salivary | NM_145252.1 | Chr 16: 2.88-2.882 Mbp | |
| | | protein 1 | | (+) | |
| | MUC7 | Mucin 7, salivary | NM_152291.1 | Chr 4: 71.587-71.598 Mbp | |
| | | | | (+) | |
| | PIP | Prolactin-induced protein | NM_002652.2 | Chr 7: 142.223-142.23 Mbp | |
| | | | | (+) | |
| | PRB1 | Proline-rich protein BstNI | NM_005039.2 | Chr 12: 11.405-11.448 Mbp | |
| | | subfamily 1 | | (−) | |
| | PRB2 | Proline-rich protein BstNI | | Chr 12: 11.435-11.437 Mbp | |
| | | subfamily 2 | | (−) | |
| | PRB3 | Proline-rich protein BstNI | NM_006249.3 | Chr 12: 11.319-11.322 Mbp | |
| | | subfamily 3 | | (−) | |
| | PRB4 | Proline-rich protein BstNI | NM_002723.3 | Chr 12: 11.36-11.363 Mbp | |
| | | subfamily 4 | | (−) | |
| | PROL1 | Proline rich 1 | NM_021225.1 | Chr 4: 71.513-71.525 Mbp | |
| | | | | (+) | |
| | PROL3 | Proline rich 3 | NM_006685.2 | Chr 4: 71.498-71.505 Mbp | |
| | | | | (+) | |
| | PROL5 | Proline rich 5 (salivary) | NM_012390.1 | Chr 4: 71.477-71.482 Mbp | |
| | | | | (+) | |
| | PRR4 | Proline rich 4 (lacrimal) | NM_007244.1 | Chr 12: 10.898-10.905 Mbp | |
| | | | | (−) | |
| | SLP1 | secretory leukocyte protease | NM_003064.2 | Chr 20: 44.519-44.521 Mbp | |
| | | inhibitor (antileukoproteinase) | | (−) | |
| | STATH | Statherin | NM_003154.1 | Chr 4: 71.111-71.118 Mbp | |
| | | | | (+) | |
| Tongue | C1orf10 | Chromosome 1 open reading | NM_016190.1 | Chr 1: 149.156-149.161 Mbp | |
| | | fram 10 | | (−) | |
| | Hs.46320 | Small proline-rich protein | | Chr 1: 150.174-150.174 Mbp | |
| | | SPRK [human, odontogenic | | (−) | |
| | | keratocysts, mRNA Partial, | | | |
| | | 317 nt] | | | |
| | KRT13 | Keratin 13 | NM_002274.2 | Chr 17: 39.565-39.57 Mbp | |
| | | | | (−) | |
| | KRT16 | Keratin 16 (foacl non- | NM_005557.2 | Chr 17: 39.674-39.677 Mbp | |
| | | epidermolytic palmoplantar | | (−) | |
| | | keratoderma) | | | |
| | KRT4 | Keratin 4 | NM_002272.1 | Chr 12: 52.917-52.925 Mbp | |
| | | | | (−) | |
| | LY6D | Lymphocyte antigen 6 | NM_003695.1 | Chr 8: 143.67-143.672 Mbp | |
| | | complex, locus D | | (−) | |
| | MYH2 | Myosin, heavy polypeptide 2, | NM_017534.2 | Chr 17: 10.367-10.394 Mbp | |
| | | skeletal muscle, adult | | (−) | |
| | PITX1 | paired-like homeodomain | NM_002653.3 | Chr 5: 134.394-134.4 Mbp | |
| | | transcription factor 1 | | (−) | |
| | PKP1 | Plakophilin 1 (ectodermal | NM_000299.1 | Chr 1: 197.719-197.765 Mbp | |
| | | dysplasia/skin fragility | | (+) | |
| | | syndrome) | | | |
| | RHCG | Rhesus blood group, C | NM_016321.1 | Chr 15: 87.601-87.627 Mbp | |
| | | glycoprotein | | (−) | |
| | S100A7 | S100 calcium binding protein | NM_002963.2 | Chr 1: 150.205-150.206 Mbp | |
| | | A7 (psoriasin 1) | | (−) | |
| | SPRR1A | small proline-rich protein 1A | NM_006945.2 | Chr 1: 149.787-149.841 Mbp | |
| | | | | (+) | |
| | SPRR2B | small proline-rich protein 2B | NM_006945.2 | Chr 1: 149.787-149.841 Mbp | |
| | | | | (+) | |
| | SPRR3 | small proline-rich protein 3 | NM_005416.1 | Chr 1: 149.749-149.751 Mbp | |
| | | | | (+) | |
| Pituitary Gland | CGA | Glycoprotein hormones, alpha | NM_000735.2 | Chr 6: 87.745-87.754 Mbp | |
| | | polypeptide | | (−) | |
| | CHGB | Chromogranin B (secretogranin | NM_001819.1 | Chr 20: 5.84-5.854 Mbp | |
| | | 1) | | (+) | |
| | DLK1 | Delta-like 1 homolog | NM_003836.3 | Chr 14: 99.183-99.191 Mbp | |
| | | (Drosophila) | | (+) | |
| | GAL | Galanin | NM_015973.2 | Chr 11: 68.702-68.708 Mbp | |
| | | | | (+) | |
| | GH1 | growth hormone 1 | NM_000515.3 | Chr 17: 62.335-62.337 Mbp | |
| | | | | (−) | |
| | GH2 | growth hormone 2 | NM_002059.3 | Chr 17: 62.298-62.314 Mbp | |
| | | | | (−) | |
| | GHRHR | growth hormone releasing | NM_000823.1 | Chr 7: 30.711-30.727 Mbp | |
| | | hormone receptor | | (+) | |
| | POMC | Proopiomelanocortin | NM_000939.1 | Chr 2: 25.341-25.349 Mbp | |
| | | (adrenocorticotropin/beta- | | (−) | |
| | | lipotropin/alpha-melanocyte | | | |
| | | stimulating horomone/beta- | | | |
| | | melanocyte stimulating | | | |
| | | horomone/beta-endorphin | | | |
| | PRL | Proactin | NM_000948.2 | Chr 6: 22.35-22.36 Mbp | |
| | | | | (−) | |
| | SCG2 | Secretogranin II (chromogranin | NM_003469.2 | Chr 2: 224.425-224.431 Mbp | |
| | | C) | | (−) | |
| | TSHB | Thyroid stimulating hormone, | NM_000549.2 | Chr 1: 114.672-114.677 Mbp | |
| | | beta | | (+) | |
| Skin | SCGB1D2 | Secretoglobin, family 1D, | NM_006551.2 | Chr 11: 62.26-62.263 Mbp | |
| | | member 2 | | (+) | |
| | UNQ467 | KIPU467 | NM_207392.1 | Chr 19: 40.654-40.657 Mbp | |
| | | | | (−) | |
| Retinoblastoma | KIAA1199 | KIAA1199 | NM_018689.1 | Chr 15: 78.647-78.819 Mbp | |
| | | | | (+) | |
| Spinal Cord | BCAS1 | breast carcinoma amplified | NM_003657.1 | Chr 20: 53.198-53.325 Mbp | |
| | | sequence 1 | | (−) | |
| | PTPRZ1 | Protein tyrosine phosphatase, | NM_002851.1 | Chr 7: 121.054-121.242 Mbp | |
| | | receptor-type, Z polypeptide 1 | | (+) | |
| | UGT8 | UDP glycosyltransferase 8 | NM_003360.2 | Chr 4: 115.936-115.99 Mbp | |
| | | (UDP-galactose ceramide | | (+) | |
| | | galactosyltransferase) | | | |
| Spleen | ECGF1 | Endothelial cell growth factor | NM_001953.2 | Chr 22: 49.096-49.1 Mbp | |
| | | 1 (platelet-derived) | | (−) | |
| | HMOX1 | Heme oxygenase (decycling) 1 | NM_002133.1 | Chr 22: 34.101-34.114 Mbp | |
| | | | | (+) | |
| Thymus | CD1E | CD1E antigen, e polypeptide | NM_030893.1 | Chr 1: 155.101-155.105 Mbp | |
| | | | | (+) | |
| | LCK | Lymphocyte-specific protein | NM_005356.2 | Chr 1: 32.143-32.178 Mbp | |
| | | tyrosine kinase | | (+) | |
| Thyroid | DIO1 | Deiodinase, iodothyronine, | NM_000792.3 | Chr 1: 53.717-53.734 Mbp | |
| | | type I | | (+) | |
| | PAX8 | paired box gene 8 | NM_003466.2 | Chr 2: 113.881-113.943 Mbp | |
| | | | | (−) | |
| | PTH | Parathyroid horomone | NM_000315.2 | Chr 11: 13.552-13.556 Mbp | |
| | | | | (−) | |
| | SLC6A4 | solute carrier family 26, | NM_000441.1 | Chr 7: 106.847-106.904 Mbp | |
| | | member 4 | | (+) | |
| | TFF3 | Trefoil factor 3 (intestinal) | NM_003226.2 | Chr 21: 42.626-42.629 Mbp | |
| | | | | (−) | |
| Trachea | AGR2 | Anterior gradient 2 homolog | NM_006408.2 | Chr 7: 16.541-16.554 Mbp | |
| | | (Xenopus laevis) | | (−) | |
| | C17 | Cytokine-like protein C17 | NM_018659.1 | Chr 4: 5.009-5.013 Mbp | |
| | | | | (−) | |
| | DMBT1 | deleted in malignant brain | NM_004406.1 | Chr | |
| | | tumors 1 | | 10_random: 0.506-0.658 Mbp | |
| | | | | (+) | |
| | LOC389429 | hypothetical LOC389429 | | Chr 6: 127.833-127.848 Mbp | |
| | | | | (+) | |
| | LTF | Lactotransferrin | NM_002343.1 | Chr 3: 46.296-46.325 Mbp | |
| | | | | (−) | |
| | MSMB | Microseminoprotein, beta- | NM_002443.2 | Chr 10: 51.441-51.455 Mbp | |
| | | | | (+) | |
| Kidney | BHMT | Betaine-homocysteine | NM_001713.1 | Chr 5: 78.446-78.466 Mbp | |
| | | methyltransferase | | (+) | |
| | CDH16 | Cadherin 16, KSP-cadherin | NM_004062.2 | Chr 16: 66.677-66.688 Mbp | |
| | | | | (−) | |
| | CYP4A11 | Cytochrome P450, family 4, | NM_000778.2 | Chr 1: 46.781-46.793 Mbp | |
| | | subfamily A, polypeptide 11 | | (−) | |
| | DDC | Dopa decarboxylase (aromatic | NM_000790.1 | Chr 7: 50.233-50.336 Mbp | |
| | | L-amino acid decarboxylase) | | (−) | |
| | GSTA2 | Glutathione S-transferase A2 | NM_000846.3 | Chr 6: 52.616-52.629 Mbp | |
| | | | | (−) | |
| | KNG1 | Kininogen 1 | NM_000893.2 | Chr 3: 187.756-187.782 Mbp | |
| | | | | (+) | |
| | NAT8 | N-acetyltransferase 8 (camello- | NM_003960.2 | Chr 2: 73.825-73.827 Mbp | |
| | | like) | | (+) | |
| | SLC12A1 | solute carrier family 12 | NM_000338.1 | Chr 15: 46.079-46.175 Mbp | |
| | | (sodium/potassium/chloride | | (+) | |
| | | transporters), member 1 | | | |
| | SLC13A3 | solute carrier family 13 | NM_022829.3 | Chr 20: 45.824-45.918 Mbp | |
| | | (sodium-dependent | | (−) | |
| | | dicarboxylate transporter), | | | |
| | | member 3 | | | |
| | UGT1A10 | UDP glycosyltransferase 1 | NM_019075.2 | Chr 2: 234.561-234.698 Mbp | |
| | | family, polypeptide A10 | | (+) | |
| | UGT2B7 | UDP glycosyltransferase 2 | NM_001074.1 | Chr 4: 70.212-70.228 Mbp | |
| | | family, polypeptide B7 | | (+) | |
| | UMOD | Uromodulin (uromucoid, | NM_003361.1 | Chr 16: 20.271-20.291 Mbp | |
| | | Tamm-Horsfall) glycoprotein | | (−) | |
| | 35460_at | Human G protein-coupled | | Chr 19: 50.769-50.769 Mbp | |
| | | receptor (GPR4) gene, | | (−) | |
| | | complete cds | | | |
| Huvec | 590_at | Human intercellular adhesion | | Chr 17: 62.42-62.422 Mbp | |
| | | molecule 2 (ICAM-2) gene | | (−) | |
| | ERG | v-ets erythroblastosis virus E26 | NM_004449.3 | Chr 21: 38.673-38.954 Mbp | |
| | | oncogene like (avian) | | (−) | |
| | ESM1 | Endothelial cell-specific | NM_007036.2 | Chr 5: 54.244-54.251 Mbp | |
| | | molecule 1 | | (−) | |
| | ICAM2 | intercellular adhesion molecule 2 | NM_000873.2 | Chr 17: 62.42-62.438 Mbp | |
| | | | | (−) | |
| | TEK | TEK tyrosine kinase, | NM_000459.1 | Chr 9: 27.099-27.22 Mbp | |
| | | endothelial (venous | | (+) | |
| | | malformations, multiple | | | |
| | | cutaneous and mucosal) | | | |
| | VEGFC | vascular endothelial growth | NM_005429.2 | Chr 4: 178.189-178.298 Mbp | |
| | | factor C | | (−) | |

What is claimed is:

1. A pluripotent stem cell containing a nucleic acid segment, wherein the nucleic acid segment comprises the structure P-I, wherein P is a transcriptional control element and I is a sequence encoding a marker, wherein the marker comprises a transformation agent.

2. The stem cell of claim 1, wherein the nucleic acid segment is a heterologous nucleic acid segment.

3. The stem cell of claim 1, wherein the nucleic acid segment is an exogenous nucleic acid segment.

4. The stem cell of claim 1, wherein the marker is heterologous.

5. The stem cells of claim 1, wherein P and I are contained in the same vector.

6. The stem cells of claim 1, wherein P and I are contained in different vectors.

7. The stem cell of claim 1, wherein I is a heterologous nucleic acid sequence.

8. The stem cell of claim 7, wherein the nucleic acid segment further comprises a suicide gene.

9. The stem cell of claim 7, wherein P is a tissue specific transcriptional control element.

10. The stem cell of claim 7, wherein P is a cell type specific transcriptional control element.

11. The stem cell of claim 7, wherein P is a cell lineage specific transcriptional control element.

12. The stem cell of claim 7, wherein P is a cell specific transcriptional control element.

13. The stem cell of claim 7, wherein P causes I to be preferentially or selectively expressed.

14. The stem cell of claim 7, wherein the marker comprises a temperature permissive immortalization agent.

15. The stem cell of claim 7, wherein the transformation agent is a temperature permissive agent.

16. The stem cell of claim 7, wherein I comprises the SV40 large T antigen.

17. The stem cell of claim 7, wherein the nucleic acid segment is flanked by a site-specific excision sequence.

18. The stem cell of claim 7, wherein I is flanked by a site-specific excision sequence.

19. The stem cell of claim 7, wherein P is flanked by a site-specific excision sequence.

20. The stem cell of claim 7, wherein the nucleic acid segment further comprises X, wherein X is a site-specific excision sequence, wherein X flanks P-I, wherein the nucleic acid segment comprises the structure X-P-I-X.

21. The stem cell of claim 20, wherein the nucleic acid segment is excised at X.

22. The stem cell of claim 21, wherein X is a loxP site.

23. A differentiated cell produced by culturing the stem cell of claim 7 under conditions in which the transcriptional control element is activated, whereby I is preferentially or selectively expressed.

24. The differentiated cell of claim 23, wherein the conditions in which the transcriptional control element is activated are conditions in which the stem cell differentiates.

25. The differentiated cell of claim 23, wherein the stem cell differentiates under the conditions in which the transcriptional control element is activated.

26. The differentiated cell of claim 23, wherein the transcriptional control element is activated by allowing the stem cells to spontaneously differentiate into an embryoid body.

27. The differentiated cell of claim 23, wherein the nucleic acid segment is excised from the differentiated cell.

28. The differentiated cell of claim 27, wherein the nucleic acid segment is excised using an adenovirus-mediated site-specific excision.

29. The differentiated cell of claim 27, wherein the nucleic acid segment is excised using a recombinase.

30. The differentiated cell of claim 29, wherein the recombinase is Cre.

31. The differentiated cell of claim 27, wherein the excision of the nucleic acid segment results in recombination of the nucleic acid molecule from which the nucleic acid segment is excised.

32. The differentiated cell of claim 23, wherein the effect of the expression of I is reversed.

33. The differentiated cell of claim 32, wherein the effect of expression of I is transformation of the differentiated cell, wherein reversal of the effect of the expression of I is reversal of transformation of the differentiated cell.

34. The differentiated cell of claim 32, wherein the effect of the expression of I is reversed by expression of a dominant negative transformation agent.

35. The differentiated cell of claim 32, wherein the effect of the expression of I is reversed by excision of the nucleic acid segment.

36. The differentiated cell of claim 23, wherein the differentiated cell is a hepatocyte.

37. The differentiated cell of claim 23, wherein the differentiated cell is a stem cell derived conditionally immortal cell.

38. A method comprising introducing the differentiated cell of claim 23 into a subject.

39. The method of claim 38, wherein the differentiated cell is introduced by administering the differentiated cell to the subject.

40. The method of claim 38, wherein the differentiated cell is introduced by transplanting the differentiated cell into the subject.

41. A method of assaying a composition for toxicity, the method comprising incubating the composition with the differentiated cell of claim 23, and assessing the differentiated cell for toxic effects.

42. A method of assaying a compound for toxicity, the method comprising incubating the compound with the differentiated cell of claim 23, and assessing the differentiated cell for toxic effects.

43. A method of assaying a composition for an effect of interest on a cell, the method comprising incubating the composition with the differentiated cell of claim 23, and assessing the differentiated cell for the effect of interest.

44. A method of assaying a compound for an effect of interest on a cell, the method comprising incubating the compound with the differentiated cell of claim 23, and assessing the differentiated cell for the effect of interest.

45. A method of deriving differentiated cells from stem cells, the method comprising:
culturing the stem cells of claim 7 under conditions in which the transcriptional control element is activated, whereby I is preferentially or selectively expressed, thereby deriving differentiated cells.

46. A method of deriving stem cell derived conditionally immortal cell types, the method comprising:
culturing the stem cells of claim 7 under conditions in which the transcriptional control element is activated, whereby I is preferentially or selectively expressed, thereby deriving stem cell derived conditionally immortal cell types.

47. A method of deriving stem cell derived conditionally immortal cell types, the method comprising:
transfecting stem cells with a nucleic acid segment comprising the structure P-I, wherein P is a transcriptional control element and I is a sequence encoding a marker, wherein the marker comprises a transformation agent;
culturing the stem cells under conditions in which the transcriptional control element is activated, whereby I is preferentially or selectively expressed, thereby deriving stem cell derived conditionally immortal cell types.

48. A method of deriving differentiated cells from stem cells, the method comprising:
transfecting stem cells with a nucleic acid segment comprising the structure P-I, wherein P is a transcriptional control element and I is a sequence encoding a marker, wherein the marker comprises a transformation agent;
culturing the stem cells under conditions in which the transcriptional control element is activated, whereby I is preferentially or selectively expressed, thereby deriving differentiated cells.

49. The method of claim 48, wherein the conditions in which the transcriptional control element is activated are conditions in which the stem cells differentiate.

50. The method of claim 48, wherein the stem cells differentiate under the conditions in which the transcriptional control element is activated.

51. The method of claim 48, wherein the transcriptional control element is activated by allowing the stem cells to spontaneously differentiate into an embryoid body.

52. The method of claim 48 further comprising selecting cells expressing I.

53. The method of claim 48 further comprising increasing the purity of the cells expressing I.

54. The method of claim 53, wherein increasing the purity comprises creating a clonal or semi-purified population of cells.

55. The method of claim 48 further comprising excising the nucleic acid segment.

56. The method of claim 48 further comprising cloning the differentiated cells.

57. The method of claim 48 further comprising culturing the differentiated cells.

58. The method of claim 48 further comprising freezing the differentiated cells.

59. The method of claim 48 further comprising adding a gene of interest to the selected cells.

60. The method of claim 48 further comprising:
excising the nucleic acid segment; and
freezing of the selected cells.

61. The method of claim 60, wherein the ends of the nucleic acid formerly containing the nucleic acid segment recombine when the nucleic acid segment is excised.

62. The method of claim 48 further comprising culturing the cells expressing I.

63. The method of claim 62, further comprising cloning the cultured cells expressing I.

64. The method of claim 48 further comprising introducing the differentiated cells into a subject.

65. The method of claim 64, wherein the differentiated cell is introduced by administering the differentiated cell to the subject.

66. The method of claim 64, wherein the differentiated cell is introduced by transplanting the differentiated cell into the subject.

67. The method of claim 48 further comprising incubating a composition with the differentiated cells, and assessing the differentiated cells for toxic effects.

68. The method of claim 48 further comprising incubating a compound with the differentiated cells, and assessing the differentiated cells for toxic effects.

69. The method of claim 48 further comprising incubating a composition with the differentiated cells, and assessing the differentiated cells for an effect of interest.

70. The method of claim 48 further comprising incubating a compound with the differentiated cells, and assessing the differentiated cells for an effect of interest.

71. A method of deriving differentiated cells from stem cells, the method comprising:
transfecting stem cells with a nucleic acid segment comprising the structure P-I, wherein P is a transcriptional control element and I is a sequence encoding a marker;
culturing the stem cells under conditions in which the transcriptional control element is activated, whereby I is preferentially or selectively expressed, wherein the conditions in which the transcriptional control element is activated are conditions in which the stem cells differentiate thereby deriving differentiated cells.

72. The method of claim 71 further comprising selecting the differentiated cells by selecting for the marker.

73. The method of claim 71 further comprising screening for the differentiated cells be identifying cells expressing the marker.

74. The method of claim 71, wherein the stem cells differentiate under the conditions in which the transcriptional control element is activated.

75. The method of claim 71, wherein the transcriptional control element is activated by allowing the stem cells to spontaneously differentiate into an embryoid body.


SEQUENCE LISTING (29 sequences)

SEQ ID NO: 1 - DNA, 202 bp, Artificial Sequence
  Description of Artificial Sequence/note = synthetic construct
cacgtcgcatggagaccaccgtgaacgcccaccaggtcttgcccaaggtcttacataagaggactcttggactctcagcaatgtcaacgaccgaccttgaggcatacttcaaagactgtgtgtttaaagactgggaggagttgggggaggagattaggctaaaggtctttgtactaggaggctgtaggcataaattggtctg

SEQ ID NO: 2 - DNA, 677 bp, Artificial Sequence
  Description of Artificial Sequence/note = synthetic construct
atgacagaatacaagcttgtggtggtgggcgctggaggcgtgggaaagagtgccctgaccatccagctgatccagaaccactttgtggacgaatacgaccccactatagaggattcctaccggaagcaggtggtcattgatggggagacgtgcctgttggacatcctggataccgccggcctggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgatgtgcccatggtgctggtggggaacaagtgtgacctggctgcacgcactgttgaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcagaacatggaggtgccggatgcagaaagaaggtgcagacgaaaggaggaggaagaaagaacggaagcaaggaagaaagaaagggctgctggag

SEQ ID NO: 3 - DNA, 530 bp, Artificial Sequence
  Description of Artificial Sequence/note = synthetic construct
tatagatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggtcggagtactgccccgcccctagcgattagccccggccccgcatagctccgccccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagacaactctgaatactttcaaaagttaccgagaaagaagaactcag

SEQ ID NO: 4 - DNA, 677 bp, Artificial Sequence; identical to SEQ ID NO: 2
  Description of Artificial Sequence/note = synthetic construct

SEQ ID NO: 5 - DNA, 37 bp, Artificial Sequence
  Description of Artificial Sequence/note = synthetic construct
attataacttcgtataatgtatgctatacgaagttat

SEQ ID NO: 6 - DNA, 40 bp, Artificial Sequence
  Description of Artificial Sequence/note = synthetic construct
ataacttcgtataatgtatgctatacgaagttatgaagac

SEQ ID NO: 7 - DNA, 677 bp, Artificial Sequence
  Description of Artificial Sequence/note = synthetic construct
atgacagaatacaagcttgtggtggtgggcgctggaggcgtgggaaagagtgccctgaccatccagctgatccagaaccactttgtggacgaatacgaccccactatagaggattgctaccggaagcaggtggtcattgatggggagacgtgcctgttggacatcctggataccgccggcctggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgatgtgcccatggtgctggtggggaacaagtgtgacctggctgcacgcactgttgaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcagaacatggaggtgccggatgcagaaagaaggtgcagacgaaaggaggaggaagaaagaacggaagcaaggaagaaagaaagggctgctggag

SEQ ID NO: 8 - DNA, 51 bp, Artificial Sequence
  Description of Artificial Sequence/note = synthetic construct
gaatacgaccccactatagaggattgctaccggaagcaggtggtcattgat

SEQ ID NO: 9 - DNA, 1084 bp, Artificial Sequence
  Description of Artificial Sequence/note = synthetic construct
tcagatcctgcccttcaaaaacaaaacatgagcgtgccaagaaagtccaaggtgttgaatgttgccacttcaagcctaaactttctaggaacacctaagtgggtggcagcttccagttctccaggctgcttctaggccagagctgggttccacaagagacagaataggcatatatatgcttaaggaactggaaaaacaggctctctctctctcacaaacacacacacacacataccaaggtagctgtcaaaatgttatccgaaattttggaaccaaaaaatcttgaaagatggtattccaatatcacattttatgtaagttttctattatattagattcaaattacgattcgaggccacaagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttctgattccataccagaggggctcagg

SEQ ID NO: 10 - DNA, 1232 bp, Artificial Sequence
  Description of Artificial Sequence/note = synthetic construct
agagccccaaggtggaggcataaatgggactggtgaatgacagaaggggcaaaaatgcactcatccattcactctgcaagtatctacggcacgtacgccagctcccaagcaggtttgcgggttgcacagcgggcgatgcaatctgatttaggcttttaaagggattgcaatcaagtggggccccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaca

SEQ ID NO: 11 - DNA, 1039 bp, Artificial Sequence
  Description of Artificial Sequence/note = synthetic construct
aaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcaca

SEQ ID NO: 12 - DNA, 1051 bp, Artificial Sequence
  Description of Artificial Sequence/note = synthetic construct
aggaatctaggggtgaaagctgagaaactggtccctgggccagcaggggctgaagagggaggctgctgaactggcctggggcgactgccctagagggagcagcatcctttctcttgccctttgtgggggttcctggagacccttggacccatagccagggctacagctgcctgccatgaccagacactcacatacagacacatgtaaacacccagacataaacacacagaagcaacacccgcagacacactcacatagacacatagacacaccacgcacacataaagacccagacccacagagacaaatgcagacacccgggccatcccttacatagacacagatacggagcacacataggcacgcacagagccccccaccacggaggcacccagacaatacacacaggtgtacacagacccccctccacacaaacacagacacagagagacccacagacacatagacaccccgacacagacacagacaacacacacagacacatgctgacttccgccttcccccacatggacacggtcatagacacagacacagacacaggggactttggagagcttaagttcagaggtgcccttagatcatcccagtccttctggccccaacacaacacgtagcatgtacttggtccacccttcttcacctcttcaacttacggacctgcagagaggcagcagggccctgtgtgggtatgtgcgggtgtgcagatgagtgtgtgtgtatgtgtgtatgtgtgtgttaagcagtggcctttgtccccctctctccctcccccttcccaacaggtgattgggaggaatggagatccctcctccactacccattcctgagcctgaacaatgccctccccaggccccaaaatagcccctaagcctagccatatccttttatggccctgtccctattgtgcactgcaggggtggggctggggtcatgaggtatccgggcaggataaaggcctgggtgaggcggctcacctaccctgctttctgcattcttctctccacatccctctctgtacttacagccccca

SEQ ID NO: 13 - DNA, 2295 bp, Artificial Sequence
  Description of Artificial Sequence/note = synthetic construct
gaacaaaacagacatcatcccacctctttccactacaggccaagcaccatgctggtctctgggaaccctgttgtgagcaagacagacccaggcttacccttgtggactcatgttacaggcagggagacgggcacaaaacacaaataaaaagcttccatgctgtcagaagcactatgcaaaaagcaagatgctgaggtactgctaagctgtgtgggatgggggctcagcccggccagggaggggccagttgtgggtcagtcttgacccaaggcatccaggacaccctccttctggccatgagggtccacgtcagaatcaaaccctcaccttaacctcattagcgttgggcataatcaccaggccaagcgccttaaactacgagaggccccatcccacccgccctgccttagccctgccacgtgtgccaaacgctgttagacccaacaccacccaggccaggtagggggctggagcccaggtgggctgcagggaagggggcactcttctgagcagacagatctgggaatcctgggtgggaagagagacagtgagagagagattaagggatatttcccaggcatcagggctttgcactctcaggggtccttccgcctggatgtccttcccctgaagcttcctcctgttgttccgttctcagctcaagctccagcttctcagagaagcctcctgtgttgggagtggctgcgactgaactgtccctactgttattcgctcttctatttgtttgtggtccctgtgccccctcaccccacaaaaacactggcttcttgtgagcaggagcttgctctttcgtgtaccctgtgtgtccccaaggaccaagcaccttgtctgggccacagtaggtgctcaatacacatgttggctggacagtggtcactgagcggccgcacgtcgggcactctcagcacttgcacaggccgccccagacaccccacttcattcctgggaggtgtcatcatgttgcttggacgacggggagagggggacctgccagtgttggcctccattttcccccagtcatctgcccccaaggctctgactactttctttctcacggtacatcctgctattctggaatcggccctcgtggggccacctggtacatggcatttgaggccctcgtggctgattaggcctcccccaacagtgccctgtctgctgcctccagggccagcctccccttcagactggagtcccctgaagggttctgcccctcccctgctctggtagccccctccatcctccctccctccactccatctttgggggcatttgagtcacctttctacaccagtgatctgcccaagccactgctcactttcctctggataaagccaggttccccggcctagcgttcaagacccattacaactgcccccagcccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagcc

SEQ ID NO: 14 - DNA, 1116 bp, Artificial Sequence
  Description of Artificial Sequence/note = synthetic construct
agagtttcactcgtttcccaggctggagtgcactggcgtgatcttggctcactgcaacctccacttcccgggttcaagcgattctcctgcctcagcctcccgagtagctgggattacaggcatgcgccaccatgcccagctaattttgtatttttagtagagatggcgtttctccatgttggtcaggctggtcttgaactcccggcctcaggtgatccgcctgcctcggcctcccaaagtggtgggattacaggcgtgagccactgtgcctggcctcctttttatttttttcactgaacaaaccatgaaactttcccagatgtaaatatctatttcccatttttctttttttaaaataaggcattattttaaccatttgagtgttagatattatttttagataatattttaatttaggcataactgccgtgcaaaatctgaagattaatatctaccttgtgagtcattcctctgtgagacagtgcatgttaaatatgttgaattggcaggtgaaaaaggaagaaaaaatgagtagtgattggttatccacagctatgaatgagaaattgaaggtagtagactatggatgacaaacctattcttggtttccttctgtttctgaaattctaattactaccacaactacatgagagacactactaacaagcaaagttttacaactttttaaagacatagactttatgttattataattaaaaatcatgcatttttgtcatattaataaaattgcatatacgatataaaggcatggacaaaggtgaagtagcttcaagagacagagtttctgacatcattgtaattttaagcatcgtggatattcccgggaaagtttttggatgccattggggatttcctctttactggatgtggacaatatcctcctattattcacaggaagcaatccctcctataaaagggcctcagccgaagtagtgttcagctgttcttggctgacttcacatcaaaactcctatactgacctgagacagaggcagcagtgatacccacctgagagatcctgtgtttgaacaactgcttcccaaaacggaaagtatttcaagcctaaacctttgggtgaaaagaactcttgaagtc

SEQ ID NO: 15 - DNA, 1081 bp, Artificial Sequence
  Description of Artificial Sequence/note = synthetic construct
caacataggcaacaaagcaagactccatctctacaaaaaatcaaaataaaaataaattagctgggtgtggtagcgcatgcctgtggtcttagctactcaggaggctgacgaaggaggatcacttgagcccaggagttcaagactgcagtgagctatgattgtactgccgcattctagcctgggcaacaaagaaagaaccccatctctaaaaaaaaaaattaaataaataaaaggagggctgtaacaacatggcaaaaagtttactgggcatctttttttattttttttttttgagatggcgtctcgctctgtcgcccaggttggagtgcagtggcacaatcttggctcactgcaagctccacctcccgggttcacaccattctcctgcctcagcctcctgaatagctgggactacaggtgcccgtcaccacacccagctaatttttttgtatttttagtagagacggggtttcaccgtgttagccaggatggtcttgatctcctgaccttgtgatccgctcgtctcagcctcccaaagtgctgggattacaggcgtgagccactgcgcctggccctgggtatctttttgcctataataaagccagccatctaaaaacagttcaagccctggctctggtacctgtcagctgtgtggccctgggcgggtccctcactctctgtgagcctcagttgcctcacctgtgaaatgtaactcagtggttgagtggatcaagtgaagggctgtatgtcaagcccctgtcaagccacctggtgctccgtgtcctgtggatggtagctgccctgaagcgggactttgcagactgaagtgctgtctcttcagagggagtgcaggtgtccggctcctggtgtgggagaggcaccctgtgctccctggcccgtgagacaagattgggtttgggggccagggcaggagtaggggcccgtcaccaggggaaacggctcgtggtagagcagctgaggtgcagctgggcctgtggcctagaaggcagtcttgtgggtgcctcctcccccagccgcaactcaggtctgcagctgggtcctgcctccttccgagtgggcc

SEQ ID NO: 16 - DNA, 1126 bp, Artificial Sequence
  Description of Artificial Sequence/note = synthetic construct
tcactcactgcctattaattaatgttaagcctgcaaagaatggagttgtcctggatatttggccaaaaaaaaaatgtatccacaaacagggacgtaatcaggcagggagcctcgttaagaagttttgttcttgtcctaggagtgatgagagatcactgaaggatttagagaggggctgtatcatcaggcttgggttccaaagcctcactgagagagttggggagctgactgatgtcagatgctcgtgcagccgccccgtagggcctgtatttcctccatggtgcctcactgcagcaccgagcttgcaaaagatcctctctctttatgggaatttcaaaacagaagcaaaatagcaccggggcttaaagcattcttgggaatttccctgtctttccctctaaataatcagcatgtaaattgcaaaaaaaaaaaaaaaaaaaaaaaaaaagacacgggcccaaaagggagcgctcagtttcaggctctttgctttccttcctcccgaggctctctggcccttacccagcctgaagacagaaagtgtgagggggagggtaggaaggtaggtcaagcagggcaatgctgagcctgggaagaaaacaacagccttgtttagggcactgtggcttacgtaactaaattgtgcccagtttccacctggccaggggcctggagtgaatgctgaagatgcaaaggtagaggctgccagaaaagccaggaaattgctggcaagaaaggccagtggtggggtgcaggagtgggaggaaggctgggaaatgcggctgagtcacatctccagaagccccccatcatcaccctagtggctcttctgctggcaggtgcctcatgaagacctgacccaaagttttcaaaactctgcggtttctcaaccctcctctggtaatccatagtactcccccgcctccacttgccagcctcgtgattccttcattgacacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccaga

SEQ ID NO: 17 - DNA, 4105 bp, Artificial Sequence
  Description of Artificial Sequence/note = synthetic construct
aaaggcacattcacagataagtcgtttttgatctctagtaattattgagccctgggaggggcagtccctccatgatcagtaaggccccaaaatgtacaagcaccaaatacagaaactagaaagcatggtgattacacttatgaatagattccaacccaaactccccattttcctgcccaagtctttgtcaagaaaactccagaagccacctggggtcactgtctttaatcctttgaagacacccataataaggtttagacaaggcctcatgaaaaaggtataggataaaaacgactagttcagaaaaaaaaaagtaaataaatgaagagataacttcccttttcatcaaagaattgcaaaaatgaacaataatgagatcgtttccacttatccttttggcaaagatgaaagcaagttaccaatgctcagaactggtgagtagtgttggtgggattgtaaaccaatttataataattctctggggtgctgtttgggaagaggagtcaatattcctacagctgtgctacctctttgacccagaatttccactccttggacataatcctgaggagatgacaacaccagtgccagtggttaccctaccactgagtgggacaggatggagaaatgtcctgaaaagagcaattcacaatagcaaagacttggaaccaactcaaatgtccatcaatgatagactggattaagaaaatgtggcacatatacaccgtggaatactatgcagccataaaaaatatgagttcatgtcctttgtagggacatggatgaagatggaaaccatcattctcagcaaactatcgcaaggacaaaaaaccaaacaccgcatgttctcactcacaggtgggaattgaacaatgagagcacttggacacaggaaggggaacatcacacccaggggcctattgtgggatgcggggagcggggagggatagcattaggagttatacctaatgtaaatgacaagttaatgggtgcagcacatcaacatggcacatgtatacatatgtaacaaacctgcacgttgtgcacttgtaccctaaaacttaaagtataaaaaaaaaaaagatcaatgcagtgatcatggtgatattttcctgctcagcccaagttcacacatattttatttttctcaacatgatgacagccactctcacactgacttttggaatgtcatgtatgttgaactgggtctgaagacatggttttaactcaggctctgtcattttctacctcagtgattgcacaacagcaaagcagaattttcactacttccatgaatataatcattactatatgactttacttgcatcatctcctttggttactattactactgtgggagatgggtattctcattttatagacaaggaaattgacctctggacctcaggaaggttaagaaatgagcccactgccacacaataaacaccagataaaggaggcagactgactccaaagtcagtctatttaagtgcaaatttatttcgcctccaaagggacctcccagtcatcagacctgattctttgttgtacagagtgggtcaggtccagtgatgtctgaactaccttctggttctgactttcagccattctcagctcctctcttgcttgtgtctggattctaaggctgatctcatgagaatgggtgtttcagaagggtgccctctccaagacaggtgcacctcccatctggggcagtgaatatcccttttgtccttatgcagcctggcttcagatactggcttctgcctggctccttgatcccaccctgcccttgtcagtgaccaagaagaagcccagcaccttggcactgctttcccagttaatttctaactatggaatctcttgctgttagaaggtgcgaaacagtgaccttgtatttccgggcacaggtgtgaccccccaatgtcaatcatttggggtctctagctattaggaaaaagaacaacaacaacctcacagcttggacaaggcaaacattatgccaggaggaaaaaatattccacccccaagaaaacaatatcaaaaaacagaactagagactaattggaggagagattgccagcctggggcaaatgtgtatatataagtatgaggcacatcatgaccagactaactctacctttctggcttcaggtaaggctatctgtagctgtcttctcctagcccagcttctccccatcctatttgagggaggtaggagaggaaattaagaccttggacactggggtcagacctggatttgagtcattattctgccaattattggcttcatgatctttagtaagttagttttcctctctttgatcctctctgtcctagtaacaatgactactattttttgacttattccatgaatattagttatgcacctattatgtgccaggtaccagggttacaacaatgaacaacttgctatgtgctgggccctgctcgacatatgtcatcttagttaatcccagtgacaatatgcagtgttttaggtcgcatcttattagcgggtcataaaatcattccttttgtgggttatatccagggacttcttttaattaaatataatagaatatgtcagtaagcaatacaaatttgaggcaagcagttagcctctttgtccccctgttttctcatctgcaaactaggataatacttatctcatagggttgtggtaagaattaaatcacttaatgtagtaaggatatcaattgtcttataaaattttatgtgtgtgtgcacatacatgcagaagcgactgcactagatcatgacataaaatgtttttctttctttctgtctttttttttttgggggggggggatggactctctgtcacccaggcaggagtgcagtggcacgatctcagctcactgcaacctctgcctcccaggttcaagcaatcctcctgtctcagcctcccgagtagctgggattacaggcgcccaccaccatgcccggctaatttttgtatttttattagagatggggtttcaccatgctggccaggctggtctcaaactcctgaccttgtgatccaccccctcagtctcccaaagtgttgggattacaggcatgagccactgcacctggcaacataaaatgtttttcttttagtacatcatggtcacgaagatctggaaaacgttgctctaggacccaatgcctttgagcacatgaatgtctttgaactgagcctggcccatcatagatattcagtttgtgaaagcaacaatctccatttcacagacaaaaacggagacttagtaaactgaaatacccacatcacagtgttagtgatcagtaaagcagggatttgaacctcaggtgcaggctgcagctcctacatacttaaccaacatgccaccctattttgctgctataaacactggggtaatactggcatccaccctggactatttttccaattggaagaaatagtaagtgaccactccagaaacatttaaatgcattgtgaaggttaattacctctcaagttgctcgtctcaagccacttctttacgatatgtgagttgcatgaaggagcagactgccatgttcattgttctccatggtgactagctccctgtttggcacaatgtagctactcaatagatgtttgttgaatgaataagtgatccaggcaaattgaaagtttccaggcagaggatcagttaaaatccaaagctgccttcagtaaagtggtgagtccagaacactattccatctggctacttcctgctccaaatgactgagttcttcaaaatgtgcaatgtgctgagaattggggagccaagactgggatgttggtgaggtaaggagggggagtacaaggggtaaagtcccagcaaaacaagggctgcagtgttatgcaattttttagtccatataagtgacacctcctggagttgtatactatacaatcaaagcactccttccagctgtggggaggagagttagatcatgcatttgtcccatccatctctgttcacaggacaccagacatcagagacagagagaaaaattcaaagggccaacccgtctttcctttgggcaggtgctatctagacctgaagtagcgggaagagcagaaagg

SEQ ID NO: 18 - DNA, 1160 bp, Artificial Sequence
  Description of Artificial Sequence/note = synthetic construct
agtgaaagctgcgagaaagacccgtgcgctctgtaactaggcgagtcagcatcacagcccaatggaatatactctaaagatgggattttcatgcaaaatatttaaaagattccatcttgatctcttgtgcggcttaagcatttaaagatataacttggggttctccctggatcctggctacatcccatgaagcccaagtgcacaattttggaatatttcctccacggtgctttgcttgcttgtttgctttcggctgggtcaggtgaggaggatgacagggcttctgtattgctaaatgggttttcacaggagatggaagataaaaatcaaaattatttgtaacgtaagacagcagggcctggtgagaggacgcttcgccgccaacaattagcaattcggcttctacacagcagccggagatcagctttgctgcatttggtccaggttggagcatctccgcagcagctgcaacagccgcacgaaggtagctccgggcggggagcgaggcgctgtcctcggtgctgaaaggccgaggcgcgcggtgggcgcgacagccccggagacccgaggtctcgcggagggacagcggctacgggccccgagctgtgctttctcagcgccgcgcacgcgacgcgtccacggtggtgcggggtgccgggcgccgtgcgggggagggggcgcgcgctcccgcctcctgccgcgagtcgcgcacgcgcgcccgggactgcctgcccctctctgtgacttgcctgtgtgtgtgcgtgtgtgtatgtgtgtgtgtgtgtgtgtgtgcgcgcgcgcgtgagtgagagaggagagagggagaagagagcgcgagagagggtgagtgtgtgtgagtgcatgggagggtgctgaatattccgagacactgggaccacagcggcagctccgctgaaaactgcattcagccagtcctccggacttctggagcggggacagggcgcagggcatcagcagccaccagcaggacctgggaaatagggattcttctgcctccacttcaggttttagcagcttggtgctaaattgctgtctcaaaatgcagaggatctaatttgcagaggaaaacagccaaagaaggaagaggaggaaaaggaaaaaaaaaggggtatattgtggatgctctacttttcttggaa

SEQ ID NO: 19 - DNA, 1084 bp, Artificial Sequence
  Description of Artificial Sequence/note = synthetic construct
gcctccccacagccaagttgctggagcttcgccccacctgcccccaacctgcactggcctgactcccccattccacacctggcccagcacccaccacatggtgtggcagacagcactggggcagctgaaagactccatgctacaaattggcactgcccagcttcctgctcctgggagtggggtccgttgtctgagcttccgctccctggaagagctccaaactcctcaatggtttgggatttttcaaaccaattccaaataagccaggggagattttcctgtctgggttcccatcattaccacctccacagttggagattccctcctaatccagatgctcaaaccaggaccctaacaactagggcttcatatatcactgggacacttagcataatcaccattgcaggttgccagacatcacccaaagtgatgttctaccaaagacagaaactgcagctctcccatgaccttccaggtgctgccctgactcctccagctgaacctgctagggcagagggaagtggaacgtgcagcatatttgcctgccactctgtccatgtgcccaccaactcggctgcaggctccagagacactggctgggggtcctggggctcagagctgagcaaggttacagggcagacaggttggcctggatcaacctgaccaacctcagctgaagcagtgacttcttcattgtctgaaactaaaccaggaacccagtggagctccctgagtgcagagtgggtgaccttagccagtgaggcatttctcttcttacagacctggtatttttctttaccaggttctgtgctcccctcaagggtcctaagtattcctgcagcctgagtgttctgcagggaaatgtgctgtgtaaacactatgcctatttcctgcttggagaacaggttttgtggtagagctctcaggggtggggaagaagcctggcagcccacatgctataaattctgttgtccacctttatgctctaacttggaggcagagacccaagcagctggaggctctgtgtgtgggtcgctgatttcttggagcctgaaaagaaggagcagcgactggacccagagcc

SEQ ID NO: 20 - DNA, 1044 bp, Artificial Sequence
  Description of Artificial Sequence/note = synthetic construct
gtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggggacaggggtctggggacaggggtcctggggacaggggtgtggggacaggggtgtggggacaggggtgtggggacaggggtcctggggacaggggtctggggacaggggtctgaggacaggggtgtggggacaggggtgtggggacaggggtgtggggacaggggtgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcagatcactgtccttctgcc

SEQ ID NO: 21 - DNA, 1027 bp, Artificial Sequence
  Description of Artificial Sequence/note = synthetic construct
ccagcacgcagtagtgctcgaggcagggagcgtgtttatcaagagggataaacttgatacgaactctgtacgaaggaaggtgtaggtggatggaggggtgtgtgctgccactgagcacaagaaccccacggggtggcctgccaaagttcaaaacgagggagacaggttgatctggacccaggaactacagtgctgaatcctaaaccggggaaagatgagacctagaagagggaggtggtaacctaattggagggtgaggagggaaagagcctgccacagatggggcatctataggggtgctgttgaataactgagagcagctgacttaagcccgaagtgggtacttctccctgggcagatgggaggtctgggacaggctcctctggcagaagggctcctggccaccctgtcctaaggtgggtcagtcacttcctccttcaccagttccacagcatcttactatgagcttggcattcgaggcttctcttggcagggccctgcactcctagcctctccttgcacattgcacccccattccagagaggtttagttaaaggcgggggttaccaagtcagtcagatcttgggcaagtcaccactcctccagagcctcagtttccttatctggaaagtggaggtcatggcaacccgccaacctggttggatgggagcctgagctgttgtgttgcaccttgcctggggcccacgactttgtagctcctgtcctgcactgggcttatgttttcattcattccagaaaccttttcagagagtccctttggggagtgtgggggacaggagggaaagaaacctggtccttgtagccgttcgtctgctccctgccctgggcagaggacgtggggactcaggccagcctgagatcactgggaccagaggaggggctggaggatactacacgcaggggtgggctgggctgggctgggctgggccaggaatgcagcggggcagggctatttaagtcaagggccggctggcaaccccagcaagctgtcctgtgagccgccagc

SEQ ID NO: 22 - DNA, 240 bp, Artificial Sequence
  Description of Artificial Sequence/note = synthetic construct
cgtcgcatggagaccaccgtgaacgcccaccaggtcttgcccaaggtcttacataagaggactcttggactctcagcaatgtcaacgaccgaccttgaggcatacttcaaagactgtgtgtttaaagactgggaggagttgggggaggagattaggctaaaggtctttgtactaggaggctgtaggcataaattggtctgttcaccagcaccatgcaactttttcacctctgcctaatca

SEQ ID NO: 23 - DNA, 2562 bp, Artificial Sequence
  Description of Artificial Sequence/note = synthetic construct
ggtgccctcctgggtctgggcttgggctgggctcttagatgtctcagagaaattgagactctattaatcatctgagtctttccatttctggtgagatgatcatgggtcatcattggcctgagtggtgggatgagctataaatagttctaaattcctggtgtaagtccttgttcaacgagatggacagaatttggccttctaggatgtcatttataacatttggctctttgccaaaatgcaagttagcccatgttttactctggggactgtgaattgtgatccctttaatagactttcccatgttcctccaaatcctggagcagttcttatgggaactgattagttttgtgaaagtctaaacttcacccataaagccatcttggcctgaagtcatctgtgagggcaattatttaataatcttaatgctttcttgaggattactgttccaattatgatttccatttctccttgagtcagctttaagttttattgctagaaaagaaaaatgccaacttgccgtcatctctgctgtcactattttgtgttcaacaattgccttctctatctgctgtatctttttcagcacagaagctgtaatgttattaaacaaagcaatgtatccagatcactcagaatctatgcctgtcacggggagcaggagaagagggtgaatgaagagccacagcatggcaggggagccactgcaaggatgctgaaactcgtgtgaacagagttgctgtaggcaggctgctatggaaccttttggggaagcactgcctcttagggatggcagtgaaaatgggagaagagggtggcattgcctccagatggaagatgtagtgctttgccttgctccttggtgcttggagagggaaagggatgctgctgtaaagttcctggctggactttggcttgataaagcacgggcacctttgggagtatgagggtgggtgggtgtgcacatcttccatgaaggaactgttaatattggggcagatgtttcaagtatggcagacaaaggatgttctgcgtggggaaatgtggtgacacccatttcacaaggacagctcacatagattgagtgctcaggaaggaccagcaccatacccagtgcctgatgtgtatcatctcaattagtccttgcctcagatgcaaaaggaaaccatcgccatcatcatcaccaccatcatcatcttcctcctgtgcagatggaaaggctgaggcatagagaggtgacggagtctgcccaggactgcaagcctgctggtggcagagccaggttccaatggaatgaaggctgtcatcctcagatggcagggtaggcaggtggctagagctcacttgggagaaggggaaaggacactgactttggctagggatggagcagagcttgggctggctttccatgcacgggcagggggcgtggctcatggctacgctccagccccgggtgtggacattgaatcttccaggtctaccctaggctatgggtctggacagcactgtgatggaaagaagacactctatgtcctgcattctgtgaccaatgatgtgactgtgggaatggcgctggcatctggctgccactctgggacgggtggccagctgccatcaggccccacccaggatgggaccaccatgcgacttcttccctcgctcctcctggtcatgtccagagccccaggaggaccagcaaagcctctcgagccgatggcagctcacgttctaccttgtcagctactcctctcctgggcaacattggctgcttgctgtggctctccccggggtatgtgactgcctctgtgctgggcacctggcctgggctttccttctgggcctgggcagctgggctcagcttggacccaggcagcagccacagaggggcccatggaggtgacagagttgcttctatgatggtgaacgggcagctgtgacacggaggaggcgaccactcctcagtttccaagtgctgcggtcagggccggggccagcaaagtccctcccatattcaaagagtgggtttgggtttgtcccaggaggacatagtcaggagcccatgctggcacatgcctcctccaaagttcagcctggatccccagcctctgccaacggccccgctccttagctaacccagcttgctcctgggttccacggcggagtcagatgtttctgggcagtttcacctttgtgccttaaatgcatgttgaggactttaaggaattgtggagaaatagggctgtggcaaaggcaagtgacaactgggaacaatgatcctgcagaggctgctgaggcctgggccccaggggcgtgggttcatccttctgcctgggctttggtgggaggggcagactctgtggtctgagacacaaaaaaacccaaaacatacgtgtgtacagacacacagcagagccacacacacacttgtgcccatgcacacactcacaggaggcccgtggactccgcacagggaagaaactcctccggtcgacagtggacggcgctgcagcagggactcacccccaagccctgcctgcctcccattgcccacctggccctggcttg

SEQ ID NO: 24 - DNA, 33 bp, Artificial Sequence
  Description of Artificial Sequence/note = synthetic construct
ataacttcgtataatgtatgctatacgaagtta

SEQ ID NO: 25 - DNA, 50 bp, Artificial Sequence
  Description of Artificial Sequence/note = synthetic construct
tgaagttcctattccgaagttcctattctctagaaagtataggaacttca

SEQ ID NO: 26 - DNA, 4829 bp, Artificial Sequence
  Description of Artificial Sequence/note = synthetic construct
ataataaacccaagcttggcactgggatctgcgaacgcagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaaccgcgctatgacggcaataaaaagacagaataaaacgcacgggtgttgggtcgtttgttcataaacgcggggttcggtcccagggctggcactctgtcgataccccaccgagaccccattgggccaatacgcccgcgtttcttccttttccccaccccaccccccaagttcgggtgaaggcccagggctcgcagccaacgtcggggcggcaggccctgccatagccactggccccgtgggttagggacggggtccactagctagttctagtatgcatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatcctcaggactctccctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgcgtatagatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggtcggagtactgccccgcccctagcgattagccccggccccgcatagctccgccccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagacaactctgaatactttcaaaagttaccgagaaagaagaactcagacacagcagaagagcaattggtaccggatccgatatcgatgcggccgctcgagactagtgagctcgtcgactctagactcttctggttctggcgactataaggatgacgatgacaagtaatagccctttagtgagggttaattgctagc

SEQ ID NO: 27 - DNA, 8433 bp, Artificial Sequence
  Description of Artificial Sequence/note = synthetic construct
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgcgtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgatctaaaatacacaaacaattagaatcagtagtttaacacattatacacttaaaaattttatatttaccttagagctttaaatctctgtaggtagtttgtccaattatgtcacaccacagaagtaaggttccttcacaaagatcgcggccgcctaagtcatttggtgcggcgcctccagcatctccataaggaaggtgtcaatgggtgtgtccccgatgagcttgaagaagaagagatgttccaggcatttgagcccgatggagcgcagagccggcaggcggagcaagagcttagcgaaccttcccggctgctctgggtacttgtgcttgcagtaggcctccaaggacgcatagaccttctccctcagcgcctccacctcggccgggttcgagagccccttggagtcagggttaaagaggacgatggcgcgcaggcagcccagctccgtcttgtccatctgcatgtcccgcatcttggacacaagctccgtcagcaccctgtcaaagatggcgcccacccctgcgctgtgggcgctgttccggtggacgtgcagcccggtggccaggaggatcccgtccttcacggcgatggagcggtgggagaaggaggcgatgagcagctcattccagcctgcccgcagcaggatgacctggtcgtccaggggcagctctgagaagtgtgggatccgcttggcccactccaccagggtgaaaagctgtttgtcggctgcttggcaaatgttggtgacagggtcgttcggcgagctggggttcagccccatgtttgcctccacgtaggtctcggtcttgggctccacggccagctcagcctccaggatcctctccaccggcatgtcctcgttggcgctgctggtcgactccacctcattctcgttccggtccttgccacgctgccgctcctcctgcacggcttcccgcttcatgcccatggccaggcacttctggtagcggcagtactggcaccggttccgctgccgcttgtcaatcaggcagtccttgttgtcgcggcaggtgtaggtcaggtccttgcgcaccgtccgcttgaagaagcccttgcacccctcgcagctgtacactccatagtgcttgcctgaggagcggtccccgcagatggcgcagatgtgcttggtgaaggaagccatgtttcctgaggggtgggcggggaccttgaggacgccattgaggcccagggggggcttgatgtcctcgctgctgctgacggggttcataggtgagctgagctgggggctgccagtgctgaagcccagggtgggtgtggtgggcaccgacatggagtgggggcccatgggggagctgatgaccgagaaaggcgggcccatgccgttgatgggggagctcagggtgctgatgggagaatgcagctgtcccggggagccgatgccaggccccagggacgggtgcagcgagggggcagccatggagcctcgccccgtcggggaggtgagggaggagttcacctgggtggagaaatcgagcggcaggaaatgtttggtgtccaagtcttcttcagaaataagtttttgttccatattatcgatcgtgtttttcaaaggaaaaccacgtccccgtggttcggggggcctagacgttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggggtaaccctatgcagtcgtcgagtgctccgacttaaccgccactcccccgataagctgctcttggtgcgaatgcagggctacacccatcaaggccatcgccgtctgggcgttcgcatacatgctgacgttgccgcccaccccaacaccgactccaactccgttgcccatcggtaccgctgatgtggtggagctagcggtaacggcagccgtgatactgctggtggttgccggcgtgatgggtcctatggccgcacttccgcccatgtattcgctgctcgtactgaccgcggacaaggaaccaggtgcggttacggaggcgggcacgggagcggagacgggaaggagctgtggctgtggttgaatctgtggctggagttgcgtctgaagctgtgggtggagctggggttgcagttgaccttgcagctgaggtggtagctgaggttgtagctgcggctgtgtctggtgctgggaatcgttctgggtcagggaggagggttggggctggggctgaggctgaggctgatgctgggccgcggctgccgccgccgaagtggaggcagagtcgcaatcaatgccggcggtaatggcgcccccaaccgatgcccgcatacgctcagcccgctcgagacgctcgttctcctcctgggtaatctgaaggtgcgactggaccgatggcgggatggcatgaacgtcccagatctcctcgaggaacttgggcagtttgcggtttttgagctttagtgagaaacacatctcggcgttctggttgcccagcgtacgcagctcggtgaggatcgagagcagctttgcgtagaagacgaggctcattgagtcgccgcagtggcggttgagtatataaatgcgtagcgtgtcgatgtagtagctctggatcgcttcgactagctgggccttctccaggcccggccggtccgagaagatcacaatggcagtgagaagcgcgtattcgacgttgtccaccttcatcgagaacatttggcggcagaaatgcagcaggtcttcaatgttatcagccattccggccattttgtaagaatcccgcgtatatgatctattattcgcgaagaatattgagtccgagctgtggtcatagcgtcgtgccatacgcagcatcatcacctccgacgagcaggcctttagtaacgtgatctggtcctcctggggtatctttgtaaacgctggtagacctttagcaaactcaacaatcaactggaccgtgagtatggttatctcggttatatgccgaaagctgacgtccgtttggctctcgttctcatcgggttgactcattatacgcctgagatcctcttcagatggctgctcatagccatcctggtaccaaattaacttgtatataacggccaactgattgtacgttaaggaaggtatattgcgcgcttgacacttggccaatatttcatcaggtagtagcggaatagtggcatgctggggcggctcgcatgtcataaggtcaagaatctccttcttaacaaagtcttggccgccaccagaggccaagctgccattgccgccatgctgagagctcggcgaagtggtcattttgtccttctccttctgggccttcttttcgcgccgcttcatcgcacattggttctccgggacgacgcattccggccgcatacccacggccaggcactttttcaggcggcactcctgacactttcgcctcatgtacatgtccatttcgcaggcgcgcccgaacttgcagcagtagacggcgctcttcgtaacgctgcgtcgaaagaacaccttgcaggatccacaggtgagggcgttgtagtggtagccggaggccctgtcgccgcaaaccaggcacagctcctcttgcacccgtggcgcaggtcccttcttgctcttcttcacatcgcagctttcgttcgccgagtatccgttcaagctgctcgaaggcgagagatcatcgcgacctgaagatatagaatttgatattcttctagatgtacctagaagcttcccaccgtactcgtcaattccaagggcatcggtaaacatctgctcaaactcgaagtcggccatatccagagcgccgtagggggcggagtcgtggggggtaaatcccggacctggggaatccccgtcccccaacatgtccagatcgaaatcgtctagcgcgtcggcatgcgccatcgccacgtcctcgccgtctaagtggagttcgtcccccaggctgacatcggtcgggggggccaagtcttcttcagaaataagtttttgttccatggtggcggccggccactagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaatgcaacggcgctgcagccactgcatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgc

SEQ ID NO: 28 - DNA, 151 bp, Artificial Sequence
  Description of Artificial Sequence/note = synthetic construct
gaactcagacacagcagaagagcaattggtaccggatccgatatcgatgcggccgctcgagactagtgagctcgtcgactctagactcttctggttctggcgactataaggatgacgatgacaagtaatagccctttagtgagggttaatt

SEQ ID NO: 29 - protein, 11 aa, Artificial Sequence
  Description of Artificial Sequence/note = synthetic construct
GSGDYKDDDDK